SEQUENCE LISTING (64 sequences)

SEQ ID NO: 1 - DNA, 23 bp, synthetic construct
  source 1..23: mol_type = other DNA
gaactcaagcargcytgctactc

SEQ ID NO: 2 - DNA, 26 bp, synthetic construct
  source 1..26: mol_type = other DNA
accatrccaccaaaaggmgtrtgaaa

SEQ ID NO: 3 - DNA, 30 bp, synthetic construct
  source 1..30: mol_type = other DNA
aaaggatcccgacatcataacggttctggc

SEQ ID NO: 4 - DNA, 37 bp, synthetic construct
  source 1..37: mol_type = other DNA
aaacgtacgggtctgtttcctgtgtgaaattgttatc

SEQ ID NO: 5 - DNA, 46 bp, synthetic construct
  source 1..46: mol_type = other DNA
ggataacaatttcacacaggaaacagaccagcgggacaccagctcg

SEQ ID NO: 6 - DNA, 50 bp, synthetic construct
  source 1..50: mol_type = other DNA
ggccgctaatacgactcactatagggagttaggagtttccaccgcccaac

SEQ ID NO: 7 - DNA, 50 bp, synthetic construct
  source 1..50: mol_type = other DNA
gttgggcggtggaaactcctaactccctatagtgagtcgtattagcggcc

SEQ ID NO: 8 - DNA, 46 bp, synthetic construct
  source 1..46: mol_type = other DNA
cgagctggtgtcccgctggtctgtttcctgtgtgaaattgttatcc

SEQ ID NO: 9 - DNA, 61 bp, synthetic construct
  source 1..61: mol_type = other DNA
ggataacaatttcacacaggaaacagaccatgctagataaaataaatcgttatgctcatgg

SEQ ID NO: 10 - DNA, 60 bp, synthetic construct
  source 1..60: mol_type = other DNA
cactcctgctgtatgaattatgcctttgagtgttggcattgatatctgaatttgtttgag

SEQ ID NO: 11 - DNA, 60 bp, synthetic construct
  source 1..60: mol_type = other DNA
ctcaaacaaattcagatatcaatgccaacactcaaaggcataattcatacagcaggagtg

SEQ ID NO: 12 - DNA, 56 bp, synthetic construct
  source 1..56: mol_type = other DNA
ctcctgcttctgagtctgggttaggtctgcaagtatccatgacttcatagccatag

SEQ ID NO: 13 - DNA, 56 bp, synthetic construct
  source 1..56: mol_type = other DNA
ctatggctatgaagtcatggatacttgcagacctaacccagactcagaagcaggag

SEQ ID NO: 14 - DNA, 50 bp, synthetic construct
  source 1..50: mol_type = other DNA
cgagctggtgtcccgcttagagtaatctcctccctatcttcattgatttc

SEQ ID NO: 15 - DNA, 50 bp, synthetic construct
  source 1..50: mol_type = other DNA
gaaatcaatgaagatagggaggagattactctaagcgggacaccagctcg

SEQ ID NO: 16 - DNA, 19 bp, synthetic construct
  source 1..19: mol_type = other DNA
cggcgctggagaataggtg

SEQ ID NO: 17 - DNA, 18 bp, synthetic construct
  source 1..18: mol_type = other DNA
atccgggtgcggtagtcg

SEQ ID NO: 18 - DNA, 61 bp, synthetic construct
  source 1..61: mol_type = other DNA
ccatgagcataacgatttattttatctagcatggtctgtttcctgtgtgaaattgttatcc

SEQ ID NO: 19 - DNA, 23 bp, synthetic construct
  source 1..23: mol_type = other DNA
ttgacagctagctcagtcctagg

SEQ ID NO: 20 - DNA, 44 bp, synthetic construct
  source 1..44: mol_type = other DNA
cacaatttgctgaattgtggcttattgcagaaagccatcccgtc

SEQ ID NO: 21 - DNA, 44 bp, synthetic construct
  source 1..44: mol_type = other DNA
gacgggatggctttctgcaataagccacaattcagcaaattgtg

SEQ ID NO: 22 - DNA, 21 bp, synthetic construct
  source 1..21: mol_type = other DNA
ttcattacgaccagtctaaaa

SEQ ID NO: 23 - DNA, 21 bp, synthetic construct
  source 1..21: mol_type = other DNA
gctttttaacttggattttta

SEQ ID NO: 24 - DNA, 21 bp, synthetic construct
  source 1..21: mol_type = other DNA
ctaaagacaagtgagatagca

SEQ ID NO: 25 - DNA, 44 bp, synthetic construct
  source 1..44: mol_type = other DNA
cgagcggccgcatacgatttattgacagctagctcagtcctagg

SEQ ID NO: 26 - DNA, 43 bp, synthetic construct
  source 1..43: mol_type = other DNA
ttatctagcatggtctgtttcttcattacgaccagtctaaaaa

SEQ ID NO: 27 - DNA, 38 bp, synthetic construct
  source 1..38: mol_type = other DNA
cgagcggccgcatacgatttagctttttaacttggatt

SEQ ID NO: 28 - DNA, 41 bp, synthetic construct
  source 1..41: mol_type = other DNA
ttatctagcatggtctgtttcctaaagacaagtgagatagc

SEQ ID NO: 29 - DNA, 23 bp, synthetic construct
  source 1..23: mol_type = other DNA
taattatttaggggatatttgct

SEQ ID NO: 30 - DNA, 24 bp, synthetic construct
  source 1..24: mol_type = other DNA
tgaacgtaaactgggcaaccatag

SEQ ID NO: 31 - DNA, 22 bp, synthetic construct
  source 1..22: mol_type = other DNA
gggtgtatttctcagagagggt

SEQ ID NO: 32 - DNA, 20 bp, synthetic construct
  source 1..20: mol_type = other DNA
agtctctggtaacgtggtga

SEQ ID NO: 33 - DNA, 20 bp, synthetic construct
  source 1..20: mol_type = other DNA
ggagcgctcaagaagtgaag

SEQ ID NO: 34 - DNA, 20 bp, synthetic construct
  source 1..20: mol_type = other DNA
aagctttctggtgtgttggc

SEQ ID NO: 35 - DNA, 25 bp, synthetic construct
  source 1..25: mol_type = other DNA
aatgatggtaatggaagagttatca

SEQ ID NO: 36 - DNA, 24 bp, synthetic construct
  source 1..24: mol_type = other DNA
tgcaaatatatcagccagttctcc

SEQ ID NO: 37 - DNA, 20 bp, synthetic construct
  source 1..20: mol_type = other DNA
aggccatttgaccaagacca

SEQ ID NO: 38 - DNA, 19 bp, synthetic construct
  source 1..19: mol_type = other DNA
gcgcggtcagttctctcta

SEQ ID NO: 39 - DNA, 20 bp, synthetic construct
  source 1..20: mol_type = other DNA
gatagcctcagcgttctgga

SEQ ID NO: 40 - DNA, 19 bp, synthetic construct
  source 1..19: mol_type = other DNA
agtgctcatagacttgcca

SEQ ID NO: 41 - DNA, 20 bp, synthetic construct
  source 1..20: mol_type = other DNA
accctcatcaggggtgaaag

SEQ ID NO: 42 - DNA, 20 bp, synthetic construct
  source 1..20: mol_type = other DNA
ctcgttgacggctaaacctt

SEQ ID NO: 43 - DNA, 20 bp, synthetic construct
  source 1..20: mol_type = other DNA
gagaggtactggctcggtaa

SEQ ID NO: 44 - DNA, 20 bp, synthetic construct
  source 1..20: mol_type = other DNA
ggctctctgatagcggaact

SEQ ID NO: 45 - DNA, 20 bp, synthetic construct
  source 1..20: mol_type = other DNA
taagcatcggctaactccgt

SEQ ID NO: 46 - DNA, 20 bp, synthetic construct
  source 1..20: mol_type = other DNA
gacgctttacgcccaatcat

SEQ ID NO: 47 - DNA, 3417 bp, synthetic construct
  source 1..3417: mol_type = other DNA
atgctagataaaataaatcgttatgctcatgggtttgtagctgtaccagtgatttgtgcttgttcagaagcaggggtattcgagttactatcacagaaaaaatcactgaaattagaagaaatagtagagcatttagcagcaaatagtggacatctaatggtagctatgcgactcctagagtcattatcattcctatatcgctctcaagcagaagaatacatattgacagaacaaagtcaacaacatcaaatcatccctaaagctctgatgagcttatataaatatccatttgagttatatttaaaaggagaagtagaaacaggaatcagcaattggattaattgttcatctcgaagatgggatacagaaaactcattactaagtgatttattagatggagtattactcatccccctgctactagaactgaaaaaacaaaacttactggatgaatcaaaaaaaatatttaacacattaacaaattcccttaaacaagaactatcaacattattcatcaatttaggatgggcagaagaaaaaacagaagggctatatttaacagatataggtagatttatgcgtgacagatccttaaacttaggaacaacagcgtcttatgctcctatgttgttacagatgaaagaattactatttggaaatcctcagagagtatttcaaagaaataagactgaaaaagaaagacacgtaaatagaacattaaatgtagtagcaagtggctttcaacacgaaaagttttttgccgatacagataaaatcatcatatctatatttaaccaacagccaatagaagagcaaccaagctatatagtagacatgggttgtggggatggaacgctactgaaacgaatatataaaatcatcaaacaattctctgctaggggaaaagtattaacagagtatcctatcatcatggtaggagtagactacaatcaagaagcattagatgtcacagataaaaatttagtagatattccccatctagtcattccaggagatataggagcaccagaaaaattattagagcaattgaaagcacaaggaatagaaccagaaaaagtattacatattcgttcattcctagatcatgaccgaccatttatagctccaaaaaatacagaaatagctcaagcacgttctcagttagattatcaagtagtagatgtagatcgagaaggaaagctgataccacctcacatagcagtacaaagtttagtagaacacttagaaagatggtcatcaataataactagacatggattattgctcctagaagtacatagtttaacgccagcagtagtcaaaaaatatatagacgaaagtgaatctctgcattttgatgcctatcatgccttctcaatgcaacatttagtagaagcagatgtgttcttgatggcagcagcagaggtgggtttattttcacggaaagaagcttttcgtaagtatccgaaaacgttgccattgactcgaatcacagttaatcattttgaaaagcgtaaatatcagatacggtatgcaactgtaaacgatataccaaatctgttaaagtgtgcgacatttaatcaacctgtaaacgaacccttctttcaagttttattgaaacaaactccaacagcacatttattattagaatatcaaggtgaattggtagctgcaatcttcacagaaactaaaaattctaatgaggtgctaggaattcgtgagtttttagtaaggacttctgtagaaaattggcaagtattagcaaaagatttactagaatttgtggaacagtggggagtagtcaaaccaggaataaaagaaatagaaggattgttaaaataccatgaagccatctcaaactttcaaaaatcaaagtggtatcaatcttcagttttaaacaaaaagcttatagaaaaaataactctacacgaattagctactttagaactatgtaatttaatggctccagaatatgagctagaagcctttgccgcacgttggcttttgcgtgtttttcaagatatgggtgtatttctcagagagggtgaatcttatcaggagtctgagttggtttcccagctaaacatctcaccacgttaccagagacttttaggtgctttgttacaaattttgcataagcgtggtattctaaaaattgagaaagatagagtgttcacattagcaagatgtaagacctttgccttagaaaatatttcgtctgaagtttctgctttttatgattatttttctgaaaaatatcctgctcatttatcctggttaacagtagtcaaaaggtgtctagagaaatatcctttgattctacgcggtgaagttgatgtcaacgaagttgtttttacagatggggatatggagctatttgctggactatttctaggacatcgtgttgctgactactttaatgagttgctagcagatggggtttgctgggaagtagaacagcggttgttagaagaaaagagggcacaacctattcggatcttggagattggagcaggaacaggaggtgttacaggaatattgctagaaaagctagcttctcatgcagagcaaattgaattttggtttactgatatttctagcgtttttacacgttatggtgagagtaagttcaagcagtttccttgggtgaaatatcaaacctttgacatagaaaaatctcttgatgctcaggggataaagtctgaaagttttgatgtggtaattgctaataacgtactccataacacaaaattaattcatcaaaccttaaataacagtaactcactattaaatactggggggttattggcattactagagtttactcaaccaattgatattcttttatactttggagggttgcttcaaggattttggttgtttgaagatccagaataccgactagaagttggttgtttactgagtataccactgtggcaaaaagttctcagtgattgtgggtttgatgaaataataccattgggattaccctgtgagatgcacgctctttctaaggcaagagaatctgttatatttgctcgaaagcatcaagtacaggagaaaacattttctgaaaaaattaaacaaaatttgacagaaaatggtaagcatgggcaggcagaattcgattttattagtattaataattcacaagaaagttcatccaaattagaaatttttgaacaggaatgtcgaaaattattaaaatctctactgggtgttcaacgtatggagagattgcctggtgacacaccactaatggagtcaggaatggattcactggagttgttagaatttcgtgctcttatagaaagaaagtttgggattaagttaaagtctaccttcttttttagttacaaaactcttatagcggtagcagagtatctttcagaacgggaagatattaattttagttag

SEQ ID NO: 48 - DNA, 8823 bp, synthetic construct
  source 1..8823: mol_type = other DNA
atgcattctaaccagagcagttcactagcaagaatagccattgtaggtgtggcttgccgttttcctggagcagatactaaagaagaattttggcagctacttaaggaaggtcgggactcaatgcagaatcttccttcagagcgttggggaaatctatttaactccatgagttcagaaatagatttatctattcaacgcggtggttttttgaaagatatagatttgtttgactcatccttttttaggattactcctagggaagcgcagttaatggacccccagcaaaggctgttgttagaattgtcctgggaagcgatggaagatgcaggatatgcaagggatacattaaagggtaaatcagtaggggtttatgtaggagtttgtcactatgactacagaagtttattagaaaaaggtttggagacagccgaaatagctcaaattgctactggtacagctccagcaacttttgccaatcgcctttcctatttttataattttcacggccctagcttaactgttgatacggcttgttccagctctttggtagcaatgtatgaagcagtaaatgcaatacgtcgtgggcagtgtcaaacagcattggtaggaggcgttaatttaatgtgttctcctgtgaataaccaagtttatagtgctgcgggtatgttatctcctgatggagtctgccgggtgtttgatgcgggtgctaatgggtttgtcaggggagaaggaggggcagtagttgtacttaaagattatcaaaaagcgttgacggatggtgattctatttatggggttgttcgcagtgtagcagtgaatcatggagggcaagcaagttcttttacagcaccaaatccccaagctcaggcaaaattgttggaacaggcttatagggaagctaatattgatatagagtcggtaggttatattgaagcacatggtacgggtacatctctaggagacccaattgaggtcgaggctttaaatgaagcgtttaaagggttgagttcaagtggtaagttacctgctaatagttgtggtttgggctcagttaaaacaaacattggtcatttggaaggggcagcaggtttggctggactaatcaaagttttgctgtgcatgaggtatgcaactcttccatgttcattaaattatcagcaacttaatcctgatattgaattagaagaaggtccgttttttgtggtggacaagcttcaatcttgggaaataaaagttgatagggtaggaaaaccttatccacttagagcagggttgagtagctttggttttggcggtacaaatgctcatgtagttcttgaggaaggagaaaataagaaggaaggaagaagaggggataaggaaccttcagttcatctattaaccctttcagctaaaactgaaacagctctttcagagttagtcagtcgttatcaaaaatatttaaacactaatgcagaatcagagttagccgatatttgttataccgctaatactgggcgagtccattttaaccatcgactagcagcgatcgcctcaaacaaacaggagttagtagaaaaacttaaggaataccaagctggggaggcaacagcgggagtattaataggggaacttgcagaacgcataagaacaccgaaattaggtttcttattcacaggtcaaggttcccagtatgtcaatatgggaaaacaactgtataaaacacagcctgtattccgtgaagtattggataagtgtgatataatattggaaacggaaatagaatgttctctattagatgttctatataacaaaactacagattctcaagattcatctttaataaaccaaacagcttacactcaaccagttctgtttgcaatagagtatgcgctatttaagttatgggaatcttggggaattaaacccagcatagtaatgggtcacagtgtaggagaatatgtggctgcttgtgtagcaggagtcttcaatcttgaagatggtttgagattaatagctgctaggggcaggctgatgcaacagttaccttcaggtggagaaatggtttctgtgatggcttcagaatctacagttagcagactcctagaacctcacaaaaaagaaatagcgttggcgaagcccgccgtaggcatcgcagcaataaatggaccagaaagcacagttatttctggagactctgtagcagtaacaggtgtagtaaatgacctggaagcaaagggaataaaaactaaaaaactagaggtatctcatgctttccattcaccattaatggaaccaatgttaggagagtttgaagctatagctaatcaactaacctacaatcaacctaaaatacctattatatctaacgttacaggtacaaaagcagataatactattgcttcccctcaatactgggttaatcatgtcggtaaacctgtaagatttgcccaaggaatggaaacgctacatcaacaagggtatgaaacctttatagaagttggaccgaaaccagtattgttaggaatgggcaggcaatgtctaccaggaaatgtaggtgtctggttgccatcattacgtccgggagtggatgagtggcaacaaatgctttatagcttgggaaaattgtatgtaacaggagtaaaaatagattggtcaggatttgagtctgactctagtcgccagaaagtagcattgccaacttatccattccagcgagaaagatattggatagaaataacagaaaacaaacataaggaacatcaaaagtcagaaaatataagtgacacttcaattgttaaactactcactcaaggaaaaacagaagctctcactcaacaactagaaacagaagccaaattttcaccagaagagcttaaacttttaccagaaatattagagacattagccaaacaacatcaagaacaattaacagcagtaaccatcaaaaactggttctacgaaatccagtggaaacccttagctcaaaacaaccccaatgcaaacattgaacctagtcattggttaattttagccgataccacaggagtagcagaaaaattagctcaaaaattacaacaacagggtcataaatacagcttagtttatcgaggagagagctatcaaagacaagcaacaggtacttatcaacttaatcctcagattcccgaagcatttgaaaagctgtatcaagaaattcaacaaagtagtgaaactgccattacgaagttaattcacttgtggagtttagatgctccccaatcaaaagacttaaccctggaaaccctagaagaagctcaattatggggatgtggcagcgtagtacacctgttacagaccttagtcaaaaactctagcattcctgaactatggttagtaacccgtgggtctcaatcagtattatcccaaacagaaaaaaatctaacaggactagcagcgtcacccttgtggggattaggtagagtagtgtctaatgaacatccccaattatggggaggattagtagatttagacccacaagctgcagcaggagacgaagtagaaatgctgtggcaattattagttaatgaacaagaagaagataatctagctgtacggggagaaaatacctatgtagctcgtctggccaggcaagaacctcaagaatttcctgaatccctatccttatcatcagatggtagttacctaataacaggagggttaggagctttagggttacatactgcccaatggttagtatccaagggagcgaaaaatattgtcttaactgggcgtcgccctccctcagaaaaagtaagtgaatctataaaaaaattagaagaaacaggatgtcaagtgaaggtgatgttgggggatgtttctgttgaagaagacataggcaaaattctcaaacaaattcagatatcaatgccaacactcaaaggcataattcatacagcaggagtgttagatgatggaaccatacaacaaatgaattgggagcgttttgccaaagtcatgtcgcccaaggtaaaaggaagttggcatttacataaattaactgaaaatcagccattagatttctttgtgtgtttctcctcaatagcttcgatgttggggactcttggccaaggaaactatgctgcagccaatgcctttatggatgctttagctagttatcgttgcagcagaggattatcaggattggcgattaattggggagcatgggcatcagggggaatggctgctcgtttagcagtggagcatcaaaataggatgcacagcagtggcataactgagatggcgaccaaagaaggaatgtatgccttagatttactattaacaaatgaatctgctacagctcaggtaggtgtagcaagtatagagtggcaggtactctcagaaagttggagtggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttaggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttatgtatagaggaattaatgggatggaagacaacagaaattgacccattatctgaagaaacaagattagagatgatggagcgctcaagaagtgaagcaatagccatcatcggcataggctgtagatttcccggaaatgccaacacaccagaaagcttctggcaattgttatccaatggcaaagactccattacagagattcccctagaacgttgggatctagattcctactacgaccccaaccctgatactccagggaaaatgtatatccgtcacgcagcattagtagaaaaagtagatcagttcgacccacgattttttggaatctctaaccgagaagcttatagtcttgatccacagcaacgcttcattttggaagtaacttgggaagctttagaaagagctggtattaaccctcaacaattagaaaacactcagacaggggtgttcctgggtatcggtcaaaatgattacgcgaatttaggttttcaccaagcagccgaagatataagcccttacgatgctacaggaaatttgttttgttttgtagcaggtaggttatcttactttttaggaacgcaaggtccatcaatggcaatagatacagcttgttcatcatctctagtagctatccatgaagcttgtgagagtctgcgtcagggtgagtccaacttggctttggctggaggagttcaactaattctctctcccgaagtaacaaccgcactatcaagattaaaggctctagcacctgatggtaagtgtaaaacttttgatgctgctgctgatggttttggtaggggagagggatgtggcattgtggtactgaagcgtttgtcggatgcgctcaaggatggggacaggatatcagcagtaattcctggttcggctgttaaccatgatggaccaagtagcggaatgacagtaccgaataaactggctcaggaaaaactgattcagaaagctctcaaagcagccaaggtaaaaccgttacaagtgagttatgtggaagctcatggtacaggaacttctctaggagatcctatggaagtgagagctttagctagggtgtttgaggagggacgtgatcaggaaaatccattgaacatcggttcagttaaaactaatatcggtcatctggaagcagcagctggaatagcaggtatgattaaggtgattttgcaattgcaacatcaggaaattgtgccccatctgcattttgctaatcctaacccctatgttgattgggagaatatgcctctacaagtaccgactcaactgactccttggttgtcgaaaggggagaaaagggtggcaggagttagttcttttggtatgagtggtacgaatgctcatatagttttagaagaggctcctattgaagtcagaaggcagaagtcagaagtcagaagtgaagaatatctagaacgtccggttcatatactaactctgtcggccaagactgaaaaagcactagaagatttagttaatagttatgaaagttatttagaagccgaaaataatgataattatttaggggatatttgctacacagccaacatcgggagagcaaaatttgaccacaaattagcagtggttacttctgataaacaagagttattagagaaactcaaacaatataaacaaggtgagaatgttgctggaatcttttcaggaaaacaaataagtgaaactagaacaaaaatagcctttatatttactggtcaaggttctcaatatctgcaaatgggaaggcagttatacgaaactcaacccacttttcataaaattattgaccagtgtagtgaaatgctggtaaaatatttagatgtttctttattagatatactttatccagttgaggttaaagatgaaagttcgactttgatagaccaaacagcttatactcaacccgctatatttgcccttgaatatgccctagctaaattatgggaatcatggggtataaagccagatgtagtcatgggtcacagtgtaggagaatatgtggcagcaacagtagcaggggtatttagtttagaagatggtttaaaattaatagccatgcggggacagttgatgcaaaagttaccctccggaggtcagatggcatctataatggcatcagaatctcaggtaatagaggcgataaaagaatatagttcccaagtaacaatagcagcagttaatggaccagaaattatagtaatttcaggtgagagtccagccatttcaaaaatttgtagtaaatttgaatcagaaggagtcaagaccaaggcgctacaagtatctcatgctttccattccccattgatggaaccaatgttaacagaatttgaagcagtagccaaagaaatatcctataatcatccccaaataccactaatatcaaatgttactggtcaagaagtaaatggagaaataaccactgctgaatattgggtgcgtcatgtgcgtcaaccagtaagatttgcggaagggatggaaactttacacaaacaaggtgctgaaattttcctagaaataggctcaaaaccgatacttttaggtatgggtcgtgagtgtctcatgggagaaaaaaaactatggttgcccagtttacgttcaggaaaaccagactggttacaaatgctacaaagtctggggcaattgtatgtacgagcaataaaaattgattggttaggatttgatcgggattatttccgtaataaggtagagttaccaacatatccttggcaacgaaaaaggtattggataacagatattagacaacggaaaagtcaagacaaaaaaagcataacatctgaaaaaaaagtacaacttgatggagtaaatattcaactaaaggaaatacaaatgaacgacaaaatcttacagcaaccaaaactaaaattatcagatccagaatcactatatttatcgaatgctgaatcaactatagaggttccaacaaaagcagttcaagtaaaacctgctgctgatattgaatctgaaaatataactcaattgaatagccttgatagagatgtgactcaaatcatagaaaccctcaaagaaagtttggcagatgctttatatgcagatataagtgaaattgaagaagacaaaaagtttgttgatttaggtttagattctattgtgggagtagaatggataactaacatcaataaaatctacaatttaaatataaaagctactaaactatacgattaccctactttgccagatttggctaaatatgtcgctcacaccctatcagctcagggtagaaatattgatgtagagcgatcgccatctgactcaagtcaagcaagcattagcaagcaatctcaactaactgatacccagagcaatttctcacaagtcaaggaaattttgaaacaacaattagcagatgctctgtatgcggatattagtgaaattgaagtaaacaagaagtttgttgatttaggtttagattctattgttggagtagaatggataactaatatcaataaaacctacaatttgaatatcaaagccactaaaatatacgactaccctaccttattagatttcgcaaaatatattaaccaggaaatttattctacaggagtaagtagattttcaacggagcataaagaatttaatcaaaaagactattcatctggaggttcgcaagaggaaatgacacaaaaattacgatcaatattgaagaaagtggccaataaagaattaacagttcaggaaggaaataaaatgattcaacaaataaaaaatcaattaaagtaa

SEQ ID NO: 49 - DNA, 261 bp, synthetic construct
  source 1..261: mol_type = other DNA
atgaacaaagagcaaatatttcaaataataaaaaaatatacctacgaaatagcaccagaattagaagaagtacctatatcacctactgatagtctcaaaaacttggggattgattcagtaaatagggcagaaattataatgatggtaatggaagagttatcattaaatatcccgcgtattgaattagctggatccaaaaatataggagaactggctgatatatttgcattcaaattagaagcaataaattcacaaagctag

SEQ ID NO: 50 - DNA, 1224 bp, synthetic construct
  source 1..1224: mol_type = other DNA
atgagtaacatagaaataacaggtatgggcattgttacttccattggtcaaggagttgctacttttaaagaagctctgttatcagggaaaactcaatttgcttatctaaaacagccaggacgtgaaagcatcaaaccatttattggtgccgaaattcccgatattgacgccaaaaccctatttcctgagtacagtggactgttacgtactgctactaagagcgcccaagttgcaatagtagctgtcgctgaagcctggcaagatgctcaactcacctccagccaagttaacccagaacgagtaggattagttgtaggtggttcaaatttacaacaacgttatcagcagcaaacctggcaacgttatcactcacgtcaggagtttatacgaccaacttatggtctgaccttttgggatacagatatattaggtctgatttcccagtgttttcagattcaaggtgagggctatagtgtaggtggagcttcggccagcggagcagttgctataattcatgctgctcgtcaaattttaatggggaacagtgatgtttgcattgctctgggagctttatccgatctatcagggtatgaattccaagcactaatgaatttaggtgcaatggggagcgaacgttttgccgatcgcccaaaccttgcttgtaggccatttgaccaagaccatgatggctttatttatggagagggttgtggagcagtgattttagagagaactgaccgcgctcaacaacggggcgctcaatcccatggccaacttaagggttggggattaactttagatgggaaccgtagtcccgaaccttcccaaaaaggggaagaacgggctataaatactgctttagccatggcagaccttcagccagagagtattgattatgtgaatacccacggtactggttccccccttggcgacaaaacagaagtggcagctttcaaatcagtaggacttcagcattgcctatttaattcaacaaaatctttaataggccactgcttaactgctgctggtgtagtggaagcgatcgccaccatattacaaatgaagtttggtttttgtcatccaactaaaaatttagttaatcctattgataccagtctcaattgggtgaaagaaacttctgttcaagctgagattaaatatgctatcagcaacagttttggttttgggggtatcaatacagctttattaattggacaggagtaa

SEQ ID NO: 51 - DNA, 1260 bp, synthetic construct
  source 1..1260: mol_type = other DNA
atgcaacaagttggaattgaagcactaaatgtatatgggggttcagctaaactagatgtgcgaatgctaggcgaagcacgtcacttagacatgactcgcttcgataatcttctgatgaaagagaagacagttgctatgccctatgaagatccagtttcttatgctattaatgcagcaaaacctattattgatagcctcagcgttctggaaaagcaacaaatcaaaatggtgatagcttgtagcgaatctggcattgattttggcaagtctatgagcacttatatccaggattatttgggactaaatcgtaactgtcgaatgtttgaaataaagcaagcttgctactcaggtacagcaggtttacaaatggcattcaatttaattttgtcccaaacctgtccaggggctaaagctttggttataggaactgatatatttcggcctgttgttgtagagggaggagaagctctcagcgaagattggtcttttgtggaaccaagcagtggtgctggagcagtagctattttagtcagtgatgttcctaaaattttccaagctgatgtaggatgtaatggttactatggctatgaagtcatggatacttgcagacctaacccagactcagaagcaggagatgcagatttatcattgctatcttacctagattgttgtgaaaatgcttaccgggaatatcaagatcgagtagaaggagtagattaccaaaaaacttttgattacttgagctttcatactccttttgggggtatggtgaaaggggctcacagaagtatgatgcgtaagtttaaaagggcaaaacctgtggagattgaagaggactttcagaaacgggtgatgccgggattagtctattgtcaacaggtgggtaatattatgggagctacagtatttttatctttagctagcatgattgataatgcagattttagcaaagcccgacgaattggtgtattctcttatggctctggttgttgttcggaattttatagtggagtcgtcaccccgcaaggaaaggaaattcaagctcaacaaaaaatttcatcacagttagcaatgcgctattccttaagtatggaagaatatgagcagttactaagtcatagttcggcagttgcctttggcactagaaatgttaccctagattacaaactatttcctggtgtgtgggaacaaattgaaggtaaaggtcgcttagtgctgaaaagaatcaaggaatttcaccgagaatatgaatgggtatag

SEQ ID NO: 52 - DNA, 777 bp, synthetic construct
  source 1..777: mol_type = other DNA
atgggtatagccatgagttatcaaaccctgaaaatcagttatcaagatgttgtacaaaggattcagatatatcgacctgaatctaacaatagcatcaatagtcaattaacgatggaattgttgtcagctttgcaagctgctgaagcagaggaagttgttaaagtagtgatattagaaggactacctgatgtattttgtacagggatggattttgaagaagtggcaacagcaaaacaatttgatccaaaagctagtgctaatggttactacaatattttgaaacaaatgtctcaaagtagcaaagtaattttgtcacttgtgcgtggtaaagtacaggcagggggagttggtttagtagcagcaagcgatctagttattgctgatgaaacggcaacttttgttttatcagaattattatttggattattaccagcttgtgtattgccttttttgattcgtcgagtgggatttcaaaaagcctaccgtttagcactaacgactcaagctatttcagtatcagaggctgataagtggggattaatagatgaatatggcagtaatattaatcagttaataagtaaatatattcgacgtttaaagtatttaccctcatcaggggtgaaagagttaaaaaattatattaatcagttatggattattcaggcagaaactcaaggtttagccgtcaacgagatttctagcttaatagcagaacctactgttcaagaaaaaattaaacgttttcaaaaagaaggattatttccatggcaaacctaa

SEQ ID NO: 53 - DNA, 9660 bp, synthetic construct
  source 1..9660: mol_type = other DNA
atggcaaacctaaatcttaatttggacttagtagagggcaactctgatgtagtacaactggtggagttgggtaatggtgtcgtgcaaatcacgatgaaagatgaagaaagctgcaatggcttttctcctggaataattgaaggattatacaaatgttttggtgcagttgctcaaaaccaaagttataaagtagtaattttgacaggctatggaaattatttctgttcgggagggacaaaagaacggttaatcagtatttggaaaggagacagcaaatgtaatgatttagatttttttagaatagcattagattgtgaaataccagtaattgcagctatgcaaggtcatagtattggcggtggtttggttttgggattgtatgcagatttagtagtgttaagtcaagaaagtatttacactactaattttatgaagtatggttttactccaggtgtcggatgcacgttaattctccctgagaaattcggtgctttagggtttgaaatgatgtatactgcccaaaattatcgagggaaagaattagctgaacggggtgtttcttttccagttgtaccgagaaaagatgtgctagaagtggctaaaaatatagcctatgaaatgtcggaaaaacccagactatctttaataactttaaaggaacacttaacttcaaaaatccgcaaaacgctaccaggatttatagataaagaagtagctatgcacgaaataacctttcaccaaccagaggtagcaagtagaatagaggaaaattttgacaaaaggacaacggcaagtaataaccctcaaaattttcctcaagaggctgtaagaaaagaaataagaacagattcattgaattgtcaaccatttcagttgaagacatttagttatggttcgttaaacaatttaactttggtacctctagaacgtagagttccaagcccaagtgaagttgaggttcaaatcaaaactgtaccagttaattttcgggatatactcaatgcacttggtatgctccaagagtattacgaaaaaacatttggcattgctaatgctgaagatctcacttttggttttgaaggtgcaggtactatcgtagctgttggggcagaagtatcgcagtggcaagtcggcgatgaagtaatggtaatgagaattcacgatgcatttagtagctttattatctgctcgccggacaaactggtgcgtaaaaattttaatctgaatatggaagatggagctagtatttgggggccgttttcgactgcatatcacgggttgattaacttagccaaaattcagccaggagatagagtattaattcacgctgcttcaggtgggattggacaagcagcaattcagttggctcaactttttggggcggaagtatttgctactactagtccaggtaagatgaattatctccgggaacagggaattaagtatgtgatgaattctcgaacgatggagtttgtaaatgatgtgatggaattcactcagggccgtggggtagatgttattctcaatagcctgactcatggagaatacatccagaaaaatctagagatccttgccgatagagggcgatatgttgaacttggtaagttgggcatttggagtcacgagcaagtctatcagaaacgcccagatatcaaatattttacttttgatttgttagaagaatttgccaaagataatcaattgttttctcagatatgggataatttggcacttgaatttgaccgcgatcgcttgaagccactaccttacaaaacatttccaatagaagatgttcttaaagcctttgattatatgcgacgcgggaagcattttggcagggtagtggtagttatgcctgactcctattctagacaggaacaagagttggatgctcggttatccattgaaaacaaaatgactaaagaggaacagatattatttcaattacaatctggtgaagtttccttagaaaatgcggaacaactattgttaggaaatacggaaacagaaacacaagataaagctatagcagaaaatcaaatagataatattcaaaacaagttaattaacatggatagctcagagaaaatcttatctttgattagttcagtagaaatatctttagaaacagcagaaaaattattattagaagtagtagaaccagaagttaaaacagaggttaatgatgaggttaatcctagtcaaaatcatataccaactacagatatagcgattattggtatttcatgtagatatccaggagcgaataactggaaagaattttgggaaaatttaaagaatggaattgacagtgtaacggaagctcctcctggaagatgggaagaaaaaaattggtatcatccagatccagataatccaggtacttcctattcaaaatgtgccggttttttagatgaaattgataaatttgaccctttattttttcatatttctccggaagaagcttggtttatggagcctgagcaaagaatatttttagaagaagcttaccacgctatagaagatgctggatatgctacagactctcttagaggtaaacaatatggagtatttgtgggagttacggtaaatggtggttatcttaagttgttgtcaatttcaggattagatgttcataggatggcggctacaggaaatggtccgtcaatgataccagcaagaattgcctatatgcttgaccttcaagggccagtagtagctattgatactgcctgctcgtcgtcattggtagctgttcatcaagcttgccaaagcatacaacgaggagagagcgaaatagcgatcgctggaggtattactctaatgccaacatcagacttccaaataatgtcaagtcagtttcaagttgtatctcctgatggacgttgtaagacttttgatgcttcagcatcgggtacagcctggagcgaaggttgtggtgttctcttattaaaaagctacagtcaagcaattcaagacaacgaccatatttatggagtaatcaaaggaacaggagttaattatgatggtaatactaacgggattagtgcccctagtagtcaatctcaagccagtttagaagaagcagtttatcagaaatttggaattaacccagaaactattagttatgtagaagctcatggtacagcaacacctctgggagacccgatagaagtagaagctttaacagaagccttttctaaatggacgaacaaaaaacagttttgtgcgattggctcggtaaaaactaatattggtcattcagcagcggctgccggagtttctggtttgatcaagacaattttgtgcctcaaaaatcaaaaattagttccatccttacattttaatcaaccaaacccacatattgactttgaaaatagccctttttatgtcaatacaaaattaaaagattgggaagtgcttgaaggccaaccaaggcaagctacagttagttcttttggttttagtggcactaatgctcatatagttatagaagaggctccttctcaagttaaaagtcagaatattgttgaacgtccgatccatctgttaactctatctgcgaaaacagaaaaggctctagaagatttagtcagtaattatcaaaattatttagaaactaatcccgagttaccactagcagatgtatgttatacagcctctacaggtagagcacattttaattatcgattaggagttattgcttctgaaccaaaagcattaatagagaaactacttgggtggaaagctcaggaagaattagtaggactattttcaggaaaacgaaatagcgaaggtcagaaaatagcattcctgttcacaggtcaaggttcccagtatgcgaatatgggaaggcaactttatgaaaaagcaccaactttccgtcaagctttagaggaatgtgaccaaattttacaaccctatctagaagtacctctattagaggtcatatactctgaggatgcacaaaagtcaagtgataatctattagaccaaacagcttacacccaaccagctgtgtttgctgttgaatatgctttggctaaattatggtcttcatggggaatcaaaccatgtgtagtcatgggtcacagcgtaggagaatatgtagcagcaacagtagccggagtattcagtttagaagatggtctgaaactaatagccatgcgaggaaagttgatgcaaaagttaccctccggtggtgagatggtatccgtaatggcatcagagtctcaggtaacagaggctataaaagaatatagttcccaagtaacaatagcagcgataaatggaccagaaagtatagtaatttctggtgagagtgtagccataaaaaatatttgtagtttatttgaatcagagggaataaagaccaagcagctacaagtatcccacgctttccattctccaatgatggaaccgatgttagcagagtttgaatcagtagctaaacaagtcacctataatcaaccccaaataccactaatatcaaacgtcacaggtactgaagtagatggagaaataacaaatgctgaatattgggtagatcatgtgcgtcaaccagtaagatttgcccagagtatgaaaactctagagtcggaaggatatgaaaccttcctagaaataggaccgagaccaatattgctaggaatgggaagacaatgtgtaacagaagatgtaggagaatggctgccatcattacgtccaggggtggatgaatgggaacaaatgctatcaagcttaggaaaattgtatgtaaaaggagccaaaatagactggtcagggtttgactctgattatactcgccagaaagtagtattacccacatatccattccagggagaacgttattgggtagaaaccaacaacaacttctggcctcaacggcaattttcccaaggggaaaacctccatcccctattaggtcaaaagctaaattgtgcaggggaacaacaaatatttgcatcacaaataggagaaaactcacccaactatctgagggaccaccgagtatttaatcaagcactatttcccataacaggctacctagaaatagcaatagcagcaggaaatcaccaattaaaaacatcccagatagtaatagaagacctaaccataactagaggatggatactaccaacaggagaattaaccaatgcccaaaccatactcaccccaatagataaccaaagctataagtttcaaatattttctcaaccagaacaacaggagtggagactccacacgacaggaaaaatcagaaaagagtcaaccccccctactcagacaaaagttgacctagaaaaatacaagagtgaatgtaatcaaacaatagaagtcaaacaacattatcaaaaatgtcaacaagtagggatagactacgggaataccttccaaggcatccaagaattgtggtcaggttcaaaccaagcattaggttatatcaaactgcccgaagaattgataacacaaacaagcgactatcatttccatccagcactattagatgcagccttgcaagtaatgttttatgcactgccagcaacggataatgacaaaacttatctgtcagcaggaatagaagaattcagactatataagactcccgggctgagtatatgggcatatgtatcagtaaccagtcaagaagtggaaactccagaaagtttgacggctattgtcaccatagtaactccagaaggagaaataattgccaacatcaaaggtttacaagtcaaactagcaacaaaacagaccctactgggaacagaaaccgaatcaatagaaaattggttatatgaagtagagtggagaaacaaaggtattttaggtaaactactcccaccagatttcctcatacctcctatacaaatcaaccaaaaattaactccaactctgacagaattagtaactcaagtagataatgaaacaacagcgtcttttgaaacaagcttagaagaattaagcagagattatatagtacaagcattacagtcaatgagttggtcatacaaaccaacagaaagctttgcatttgatgtagcagcccaaaaattaggtatagttcctacccatcgaccactgtttaagcgtttgctgcaaatattaacagagtcaggaatactcaactcaaagaatcagcagtgggaagtagcacaaaccttacctgaagtcaagcctacggaaaaaatcagcagtttacagaagaaatatccagaagaaacagcagcattgacactactctctcgttgtgggtctaaactaagtggggtattacgaggagcaatagacccaggagagttagtgttcccccaaggagatttgacagcagcgactcaactttatgaagactcaacagtagcgaaagtgatgaacacaatagtagaaaaatccatcaccaaagctatagaaaaatccccgaaaagccgggggctcaggttgttggaaataggagggggaacaggagggactacaagctatatcctacctcatctaactcctcagcaaaccgaatatacattcacggatataggggcattatttacagccaaagctcaagagaaattccgggattataagttcataaagtatcaaactttagacatagaagtagacccgacaactcaaggatttgaggctcatcaatatgatgtaattattgcagctaatgtacttcatgcaacgacagatatgaagcagacattatctcatgtgcgagaactgttagcagatgggggaatgttggtgttatctgaagcaacagctaaaacactatgggtagatttagtatttgggttgttagaaggatggtggaaattcagggattatgaattacgaccagattatcctttgttgagtcgtgaaaaatggcatcatgtcttgagagaaacgggttttactgaagtagttactatgccagaagtggagggaatggcagaaacattgtcagcacaaacggtaattgtagctaaaagctctcaaacgaagttagaacaaaggaatgatgattctaagagttggttgatactggcagactctgagggagtagggcaacagttagcgaccttactccgctcagtaggagaggtttgtactttagtatttgccggagaaaggtatcaacagatagctccgggagaatttagtattaatcctaatcaagctaaagattttgaggaggtaatagagacagtggcagggaagtcaccatcattgtatggagtggtacaatgttggacgactgaagccggagtggggaatgggattaattctgaggagttaggaagtttatcgaagttagggtgtggcacaactctatctttagtacaagcattggtgaaaggcgggttatcaactgtccctcgattatggttggtgacaaatggtgctcaggcggtgccgaataatcatccggtgataccaggagtagctcaatcttcggtatggggaatggggaaagtgattagctgggaacatccagagttgaactgtactcgtatagatttggacccagaggagactttagagggtaaagttgatgccctatttaaagaaatttggtcggaggatagggaagaccaggtagcatggcgtggggatagtcgttatgtagctcggttggtgggtagtcatcatcggcaattagtggcacaacaagctgatggtaaaactcaaaagcccttaagtttccgttctgaagcaagctatttgattacaggaggtatgggaggtttgggtttgctggtagctagttggatggtgtcaaagggagctaaacatttgatattgttaggacgccgttcaccggatgatgctacgaggaaaaaaataaccgagttagaaatggcaggagcgtccgtggtagtggaaaaagccgatgtgactgatttggagtcgatgaaaggggtgttgcagaggattgaggagtcaaagcgaccgttagtaggagtgattcattctgtgatggtgctatcaaatggagtgctacgaaatcagacttggtctagttttgaacaggtgatggaaccgaaagttcaaggtgcttggcacttgcatcaattgactcaaagtcaaccattagacttctttgtgctgttttcttcagcaacatctctgttgggttcactgggtcagggcatggcaaattattctgctgctaatgggtttcttgatggtttagctcattatcgtcgaactatgggattaccgggattgagtatccattggggagcagtttctcaagtgggacgagcgctcgggcgagatacagagacagccgctatgctcagtaagaatgggatgggtttaatatctccggctcaggtattggagtctttggaactattgatgagtagttcggatgtggaagtaggggtgatgcctattgagtggtcagggtggcaggagagagtggcacagtggccgtttttagtggattggcaggaaactatattggaggtagcccaaccatcgaagtcagattttctgttaaagttggaggctacaccacctaatgagcttcgcttgttgttggtggctcatgtgcgtcgtcaggtggctcaagtgttgagaattagtcatcctgaatcaattgaaatggatacagggttttttgatttgggtatagactctttgacttctgtggagttgaggaataagttgcaaggtagtttaaagtgttcagtaccttctactgtaactttggactaccctaccattaaggcattggtagaatatttatatcaacagctattgttagaacaagttagttactcaaatactgtaccaactgaagaaatcaatgaagatagggaggagattactctaagcgggacaccagctcgggaagcaagtagtgccttacgggatggctatcgtcaagcaggagtcagcgggcgggttcggagttatttggatttattggctggtttaagtgattttcgggaacattttgatggtagtgatggtttttctttagatttggtggatatggctgatggtcccggtgaagtgaccgttatttgttgtgccggtactgccgctatttctggtccccatgaatttacccgcttagccggtgctttgcgtggtattgctcccgtgcgggctgttccacaaccaggttatgaagaaggtgaacccttacccagttctatggccgctgtggccgctgttcaagccgatgctgttattcgcacccaaggtgataaaccctttgtggttgctggtcatagtgccggtgctttaatggcctatgctttggccactgaattattggatcgtggtcatcctccccgcggtgtggttttaattgatgtgtatcctcccggtcatcaagatgctatgaatgcctggttagaagaattgaccgccactttgtttgatcgggaaaccgttcgcatggatgatacccgcttaactgctttgggtgcctatgatcgtttaactggtcaatggcgcccccgtgaaaccggtttgcccactttattggtgagtgctggtgaacctatgggtccctggcccgatgattcttggaaacccacctggccctttgaacatgatactgtggccgttcccggtgatcattttactatggttcaagaacacgctgatgctattgcacgacacattgatgcctggttgggcggtggaaactcctaa

SEQ ID NO: 54 - DNA, 8826 bp, synthetic construct
  source 1..8826: mol_type = other DNA
atggcaaacctaaatcttaatttggacttagtagagggcaactctgatgtagtacaactggtggagttgggtaatggtgtcgtgcaaatcacgatgaaagatgaagaaagctgcaatggcttttctcctggaataattgaaggattatacaaatgttttggtgcagttgctcaaaaccaaagttataaagtagtaattttgacaggctatggaaattatttctgttcgggagggacaaaagaacggttaatcagtatttggaaaggagacagcaaatgtaatgatttagatttttttagaatagcattagattgtgaaataccagtaattgcagctatgcaaggtcatagtattggcggtggtttggttttgggattgtatgcagatttagtagtgttaagtcaagaaagtatttacactactaattttatgaagtatggttttactccaggtgtcggatgcacgttaattctccctgagaaattcggtgctttagggtttgaaatgatgtatactgcccaaaattatcgagggaaagaattagctgaacggggtgtttcttttccagttgtaccgagaaaagatgtgctagaagtggctaaaaatatagcctatgaaatgtcggaaaaacccagactatctttaataactttaaaggaacacttaacttcaaaaatccgcaaaacgctaccaggatttatagataaagaagtagctatgcacgaaataacctttcaccaaccagaggtagcaagtagaatagaggaaaattttgacaaaaggacaacggcaagtaataaccctcaaaattttcctcaagaggctgtaagaaaagaaataagaacagattcattgaattgtcaaccatttcagttgaagacatttagttatggttcgttaaacaatttaactttggtacctctagaacgtagagttccaagcccaagtgaagttgaggttcaaatcaaaactgtaccagttaattttcgggatatactcaatgcacttggtatgctccaagagtattacgaaaaaacatttggcattgctaatgctgaagatctcacttttggttttgaaggtgcaggtactatcgtagctgttggggcagaagtatcgcagtggcaagtcggcgatgaagtaatggtaatgagaattcacgatgcatttagtagctttattatctgctcgccggacaaactggtgcgtaaaaattttaatctgaatatggaagatggagctagtatttgggggccgttttcgactgcatatcacgggttgattaacttagccaaaattcagccaggagatagagtattaattcacgctgcttcaggtgggattggacaagcagcaattcagttggctcaactttttggggcggaagtatttgctactactagtccaggtaagatgaattatctccgggaacagggaattaagtatgtgatgaattctcgaacgatggagtttgtaaatgatgtgatggaattcactcagggccgtggggtagatgttattctcaatagcctgactcatggagaatacatccagaaaaatctagagatccttgccgatagagggcgatatgttgaacttggtaagttgggcatttggagtcacgagcaagtctatcagaaacgcccagatatcaaatattttacttttgatttgttagaagaatttgccaaagataatcaattgttttctcagatatgggataatttggcacttgaatttgaccgcgatcgcttgaagccactaccttacaaaacatttccaatagaagatgttcttaaagcctttgattatatgcgacgcgggaagcattttggcagggtagtggtagttatgcctgactcctattctagacaggaacaagagttggatgctcggttatccattgaaaacaaaatgactaaagaggaacagatattatttcaattacaatctggtgaagtttccttagaaaatgcggaacaactattgttaggaaatacggaaacagaaacacaagataaagctatagcagaaaatcaaatagataatattcaaaacaagttaattaacatggatagctcagagaaaatcttatctttgattagttcagtagaaatatctttagaaacagcagaaaaattattattagaagtagtagaaccagaagttaaaacagaggttaatgatgaggttaatcctagtcaaaatcatataccaactacagatatagcgattattggtatttcatgtagatatccaggagcgaataactggaaagaattttgggaaaatttaaagaatggaattgacagtgtaacggaagctcctcctggaagatgggaagaaaaaaattggtatcatccagatccagataatccaggtacttcctattcaaaatgtgccggttttttagatgaaattgataaatttgaccctttattttttcatatttctccggaagaagcttggtttatggagcctgagcaaagaatatttttagaagaagcttaccacgctatagaagatgctggatatgctacagactctcttagaggtaaacaatatggagtatttgtgggagttacggtaaatggtggttatcttaagttgttgtcaatttcaggattagatgttcataggatggcggctacaggaaatggtccgtcaatgataccagcaagaattgcctatatgcttgaccttcaagggccagtagtagctattgatactgcctgctcgtcgtcattggtagctgttcatcaagcttgccaaagcatacaacgaggagagagcgaaatagcgatcgctggaggtattactctaatgccaacatcagacttccaaataatgtcaagtcagtttcaagttgtatctcctgatggacgttgtaagacttttgatgcttcagcatcgggtacagcctggagcgaaggttgtggtgttctcttattaaaaagctacagtcaagcaattcaagacaacgaccatatttatggagtaatcaaaggaacaggagttaattatgatggtaatactaacgggattagtgcccctagtagtcaatctcaagccagtttagaagaagcagtttatcagaaatttggaattaacccagaaactattagttatgtagaagctcatggtacagcaacacctctgggagacccgatagaagtagaagctttaacagaagccttttctaaatggacgaacaaaaaacagttttgtgcgattggctcggtaaaaactaatattggtcattcagcagcggctgccggagtttctggtttgatcaagacaattttgtgcctcaaaaatcaaaaattagttccatccttacattttaatcaaccaaacccacatattgactttgaaaatagccctttttatgtcaatacaaaattaaaagattgggaagtgcttgaaggccaaccaaggcaagctacagttagttcttttggttttagtggcactaatgctcatatagttatagaagaggctccttctcaagttaaaagtcagaatattgttgaacgtccgatccatctgttaactctatctgcgaaaacagaaaaggctctagaagatttagtcagtaattatcaaaattatttagaaactaatcccgagttaccactagcagatgtatgttatacagcctctacaggtagagcacattttaattatcgattaggagttattgcttctgaaccaaaagcattaatagagaaactacttgggtggaaagctcaggaagaattagtaggactattttcaggaaaacgaaatagcgaaggtcagaaaatagcattcctgttcacaggtcaaggttcccagtatgcgaatatgggaaggcaactttatgaaaaagcaccaactttccgtcaagctttagaggaatgtgaccaaattttacaaccctatctagaagtacctctattagaggtcatatactctgaggatgcacaaaagtcaagtgataatctattagaccaaacagcttacacccaaccagctgtgtttgctgttgaatatgctttggctaaattatggtcttcatggggaatcaaaccatgtgtagtcatgggtcacagcgtaggagaatatgtagcagcaacagtagccggagtattcagtttagaagatggtctgaaactaatagccatgcgaggaaagttgatgcaaaagttaccctccggtggtgagatggtatccgtaatggcatcagagtctcaggtaacagaggctataaaagaatatagttcccaagtaacaatagcagcgataaatggaccagaaagtatagtaatttctggtgagagtgtagccataaaaaatatttgtagtttatttgaatcagagggaataaagaccaagcagctacaagtatcccacgctttccattctccaatgatggaaccgatgttagcagagtttgaatcagtagctaaacaagtcacctataatcaaccccaaataccactaatatcaaacgtcacaggtactgaagtagatggagaaataacaaatgctgaatattgggtagatcatgtgcgtcaaccagtaagatttgcccagagtatgaaaactctagagtcggaaggatatgaaaccttcctagaaataggaccgagaccaatattgctaggaatgggaagacaatgtgtaacagaagatgtaggagaatggctgccatcattacgtccaggggtggatgaatgggaacaaatgctatcaagcttaggaaaattgtatgtaaaaggagccaaaatagactggtcagggtttgactctgattatactcgccagaaagtagtattacccacatatccattccagggagaacgttattgggtagaaaccaacaacaacttctggcctcaacggcaattttcccaaggggaaaacctccatcccctattaggtcaaaagctaaattgtgcaggggaacaacaaatatttgcatcacaaataggagaaaactcacccaactatctgagggaccaccgagtatttaatcaagcactatttcccataacaggctacctagaaatagcaatagcagcaggaaatcaccaattaaaaacatcccagatagtaatagaagacctaaccataactagaggatggatactaccaacaggagaattaaccaatgcccaaaccatactcaccccaatagataaccaaagctataagtttcaaatattttctcaaccagaacaacaggagtggagactccacacgacaggaaaaatcagaaaagagtcaaccccccctactcagacaaaagttgacctagaaaaatacaagagtgaatgtaatcaaacaatagaagtcaaacaacattatcaaaaatgtcaacaagtagggatagactacgggaataccttccaaggcatccaagaattgtggtcaggttcaaaccaagcattaggttatatcaaactgcccgaagaattgataacacaaacaagcgactatcatttccatccagcactattagatgcagccttgcaagtaatgttttatgcactgccagcaacggataatgacaaaacttatctgtcagcaggaatagaagaattcagactatataagactcccgggctgagtatatgggcatatgtatcagtaaccagtcaagaagtggaaactccagaaagtttgacggctattgtcaccatagtaactccagaaggagaaataattgccaacatcaaaggtttacaagtcaaactagcaacaaaacagaccctactgggaacagaaaccgaatcaatagaaaattggttatatgaagtagagtggagaaacaaaggtattttaggtaaactactcccaccagatttcctcatacctcctatacaaatcaaccaaaaattaactccaactctgacagaattagtaactcaagtagataatgaaacaacagcgtcttttgaaacaagcttagaagaattaagcagagattatatagtacaagcattacagtcaatgagttggtcatacaaaccaacagaaagctttgcatttgatgtagcagcccaaaaattaggtatagttcctacccatcgaccactgtttaagcgtttgctgcaaatattaacagagtcaggaatactcaactcaaagaatcagcagtgggaagtagcacaaaccttacctgaagtcaagcctacggaaaaaatcagcagtttacagaagaaatatccagaagaaacagcagcattgacactactctctcgttgtgggtctaaactaagtggggtattacgaggagcaatagacccaggagagttagtgttcccccaaggagatttgacagcagcgactcaactttatgaagactcaacagtagcgaaagtgatgaacacaatagtagaaaaatccatcaccaaagctatagaaaaatccccgaaaagccgggggctcaggttgttggaaataggagggggaacaggagggactacaagctatatcctacctcatctaactcctcagcaaaccgaatatacattcacggatataggggcattatttacagccaaagctcaagagaaattccgggattataagttcataaagtatcaaactttagacatagaagtagacccgacaactcaaggatttgaggctcatcaatatgatgtaattattgcagctaatgtacttcatgcaacgacagatatgaagcagacattatctcatgtgcgagaactgttagcagatgggggaatgttggtgttatctgaagcaacagctaaaacactatgggtagatttagtatttgggttgttagaaggatggtggaaattcagggattatgaattacgaccagattatcctttgttgagtcgtgaaaaatggcatcatgtcttgagagaaacgggttttactgaagtagttactatgccagaagtggagggaatggcagaaacattgtcagcacaaacggtaattgtagctaaaagctctcaaacgaagttagaacaaaggaatgatgattctaagagttggttgatactggcagactctgagggagtagggcaacagttagcgaccttactccgctcagtaggagaggtttgtactttagtatttgccggagaaaggtatcaacagatagctccgggagaatttagtattaatcctaatcaagctaaagattttgaggaggtaatagagacagtggcagggaagtcaccatcattgtatggagtggtacaatgttggacgactgaagccggagtggggaatgggattaattctgaggagttaggaagtttatcgaagttagggtgtggcacaactctatctttagtacaagcattggtgaaaggcgggttatcaactgtccctcgattatggttggtgacaaatggtgctcaggcggtgccgaataatcatccggtgataccaggagtagctcaatcttcggtatggggaatggggaaagtgattagctgggaacatccagagttgaactgtactcgtatagatttggacccagaggagactttagagggtaaagttgatgccctatttaaagaaatttggtcggaggatagggaagaccaggtagcatggcgtggggatagtcgttatgtagctcggttggtgggtagtcatcatcggcaattagtggcacaacaagctgatggtaaaactcaaaagcccttaagtttccgttctgaagcaagctatttgattacaggaggtatgggaggtttgggtttgctggtagctagttggatggtgtcaaagggagctaaacatttgatattgttaggacgccgttcaccggatgatgctacgaggaaaaaaataaccgagttagaaatggcaggagcgtccgtggtagtggaaaaagccgatgtgactgatttggagtcgatgaaaggggtgttgcagaggattgaggagtcaaagcgaccgttagtaggagtgattcattctgtgatggtgctatcaaatggagtgctacgaaatcagacttggtctagttttgaacaggtgatggaaccgaaagttcaaggtgcttggcacttgcatcaattgactcaaagtcaaccattagacttctttgtgctgttttcttcagcaacatctctgttgggttcactgggtcagggcatggcaaattattctgctgctaatgggtttcttgatggtttagctcattatcgtcgaactatgggattaccgggattgagtatccattggggagcagtttctcaagtgggacgagcgctcgggcgagatacagagacagccgctatgctcagtaagaatgggatgggtttaatatctccggctcaggtattggagtctttggaactattgatgagtagttcggatgtggaagtaggggtgatgcctattgagtggtcagggtggcaggagagagtggcacagtggccgtttttagtggattggcaggaaactatattggaggtagcccaaccatcgaagtcagattttctgttaaagttggaggctacaccacctaatgagcttcgcttgttgttggtggctcatgtgcgtcgtcaggtggctcaagtgttgagaattagtcatcctgaatcaattgaaatggatacagggttttttgatttgggtatagactctttgacttctgtggagttgaggaataagttgcaaggtagtttaaagtgttcagtaccttctactgtaactttggactaccctaccattaaggcattggtagaatatttatatcaacagctattgttagaacaagttagttactcaaatactgtaccaactgaagaaatcaatgaagatagggaggagattactcta

SEQ ID NO: 55 - DNA, 995 bp, synthetic construct
  source 1..995: mol_type = other DNA
ttgacagctagctcagtcctaggtataatgctagcatgaccgtattacatagtgtggatttttttccgtctggtaacgcgtccgtggcgatagaaccccggctcccgcaggcggattttcctgaacatcatcatgattttcatgaaattgtgattgtcgaacatggcacgggtattcatgtgtttaatgggcagccctataccatcaccggtggcacggtctgtttcgtacgcgatcatgatcggcatctgtatgaacataccgataatctgtgtctgaccaatgtgctgtatcgctcgccggatcgatttcagtttctcgccgggctgaatcagttgctgccacaagagctggatgggcagtatccgtctcactggcgcgttaaccacagcgtattgcagcaggtgcgacagctggttgcacagatggaacagcaggaaggggaaaatgatttaccctcgaccgccagtcgcgagatcttgtttatgcaattactgctcttgctgcgtaaaagcagtttgcaggagaacctggaaaacagcgcatcacgtctcaacttgcttctggcctggctggaggaccattttgccgatgaggtgaattgggatgccgtggcggatcaattttctctttcactgcgtacgctacatcggcagcttaagcagcaaacgggactgacgcctcagcgatacctgaaccgcctgcgactgatgaaagcccgacatctgctacgccacagcgaggccagcgttactgacatcgcctatcgctgtggattcagcgacagtaaccacttttcgacgctttttcgccgagagtttaactggtcaccgcgtgatattcgccagggacgggatggctttctgcaataagccacaattcagcaaattgtgaacatcatcacgttcatctttccctggttgccaatggcccattttcctgtcagtaacgagaaggtcgcgaattcaggcgctttttagactggtcgtaatgaa

SEQ ID NO: 56 - DNA, 1194 bp, synthetic construct
  source 1..1194: mol_type = other DNA
gctttttaacttggatttttaccttctcagcctaaaccttaacattagtgtcaatgtcaaggttttgagtaaactaaagggatgaagactaatcacttaacgattaaagaactcacagatgcagtgggaggtggcgttacgcctcgcatggtgcgccattaccacaccctgggattgcttccccccgttcaacgctcagagggcaactaccgcctttatactcagcaggacgtacaacggctccaacgagtcattgccctcaaacagcagggctttcagttgtctcatattcggcaactgctggatagccattctgaagagagccttgatcccaccctaatggtgcagttgcaacagcaatatcaggctgtgattcagcagattactcgactccgccaaaccgcatctgctttagaaggattactgggacgcgaccagagttgccagattacccaagcggaagctctggcccaactgaaacagcttgatgtggatgtccaggaggggttaggaaaactcgatcagttgtggacgaacctagatgcggagacaacaactcatccagaagcctttcaggaatccctcaaacacctgctaccggatttatcggcttactctgaaattaccattcacttactacatcaattagtgctggcctgtggtgatgttagcttggtaaacgccgttcgattgagtcagggagcgatcgcctcggcacgagatgcactgaaagcagggtgtccagtcgtcaccgatgttccggttgtggctgcggctcttgatcaaactcggttagctcatttaggatgtacggttaaaacgctgattgacgaccctcacatcacagggcttagggaagccgagcaagctttttggcaccatgaccattggcaacagcggttacaacagattccccaaggatgtgtgctggcgatcggctatgccccttctgttttactcactgcctgtaagctgatagagcaacaacatattcagccggctcttgtgatcggaatgccgatcggttttagtcatgctccgggggcaaaacgacgactgatgaccagtcccattccccatatcaccattcaggggagcctcggtggaggacttctagccgcagtaacgctaaacgctttagtggaaacattgattgcaaagccagattgccactgctatctcacttgtctttag

SEQ ID NO: 57 - DNA, 32767 bp, synthetic construct
  source 1..32767: mol_type = other DNA
atcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgcgaattcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttaggtgacactataggatcccgacatcataacggttctggcaaatattctgaaatgagctgttgacaattaatcatccggctcgtataatgtgtggaattgtgagcggataacaatttcacacaggaaacagaccatgctagataaaataaatcgttatgctcatgggtttgtagctgtaccagtgatttgtgcttgttcagaagcaggggtattcgagttactatcacagaaaaaatcactgaaattagaagaaatagtagagcatttagcagcaaatagtggacatctaatggtagctatgcgactcctagagtcattatcattcctatatcgctctcaagcagaagaatacatattgacagaacaaagtcaacaacatcaaatcatccctaaagctctgatgagcttatataaatatccatttgagttatatttaaaaggagaagtagaaacaggaatcagcaattggattaattgttcatctcgaagatgggatacagaaaactcattactaagtgatttattagatggagtattactcatccccctgctactagaactgaaaaaacaaaacttactggatgaatcaaaaaaaatatttaacacattaacaaattcccttaaacaagaactatcaacattattcatcaatttaggatgggcagaagaaaaaacagaagggctatatttaacagatataggtagatttatgcgtgacagatccttaaacttaggaacaacagcgtcttatgctcctatgttgttacagatgaaagaattactatttggaaatcctcagagagtatttcaaagaaataagactgaaaaagaaagacacgtaaatagaacattaaatgtagtagcaagtggctttcaacacgaaaagttttttgccgatacagataaaatcatcatatctatatttaaccaacagccaatagaagagcaaccaagctatatagtagacatgggttgtggggatggaacgctactgaaacgaatatataaaatcatcaaacaattctctgctaggggaaaagtattaacagagtatcctatcatcatggtaggagtagactacaatcaagaagcattagatgtcacagataaaaatttagtagatattccccatctagtcattccaggagatataggagcaccagaaaaattattagagcaattgaaagcacaaggaatagaaccagaaaaagtattacatattcgttcattcctagatcatgaccgaccatttatagctccaaaaaatacagaaatagctcaagcacgttctcagttagattatcaagtagtagatgtagatcgagaaggaaagctgataccacctcacatagcagtacaaagtttagtagaacacttagaaagatggtcatcaataataactagacatggattattgctcctagaagtacatagtttaacgccagcagtagtcaaaaaatatatagacgaaagtgaatctctgcattttgatgcctatcatgccttctcaatgcaacatttagtagaagcagatgtgttcttgatggcagcagcagaggtgggtttattttcacggaaagaagcttttcgtaagtatccgaaaacgttgccattgactcgaatcacagttaatcattttgaaaagcgtaaatatcagatacggtatgcaactgtaaacgatataccaaatctgttaaagtgtgcgacatttaatcaacctgtaaacgaacccttctttcaagttttattgaaacaaactccaacagcacatttattattagaatatcaaggtgaattggtagctgcaatcttcacagaaactaaaaattctaatgaggtgctaggaattcgtgagtttttagtaaggacttctgtagaaaattggcaagtattagcaaaagatttactagaatttgtggaacagtggggagtagtcaaaccaggaataaaagaaatagaaggattgttaaaataccatgaagccatctcaaactttcaaaaatcaaagtggtatcaatcttcagttttaaacaaaaagcttatagaaaaaataactctacacgaattagctactttagaactatgtaatttaatggctccagaatatgagctagaagcctttgccgcacgttggcttttgcgtgtttttcaagatatgggtgtatttctcagagagggtgaatcttatcaggagtctgagttggtttcccagctaaacatctcaccacgttaccagagacttttaggtgctttgttacaaattttgcataagcgtggtattctaaaaattgagaaagatagagtgttcacattagcaagatgtaagacctttgccttagaaaatatttcgtctgaagtttctgctttttatgattatttttctgaaaaatatcctgctcatttatcctggttaacagtagtcaaaaggtgtctagagaaatatcctttgattctacgcggtgaagttgatgtcaacgaagttgtttttacagatggggatatggagctatttgctggactatttctaggacatcgtgttgctgactactttaatgagttgctagcagatggggtttgctgggaagtagaacagcggttgttagaagaaaagagggcacaacctattcggatcttggagattggagcaggaacaggaggtgttacaggaatattgctagaaaagctagcttctcatgcagagcaaattgaattttggtttactgatatttctagcgtttttacacgttatggtgagagtaagttcaagcagtttccttgggtgaaatatcaaacctttgacatagaaaaatctcttgatgctcaggggataaagtctgaaagttttgatgtggtaattgctaataacgtactccataacacaaaattaattcatcaaaccttaaataacagtaactcactattaaatactggggggttattggcattactagagtttactcaaccaattgatattcttttatactttggagggttgcttcaaggattttggttgtttgaagatccagaataccgactagaagttggttgtttactgagtataccactgtggcaaaaagttctcagtgattgtgggtttgatgaaataataccattgggattaccctgtgagatgcacgctctttctaaggcaagagaatctgttatatttgctcgaaagcatcaagtacaggagaaaacattttctgaaaaaattaaacaaaatttgacagaaaatggtaagcatgggcaggcagaattcgattttattagtattaataattcacaagaaagttcatccaaattagaaatttttgaacaggaatgtcgaaaattattaaaatctctactgggtgttcaacgtatggagagattgcctggtgacacaccactaatggagtcaggaatggattcactggagttgttagaatttcgtgctcttatagaaagaaagtttgggattaagttaaagtctaccttcttttttagttacaaaactcttatagcggtagcagagtatctttcagaacgggaagatattaattttagttagtaggactatgactgaaaaaaaaacaacgttttcacaggtccacagggggaaattttgaatcagggggaaaatttgaatgcattctaaccagagcagttcactagcaagaatagccattgtaggtgtggcttgccgttttcctggagcagatactaaagaagaattttggcagctacttaaggaaggtcgggactcaatgcagaatcttccttcagagcgttggggaaatctatttaactccatgagttcagaaatagatttatctattcaacgcggtggttttttgaaagatatagatttgtttgactcatccttttttaggattactcctagggaagcgcagttaatggacccccagcaaaggctgttgttagaattgtcctgggaagcgatggaagatgcaggatatgcaagggatacattaaagggtaaatcagtaggggtttatgtaggagtttgtcactatgactacagaagtttattagaaaaaggtttggagacagccgaaatagctcaaattgctactggtacagctccagcaacttttgccaatcgcctttcctatttttataattttcacggccctagcttaactgttgatacggcttgttccagctctttggtagcaatgtatgaagcagtaaatgcaatacgtcgtgggcagtgtcaaacagcattggtaggaggcgttaatttaatgtgttctcctgtgaataaccaagtttatagtgctgcgggtatgttatctcctgatggagtctgccgggtgtttgatgcgggtgctaatgggtttgtcaggggagaaggaggggcagtagttgtacttaaagattatcaaaaagcgttgacggatggtgattctatttatggggttgttcgcagtgtagcagtgaatcatggagggcaagcaagttcttttacagcaccaaatccccaagctcaggcaaaattgttggaacaggcttatagggaagctaatattgatatagagtcggtaggttatattgaagcacatggtacgggtacatctctaggagacccaattgaggtcgaggctttaaatgaagcgtttaaagggttgagttcaagtggtaagttacctgctaatagttgtggtttgggctcagttaaaacaaacattggtcatttggaaggggcagcaggtttggctggactaatcaaagttttgctgtgcatgaggtatgcaactcttccatgttcattaaattatcagcaacttaatcctgatattgaattagaagaaggtccgttttttgtggtggacaagcttcaatcttgggaaataaaagttgatagggtaggaaaaccttatccacttagagcagggttgagtagctttggttttggcggtacaaatgctcatgtagttcttgaggaaggagaaaataagaaggaaggaagaagaggggataaggaaccttcagttcatctattaaccctttcagctaaaactgaaacagctctttcagagttagtcagtcgttatcaaaaatatttaaacactaatgcagaatcagagttagccgatatttgttataccgctaatactgggcgagtccattttaaccatcgactagcagcgatcgcctcaaacaaacaggagttagtagaaaaacttaaggaataccaagctggggaggcaacagcgggagtattaataggggaacttgcagaacgcataagaacaccgaaattaggtttcttattcacaggtcaaggttcccagtatgtcaatatgggaaaacaactgtataaaacacagcctgtattccgtgaagtattggataagtgtgatataatattggaaacggaaatagaatgttctctattagatgttctatataacaaaactacagattctcaagattcatctttaataaaccaaacagcttacactcaaccagttctgtttgcaatagagtatgcgctatttaagttatgggaatcttggggaattaaacccagcatagtaatgggtcacagtgtaggagaatatgtggctgcttgtgtagcaggagtcttcaatcttgaagatggtttgagattaatagctgctaggggcaggctgatgcaacagttaccttcaggtggagaaatggtttctgtgatggcttcagaatctacagttagcagactcctagaacctcacaaaaaagaaatagcgttggcgaagcccgccgtaggcatcgcagcaataaatggaccagaaagcacagttatttctggagactctgtagcagtaacaggtgtagtaaatgacctggaagcaaagggaataaaaactaaaaaactagaggtatctcatgctttccattcaccattaatggaaccaatgttaggagagtttgaagctatagctaatcaactaacctacaatcaacctaaaatacctattatatctaacgttacaggtacaaaagcagataatactattgcttcccctcaatactgggttaatcatgtcggtaaacctgtaagatttgcccaaggaatggaaacgctacatcaacaagggtatgaaacctttatagaagttggaccgaaaccagtattgttaggaatgggcaggcaatgtctaccaggaaatgtaggtgtctggttgccatcattacgtccgggagtggatgagtggcaacaaatgctttatagcttgggaaaattgtatgtaacaggagtaaaaatagattggtcaggatttgagtctgactctagtcgccagaaagtagcattgccaacttatccattccagcgagaaagatattggatagaaataacagaaaacaaacataaggaacatcaaaagtcagaaaatataagtgacacttcaattgttaaactactcactcaaggaaaaacagaagctctcactcaacaactagaaacagaagccaaattttcaccagaagagcttaaacttttaccagaaatattagagacattagccaaacaacatcaagaacaattaacagcagtaaccatcaaaaactggttctacgaaatccagtggaaacccttagctcaaaacaaccccaatgcaaacattgaacctagtcattggttaattttagccgataccacaggagtagcagaaaaattagctcaaaaattacaacaacagggtcataaatacagcttagtttatcgaggagagagctatcaaagacaagcaacaggtacttatcaacttaatcctcagattcccgaagcatttgaaaagctgtatcaagaaattcaacaaagtagtgaaactgccattacgaagttaattcacttgtggagtttagatgctccccaatcaaaagacttaaccctggaaaccctagaagaagctcaattatggggatgtggcagcgtagtacacctgttacagaccttagtcaaaaactctagcattcctgaactatggttagtaacccgtgggtctcaatcagtattatcccaaacagaaaaaaatctaacaggactagcagcgtcacccttgtggggattaggtagagtagtgtctaatgaacatccccaattatggggaggattagtagatttagacccacaagctgcagcaggagacgaagtagaaatgctgtggcaattattagttaatgaacaagaagaagataatctagctgtacggggagaaaatacctatgtagctcgtctggccaggcaagaacctcaagaatttcctgaatccctatccttatcatcagatggtagttacctaataacaggagggttaggagctttagggttacatactgcccaatggttagtatccaagggagcgaaaaatattgtcttaactgggcgtcgccctccctcagaaaaagtaagtgaatctataaaaaaattagaagaaacaggatgtcaagtgaaggtgatgttgggggatgtttctgttgaagaagacataggcaaaattctcaaacaaattcagatatcaatgccaacactcaaaggcataattcatacagcaggagtgttagatgatggaaccatacaacaaatgaattgggagcgttttgccaaagtcatgtcgcccaaggtaaaaggaagttggcatttacataaattaactgaaaatcagccattagatttctttgtgtgtttctcctcaatagcttcgatgttggggactcttggccaaggaaactatgctgcagccaatgcctttatggatgctttagctagttatcgttgcagcagaggattatcaggattggcgattaattggggagcatgggcatcagggggaatggctgctcgtttagcagtggagcatcaaaataggatgcacagcagtggcataactgagatggcgaccaaagaaggaatgtatgccttagatttactattaacaaatgaatctgctacagctcaggtaggtgtagcaagtatagagtggcaggtactctcagaaagttggagtggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttaggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttatgtatagaggaattaatgggatggaagacaacagaaattgacccattatctgaagaaacaagattagagatgatggagcgctcaagaagtgaagcaatagccatcatcggcataggctgtagatttcccggaaatgccaacacaccagaaagcttctggcaattgttatccaatggcaaagactccattacagagattcccctagaacgttgggatctagattcctactacgaccccaaccctgatactccagggaaaatgtatatccgtcacgcagcattagtagaaaaagtagatcagttcgacccacgattttttggaatctctaaccgagaagcttatagtcttgatccacagcaacgcttcattttggaagtaacttgggaagctttagaaagagctggtattaaccctcaacaattagaaaacactcagacaggggtgttcctgggtatcggtcaaaatgattacgcgaatttaggttttcaccaagcagccgaagatataagcccttacgatgctacaggaaatttgttttgttttgtagcaggtaggttatcttactttttaggaacgcaaggtccatcaatggcaatagatacagcttgttcatcatctctagtagctatccatgaagcttgtgagagtctgcgtcagggtgagtccaacttggctttggctggaggagttcaactaattctctctcccgaagtaacaaccgcactatcaagattaaaggctctagcacctgatggtaagtgtaaaacttttgatgctgctgctgatggttttggtaggggagagggatgtggcattgtggtactgaagcgtttgtcggatgcgctcaaggatggggacaggatatcagcagtaattcctggttcggctgttaaccatgatggaccaagtagcggaatgacagtaccgaataaactggctcaggaaaaactgattcagaaagctctcaaagcagccaaggtaaaaccgttacaagtgagttatgtggaagctcatggtacaggaacttctctaggagatcctatggaagtgagagctttagctagggtgtttgaggagggacgtgatcaggaaaatccattgaacatcggttcagttaaaactaatatcggtcatctggaagcagcagctggaatagcaggtatgattaaggtgattttgcaattgcaacatcaggaaattgtgccccatctgcattttgctaatcctaacccctatgttgattgggagaatatgcctctacaagtaccgactcaactgactccttggttgtcgaaaggggagaaaagggtggcaggagttagttcttttggtatgagtggtacgaatgctcatatagttttagaagaggctcctattgaagtcagaaggcagaagtcagaagtcagaagtgaagaatatctagaacgtccggttcatatactaactctgtcggccaagactgaaaaagcactagaagatttagttaatagttatgaaagttatttagaagccgaaaataatgataattatttaggggatatttgctacacagccaacatcgggagagcaaaatttgaccacaaattagcagtggttacttctgataaacaagagttattagagaaactcaaacaatataaacaaggtgagaatgttgctggaatcttttcaggaaaacaaataagtgaaactagaacaaaaatagcctttatatttactggtcaaggttctcaatatctgcaaatgggaaggcagttatacgaaactcaacccacttttcataaaattattgaccagtgtagtgaaatgctggtaaaatatttagatgtttctttattagatatactttatccagttgaggttaaagatgaaagttcgactttgatagaccaaacagcttatactcaacccgctatatttgcccttgaatatgccctagctaaattatgggaatcatggggtataaagccagatgtagtcatgggtcacagtgtaggagaatatgtggcagcaacagtagcaggggtatttagtttagaagatggtttaaaattaatagccatgcggggacagttgatgcaaaagttaccctccggaggtcagatggcatctataatggcatcagaatctcaggtaatagaggcgataaaagaatatagttcccaagtaacaatagcagcagttaatggaccagaaattatagtaatttcaggtgagagtccagccatttcaaaaatttgtagtaaatttgaatcagaaggagtcaagaccaaggcgctacaagtatctcatgctttccattccccattgatggaaccaatgttaacagaatttgaagcagtagccaaagaaatatcctataatcatccccaaataccactaatatcaaatgttactggtcaagaagtaaatggagaaataaccactgctgaatattgggtgcgtcatgtgcgtcaaccagtaagatttgcggaagggatggaaactttacacaaacaaggtgctgaaattttcctagaaataggctcaaaaccgatacttttaggtatgggtcgtgagtgtctcatgggagaaaaaaaactatggttgcccagtttacgttcaggaaaaccagactggttacaaatgctacaaagtctggggcaattgtatgtacgagcaataaaaattgattggttaggatttgatcgggattatttccgtaataaggtagagttaccaacatatccttggcaacgaaaaaggtattggataacagatattagacaacggaaaagtcaagacaaaaaaagcataacatctgaaaaaaaagtacaacttgatggagtaaatattcaactaaaggaaatacaaatgaacgacaaaatcttacagcaaccaaaactaaaattatcagatccagaatcactatatttatcgaatgctgaatcaactatagaggttccaacaaaagcagttcaagtaaaacctgctgctgatattgaatctgaaaatataactcaattgaatagccttgatagagatgtgactcaaatcatagaaaccctcaaagaaagtttggcagatgctttatatgcagatataagtgaaattgaagaagacaaaaagtttgttgatttaggtttagattctattgtgggagtagaatggataactaacatcaataaaatctacaatttaaatataaaagctactaaactatacgattaccctactttgccagatttggctaaatatgtcgctcacaccctatcagctcagggtagaaatattgatgtagagcgatcgccatctgactcaagtcaagcaagcattagcaagcaatctcaactaactgatacccagagcaatttctcacaagtcaaggaaattttgaaacaacaattagcagatgctctgtatgcggatattagtgaaattgaagtaaacaagaagtttgttgatttaggtttagattctattgttggagtagaatggataactaatatcaataaaacctacaatttgaatatcaaagccactaaaatatacgactaccctaccttattagatttcgcaaaatatattaaccaggaaatttattctacaggagtaagtagattttcaacggagcataaagaatttaatcaaaaagactattcatctggaggttcgcaagaggaaatgacacaaaaattacgatcaatattgaagaaagtggccaataaagaattaacagttcaggaaggaaataaaatgattcaacaaataaaaaatcaattaaagtaaaaatatagtataatttttaaggaaataaaattttatgaacaaagagcaaatatttcaaataataaaaaaatatacctacgaaatagcaccagaattagaagaagtacctatatcacctactgatagtctcaaaaacttggggattgattcagtaaatagggcagaaattataatgatggtaatggaagagttatcattaaatatcccgcgtattgaattagctggatccaaaaatataggagaactggctgatatatttgcattcaaattagaagcaataaattcacaaagctagaaggcctatcaatgagtaacatagaaataacaggtatgggcattgttacttccattggtcaaggagttgctacttttaaagaagctctgttatcagggaaaactcaatttgcttatctaaaacagccaggacgtgaaagcatcaaaccatttattggtgccgaaattcccgatattgacgccaaaaccctatttcctgagtacagtggactgttacgtactgctactaagagcgcccaagttgcaatagtagctgtcgctgaagcctggcaagatgctcaactcacctccagccaagttaacccagaacgagtaggattagttgtaggtggttcaaatttacaacaacgttatcagcagcaaacctggcaacgttatcactcacgtcaggagtttatacgaccaacttatggtctgaccttttgggatacagatatattaggtctgatttcccagtgttttcagattcaaggtgagggctatagtgtaggtggagcttcggccagcggagcagttgctataattcatgctgctcgtcaaattttaatggggaacagtgatgtttgcattgctctgggagctttatccgatctatcagggtatgaattccaagcactaatgaatttaggtgcaatggggagcgaacgttttgccgatcgcccaaaccttgcttgtaggccatttgaccaagaccatgatggctttatttatggagagggttgtggagcagtgattttagagagaactgaccgcgctcaacaacggggcgctcaatcccatggccaacttaagggttggggattaactttagatgggaaccgtagtcccgaaccttcccaaaaaggggaagaacgggctataaatactgctttagccatggcagaccttcagccagagagtattgattatgtgaatacccacggtactggttccccccttggcgacaaaacagaagtggcagctttcaaatcagtaggacttcagcattgcctatttaattcaacaaaatctttaataggccactgcttaactgctgctggtgtagtggaagcgatcgccaccatattacaaatgaagtttggtttttgtcatccaactaaaaatttagttaatcctattgataccagtctcaattgggtgaaagaaacttctgttcaagctgagattaaatatgctatcagcaacagttttggttttgggggtatcaatacagctttattaattggacaggagtaaactcatgcaacaagttggaattgaagcactaaatgtatatgggggttcagctaaactagatgtgcgaatgctaggcgaagcacgtcacttagacatgactcgcttcgataatcttctgatgaaagagaagacagttgctatgccctatgaagatccagtttcttatgctattaatgcagcaaaacctattattgatagcctcagcgttctggaaaagcaacaaatcaaaatggtgatagcttgtagcgaatctggcattgattttggcaagtctatgagcacttatatccaggattatttgggactaaatcgtaactgtcgaatgtttgaaataaagcaagcttgctactcaggtacagcaggtttacaaatggcattcaatttaattttgtcccaaacctgtccaggggctaaagctttggttataggaactgatatatttcggcctgttgttgtagagggaggagaagctctcagcgaagattggtcttttgtggaaccaagcagtggtgctggagcagtagctattttagtcagtgatgttcctaaaattttccaagctgatgtaggatgtaatggttactatggctatgaagtcatggatacttgcagacctaacccagactcagaagcaggagatgcagatttatcattgctatcttacctagattgttgtgaaaatgcttaccgggaatatcaagatcgagtagaaggagtagattaccaaaaaacttttgattacttgagctttcatactccttttgggggtatggtgaaaggggctcacagaagtatgatgcgtaagtttaaaagggcaaaacctgtggagattgaagaggactttcagaaacgggtgatgccgggattagtctattgtcaacaggtgggtaatattatgggagctacagtatttttatctttagctagcatgattgataatgcagattttagcaaagcccgacgaattggtgtattctcttatggctctggttgttgttcggaattttatagtggagtcgtcaccccgcaaggaaaggaaattcaagctcaacaaaaaatttcatcacagttagcaatgcgctattccttaagtatggaagaatatgagcagttactaagtcatagttcggcagttgcctttggcactagaaatgttaccctagattacaaactatttcctggtgtgtgggaacaaattgaaggtaaaggtcgcttagtgctgaaaagaatcaaggaatttcaccgagaatatgaatgggtatagccatgagttatcaaaccctgaaaatcagttatcaagatgttgtacaaaggattcagatatatcgacctgaatctaacaatagcatcaatagtcaattaacgatggaattgttgtcagctttgcaagctgctgaagcagaggaagttgttaaagtagtgatattagaaggactacctgatgtattttgtacagggatggattttgaagaagtggcaacagcaaaacaatttgatccaaaagctagtgctaatggttactacaatattttgaaacaaatgtctcaaagtagcaaagtaattttgtcacttgtgcgtggtaaagtacaggcagggggagttggtttagtagcagcaagcgatctagttattgctgatgaaacggcaacttttgttttatcagaattattatttggattattaccagcttgtgtattgccttttttgattcgtcgagtgggatttcaaaaagcctaccgtttagcactaacgactcaagctatttcagtatcagaggctgataagtggggattaatagatgaatatggcagtaatattaatcagttaataagtaaatatattcgacgtttaaagtatttaccctcatcaggggtgaaagagttaaaaaattatattaatcagttatggattattcaggcagaaactcaaggtttagccgtcaacgagatttctagcttaatagcagaacctactgttcaagaaaaaattaaacgttttcaaaaagaaggattatttccatggcaaacctaaatcttaatttggacttagtagagggcaactctgatgtagtacaactggtggagttgggtaatggtgtcgtgcaaatcacgatgaaagatgaagaaagctgcaatggcttttctcctggaataattgaaggattatacaaatgttttggtgcagttgctcaaaaccaaagttataaagtagtaattttgacaggctatggaaattatttctgttcgggagggacaaaagaacggttaatcagtatttggaaaggagacagcaaatgtaatgatttagatttttttagaatagcattagattgtgaaataccagtaattgcagctatgcaaggtcatagtattggcggtggtttggttttgggattgtatgcagatttagtagtgttaagtcaagaaagtatttacactactaattttatgaagtatggttttactccaggtgtcggatgcacgttaattctccctgagaaattcggtgctttagggtttgaaatgatgtatactgcccaaaattatcgagggaaagaattagctgaacggggtgtttcttttccagttgtaccgagaaaagatgtgctagaagtggctaaaaatatagcctatgaaatgtcggaaaaacccagactatctttaataactttaaaggaacacttaacttcaaaaatccgcaaaacgctaccaggatttatagataaagaagtagctatgcacgaaataacctttcaccaaccagaggtagcaagtagaatagaggaaaattttgacaaaaggacaacggcaagtaataaccctcaaaattttcctcaagaggctgtaagaaaagaaataagaacagattcattgaattgtcaaccatttcagttgaagacatttagttatggttcgttaaacaatttaactttggtacctctagaacgtagagttccaagcccaagtgaagttgaggttcaaatcaaaactgtaccagttaattttcgggatatactcaatgcacttggtatgctccaagagtattacgaaaaaacatttggcattgctaatgctgaagatctcacttttggttttgaaggtgcaggtactatcgtagctgttggggcagaagtatcgcagtggcaagtcggcgatgaagtaatggtaatgagaattcacgatgcatttagtagctttattatctgctcgccggacaaactggtgcgtaaaaattttaatctgaatatggaagatggagctagtatttgggggccgttttcgactgcatatcacgggttgattaacttagccaaaattcagccaggagatagagtattaattcacgctgcttcaggtgggattggacaagcagcaattcagttggctcaactttttggggcggaagtatttgctactactagtccaggtaagatgaattatctccgggaacagggaattaagtatgtgatgaattctcgaacgatggagtttgtaaatgatgtgatggaattcactcagggccgtggggtagatgttattctcaatagcctgactcatggagaatacatccagaaaaatctagagatccttgccgatagagggcgatatgttgaacttggtaagttgggcatttggagtcacgagcaagtctatcagaaacgcccagatatcaaatattttacttttgatttgttagaagaatttgccaaagataatcaattgttttctcagatatgggataatttggcacttgaatttgaccgcgatcgcttgaagccactaccttacaaaacatttccaatagaagatgttcttaaagcctttgattatatgcgacgcgggaagcattttggcagggtagtggtagttatgcctgactcctattctagacaggaacaagagttggatgctcggttatccattgaaaacaaaatgactaaagaggaacagatattatttcaattacaatctggtgaagtttccttagaaaatgcggaacaactattgttaggaaatacggaaacagaaacacaagataaagctatagcagaaaatcaaatagataatattcaaaacaagttaattaacatggatagctcagagaaaatcttatctttgattagttcagtagaaatatctttagaaacagcagaaaaattattattagaagtagtagaaccagaagttaaaacagaggttaatgatgaggttaatcctagtcaaaatcatataccaactacagatatagcgattattggtatttcatgtagatatccaggagcgaataactggaaagaattttgggaaaatttaaagaatggaattgacagtgtaacggaagctcctcctggaagatgggaagaaaaaaattggtatcatccagatccagataatccaggtacttcctattcaaaatgtgccggttttttagatgaaattgataaatttgaccctttattttttcatatttctccggaagaagcttggtttatggagcctgagcaaagaatatttttagaagaagcttaccacgctatagaagatgctggatatgctacagactctcttagaggtaaacaatatggagtatttgtgggagttacggtaaatggtggttatcttaagttgttgtcaatttcaggattagatgttcataggatggcggctacaggaaatggtccgtcaatgataccagcaagaattgcctatatgcttgaccttcaagggccagtagtagctattgatactgcctgctcgtcgtcattggtagctgttcatcaagcttgccaaagcatacaacgaggagagagcgaaatagcgatcgctggaggtattactctaatgccaacatcagacttccaaataatgtcaagtcagtttcaagttgtatctcctgatggacgttgtaagacttttgatgcttcagcatcgggtacagcctggagcgaaggttgtggtgttctcttattaaaaagctacagtcaagcaattcaagacaacgaccatatttatggagtaatcaaaggaacaggagttaattatgatggtaatactaacgggattagtgcccctagtagtcaatctcaagccagtttagaagaagcagtttatcagaaatttggaattaacccagaaactattagttatgtagaagctcatggtacagcaacacctctgggagacccgatagaagtagaagctttaacagaagccttttctaaatggacgaacaaaaaacagttttgtgcgattggctcggtaaaaactaatattggtcattcagcagcggctgccggagtttctggtttgatcaagacaattttgtgcctcaaaaatcaaaaattagttccatccttacattttaatcaaccaaacccacatattgactttgaaaatagccctttttatgtcaatacaaaattaaaagattgggaagtgcttgaaggccaaccaaggcaagctacagttagttcttttggttttagtggcactaatgctcatatagttatagaagaggctccttctcaagttaaaagtcagaatattgttgaacgtccgatccatctgttaactctatctgcgaaaacagaaaaggctctagaagatttagtcagtaattatcaaaattatttagaaactaatcccgagttaccactagcagatgtatgttatacagcctctacaggtagagcacattttaattatcgattaggagttattgcttctgaaccaaaagcattaatagagaaactacttgggtggaaagctcaggaagaattagtaggactattttcaggaaaacgaaatagcgaaggtcagaaaatagcattcctgttcacaggtcaaggttcccagtatgcgaatatgggaaggcaactttatgaaaaagcaccaactttccgtcaagctttagaggaatgtgaccaaattttacaaccctatctagaagtacctctattagaggtcatatactctgaggatgcacaaaagtcaagtgataatctattagaccaaacagcttacacccaaccagctgtgtttgctgttgaatatgctttggctaaattatggtcttcatggggaatcaaaccatgtgtagtcatgggtcacagcgtaggagaatatgtagcagcaacagtagccggagtattcagtttagaagatggtctgaaactaatagccatgcgaggaaagttgatgcaaaagttaccctccggtggtgagatggtatccgtaatggcatcagagtctcaggtaacagaggctataaaagaatatagttcccaagtaacaatagcagcgataaatggaccagaaagtatagtaatttctggtgagagtgtagccataaaaaatatttgtagtttatttgaatcagagggaataaagaccaagcagctacaagtatcccacgctttccattctccaatgatggaaccgatgttagcagagtttgaatcagtagctaaacaagtcacctataatcaaccccaaataccactaatatcaaacgtcacaggtactgaagtagatggagaaataacaaatgctgaatattgggtagatcatgtgcgtcaaccagtaagatttgcccagagtatgaaaactctagagtcggaaggatatgaaaccttcctagaaataggaccgagaccaatattgctaggaatgggaagacaatgtgtaacagaagatgtaggagaatggctgccatcattacgtccaggggtggatgaatgggaacaaatgctatcaagcttaggaaaattgtatgtaaaaggagccaaaatagactggtcagggtttgactctgattatactcgccagaaagtagtattacccacatatccattccagggagaacgttattgggtagaaaccaacaacaacttctggcctcaacggcaattttcccaaggggaaaacctccatcccctattaggtcaaaagctaaattgtgcaggggaacaacaaatatttgcatcacaaataggagaaaactcacccaactatctgagggaccaccgagtatttaatcaagcactatttcccataacaggctacctagaaatagcaatagcagcaggaaatcaccaattaaaaacatcccagatagtaatagaagacctaaccataactagaggatggatactaccaacaggagaattaaccaatgcccaaaccatactcaccccaatagataaccaaagctataagtttcaaatattttctcaaccagaacaacaggagtggagactccacacgacaggaaaaatcagaaaagagtcaaccccccctactcagacaaaagttgacctagaaaaatacaagagtgaatgtaatcaaacaatagaagtcaaacaacattatcaaaaatgtcaacaagtagggatagactacgggaataccttccaaggcatccaagaattgtggtcaggttcaaaccaagcattaggttatatcaaactgcccgaagaattgataacacaaacaagcgactatcatttccatccagcactattagatgcagccttgcaagtaatgttttatgcactgccagcaacggataatgacaaaacttatctgtcagcaggaatagaagaattcagactatataagactcccgggctgagtatatgggcatatgtatcagtaaccagtcaagaagtggaaactccagaaagtttgacggctattgtcaccatagtaactccagaaggagaaataattgccaacatcaaaggtttacaagtcaaactagcaacaaaacagaccctactgggaacagaaaccgaatcaatagaaaattggttatatgaagtagagtggagaaacaaaggtattttaggtaaactactcccaccagatttcctcatacctcctatacaaatcaaccaaaaattaactccaactctgacagaattagtaactcaagtagataatgaaacaacagcgtcttttgaaacaagcttagaagaattaagcagagattatatagtacaagcattacagtcaatgagttggtcatacaaaccaacagaaagctttgcatttgatgtagcagcccaaaaattaggtatagttcctacccatcgaccactgtttaagcgtttgctgcaaatattaacagagtcaggaatactcaactcaaagaatcagcagtgggaagtagcacaaaccttacctgaagtcaagcctacggaaaaaatcagcagtttacagaagaaatatccagaagaaacagcagcattgacactactctctcgttgtgggtctaaactaagtggggtattacgaggagcaatagacccaggagagttagtgttcccccaaggagatttgacagcagcgactcaactttatgaagactcaacagtagcgaaagtgatgaacacaatagtagaaaaatccatcaccaaagctatagaaaaatccccgaaaagccgggggctcaggttgttggaaataggagggggaacaggagggactacaagctatatcctacctcatctaactcctcagcaaaccgaatatacattcacggatataggggcattatttacagccaaagctcaagagaaattccgggattataagttcataaagtatcaaactttagacatagaagtagacccgacaactcaaggatttgaggctcatcaatatgatgtaattattgcagctaatgtacttcatgcaacgacagatatgaagcagacattatctcatgtgcgagaactgttagcagatgggggaatgttggtgttatctgaagcaacagctaaaacactatgggtagatttagtatttgggttgttagaaggatggtggaaattcagggattatgaattacgaccagattatcctttgttgagtcgtgaaaaatggcatcatgtcttgagagaaacgggttttactgaagtagttactatgccagaagtggagggaatggcagaaacattgtcagcacaaacggtaattgtagctaaaagctctcaaacgaagttagaacaaaggaatgatgattctaagagttggttgatactggcagactctgagggagtagggcaacagttagcgaccttactccgctcagtaggagaggtttgtactttagtatttgccggagaaaggtatcaacagatagctccgggagaatttagtattaatcctaatcaagctaaagattttgaggaggtaatagagacagtggcagggaagtcaccatcattgtatggagtggtacaatgttggacgactgaagccggagtggggaatgggattaattctgaggagttaggaagtttatcgaagttagggtgtggcacaactctatctttagtacaagcattggtgaaaggcgggttatcaactgtccctcgattatggttggtgacaaatggtgctcaggcggtgccgaataatcatccggtgataccaggagtagctcaatcttcggtatggggaatggggaaagtgattagctgggaacatccagagttgaactgtactcgtatagatttggacccagaggagactttagagggtaaagttgatgccctatttaaagaaatttggtcggaggatagggaagaccaggtagcatggcgtggggatagtcgttatgtagctcggttggtgggtagtcatcatcggcaattagtggcacaacaagctgatggtaaaactcaaaagcccttaagtttccgttctgaagcaagctatttgattacaggaggtatgggaggtttgggtttgctggtagctagttggatggtgtcaaagggagctaaacatttgatattgttaggacgccgttcaccggatgatgctacgaggaaaaaaataaccgagttagaaatggcaggagcgtccgtggtagtggaaaaagccgatgtgactgatttggagtcgatgaaaggggtgttgcagaggattgaggagtcaaagcgaccgttagtaggagtgattcattctgtgatggtgctatcaaatggagtgctacgaaatcagacttggtctagttttgaacaggtgatggaaccgaaagttcaaggtgcttggcacttgcatcaattgactcaaagtcaaccattagacttctttgtgctgttttcttcagcaacatctctgttgggttcactgggtcagggcatggcaaattattctgctgctaatgggtttcttgatggtttagctcattatcgtcgaactatgggattaccgggattgagtatccattggggagcagtttctcaagtgggacgagcgctcgggcgagatacagagacagccgctatgctcagtaagaatgggatgggtttaatatctccggctcaggtattggagtctttggaactattgatgagtagttcggatgtggaagtaggggtgatgcctattgagtggtcagggtggcaggagagagtggcacagtggccgtttttagtggattggcaggaaactatattggaggtagcccaaccatcgaagtcagattttctgttaaagttggaggctacaccacctaatgagcttcgcttgttgttggtggctcatgtgcgtcgtcaggtggctcaagtgttgagaattagtcatcctgaatcaattgaaatggatacagggttttttgatttgggtatagactctttgacttctgtggagttgaggaataagttgcaaggtagtttaaagtgttcagtaccttctactgtaactttggactaccctaccattaaggcattggtagaatatttatatcaacagctattgttagaacaagttagttactcaaatactgtaccaactgaagaaatcaatgaagatagggaggagattactctaagcgggacaccagctcgggaagcaagtagtgccttacgggatggctatcgtcaagcaggagtcagcgggcgggttcggagttatttggatttattggctggtttaagtgattttcgggaacattttgatggtagtgatggtttttctttagatttggtggatatggctgatggtcccggtgaagtgaccgttatttgttgtgccggtactgccgctatttctggtccccatgaatttacccgcttagccggtgctttgcgtggtattgctcccgtgcgggctgttccacaaccaggttatgaagaaggtgaacccttacccagttctatggccgctgtggccgctgttcaagccgatgctgttattcgcacccaaggtgataaaccctttgtggttgctggtcatagtgccggtgctttaatggcctatgctttggccactgaattattggatcgtggtcatcctccccgcggtgtggttttaattgatgtgtatcctcccggtcatcaagatgctatgaatgcctggttagaagaattgaccgccactttgtttgatcgggaaaccgttcgcatggatgatacccgcttaactgctttgggtgcctatgatcgtttaactggtcaatggcgcccccgtgaaaccggtttgcccactttattggtgagtgctggtgaacctatgggtccctggcccgatgattcttggaaacccacctggccctttgaacatgatactgtggccgttcccggtgatcattttactatggttcaagaacacgctgatgctattgcacgacacattgatgcctggttgggcggtggaaactcctaactccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtcgaggacgatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagacgatcccctcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaact

SEQ ID NO: 58 - DNA, 32767 bp, synthetic construct
  source 1..32767: mol_type = other DNA
atcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgcgaattcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttagctttttaacttggatttttaccttctcagcctaaaccttaacattagtgtcaatgtcaaggttttgagtaaactaaagggatgaagactaatcacttaacgattaaagaactcacagatgcagtgggaggtggcgttacgcctcgcatggtgcgccattaccacaccctgggattgcttccccccgttcaacgctcagagggcaactaccgcctttatactcagcaggacgtacaacggctccaacgagtcattgccctcaaacagcagggctttcagttgtctcatattcggcaactgctggatagccattctgaagagagccttgatcccaccctaatggtgcagttgcaacagcaatatcaggctgtgattcagcagattactcgactccgccaaaccgcatctgctttagaaggattactgggacgcgaccagagttgccagattacccaagcggaagctctggcccaactgaaacagcttgatgtggatgtccaggaggggttaggaaaactcgatcagttgtggacgaacctagatgcggagacaacaactcatccagaagcctttcaggaatccctcaaacacctgctaccggatttatcggcttactctgaaattaccattcacttactacatcaattagtgctggcctgtggtgatgttagcttggtaaacgccgttcgattgagtcagggagcgatcgcctcggcacgagatgcactgaaagcagggtgtccagtcgtcaccgatgttccggttgtggctgcggctcttgatcaaactcggttagctcatttaggatgtacggttaaaacgctgattgacgaccctcacatcacagggcttagggaagccgagcaagctttttggcaccatgaccattggcaacagcggttacaacagattccccaaggatgtgtgctggcgatcggctatgccccttctgttttactcactgcctgtaagctgatagagcaacaacatattcagccggctcttgtgatcggaatgccgatcggttttagtcatgctccgggggcaaaacgacgactgatgaccagtcccattccccatatcaccattcaggggagcctcggtggaggacttctagccgcagtaacgctaaacgctttagtggaaacattgattgcaaagccagattgccactgctatctcacttgtctttaggaaacagaccatgctagataaaataaatcgttatgctcatgggtttgtagctgtaccagtgatttgtgcttgttcagaagcaggggtattcgagttactatcacagaaaaaatcactgaaattagaagaaatagtagagcatttagcagcaaatagtggacatctaatggtagctatgcgactcctagagtcattatcattcctatatcgctctcaagcagaagaatacatattgacagaacaaagtcaacaacatcaaatcatccctaaagctctgatgagcttatataaatatccatttgagttatatttaaaaggagaagtagaaacaggaatcagcaattggattaattgttcatctcgaagatgggatacagaaaactcattactaagtgatttattagatggagtattactcatccccctgctactagaactgaaaaaacaaaacttactggatgaatcaaaaaaaatatttaacacattaacaaattcccttaaacaagaactatcaacattattcatcaatttaggatgggcagaagaaaaaacagaagggctatatttaacagatataggtagatttatgcgtgacagatccttaaacttaggaacaacagcgtcttatgctcctatgttgttacagatgaaagaattactatttggaaatcctcagagagtatttcaaagaaataagactgaaaaagaaagacacgtaaatagaacattaaatgtagtagcaagtggctttcaacacgaaaagttttttgccgatacagataaaatcatcatatctatatttaaccaacagccaatagaagagcaaccaagctatatagtagacatgggttgtggggatggaacgctactgaaacgaatatataaaatcatcaaacaattctctgctaggggaaaagtattaacagagtatcctatcatcatggtaggagtagactacaatcaagaagcattagatgtcacagataaaaatttagtagatattccccatctagtcattccaggagatataggagcaccagaaaaattattagagcaattgaaagcacaaggaatagaaccagaaaaagtattacatattcgttcattcctagatcatgaccgaccatttatagctccaaaaaatacagaaatagctcaagcacgttctcagttagattatcaagtagtagatgtagatcgagaaggaaagctgataccacctcacatagcagtacaaagtttagtagaacacttagaaagatggtcatcaataataactagacatggattattgctcctagaagtacatagtttaacgccagcagtagtcaaaaaatatatagacgaaagtgaatctctgcattttgatgcctatcatgccttctcaatgcaacatttagtagaagcagatgtgttcttgatggcagcagcagaggtgggtttattttcacggaaagaagcttttcgtaagtatccgaaaacgttgccattgactcgaatcacagttaatcattttgaaaagcgtaaatatcagatacggtatgcaactgtaaacgatataccaaatctgttaaagtgtgcgacatttaatcaacctgtaaacgaacccttctttcaagttttattgaaacaaactccaacagcacatttattattagaatatcaaggtgaattggtagctgcaatcttcacagaaactaaaaattctaatgaggtgctaggaattcgtgagtttttagtaaggacttctgtagaaaattggcaagtattagcaaaagatttactagaatttgtggaacagtggggagtagtcaaaccaggaataaaagaaatagaaggattgttaaaataccatgaagccatctcaaactttcaaaaatcaaagtggtatcaatcttcagttttaaacaaaaagcttatagaaaaaataactctacacgaattagctactttagaactatgtaatttaatggctccagaatatgagctagaagcctttgccgcacgttggcttttgcgtgtttttcaagatatgggtgtatttctcagagagggtgaatcttatcaggagtctgagttggtttcccagctaaacatctcaccacgttaccagagacttttaggtgctttgttacaaattttgcataagcgtggtattctaaaaattgagaaagatagagtgttcacattagcaagatgtaagacctttgccttagaaaatatttcgtctgaagtttctgctttttatgattatttttctgaaaaatatcctgctcatttatcctggttaacagtagtcaaaaggtgtctagagaaatatcctttgattctacgcggtgaagttgatgtcaacgaagttgtttttacagatggggatatggagctatttgctggactatttctaggacatcgtgttgctgactactttaatgagttgctagcagatggggtttgctgggaagtagaacagcggttgttagaagaaaagagggcacaacctattcggatcttggagattggagcaggaacaggaggtgttacaggaatattgctagaaaagctagcttctcatgcagagcaaattgaattttggtttactgatatttctagcgtttttacacgttatggtgagagtaagttcaagcagtttccttgggtgaaatatcaaacctttgacatagaaaaatctcttgatgctcaggggataaagtctgaaagttttgatgtggtaattgctaataacgtactccataacacaaaattaattcatcaaaccttaaataacagtaactcactattaaatactggggggttattggcattactagagtttactcaaccaattgatattcttttatactttggagggttgcttcaaggattttggttgtttgaagatccagaataccgactagaagttggttgtttactgagtataccactgtggcaaaaagttctcagtgattgtgggtttgatgaaataataccattgggattaccctgtgagatgcacgctctttctaaggcaagagaatctgttatatttgctcgaaagcatcaagtacaggagaaaacattttctgaaaaaattaaacaaaatttgacagaaaatggtaagcatgggcaggcagaattcgattttattagtattaataattcacaagaaagttcatccaaattagaaatttttgaacaggaatgtcgaaaattattaaaatctctactgggtgttcaacgtatggagagattgcctggtgacacaccactaatggagtcaggaatggattcactggagttgttagaatttcgtgctcttatagaaagaaagtttgggattaagttaaagtctaccttcttttttagttacaaaactcttatagcggtagcagagtatctttcagaacgggaagatattaattttagttagtaggactatgactgaaaaaaaaacaacgttttcacaggtccacagggggaaattttgaatcagggggaaaatttgaatgcattctaaccagagcagttcactagcaagaatagccattgtaggtgtggcttgccgttttcctggagcagatactaaagaagaattttggcagctacttaaggaaggtcgggactcaatgcagaatcttccttcagagcgttggggaaatctatttaactccatgagttcagaaatagatttatctattcaacgcggtggttttttgaaagatatagatttgtttgactcatccttttttaggattactcctagggaagcgcagttaatggacccccagcaaaggctgttgttagaattgtcctgggaagcgatggaagatgcaggatatgcaagggatacattaaagggtaaatcagtaggggtttatgtaggagtttgtcactatgactacagaagtttattagaaaaaggtttggagacagccgaaatagctcaaattgctactggtacagctccagcaacttttgccaatcgcctttcctatttttataattttcacggccctagcttaactgttgatacggcttgttccagctctttggtagcaatgtatgaagcagtaaatgcaatacgtcgtgggcagtgtcaaacagcattggtaggaggcgttaatttaatgtgttctcctgtgaataaccaagtttatagtgctgcgggtatgttatctcctgatggagtctgccgggtgtttgatgcgggtgctaatgggtttgtcaggggagaaggaggggcagtagttgtacttaaagattatcaaaaagcgttgacggatggtgattctatttatggggttgttcgcagtgtagcagtgaatcatggagggcaagcaagttcttttacagcaccaaatccccaagctcaggcaaaattgttggaacaggcttatagggaagctaatattgatatagagtcggtaggttatattgaagcacatggtacgggtacatctctaggagacccaattgaggtcgaggctttaaatgaagcgtttaaagggttgagttcaagtggtaagttacctgctaatagttgtggtttgggctcagttaaaacaaacattggtcatttggaaggggcagcaggtttggctggactaatcaaagttttgctgtgcatgaggtatgcaactcttccatgttcattaaattatcagcaacttaatcctgatattgaattagaagaaggtccgttttttgtggtggacaagcttcaatcttgggaaataaaagttgatagggtaggaaaaccttatccacttagagcagggttgagtagctttggttttggcggtacaaatgctcatgtagttcttgaggaaggagaaaataagaaggaaggaagaagaggggataaggaaccttcagttcatctattaaccctttcagctaaaactgaaacagctctttcagagttagtcagtcgttatcaaaaatatttaaacactaatgcagaatcagagttagccgatatttgttataccgctaatactgggcgagtccattttaaccatcgactagcagcgatcgcctcaaacaaacaggagttagtagaaaaacttaaggaataccaagctggggaggcaacagcgggagtattaataggggaacttgcagaacgcataagaacaccgaaattaggtttcttattcacaggtcaaggttcccagtatgtcaatatgggaaaacaactgtataaaacacagcctgtattccgtgaagtattggataagtgtgatataatattggaaacggaaatagaatgttctctattagatgttctatataacaaaactacagattctcaagattcatctttaataaaccaaacagcttacactcaaccagttctgtttgcaatagagtatgcgctatttaagttatgggaatcttggggaattaaacccagcatagtaatgggtcacagtgtaggagaatatgtggctgcttgtgtagcaggagtcttcaatcttgaagatggtttgagattaatagctgctaggggcaggctgatgcaacagttaccttcaggtggagaaatggtttctgtgatggcttcagaatctacagttagcagactcctagaacctcacaaaaaagaaatagcgttggcgaagcccgccgtaggcatcgcagcaataaatggaccagaaagcacagttatttctggagactctgtagcagtaacaggtgtagtaaatgacctggaagcaaagggaataaaaactaaaaaactagaggtatctcatgctttccattcaccattaatggaaccaatgttaggagagtttgaagctatagctaatcaactaacctacaatcaacctaaaatacctattatatctaacgttacaggtacaaaagcagataatactattgcttcccctcaatactgggttaatcatgtcggtaaacctgtaagatttgcccaaggaatggaaacgctacatcaacaagggtatgaaacctttatagaagttggaccgaaaccagtattgttaggaatgggcaggcaatgtctaccaggaaatgtaggtgtctggttgccatcattacgtccgggagtggatgagtggcaacaaatgctttatagcttgggaaaattgtatgtaacaggagtaaaaatagattggtcaggatttgagtctgactctagtcgccagaaagtagcattgccaacttatccattccagcgagaaagatattggatagaaataacagaaaacaaacataaggaacatcaaaagtcagaaaatataagtgacacttcaattgttaaactactcactcaaggaaaaacagaagctctcactcaacaactagaaacagaagccaaattttcaccagaagagcttaaacttttaccagaaatattagagacattagccaaacaacatcaagaacaattaacagcagtaaccatcaaaaactggttctacgaaatccagtggaaacccttagctcaaaacaaccccaatgcaaacattgaacctagtcattggttaattttagccgataccacaggagtagcagaaaaattagctcaaaaattacaacaacagggtcataaatacagcttagtttatcgaggagagagctatcaaagacaagcaacaggtacttatcaacttaatcctcagattcccgaagcatttgaaaagctgtatcaagaaattcaacaaagtagtgaaactgccattacgaagttaattcacttgtggagtttagatgctccccaatcaaaagacttaaccctggaaaccctagaagaagctcaattatggggatgtggcagcgtagtacacctgttacagaccttagtcaaaaactctagcattcctgaactatggttagtaacccgtgggtctcaatcagtattatcccaaacagaaaaaaatctaacaggactagcagcgtcacccttgtggggattaggtagagtagtgtctaatgaacatccccaattatggggaggattagtagatttagacccacaagctgcagcaggagacgaagtagaaatgctgtggcaattattagttaatgaacaagaagaagataatctagctgtacggggagaaaatacctatgtagctcgtctggccaggcaagaacctcaagaatttcctgaatccctatccttatcatcagatggtagttacctaataacaggagggttaggagctttagggttacatactgcccaatggttagtatccaagggagcgaaaaatattgtcttaactgggcgtcgccctccctcagaaaaagtaagtgaatctataaaaaaattagaagaaacaggatgtcaagtgaaggtgatgttgggggatgtttctgttgaagaagacataggcaaaattctcaaacaaattcagatatcaatgccaacactcaaaggcataattcatacagcaggagtgttagatgatggaaccatacaacaaatgaattgggagcgttttgccaaagtcatgtcgcccaaggtaaaaggaagttggcatttacataaattaactgaaaatcagccattagatttctttgtgtgtttctcctcaatagcttcgatgttggggactcttggccaaggaaactatgctgcagccaatgcctttatggatgctttagctagttatcgttgcagcagaggattatcaggattggcgattaattggggagcatgggcatcagggggaatggctgctcgtttagcagtggagcatcaaaataggatgcacagcagtggcataactgagatggcgaccaaagaaggaatgtatgccttagatttactattaacaaatgaatctgctacagctcaggtaggtgtagcaagtatagagtggcaggtactctcagaaagttggagtggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttaggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttatgtatagaggaattaatgggatggaagacaacagaaattgacccattatctgaagaaacaagattagagatgatggagcgctcaagaagtgaagcaatagccatcatcggcataggctgtagatttcccggaaatgccaacacaccagaaagcttctggcaattgttatccaatggcaaagactccattacagagattcccctagaacgttgggatctagattcctactacgaccccaaccctgatactccagggaaaatgtatatccgtcacgcagcattagtagaaaaagtagatcagttcgacccacgattttttggaatctctaaccgagaagcttatagtcttgatccacagcaacgcttcattttggaagtaacttgggaagctttagaaagagctggtattaaccctcaacaattagaaaacactcagacaggggtgttcctgggtatcggtcaaaatgattacgcgaatttaggttttcaccaagcagccgaagatataagcccttacgatgctacaggaaatttgttttgttttgtagcaggtaggttatcttactttttaggaacgcaaggtccatcaatggcaatagatacagcttgttcatcatctctagtagctatccatgaagcttgtgagagtctgcgtcagggtgagtccaacttggctttggctggaggagttcaactaattctctctcccgaagtaacaaccgcactatcaagattaaaggctctagcacctgatggtaagtgtaaaacttttgatgctgctgctgatggttttggtaggggagagggatgtggcattgtggtactgaagcgtttgtcggatgcgctcaaggatggggacaggatatcagcagtaattcctggttcggctgttaaccatgatggaccaagtagcggaatgacagtaccgaataaactggctcaggaaaaactgattcagaaagctctcaaagcagccaaggtaaaaccgttacaagtgagttatgtggaagctcatggtacaggaacttctctaggagatcctatggaagtgagagctttagctagggtgtttgaggagggacgtgatcaggaaaatccattgaacatcggttcagttaaaactaatatcggtcatctggaagcagcagctggaatagcaggtatgattaaggtgattttgcaattgcaacatcaggaaattgtgccccatctgcattttgctaatcctaacccctatgttgattgggagaatatgcctctacaagtaccgactcaactgactccttggttgtcgaaaggggagaaaagggtggcaggagttagttcttttggtatgagtggtacgaatgctcatatagttttagaagaggctcctattgaagtcagaaggcagaagtcagaagtcagaagtgaagaatatctagaacgtccggttcatatactaactctgtcggccaagactgaaaaagcactagaagatttagttaatagttatgaaagttatttagaagccgaaaataatgataattatttaggggatatttgctacacagccaacatcgggagagcaaaatttgaccacaaattagcagtggttacttctgataaacaagagttattagagaaactcaaacaatataaacaaggtgagaatgttgctggaatcttttcaggaaaacaaataagtgaaactagaacaaaaatagcctttatatttactggtcaaggttctcaatatctgcaaatgggaaggcagttatacgaaactcaacccacttttcataaaattattgaccagtgtagtgaaatgctggtaaaatatttagatgtttctttattagatatactttatccagttgaggttaaagatgaaagttcgactttgatagaccaaacagcttatactcaacccgctatatttgcccttgaatatgccctagctaaattatgggaatcatggggtataaagccagatgtagtcatgggtcacagtgtaggagaatatgtggcagcaacagtagcaggggtatttagtttagaagatggtttaaaattaatagccatgcggggacagttgatgcaaaagttaccctccggaggtcagatggcatctataatggcatcagaatctcaggtaatagaggcgataaaagaatatagttcccaagtaacaatagcagcagttaatggaccagaaattatagtaatttcaggtgagagtccagccatttcaaaaatttgtagtaaatttgaatcagaaggagtcaagaccaaggcgctacaagtatctcatgctttccattccccattgatggaaccaatgttaacagaatttgaagcagtagccaaagaaatatcctataatcatccccaaataccactaatatcaaatgttactggtcaagaagtaaatggagaaataaccactgctgaatattgggtgcgtcatgtgcgtcaaccagtaagatttgcggaagggatggaaactttacacaaacaaggtgctgaaattttcctagaaataggctcaaaaccgatacttttaggtatgggtcgtgagtgtctcatgggagaaaaaaaactatggttgcccagtttacgttcaggaaaaccagactggttacaaatgctacaaagtctggggcaattgtatgtacgagcaataaaaattgattggttaggatttgatcgggattatttccgtaataaggtagagttaccaacatatccttggcaacgaaaaaggtattggataacagatattagacaacggaaaagtcaagacaaaaaaagcataacatctgaaaaaaaagtacaacttgatggagtaaatattcaactaaaggaaatacaaatgaacgacaaaatcttacagcaaccaaaactaaaattatcagatccagaatcactatatttatcgaatgctgaatcaactatagaggttccaacaaaagcagttcaagtaaaacctgctgctgatattgaatctgaaaatataactcaattgaatagccttgatagagatgtgactcaaatcatagaaaccctcaaagaaagtttggcagatgctttatatgcagatataagtgaaattgaagaagacaaaaagtttgttgatttaggtttagattctattgtgggagtagaatggataactaacatcaataaaatctacaatttaaatataaaagctactaaactatacgattaccctactttgccagatttggctaaatatgtcgctcacaccctatcagctcagggtagaaatattgatgtagagcgatcgccatctgactcaagtcaagcaagcattagcaagcaatctcaactaactgatacccagagcaatttctcacaagtcaaggaaattttgaaacaacaattagcagatgctctgtatgcggatattagtgaaattgaagtaaacaagaagtttgttgatttaggtttagattctattgttggagtagaatggataactaatatcaataaaacctacaatttgaatatcaaagccactaaaatatacgactaccctaccttattagatttcgcaaaatatattaaccaggaaatttattctacaggagtaagtagattttcaacggagcataaagaatttaatcaaaaagactattcatctggaggttcgcaagaggaaatgacacaaaaattacgatcaatattgaagaaagtggccaataaagaattaacagttcaggaaggaaataaaatgattcaacaaataaaaaatcaattaaagtaaaaatatagtataatttttaaggaaataaaattttatgaacaaagagcaaatatttcaaataataaaaaaatatacctacgaaatagcaccagaattagaagaagtacctatatcacctactgatagtctcaaaaacttggggattgattcagtaaatagggcagaaattataatgatggtaatggaagagttatcattaaatatcccgcgtattgaattagctggatccaaaaatataggagaactggctgatatatttgcattcaaattagaagcaataaattcacaaagctagaaggcctatcaatgagtaacatagaaataacaggtatgggcattgttacttccattggtcaaggagttgctacttttaaagaagctctgttatcagggaaaactcaatttgcttatctaaaacagccaggacgtgaaagcatcaaaccatttattggtgccgaaattcccgatattgacgccaaaaccctatttcctgagtacagtggactgttacgtactgctactaagagcgcccaagttgcaatagtagctgtcgctgaagcctggcaagatgctcaactcacctccagccaagttaacccagaacgagtaggattagttgtaggtggttcaaatttacaacaacgttatcagcagcaaacctggcaacgttatcactcacgtcaggagtttatacgaccaacttatggtctgaccttttgggatacagatatattaggtctgatttcccagtgttttcagattcaaggtgagggctatagtgtaggtggagcttcggccagcggagcagttgctataattcatgctgctcgtcaaattttaatggggaacagtgatgtttgcattgctctgggagctttatccgatctatcagggtatgaattccaagcactaatgaatttaggtgcaatggggagcgaacgttttgccgatcgcccaaaccttgcttgtaggccatttgaccaagaccatgatggctttatttatggagagggttgtggagcagtgattttagagagaactgaccgcgctcaacaacggggcgctcaatcccatggccaacttaagggttggggattaactttagatgggaaccgtagtcccgaaccttcccaaaaaggggaagaacgggctataaatactgctttagccatggcagaccttcagccagagagtattgattatgtgaatacccacggtactggttccccccttggcgacaaaacagaagtggcagctttcaaatcagtaggacttcagcattgcctatttaattcaacaaaatctttaataggccactgcttaactgctgctggtgtagtggaagcgatcgccaccatattacaaatgaagtttggtttttgtcatccaactaaaaatttagttaatcctattgataccagtctcaattgggtgaaagaaacttctgttcaagctgagattaaatatgctatcagcaacagttttggttttgggggtatcaatacagctttattaattggacaggagtaaactcatgcaacaagttggaattgaagcactaaatgtatatgggggttcagctaaactagatgtgcgaatgctaggcgaagcacgtcacttagacatgactcgcttcgataatcttctgatgaaagagaagacagttgctatgccctatgaagatccagtttcttatgctattaatgcagcaaaacctattattgatagcctcagcgttctggaaaagcaacaaatcaaaatggtgatagcttgtagcgaatctggcattgattttggcaagtctatgagcacttatatccaggattatttgggactaaatcgtaactgtcgaatgtttgaaataaagcaagcttgctactcaggtacagcaggtttacaaatggcattcaatttaattttgtcccaaacctgtccaggggctaaagctttggttataggaactgatatatttcggcctgttgttgtagagggaggagaagctctcagcgaagattggtcttttgtggaaccaagcagtggtgctggagcagtagctattttagtcagtgatgttcctaaaattttccaagctgatgtaggatgtaatggttactatggctatgaagtcatggatacttgcagacctaacccagactcagaagcaggagatgcagatttatcattgctatcttacctagattgttgtgaaaatgcttaccgggaatatcaagatcgagtagaaggagtagattaccaaaaaacttttgattacttgagctttcatactccttttgggggtatggtgaaaggggctcacagaagtatgatgcgtaagtttaaaagggcaaaacctgtggagattgaagaggactttcagaaacgggtgatgccgggattagtctattgtcaacaggtgggtaatattatgggagctacagtatttttatctttagctagcatgattgataatgcagattttagcaaagcccgacgaattggtgtattctcttatggctctggttgttgttcggaattttatagtggagtcgtcaccccgcaaggaaaggaaattcaagctcaacaaaaaatttcatcacagttagcaatgcgctattccttaagtatggaagaatatgagcagttactaagtcatagttcggcagttgcctttggcactagaaatgttaccctagattacaaactatttcctggtgtgtgggaacaaattgaaggtaaaggtcgcttagtgctgaaaagaatcaaggaatttcaccgagaatatgaatgggtatagccatgagttatcaaaccctgaaaatcagttatcaagatgttgtacaaaggattcagatatatcgacctgaatctaacaatagcatcaatagtcaattaacgatggaattgttgtcagctttgcaagctgctgaagcagaggaagttgttaaagtagtgatattagaaggactacctgatgtattttgtacagggatggattttgaagaagtggcaacagcaaaacaatttgatccaaaagctagtgctaatggttactacaatattttgaaacaaatgtctcaaagtagcaaagtaattttgtcacttgtgcgtggtaaagtacaggcagggggagttggtttagtagcagcaagcgatctagttattgctgatgaaacggcaacttttgttttatcagaattattatttggattattaccagcttgtgtattgccttttttgattcgtcgagtgggatttcaaaaagcctaccgtttagcactaacgactcaagctatttcagtatcagaggctgataagtggggattaatagatgaatatggcagtaatattaatcagttaataagtaaatatattcgacgtttaaagtatttaccctcatcaggggtgaaagagttaaaaaattatattaatcagttatggattattcaggcagaaactcaaggtttagccgtcaacgagatttctagcttaatagcagaacctactgttcaagaaaaaattaaacgttttcaaaaagaaggattatttccatggcaaacctaaatcttaatttggacttagtagagggcaactctgatgtagtacaactggtggagttgggtaatggtgtcgtgcaaatcacgatgaaagatgaagaaagctgcaatggcttttctcctggaataattgaaggattatacaaatgttttggtgcagttgctcaaaaccaaagttataaagtagtaattttgacaggctatggaaattatttctgttcgggagggacaaaagaacggttaatcagtatttggaaaggagacagcaaatgtaatgatttagatttttttagaatagcattagattgtgaaataccagtaattgcagctatgcaaggtcatagtattggcggtggtttggttttgggattgtatgcagatttagtagtgttaagtcaagaaagtatttacactactaattttatgaagtatggttttactccaggtgtcggatgcacgttaattctccctgagaaattcggtgctttagggtttgaaatgatgtatactgcccaaaattatcgagggaaagaattagctgaacggggtgtttcttttccagttgtaccgagaaaagatgtgctagaagtggctaaaaatatagcctatgaaatgtcggaaaaacccagactatctttaataactttaaaggaacacttaacttcaaaaatccgcaaaacgctaccaggatttatagataaagaagtagctatgcacgaaataacctttcaccaaccagaggtagcaagtagaatagaggaaaattttgacaaaaggacaacggcaagtaataaccctcaaaattttcctcaagaggctgtaagaaaagaaataagaacagattcattgaattgtcaaccatttcagttgaagacatttagttatggttcgttaaacaatttaactttggtacctctagaacgtagagttccaagcccaagtgaagttgaggttcaaatcaaaactgtaccagttaattttcgggatatactcaatgcacttggtatgctccaagagtattacgaaaaaacatttggcattgctaatgctgaagatctcacttttggttttgaaggtgcaggtactatcgtagctgttggggcagaagtatcgcagtggcaagtcggcgatgaagtaatggtaatgagaattcacgatgcatttagtagctttattatctgctcgccggacaaactggtgcgtaaaaattttaatctgaatatggaagatggagctagtatttgggggccgttttcgactgcatatcacgggttgattaacttagccaaaattcagccaggagatagagtattaattcacgctgcttcaggtgggattggacaagcagcaattcagttggctcaactttttggggcggaagtatttgctactactagtccaggtaagatgaattatctccgggaacagggaattaagtatgtgatgaattctcgaacgatggagtttgtaaatgatgtgatggaattcactcagggccgtggggtagatgttattctcaatagcctgactcatggagaatacatccagaaaaatctagagatccttgccgatagagggcgatatgttgaacttggtaagttgggcatttggagtcacgagcaagtctatcagaaacgcccagatatcaaatattttacttttgatttgttagaagaatttgccaaagataatcaattgttttctcagatatgggataatttggcacttgaatttgaccgcgatcgcttgaagccactaccttacaaaacatttccaatagaagatgttcttaaagcctttgattatatgcgacgcgggaagcattttggcagggtagtggtagttatgcctgactcctattctagacaggaacaagagttggatgctcggttatccattgaaaacaaaatgactaaagaggaacagatattatttcaattacaatctggtgaagtttccttagaaaatgcggaacaactattgttaggaaatacggaaacagaaacacaagataaagctatagcagaaaatcaaatagataatattcaaaacaagttaattaacatggatagctcagagaaaatcttatctttgattagttcagtagaaatatctttagaaacagcagaaaaattattattagaagtagtagaaccagaagttaaaacagaggttaatgatgaggttaatcctagtcaaaatcatataccaactacagatatagcgattattggtatttcatgtagatatccaggagcgaataactggaaagaattttgggaaaatttaaagaatggaattgacagtgtaacggaagctcctcctggaagatgggaagaaaaaaattggtatcatccagatccagataatccaggtacttcctattcaaaatgtgccggttttttagatgaaattgataaatttgaccctttattttttcatatttctccggaagaagcttggtttatggagcctgagcaaagaatatttttagaagaagcttaccacgctatagaagatgctggatatgctacagactctcttagaggtaaacaatatggagtatttgtgggagttacggtaaatggtggttatcttaagttgttgtcaatttcaggattagatgttcataggatggcggctacaggaaatggtccgtcaatgataccagcaagaattgcctatatgcttgaccttcaagggccagtagtagctattgatactgcctgctcgtcgtcattggtagctgttcatcaagcttgccaaagcatacaacgaggagagagcgaaatagcgatcgctggaggtattactctaatgccaacatcagacttccaaataatgtcaagtcagtttcaagttgtatctcctgatggacgttgtaagacttttgatgcttcagcatcgggtacagcctggagcgaaggttgtggtgttctcttattaaaaagctacagtcaagcaattcaagacaacgaccatatttatggagtaatcaaaggaacaggagttaattatgatggtaatactaacgggattagtgcccctagtagtcaatctcaagccagtttagaagaagcagtttatcagaaatttggaattaacccagaaactattagttatgtagaagctcatggtacagcaacacctctgggagacccgatagaagtagaagctttaacagaagccttttctaaatggacgaacaaaaaacagttttgtgcgattggctcggtaaaaactaatattggtcattcagcagcggctgccggagtttctggtttgatcaagacaattttgtgcctcaaaaatcaaaaattagttccatccttacattttaatcaaccaaacccacatattgactttgaaaatagccctttttatgtcaatacaaaattaaaagattgggaagtgcttgaaggccaaccaaggcaagctacagttagttcttttggttttagtggcactaatgctcatatagttatagaagaggctccttctcaagttaaaagtcagaatattgttgaacgtccgatccatctgttaactctatctgcgaaaacagaaaaggctctagaagatttagtcagtaattatcaaaattatttagaaactaatcccgagttaccactagcagatgtatgttatacagcctctacaggtagagcacattttaattatcgattaggagttattgcttctgaaccaaaagcattaatagagaaactacttgggtggaaagctcaggaagaattagtaggactattttcaggaaaacgaaatagcgaaggtcagaaaatagcattcctgttcacaggtcaaggttcccagtatgcgaatatgggaaggcaactttatgaaaaagcaccaactttccgtcaagctttagaggaatgtgaccaaattttacaaccctatctagaagtacctctattagaggtcatatactctgaggatgcacaaaagtcaagtgataatctattagaccaaacagcttacacccaaccagctgtgtttgctgttgaatatgctttggctaaattatggtcttcatggggaatcaaaccatgtgtagtcatgggtcacagcgtaggagaatatgtagcagcaacagtagccggagtattcagtttagaagatggtctgaaactaatagccatgcgaggaaagttgatgcaaaagttaccctccggtggtgagatggtatccgtaatggcatcagagtctcaggtaacagaggctataaaagaatatagttcccaagtaacaatagcagcgataaatggaccagaaagtatagtaatttctggtgagagtgtagccataaaaaatatttgtagtttatttgaatcagagggaataaagaccaagcagctacaagtatcccacgctttccattctccaatgatggaaccgatgttagcagagtttgaatcagtagctaaacaagtcacctataatcaaccccaaataccactaatatcaaacgtcacaggtactgaagtagatggagaaataacaaatgctgaatattgggtagatcatgtgcgtcaaccagtaagatttgcccagagtatgaaaactctagagtcggaaggatatgaaaccttcctagaaataggaccgagaccaatattgctaggaatgggaagacaatgtgtaacagaagatgtaggagaatggctgccatcattacgtccaggggtggatgaatgggaacaaatgctatcaagcttaggaaaattgtatgtaaaaggagccaaaatagactggtcagggtttgactctgattatactcgccagaaagtagtattacccacatatccattccagggagaacgttattgggtagaaaccaacaacaacttctggcctcaacggcaattttcccaaggggaaaacctccatcccctattaggtcaaaagctaaattgtgcaggggaacaacaaatatttgcatcacaaataggagaaaactcacccaactatctgagggaccaccgagtatttaatcaagcactatttcccataacaggctacctagaaatagcaatagcagcaggaaatcaccaattaaaaacatcccagatagtaatagaagacctaaccataactagaggatggatactaccaacaggagaattaaccaatgcccaaaccatactcaccccaatagataaccaaagctataagtttcaaatattttctcaaccagaacaacaggagtggagactccacacgacaggaaaaatcagaaaagagtcaaccccccctactcagacaaaagttgacctagaaaaatacaagagtgaatgtaatcaaacaatagaagtcaaacaacattatcaaaaatgtcaacaagtagggatagactacgggaataccttccaaggcatccaagaattgtggtcaggttcaaaccaagcattaggttatatcaaactgcccgaagaattgataacacaaacaagcgactatcatttccatccagcactattagatgcagccttgcaagtaatgttttatgcactgccagcaacggataatgacaaaacttatctgtcagcaggaatagaagaattcagactatataagactcccgggctgagtatatgggcatatgtatcagtaaccagtcaagaagtggaaactccagaaagtttgacggctattgtcaccatagtaactccagaaggagaaataattgccaacatcaaaggtttacaagtcaaactagcaacaaaacagaccctactgggaacagaaaccgaatcaatagaaaattggttatatgaagtagagtggagaaacaaaggtattttaggtaaactactcccaccagatttcctcatacctcctatacaaatcaaccaaaaattaactccaactctgacagaattagtaactcaagtagataatgaaacaacagcgtcttttgaaacaagcttagaagaattaagcagagattatatagtacaagcattacagtcaatgagttggtcatacaaaccaacagaaagctttgcatttgatgtagcagcccaaaaattaggtatagttcctacccatcgaccactgtttaagcgtttgctgcaaatattaacagagtcaggaatactcaactcaaagaatcagcagtgggaagtagcacaaaccttacctgaagtcaagcctacggaaaaaatcagcagtttacagaagaaatatccagaagaaacagcagcattgacactactctctcgttgtgggtctaaactaagtggggtattacgaggagcaatagacccaggagagttagtgttcccccaaggagatttgacagcagcgactcaactttatgaagactcaacagtagcgaaagtgatgaacacaatagtagaaaaatccatcaccaaagctatagaaaaatccccgaaaagccgggggctcaggttgttggaaataggagggggaacaggagggactacaagctatatcctacctcatctaactcctcagcaaaccgaatatacattcacggatataggggcattatttacagccaaagctcaagagaaattccgggattataagttcataaagtatcaaactttagacatagaagtagacccgacaactcaaggatttgaggctcatcaatatgatgtaattattgcagctaatgtacttcatgcaacgacagatatgaagcagacattatctcatgtgcgagaactgttagcagatgggggaatgttggtgttatctgaagcaacagctaaaacactatgggtagatttagtatttgggttgttagaaggatggtggaaattcagggattatgaattacgaccagattatcctttgttgagtcgtgaaaaatggcatcatgtcttgagagaaacgggttttactgaagtagttactatgccagaagtggagggaatggcagaaacattgtcagcacaaacggtaattgtagctaaaagctctcaaacgaagttagaacaaaggaatgatgattctaagagttggttgatactggcagactctgagggagtagggcaacagttagcgaccttactccgctcagtaggagaggtttgtactttagtatttgccggagaaaggtatcaacagatagctccgggagaatttagtattaatcctaatcaagctaaagattttgaggaggtaatagagacagtggcagggaagtcaccatcattgtatggagtggtacaatgttggacgactgaagccggagtggggaatgggattaattctgaggagttaggaagtttatcgaagttagggtgtggcacaactctatctttagtacaagcattggtgaaaggcgggttatcaactgtccctcgattatggttggtgacaaatggtgctcaggcggtgccgaataatcatccggtgataccaggagtagctcaatcttcggtatggggaatggggaaagtgattagctgggaacatccagagttgaactgtactcgtatagatttggacccagaggagactttagagggtaaagttgatgccctatttaaagaaatttggtcggaggatagggaagaccaggtagcatggcgtggggatagtcgttatgtagctcggttggtgggtagtcatcatcggcaattagtggcacaacaagctgatggtaaaactcaaaagcccttaagtttccgttctgaagcaagctatttgattacaggaggtatgggaggtttgggtttgctggtagctagttggatggtgtcaaagggagctaaacatttgatattgttaggacgccgttcaccggatgatgctacgaggaaaaaaataaccgagttagaaatggcaggagcgtccgtggtagtggaaaaagccgatgtgactgatttggagtcgatgaaaggggtgttgcagaggattgaggagtcaaagcgaccgttagtaggagtgattcattctgtgatggtgctatcaaatggagtgctacgaaatcagacttggtctagttttgaacaggtgatggaaccgaaagttcaaggtgcttggcacttgcatcaattgactcaaagtcaaccattagacttctttgtgctgttttcttcagcaacatctctgttgggttcactgggtcagggcatggcaaattattctgctgctaatgggtttcttgatggtttagctcattatcgtcgaactatgggattaccgggattgagtatccattggggagcagtttctcaagtgggacgagcgctcgggcgagatacagagacagccgctatgctcagtaagaatgggatgggtttaatatctccggctcaggtattggagtctttggaactattgatgagtagttcggatgtggaagtaggggtgatgcctattgagtggtcagggtggcaggagagagtggcacagtggccgtttttagtggattggcaggaaactatattggaggtagcccaaccatcgaagtcagattttctgttaaagttggaggctacaccacctaatgagcttcgcttgttgttggtggctcatgtgcgtcgtcaggtggctcaagtgttgagaattagtcatcctgaatcaattgaaatggatacagggttttttgatttgggtatagactctttgacttctgtggagttgaggaataagttgcaaggtagtttaaagtgttcagtaccttctactgtaactttggactaccctaccattaaggcattggtagaatatttatatcaacagctattgttagaacaagttagttactcaaatactgtaccaactgaagaaatcaatgaagatagggaggagattactctaagcgggacaccagctcgggaagcaagtagtgccttacgggatggctatcgtcaagcaggagtcagcgggcgggttcggagttatttggatttattggctggtttaagtgattttcgggaacattttgatggtagtgatggtttttctttagatttggtggatatggctgatggtcccggtgaagtgaccgttatttgttgtgccggtactgccgctatttctggtccccatgaatttacccgcttagccggtgctttgcgtggtattgctcccgtgcgggctgttccacaaccaggttatgaagaaggtgaacccttacccagttctatggccgctgtggccgctgttcaagccgatgctgttattcgcacccaaggtgataaaccctttgtggttgctggtcatagtgccggtgctttaatggcctatgctttggccactgaattattggatcgtggtcatcctccccgcggtgtggttttaattgatgtgtatcctcccggtcatcaagatgctatgaatgcctggttagaagaattgaccgccactttgtttgatcgggaaaccgttcgcatggatgatacccgcttaactgctttgggtgcctatgatcgtttaactggtcaatggcgcccccgtgaaaccggtttgcccactttattggtgagtgctggtgaacctatgggtccctggcccgatgattcttggaaacccacctggccctttgaacatgatactgtggccgttcccggtgatcattttactatggttcaagaacacgctgatgctattgcacgacacattgatgcctggttgggcggtggaaactcctaactccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtcgaggacgatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagacgatcccctcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatct

SEQ ID NO: 59 - DNA, 32767 bp, synthetic construct
  source 1..32767: mol_type = other DNA
atcgcgagaattaattcagataaaaaaaatccttagctttcgctaaggatgatttctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacgggtactcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgcgaattcatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgagcggccgcatacgatttattgacagctagctcagtcctaggtataatgctagcatgaccgtattacatagtgtggatttttttccgtctggtaacgcgtccgtggcgatagaaccccggctcccgcaggcggattttcctgaacatcatcatgattttcatgaaattgtgattgtcgaacatggcacgggtattcatgtgtttaatgggcagccctataccatcaccggtggcacggtctgtttcgtacgcgatcatgatcggcatctgtatgaacataccgataatctgtgtctgaccaatgtgctgtatcgctcgccggatcgatttcagtttctcgccgggctgaatcagttgctgccacaagagctggatgggcagtatccgtctcactggcgcgttaaccacagcgtattgcagcaggtgcgacagctggttgcacagatggaacagcaggaaggggaaaatgatttaccctcgaccgccagtcgcgagatcttgtttatgcaattactgctcttgctgcgtaaaagcagtttgcaggagaacctggaaaacagcgcatcacgtctcaacttgcttctggcctggctggaggaccattttgccgatgaggtgaattgggatgccgtggcggatcaattttctctttcactgcgtacgctacatcggcagcttaagcagcaaacgggactgacgcctcagcgatacctgaaccgcctgcgactgatgaaagcccgacatctgctacgccacagcgaggccagcgttactgacatcgcctatcgctgtggattcagcgacagtaaccacttttcgacgctttttcgccgagagtttaactggtcaccgcgtgatattcgccagggacgggatggctttctgcaataagccacaattcagcaaattgtgaacatcatcacgttcatctttccctggttgccaatggcccattttcctgtcagtaacgagaaggtcgcgaattcaggcgctttttagactggtcgtaatgaagaaacagaccatgctagataaaataaatcgttatgctcatgggtttgtagctgtaccagtgatttgtgcttgttcagaagcaggggtattcgagttactatcacagaaaaaatcactgaaattagaagaaatagtagagcatttagcagcaaatagtggacatctaatggtagctatgcgactcctagagtcattatcattcctatatcgctctcaagcagaagaatacatattgacagaacaaagtcaacaacatcaaatcatccctaaagctctgatgagcttatataaatatccatttgagttatatttaaaaggagaagtagaaacaggaatcagcaattggattaattgttcatctcgaagatgggatacagaaaactcattactaagtgatttattagatggagtattactcatccccctgctactagaactgaaaaaacaaaacttactggatgaatcaaaaaaaatatttaacacattaacaaattcccttaaacaagaactatcaacattattcatcaatttaggatgggcagaagaaaaaacagaagggctatatttaacagatataggtagatttatgcgtgacagatccttaaacttaggaacaacagcgtcttatgctcctatgttgttacagatgaaagaattactatttggaaatcctcagagagtatttcaaagaaataagactgaaaaagaaagacacgtaaatagaacattaaatgtagtagcaagtggctttcaacacgaaaagttttttgccgatacagataaaatcatcatatctatatttaaccaacagccaatagaagagcaaccaagctatatagtagacatgggttgtggggatggaacgctactgaaacgaatatataaaatcatcaaacaattctctgctaggggaaaagtattaacagagtatcctatcatcatggtaggagtagactacaatcaagaagcattagatgtcacagataaaaatttagtagatattccccatctagtcattccaggagatataggagcaccagaaaaattattagagcaattgaaagcacaaggaatagaaccagaaaaagtattacatattcgttcattcctagatcatgaccgaccatttatagctccaaaaaatacagaaatagctcaagcacgttctcagttagattatcaagtagtagatgtagatcgagaaggaaagctgataccacctcacatagcagtacaaagtttagtagaacacttagaaagatggtcatcaataataactagacatggattattgctcctagaagtacatagtttaacgccagcagtagtcaaaaaatatatagacgaaagtgaatctctgcattttgatgcctatcatgccttctcaatgcaacatttagtagaagcagatgtgttcttgatggcagcagcagaggtgggtttattttcacggaaagaagcttttcgtaagtatccgaaaacgttgccattgactcgaatcacagttaatcattttgaaaagcgtaaatatcagatacggtatgcaactgtaaacgatataccaaatctgttaaagtgtgcgacatttaatcaacctgtaaacgaacccttctttcaagttttattgaaacaaactccaacagcacatttattattagaatatcaaggtgaattggtagctgcaatcttcacagaaactaaaaattctaatgaggtgctaggaattcgtgagtttttagtaaggacttctgtagaaaattggcaagtattagcaaaagatttactagaatttgtggaacagtggggagtagtcaaaccaggaataaaagaaatagaaggattgttaaaataccatgaagccatctcaaactttcaaaaatcaaagtggtatcaatcttcagttttaaacaaaaagcttatagaaaaaataactctacacgaattagctactttagaactatgtaatttaatggctccagaatatgagctagaagcctttgccgcacgttggcttttgcgtgtttttcaagatatgggtgtatttctcagagagggtgaatcttatcaggagtctgagttggtttcccagctaaacatctcaccacgttaccagagacttttaggtgctttgttacaaattttgcataagcgtggtattctaaaaattgagaaagatagagtgttcacattagcaagatgtaagacctttgccttagaaaatatttcgtctgaagtttctgctttttatgattatttttctgaaaaatatcctgctcatttatcctggttaacagtagtcaaaaggtgtctagagaaatatcctttgattctacgcggtgaagttgatgtcaacgaagttgtttttacagatggggatatggagctatttgctggactatttctaggacatcgtgttgctgactactttaatgagttgctagcagatggggtttgctgggaagtagaacagcggttgttagaagaaaagagggcacaacctattcggatcttggagattggagcaggaacaggaggtgttacaggaatattgctagaaaagctagcttctcatgcagagcaaattgaattttggtttactgatatttctagcgtttttacacgttatggtgagagtaagttcaagcagtttccttgggtgaaatatcaaacctttgacatagaaaaatctcttgatgctcaggggataaagtctgaaagttttgatgtggtaattgctaataacgtactccataacacaaaattaattcatcaaaccttaaataacagtaactcactattaaatactggggggttattggcattactagagtttactcaaccaattgatattcttttatactttggagggttgcttcaaggattttggttgtttgaagatccagaataccgactagaagttggttgtttactgagtataccactgtggcaaaaagttctcagtgattgtgggtttgatgaaataataccattgggattaccctgtgagatgcacgctctttctaaggcaagagaatctgttatatttgctcgaaagcatcaagtacaggagaaaacattttctgaaaaaattaaacaaaatttgacagaaaatggtaagcatgggcaggcagaattcgattttattagtattaataattcacaagaaagttcatccaaattagaaatttttgaacaggaatgtcgaaaattattaaaatctctactgggtgttcaacgtatggagagattgcctggtgacacaccactaatggagtcaggaatggattcactggagttgttagaatttcgtgctcttatagaaagaaagtttgggattaagttaaagtctaccttcttttttagttacaaaactcttatagcggtagcagagtatctttcagaacgggaagatattaattttagttagtaggactatgactgaaaaaaaaacaacgttttcacaggtccacagggggaaattttgaatcagggggaaaatttgaatgcattctaaccagagcagttcactagcaagaatagccattgtaggtgtggcttgccgttttcctggagcagatactaaagaagaattttggcagctacttaaggaaggtcgggactcaatgcagaatcttccttcagagcgttggggaaatctatttaactccatgagttcagaaatagatttatctattcaacgcggtggttttttgaaagatatagatttgtttgactcatccttttttaggattactcctagggaagcgcagttaatggacccccagcaaaggctgttgttagaattgtcctgggaagcgatggaagatgcaggatatgcaagggatacattaaagggtaaatcagtaggggtttatgtaggagtttgtcactatgactacagaagtttattagaaaaaggtttggagacagccgaaatagctcaaattgctactggtacagctccagcaacttttgccaatcgcctttcctatttttataattttcacggccctagcttaactgttgatacggcttgttccagctctttggtagcaatgtatgaagcagtaaatgcaatacgtcgtgggcagtgtcaaacagcattggtaggaggcgttaatttaatgtgttctcctgtgaataaccaagtttatagtgctgcgggtatgttatctcctgatggagtctgccgggtgtttgatgcgggtgctaatgggtttgtcaggggagaaggaggggcagtagttgtacttaaagattatcaaaaagcgttgacggatggtgattctatttatggggttgttcgcagtgtagcagtgaatcatggagggcaagcaagttcttttacagcaccaaatccccaagctcaggcaaaattgttggaacaggcttatagggaagctaatattgatatagagtcggtaggttatattgaagcacatggtacgggtacatctctaggagacccaattgaggtcgaggctttaaatgaagcgtttaaagggttgagttcaagtggtaagttacctgctaatagttgtggtttgggctcagttaaaacaaacattggtcatttggaaggggcagcaggtttggctggactaatcaaagttttgctgtgcatgaggtatgcaactcttccatgttcattaaattatcagcaacttaatcctgatattgaattagaagaaggtccgttttttgtggtggacaagcttcaatcttgggaaataaaagttgatagggtaggaaaaccttatccacttagagcagggttgagtagctttggttttggcggtacaaatgctcatgtagttcttgaggaaggagaaaataagaaggaaggaagaagaggggataaggaaccttcagttcatctattaaccctttcagctaaaactgaaacagctctttcagagttagtcagtcgttatcaaaaatatttaaacactaatgcagaatcagagttagccgatatttgttataccgctaatactgggcgagtccattttaaccatcgactagcagcgatcgcctcaaacaaacaggagttagtagaaaaacttaaggaataccaagctggggaggcaacagcgggagtattaataggggaacttgcagaacgcataagaacaccgaaattaggtttcttattcacaggtcaaggttcccagtatgtcaatatgggaaaacaactgtataaaacacagcctgtattccgtgaagtattggataagtgtgatataatattggaaacggaaatagaatgttctctattagatgttctatataacaaaactacagattctcaagattcatctttaataaaccaaacagcttacactcaaccagttctgtttgcaatagagtatgcgctatttaagttatgggaatcttggggaattaaacccagcatagtaatgggtcacagtgtaggagaatatgtggctgcttgtgtagcaggagtcttcaatcttgaagatggtttgagattaatagctgctaggggcaggctgatgcaacagttaccttcaggtggagaaatggtttctgtgatggcttcagaatctacagttagcagactcctagaacctcacaaaaaagaaatagcgttggcgaagcccgccgtaggcatcgcagcaataaatggaccagaaagcacagttatttctggagactctgtagcagtaacaggtgtagtaaatgacctggaagcaaagggaataaaaactaaaaaactagaggtatctcatgctttccattcaccattaatggaaccaatgttaggagagtttgaagctatagctaatcaactaacctacaatcaacctaaaatacctattatatctaacgttacaggtacaaaagcagataatactattgcttcccctcaatactgggttaatcatgtcggtaaacctgtaagatttgcccaaggaatggaaacgctacatcaacaagggtatgaaacctttatagaagttggaccgaaaccagtattgttaggaatgggcaggcaatgtctaccaggaaatgtaggtgtctggttgccatcattacgtccgggagtggatgagtggcaacaaatgctttatagcttgggaaaattgtatgtaacaggagtaaaaatagattggtcaggatttgagtctgactctagtcgccagaaagtagcattgccaacttatccattccagcgagaaagatattggatagaaataacagaaaacaaacataaggaacatcaaaagtcagaaaatataagtgacacttcaattgttaaactactcactcaaggaaaaacagaagctctcactcaacaactagaaacagaagccaaattttcaccagaagagcttaaacttttaccagaaatattagagacattagccaaacaacatcaagaacaattaacagcagtaaccatcaaaaactggttctacgaaatccagtggaaacccttagctcaaaacaaccccaatgcaaacattgaacctagtcattggttaattttagccgataccacaggagtagcagaaaaattagctcaaaaattacaacaacagggtcataaatacagcttagtttatcgaggagagagctatcaaagacaagcaacaggtacttatcaacttaatcctcagattcccgaagcatttgaaaagctgtatcaagaaattcaacaaagtagtgaaactgccattacgaagttaattcacttgtggagtttagatgctccccaatcaaaagacttaaccctggaaaccctagaagaagctcaattatggggatgtggcagcgtagtacacctgttacagaccttagtcaaaaactctagcattcctgaactatggttagtaacccgtgggtctcaatcagtattatcccaaacagaaaaaaatctaacaggactagcagcgtcacccttgtggggattaggtagagtagtgtctaatgaacatccccaattatggggaggattagtagatttagacccacaagctgcagcaggagacgaagtagaaatgctgtggcaattattagttaatgaacaagaagaagataatctagctgtacggggagaaaatacctatgtagctcgtctggccaggcaagaacctcaagaatttcctgaatccctatccttatcatcagatggtagttacctaataacaggagggttaggagctttagggttacatactgcccaatggttagtatccaagggagcgaaaaatattgtcttaactgggcgtcgccctccctcagaaaaagtaagtgaatctataaaaaaattagaagaaacaggatgtcaagtgaaggtgatgttgggggatgtttctgttgaagaagacataggcaaaattctcaaacaaattcagatatcaatgccaacactcaaaggcataattcatacagcaggagtgttagatgatggaaccatacaacaaatgaattgggagcgttttgccaaagtcatgtcgcccaaggtaaaaggaagttggcatttacataaattaactgaaaatcagccattagatttctttgtgtgtttctcctcaatagcttcgatgttggggactcttggccaaggaaactatgctgcagccaatgcctttatggatgctttagctagttatcgttgcagcagaggattatcaggattggcgattaattggggagcatgggcatcagggggaatggctgctcgtttagcagtggagcatcaaaataggatgcacagcagtggcataactgagatggcgaccaaagaaggaatgtatgccttagatttactattaacaaatgaatctgctacagctcaggtaggtgtagcaagtatagagtggcaggtactctcagaaagttggagtggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttaggtatcaaaaaaaattcattgctacgagaattattagagaaggaggaatgggcagcaaaagatacacgacaccaaaaagttaaagccgaatttttagcaaaattagaggaggcatcactagagaaacgtcaggaaattttaactgaacacattcgagtgcaagtatctcaggtactaggtttaagttcatctaaattaccagaagtaaatgtaggttttgcctctatgggaatggactctttgatgacaatagaattaaaaaatcgactgcaaaatcaactaggaaccaagttgcctgagacaatagctatcgaatatccaactattgcaaaattgtccttatgtatagaggaattaatgggatggaagacaacagaaattgacccattatctgaagaaacaagattagagatgatggagcgctcaagaagtgaagcaatagccatcatcggcataggctgtagatttcccggaaatgccaacacaccagaaagcttctggcaattgttatccaatggcaaagactccattacagagattcccctagaacgttgggatctagattcctactacgaccccaaccctgatactccagggaaaatgtatatccgtcacgcagcattagtagaaaaagtagatcagttcgacccacgattttttggaatctctaaccgagaagcttatagtcttgatccacagcaacgcttcattttggaagtaacttgggaagctttagaaagagctggtattaaccctcaacaattagaaaacactcagacaggggtgttcctgggtatcggtcaaaatgattacgcgaatttaggttttcaccaagcagccgaagatataagcccttacgatgctacaggaaatttgttttgttttgtagcaggtaggttatcttactttttaggaacgcaaggtccatcaatggcaatagatacagcttgttcatcatctctagtagctatccatgaagcttgtgagagtctgcgtcagggtgagtccaacttggctttggctggaggagttcaactaattctctctcccgaagtaacaaccgcactatcaagattaaaggctctagcacctgatggtaagtgtaaaacttttgatgctgctgctgatggttttggtaggggagagggatgtggcattgtggtactgaagcgtttgtcggatgcgctcaaggatggggacaggatatcagcagtaattcctggttcggctgttaaccatgatggaccaagtagcggaatgacagtaccgaataaactggctcaggaaaaactgattcagaaagctctcaaagcagccaaggtaaaaccgttacaagtgagttatgtggaagctcatggtacaggaacttctctaggagatcctatggaagtgagagctttagctagggtgtttgaggagggacgtgatcaggaaaatccattgaacatcggttcagttaaaactaatatcggtcatctggaagcagcagctggaatagcaggtatgattaaggtgattttgcaattgcaacatcaggaaattgtgccccatctgcattttgctaatcctaacccctatgttgattgggagaatatgcctctacaagtaccgactcaactgactccttggttgtcgaaaggggagaaaagggtggcaggagttagttcttttggtatgagtggtacgaatgctcatatagttttagaagaggctcctattgaagtcagaaggcagaagtcagaagtcagaagtgaagaatatctagaacgtccggttcatatactaactctgtcggccaagactgaaaaagcactagaagatttagttaatagttatgaaagttatttagaagccgaaaataatgataattatttaggggatatttgctacacagccaacatcgggagagcaaaatttgaccacaaattagcagtggttacttctgataaacaagagttattagagaaactcaaacaatataaacaaggtgagaatgttgctggaatcttttcaggaaaacaaataagtgaaactagaacaaaaatagcctttatatttactggtcaaggttctcaatatctgcaaatgggaaggcagttatacgaaactcaacccacttttcataaaattattgaccagtgtagtgaaatgctggtaaaatatttagatgtttctttattagatatactttatccagttgaggttaaagatgaaagttcgactttgatagaccaaacagcttatactcaacccgctatatttgcccttgaatatgccctagctaaattatgggaatcatggggtataaagccagatgtagtcatgggtcacagtgtaggagaatatgtggcagcaacagtagcaggggtatttagtttagaagatggtttaaaattaatagccatgcggggacagttgatgcaaaagttaccctccggaggtcagatggcatctataatggcatcagaatctcaggtaatagaggcgataaaagaatatagttcccaagtaacaatagcagcagttaatggaccagaaattatagtaatttcaggtgagagtccagccatttcaaaaatttgtagtaaatttgaatcagaaggagtcaagaccaaggcgctacaagtatctcatgctttccattccccattgatggaaccaatgttaacagaatttgaagcagtagccaaagaaatatcctataatcatccccaaataccactaatatcaaatgttactggtcaagaagtaaatggagaaataaccactgctgaatattgggtgcgtcatgtgcgtcaaccagtaagatttgcggaagggatggaaactttacacaaacaaggtgctgaaattttcctagaaataggctcaaaaccgatacttttaggtatgggtcgtgagtgtctcatgggagaaaaaaaactatggttgcccagtttacgttcaggaaaaccagactggttacaaatgctacaaagtctggggcaattgtatgtacgagcaataaaaattgattggttaggatttgatcgggattatttccgtaataaggtagagttaccaacatatccttggcaacgaaaaaggtattggataacagatattagacaacggaaaagtcaagacaaaaaaagcataacatctgaaaaaaaagtacaacttgatggagtaaatattcaactaaaggaaatacaaatgaacgacaaaatcttacagcaaccaaaactaaaattatcagatccagaatcactatatttatcgaatgctgaatcaactatagaggttccaacaaaagcagttcaagtaaaacctgctgctgatattgaatctgaaaatataactcaattgaatagccttgatagagatgtgactcaaatcatagaaaccctcaaagaaagtttggcagatgctttatatgcagatataagtgaaattgaagaagacaaaaagtttgttgatttaggtttagattctattgtgggagtagaatggataactaacatcaataaaatctacaatttaaatataaaagctactaaactatacgattaccctactttgccagatttggctaaatatgtcgctcacaccctatcagctcagggtagaaatattgatgtagagcgatcgccatctgactcaagtcaagcaagcattagcaagcaatctcaactaactgatacccagagcaatttctcacaagtcaaggaaattttgaaacaacaattagcagatgctctgtatgcggatattagtgaaattgaagtaaacaagaagtttgttgatttaggtttagattctattgttggagtagaatggataactaatatcaataaaacctacaatttgaatatcaaagccactaaaatatacgactaccctaccttattagatttcgcaaaatatattaaccaggaaatttattctacaggagtaagtagattttcaacggagcataaagaatttaatcaaaaagactattcatctggaggttcgcaagaggaaatgacacaaaaattacgatcaatattgaagaaagtggccaataaagaattaacagttcaggaaggaaataaaatgattcaacaaataaaaaatcaattaaagtaaaaatatagtataatttttaaggaaataaaattttatgaacaaagagcaaatatttcaaataataaaaaaatatacctacgaaatagcaccagaattagaagaagtacctatatcacctactgatagtctcaaaaacttggggattgattcagtaaatagggcagaaattataatgatggtaatggaagagttatcattaaatatcccgcgtattgaattagctggatccaaaaatataggagaactggctgatatatttgcattcaaattagaagcaataaattcacaaagctagaaggcctatcaatgagtaacatagaaataacaggtatgggcattgttacttccattggtcaaggagttgctacttttaaagaagctctgttatcagggaaaactcaatttgcttatctaaaacagccaggacgtgaaagcatcaaaccatttattggtgccgaaattcccgatattgacgccaaaaccctatttcctgagtacagtggactgttacgtactgctactaagagcgcccaagttgcaatagtagctgtcgctgaagcctggcaagatgctcaactcacctccagccaagttaacccagaacgagtaggattagttgtaggtggttcaaatttacaacaacgttatcagcagcaaacctggcaacgttatcactcacgtcaggagtttatacgaccaacttatggtctgaccttttgggatacagatatattaggtctgatttcccagtgttttcagattcaaggtgagggctatagtgtaggtggagcttcggccagcggagcagttgctataattcatgctgctcgtcaaattttaatggggaacagtgatgtttgcattgctctgggagctttatccgatctatcagggtatgaattccaagcactaatgaatttaggtgcaatggggagcgaacgttttgccgatcgcccaaaccttgcttgtaggccatttgaccaagaccatgatggctttatttatggagagggttgtggagcagtgattttagagagaactgaccgcgctcaacaacggggcgctcaatcccatggccaacttaagggttggggattaactttagatgggaaccgtagtcccgaaccttcccaaaaaggggaagaacgggctataaatactgctttagccatggcagaccttcagccagagagtattgattatgtgaatacccacggtactggttccccccttggcgacaaaacagaagtggcagctttcaaatcagtaggacttcagcattgcctatttaattcaacaaaatctttaataggccactgcttaactgctgctggtgtagtggaagcgatcgccaccatattacaaatgaagtttggtttttgtcatccaactaaaaatttagttaatcctattgataccagtctcaattgggtgaaagaaacttctgttcaagctgagattaaatatgctatcagcaacagttttggttttgggggtatcaatacagctttattaattggacaggagtaaactcatgcaacaagttggaattgaagcactaaatgtatatgggggttcagctaaactagatgtgcgaatgctaggcgaagcacgtcacttagacatgactcgcttcgataatcttctgatgaaagagaagacagttgctatgccctatgaagatccagtttcttatgctattaatgcagcaaaacctattattgatagcctcagcgttctggaaaagcaacaaatcaaaatggtgatagcttgtagcgaatctggcattgattttggcaagtctatgagcacttatatccaggattatttgggactaaatcgtaactgtcgaatgtttgaaataaagcaagcttgctactcaggtacagcaggtttacaaatggcattcaatttaattttgtcccaaacctgtccaggggctaaagctttggttataggaactgatatatttcggcctgttgttgtagagggaggagaagctctcagcgaagattggtcttttgtggaaccaagcagtggtgctggagcagtagctattttagtcagtgatgttcctaaaattttccaagctgatgtaggatgtaatggttactatggctatgaagtcatggatacttgcagacctaacccagactcagaagcaggagatgcagatttatcattgctatcttacctagattgttgtgaaaatgcttaccgggaatatcaagatcgagtagaaggagtagattaccaaaaaacttttgattacttgagctttcatactccttttgggggtatggtgaaaggggctcacagaagtatgatgcgtaagtttaaaagggcaaaacctgtggagattgaagaggactttcagaaacgggtgatgccgggattagtctattgtcaacaggtgggtaatattatgggagctacagtatttttatctttagctagcatgattgataatgcagattttagcaaagcccgacgaattggtgtattctcttatggctctggttgttgttcggaattttatagtggagtcgtcaccccgcaaggaaaggaaattcaagctcaacaaaaaatttcatcacagttagcaatgcgctattccttaagtatggaagaatatgagcagttactaagtcatagttcggcagttgcctttggcactagaaatgttaccctagattacaaactatttcctggtgtgtgggaacaaattgaaggtaaaggtcgcttagtgctgaaaagaatcaaggaatttcaccgagaatatgaatgggtatagccatgagttatcaaaccctgaaaatcagttatcaagatgttgtacaaaggattcagatatatcgacctgaatctaacaatagcatcaatagtcaattaacgatggaattgttgtcagctttgcaagctgctgaagcagaggaagttgttaaagtagtgatattagaaggactacctgatgtattttgtacagggatggattttgaagaagtggcaacagcaaaacaatttgatccaaaagctagtgctaatggttactacaatattttgaaacaaatgtctcaaagtagcaaagtaattttgtcacttgtgcgtggtaaagtacaggcagggggagttggtttagtagcagcaagcgatctagttattgctgatgaaacggcaacttttgttttatcagaattattatttggattattaccagcttgtgtattgccttttttgattcgtcgagtgggatttcaaaaagcctaccgtttagcactaacgactcaagctatttcagtatcagaggctgataagtggggattaatagatgaatatggcagtaatattaatcagttaataagtaaatatattcgacgtttaaagtatttaccctcatcaggggtgaaagagttaaaaaattatattaatcagttatggattattcaggcagaaactcaaggtttagccgtcaacgagatttctagcttaatagcagaacctactgttcaagaaaaaattaaacgttttcaaaaagaaggattatttccatggcaaacctaaatcttaatttggacttagtagagggcaactctgatgtagtacaactggtggagttgggtaatggtgtcgtgcaaatcacgatgaaagatgaagaaagctgcaatggcttttctcctggaataattgaaggattatacaaatgttttggtgcagttgctcaaaaccaaagttataaagtagtaattttgacaggctatggaaattatttctgttcgggagggacaaaagaacggttaatcagtatttggaaaggagacagcaaatgtaatgatttagatttttttagaatagcattagattgtgaaataccagtaattgcagctatgcaaggtcatagtattggcggtggtttggttttgggattgtatgcagatttagtagtgttaagtcaagaaagtatttacactactaattttatgaagtatggttttactccaggtgtcggatgcacgttaattctccctgagaaattcggtgctttagggtttgaaatgatgtatactgcccaaaattatcgagggaaagaattagctgaacggggtgtttcttttccagttgtaccgagaaaagatgtgctagaagtggctaaaaatatagcctatgaaatgtcggaaaaacccagactatctttaataactttaaaggaacacttaacttcaaaaatccgcaaaacgctaccaggatttatagataaagaagtagctatgcacgaaataacctttcaccaaccagaggtagcaagtagaatagaggaaaattttgacaaaaggacaacggcaagtaataaccctcaaaattttcctcaagaggctgtaagaaaagaaataagaacagattcattgaattgtcaaccatttcagttgaagacatttagttatggttcgttaaacaatttaactttggtacctctagaacgtagagttccaagcccaagtgaagttgaggttcaaatcaaaactgtaccagttaattttcgggatatactcaatgcacttggtatgctccaagagtattacgaaaaaacatttggcattgctaatgctgaagatctcacttttggttttgaaggtgcaggtactatcgtagctgttggggcagaagtatcgcagtggcaagtcggcgatgaagtaatggtaatgagaattcacgatgcatttagtagctttattatctgctcgccggacaaactggtgcgtaaaaattttaatctgaatatggaagatggagctagtatttgggggccgttttcgactgcatatcacgggttgattaacttagccaaaattcagccaggagatagagtattaattcacgctgcttcaggtgggattggacaagcagcaattcagttggctcaactttttggggcggaagtatttgctactactagtccaggtaagatgaattatctccgggaacagggaattaagtatgtgatgaattctcgaacgatggagtttgtaaatgatgtgatggaattcactcagggccgtggggtagatgttattctcaatagcctgactcatggagaatacatccagaaaaatctagagatccttgccgatagagggcgatatgttgaacttggtaagttgggcatttggagtcacgagcaagtctatcagaaacgcccagatatcaaatattttacttttgatttgttagaagaatttgccaaagataatcaattgttttctcagatatgggataatttggcacttgaatttgaccgcgatcgcttgaagccactaccttacaaaacatttccaatagaagatgttcttaaagcctttgattatatgcgacgcgggaagcattttggcagggtagtggtagttatgcctgactcctattctagacaggaacaagagttggatgctcggttatccattgaaaacaaaatgactaaagaggaacagatattatttcaattacaatctggtgaagtttccttagaaaatgcggaacaactattgttaggaaatacggaaacagaaacacaagataaagctatagcagaaaatcaaatagataatattcaaaacaagttaattaacatggatagctcagagaaaatcttatctttgattagttcagtagaaatatctttagaaacagcagaaaaattattattagaagtagtagaaccagaagttaaaacagaggttaatgatgaggttaatcctagtcaaaatcatataccaactacagatatagcgattattggtatttcatgtagatatccaggagcgaataactggaaagaattttgggaaaatttaaagaatggaattgacagtgtaacggaagctcctcctggaagatgggaagaaaaaaattggtatcatccagatccagataatccaggtacttcctattcaaaatgtgccggttttttagatgaaattgataaatttgaccctttattttttcatatttctccggaagaagcttggtttatggagcctgagcaaagaatatttttagaagaagcttaccacgctatagaagatgctggatatgctacagactctcttagaggtaaacaatatggagtatttgtgggagttacggtaaatggtggttatcttaagttgttgtcaatttcaggattagatgttcataggatggcggctacaggaaatggtccgtcaatgataccagcaagaattgcctatatgcttgaccttcaagggccagtagtagctattgatactgcctgctcgtcgtcattggtagctgttcatcaagcttgccaaagcatacaacgaggagagagcgaaatagcgatcgctggaggtattactctaatgccaacatcagacttccaaataatgtcaagtcagtttcaagttgtatctcctgatggacgttgtaagacttttgatgcttcagcatcgggtacagcctggagcgaaggttgtggtgttctcttattaaaaagctacagtcaagcaattcaagacaacgaccatatttatggagtaatcaaaggaacaggagttaattatgatggtaatactaacgggattagtgcccctagtagtcaatctcaagccagtttagaagaagcagtttatcagaaatttggaattaacccagaaactattagttatgtagaagctcatggtacagcaacacctctgggagacccgatagaagtagaagctttaacagaagccttttctaaatggacgaacaaaaaacagttttgtgcgattggctcggtaaaaactaatattggtcattcagcagcggctgccggagtttctggtttgatcaagacaattttgtgcctcaaaaatcaaaaattagttccatccttacattttaatcaaccaaacccacatattgactttgaaaatagccctttttatgtcaatacaaaattaaaagattgggaagtgcttgaaggccaaccaaggcaagctacagttagttcttttggttttagtggcactaatgctcatatagttatagaagaggctccttctcaagttaaaagtcagaatattgttgaacgtccgatccatctgttaactctatctgcgaaaacagaaaaggctctagaagatttagtcagtaattatcaaaattatttagaaactaatcccgagttaccactagcagatgtatgttatacagcctctacaggtagagcacattttaattatcgattaggagttattgcttctgaaccaaaagcattaatagagaaactacttgggtggaaagctcaggaagaattagtaggactattttcaggaaaacgaaatagcgaaggtcagaaaatagcattcctgttcacaggtcaaggttcccagtatgcgaatatgggaaggcaactttatgaaaaagcaccaactttccgtcaagctttagaggaatgtgaccaaattttacaaccctatctagaagtacctctattagaggtcatatactctgaggatgcacaaaagtcaagtgataatctattagaccaaacagcttacacccaaccagctgtgtttgctgttgaatatgctttggctaaattatggtcttcatggggaatcaaaccatgtgtagtcatgggtcacagcgtaggagaatatgtagcagcaacagtagccggagtattcagtttagaagatggtctgaaactaatagccatgcgaggaaagttgatgcaaaagttaccctccggtggtgagatggtatccgtaatggcatcagagtctcaggtaacagaggctataaaagaatatagttcccaagtaacaatagcagcgataaatggaccagaaagtatagtaatttctggtgagagtgtagccataaaaaatatttgtagtttatttgaatcagagggaataaagaccaagcagctacaagtatcccacgctttccattctccaatgatggaaccgatgttagcagagtttgaatcagtagctaaacaagtcacctataatcaaccccaaataccactaatatcaaacgtcacaggtactgaagtagatggagaaataacaaatgctgaatattgggtagatcatgtgcgtcaaccagtaagatttgcccagagtatgaaaactctagagtcggaaggatatgaaaccttcctagaaataggaccgagaccaatattgctaggaatgggaagacaatgtgtaacagaagatgtaggagaatggctgccatcattacgtccaggggtggatgaatgggaacaaatgctatcaagcttaggaaaattgtatgtaaaaggagccaaaatagactggtcagggtttgactctgattatactcgccagaaagtagtattacccacatatccattccagggagaacgttattgggtagaaaccaacaacaacttctggcctcaacggcaattttcccaaggggaaaacctccatcccctattaggtcaaaagctaaattgtgcaggggaacaacaaatatttgcatcacaaataggagaaaactcacccaactatctgagggaccaccgagtatttaatcaagcactatttcccataacaggctacctagaaatagcaatagcagcaggaaatcaccaattaaaaacatcccagatagtaatagaagacctaaccataactagaggatggatactaccaacaggagaattaaccaatgcccaaaccatactcaccccaatagataaccaaagctataagtttcaaatattttctcaaccagaacaacaggagtggagactccacacgacaggaaaaatcagaaaagagtcaaccccccctactcagacaaaagttgacctagaaaaatacaagagtgaatgtaatcaaacaatagaagtcaaacaacattatcaaaaatgtcaacaagtagggatagactacgggaataccttccaaggcatccaagaattgtggtcaggttcaaaccaagcattaggttatatcaaactgcccgaagaattgataacacaaacaagcgactatcatttccatccagcactattagatgcagccttgcaagtaatgttttatgcactgccagcaacggataatgacaaaacttatctgtcagcaggaatagaagaattcagactatataagactcccgggctgagtatatgggcatatgtatcagtaaccagtcaagaagtggaaactccagaaagtttgacggctattgtcaccatagtaactccagaaggagaaataattgccaacatcaaaggtttacaagtcaaactagcaacaaaacagaccctactgggaacagaaaccgaatcaatagaaaattggttatatgaagtagagtggagaaacaaaggtattttaggtaaactactcccaccagatttcctcatacctcctatacaaatcaaccaaaaattaactccaactctgacagaattagtaactcaagtagataatgaaacaacagcgtcttttgaaacaagcttagaagaattaagcagagattatatagtacaagcattacagtcaatgagttggtcatacaaaccaacagaaagctttgcatttgatgtagcagcccaaaaattaggtatagttcctacccatcgaccactgtttaagcgtttgctgcaaatattaacagagtcaggaatactcaactcaaagaatcagcagtgggaagtagcacaaaccttacctgaagtcaagcctacggaaaaaatcagcagtttacagaagaaatatccagaagaaacagcagcattgacactactctctcgttgtgggtctaaactaagtggggtattacgaggagcaatagacccaggagagttagtgttcccccaaggagatttgacagcagcgactcaactttatgaagactcaacagtagcgaaagtgatgaacacaatagtagaaaaatccatcaccaaagctatagaaaaatccccgaaaagccgggggctcaggttgttggaaataggagggggaacaggagggactacaagctatatcctacctcatctaactcctcagcaaaccgaatatacattcacggatataggggcattatttacagccaaagctcaagagaaattccgggattataagttcataaagtatcaaactttagacatagaagtagacccgacaactcaaggatttgaggctcatcaatatgatgtaattattgcagctaatgtacttcatgcaacgacagatatgaagcagacattatctcatgtgcgagaactgttagcagatgggggaatgttggtgttatctgaagcaacagctaaaacactatgggtagatttagtatttgggttgttagaaggatggtggaaattcagggattatgaattacgaccagattatcctttgttgagtcgtgaaaaatggcatcatgtcttgagagaaacgggttttactgaagtagttactatgccagaagtggagggaatggcagaaacattgtcagcacaaacggtaattgtagctaaaagctctcaaacgaagttagaacaaaggaatgatgattctaagagttggttgatactggcagactctgagggagtagggcaacagttagcgaccttactccgctcagtaggagaggtttgtactttagtatttgccggagaaaggtatcaacagatagctccgggagaatttagtattaatcctaatcaagctaaagattttgaggaggtaatagagacagtggcagggaagtcaccatcattgtatggagtggtacaatgttggacgactgaagccggagtggggaatgggattaattctgaggagttaggaagtttatcgaagttagggtgtggcacaactctatctttagtacaagcattggtgaaaggcgggttatcaactgtccctcgattatggttggtgacaaatggtgctcaggcggtgccgaataatcatccggtgataccaggagtagctcaatcttcggtatggggaatggggaaagtgattagctgggaacatccagagttgaactgtactcgtatagatttggacccagaggagactttagagggtaaagttgatgccctatttaaagaaatttggtcggaggatagggaagaccaggtagcatggcgtggggatagtcgttatgtagctcggttggtgggtagtcatcatcggcaattagtggcacaacaagctgatggtaaaactcaaaagcccttaagtttccgttctgaagcaagctatttgattacaggaggtatgggaggtttgggtttgctggtagctagttggatggtgtcaaagggagctaaacatttgatattgttaggacgccgttcaccggatgatgctacgaggaaaaaaataaccgagttagaaatggcaggagcgtccgtggtagtggaaaaagccgatgtgactgatttggagtcgatgaaaggggtgttgcagaggattgaggagtcaaagcgaccgttagtaggagtgattcattctgtgatggtgctatcaaatggagtgctacgaaatcagacttggtctagttttgaacaggtgatggaaccgaaagttcaaggtgcttggcacttgcatcaattgactcaaagtcaaccattagacttctttgtgctgttttcttcagcaacatctctgttgggttcactgggtcagggcatggcaaattattctgctgctaatgggtttcttgatggtttagctcattatcgtcgaactatgggattaccgggattgagtatccattggggagcagtttctcaagtgggacgagcgctcgggcgagatacagagacagccgctatgctcagtaagaatgggatgggtttaatatctccggctcaggtattggagtctttggaactattgatgagtagttcggatgtggaagtaggggtgatgcctattgagtggtcagggtggcaggagagagtggcacagtggccgtttttagtggattggcaggaaactatattggaggtagcccaaccatcgaagtcagattttctgttaaagttggaggctacaccacctaatgagcttcgcttgttgttggtggctcatgtgcgtcgtcaggtggctcaagtgttgagaattagtcatcctgaatcaattgaaatggatacagggttttttgatttgggtatagactctttgacttctgtggagttgaggaataagttgcaaggtagtttaaagtgttcagtaccttctactgtaactttggactaccctaccattaaggcattggtagaatatttatatcaacagctattgttagaacaagttagttactcaaatactgtaccaactgaagaaatcaatgaagatagggaggagattactctaagcgggacaccagctcgggaagcaagtagtgccttacgggatggctatcgtcaagcaggagtcagcgggcgggttcggagttatttggatttattggctggtttaagtgattttcgggaacattttgatggtagtgatggtttttctttagatttggtggatatggctgatggtcccggtgaagtgaccgttatttgttgtgccggtactgccgctatttctggtccccatgaatttacccgcttagccggtgctttgcgtggtattgctcccgtgcgggctgttccacaaccaggttatgaagaaggtgaacccttacccagttctatggccgctgtggccgctgttcaagccgatgctgttattcgcacccaaggtgataaaccctttgtggttgctggtcatagtgccggtgctttaatggcctatgctttggccactgaattattggatcgtggtcatcctccccgcggtgtggttttaattgatgtgtatcctcccggtcatcaagatgctatgaatgcctggttagaagaattgaccgccactttgtttgatcgggaaaccgttcgcatggatgatacccgcttaactgctttgggtgcctatgatcgtttaactggtcaatggcgcccccgtgaaaccggtttgcccactttattggtgagtgctggtgaacctatgggtccctggcccgatgattcttggaaacccacctggccctttgaacatgatactgtggccgttcccggtgatcattttactatggttcaagaacacgctgatgctattgcacgacacattgatgcctggttgggcggtggaaactcctaactccctatagtgagtcgtattagcggccgcatcgaatataacttcgtataatgtatgctatacgaagttattagcgatgaggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcctcgtcgaggacgatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagacgatcccctcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcg

SEQ ID NO: 60 - DNA, 540 bp, synthetic construct
  source 1..540: mol_type = other DNA
tacttggggctgaaaaaaaactgccgggttttcgaactcaagcaggcctgctactcgggcactgccggttttcatagcgcggtgcagttcgtgttgtcccagacctcgcccaatgccaaggcactggtggtcgcagtggacctgtgtcgcttcctgatgattggtgaaggcggcgccgcggcgcaggactgggctttttcggaacccagtggcggtgcgggcgcagtggccatgttgatcagtgacaagccgcatgtattcagcctcgacatcggcgccagtggttattacagcttcgaggtgatggacacctgtcggccggagcccgacagcgaagccggggacgccgatctatcgttgatgtcgtacctggattgctgtgagcaaaccttcaatgaatatcgccttcgggtcgacgacgtggactatgctcgcacgttcgactacctggcgtttcacacgccttttggtggcatggtcaaaggcgcgcaccgcttgatgatgcgcaagttcgcgccagccgacccgccagccatcgaa

SEQ ID NO: 61 - DNA, 540 bp, synthetic construct
  source 1..540: mol_type = other DNA
tatttgggcttaagtcgtaactgtcgcatgtttgaactcaagcaagcttgctactcaggaaccgctggcttgcaaatggcaatcaacttgattctgtcccaaacctttcctggagccaaagccttagttatcgccaccgatatctctcggtttctggttgcagagggaggggaagctcttagtgaagattggtcttttgcggaaccaagtagtggtgctggagcagtggctcttttagtcagcgatactccttacatttttcaagttgatgtaggctgcaacggttactatggttatgaagtcatggatacttgcagacctaatcccgattccgaagcaggagatgccgacttatccttactttcttacctagattgttgcgaaaatgcctaccgacattatcaaaaccgagtagaaggggtagactaccaagaaagttttgattatttgagttttcatactccttttggtggtatggttaaaggggctcatagaagtatgatgcgtaggttgaaaagagctaaacctgcagacattgaa

SEQ ID NO: 62 - DNA, 540 bp, synthetic construct
  source 1..540: mol_type = other DNA
tatttgggcttaagtcgtaactgccgcatgtttgaactcaagcaagcttgctactcaggaaccgctggcttacagatggcaatcaacttgattttgtcccaaacctttccgggagccaaagccttagttatcgccaccgatatctctcggtttctggttgccgagggaggagaagcaatcaattacgattggtcttttgctgaacccagtagtggtgctggagccgtggctcttttagtcagtgatactccccacatttttcaaattgatgtaggctgcaacggttactatggttatgaagtcatggatacctgtagacctaatcccgattcagaagcaggagatgccgacttatccttgctttcttacctagactgttgcgaaaatgcctaccgacattatcaaaaccgagtcgaaggagtagactaccgagaaagttttgattatttgagttttcatactccttttggtggtatggttaaaggcgctcatagaaatatgatgcgtaggttgaaaagagctaaacctgcagagattgaa

SEQ ID NO: 63 - DNA, 26 bp, synthetic construct
  source 1..26: mol_type = other DNA
tttcayackccttttggtggyatggt

SEQ ID NO: 64 - DNA, 834 bp, synthetic construct
  source 1..834: mol_type = other DNA
agcgggacaccagctcgggaagcaagtagtgccttacgggatggctatcgtcaagcaggagtcagcgggcgggttcggagttatttggatttattggctggtttaagtgattttcgggaacattttgatggtagtgatggtttttctttagatttggtggatatggctgatggtcccggtgaagtgaccgttatttgttgtgccggtactgccgctatttctggtccccatgaatttacccgcttagccggtgctttgcgtggtattgctcccgtgcgggctgttccacaaccaggttatgaagaaggtgaacccttacccagttctatggccgctgtggccgctgttcaagccgatgctgttattcgcacccaaggtgataaaccctttgtggttgctggtcatagtgccggtgctttaatggcctatgctttggccactgaattattggatcgtggtcatcctccccgcggtgtggttttaattgatgtgtatcctcccggtcatcaagatgctatgaatgcctggttagaagaattgaccgccactttgtttgatcgggaaaccgttcgcatggatgatacccgcttaactgctttgggtgcctatgatcgtttaactggtcaatggcgcccccgtgaaaccggtttgcccactttattggtgagtgctggtgaacctatgggtccctggcccgatgattcttggaaacccacctggccctttgaacatgatactgtggccgttcccggtgatcattttactatggttcaagaacacgctgatgctattgcacgacacattgatgcctggttgggcggtggaaactcctaa

(19) United States
(12) Patent Application Publication
(10) Pub. No.: US 2026/0218211 A1
(43) Pub. Date: Jul. 30, 2026

(54) HETEROLOGOUS PRODUCTION OF THE C33–C45 POLYKETIDE FRAGMENT OF ANTICANCER APRATOXINS IN A CYANOBACTERIAL HOST

(71) Applicants: University of Florida Research Foundation, Incorporated, Gainesville, FL (US); Smithsonian Institution, Washington, DC (US)

(72) Inventors: Yousong Ding, Gainesville, FL (US); Hendrik Luesch, Gainesville, FL (US); Valerie J. Paul, Fort Pierce, FL (US)

(73) Assignees: University of Florida Research Foundation, Incorporated, Gainesville, FL (US); Smithsonian Institution, Washington, DC (US)

(21) Appl. No.: 19/145,645

(22) PCT Filed: Jan. 5, 2024
(86) PCT No.: PCT/US2024/010479

§ 371 Date: Jul. 3, 2025

Related U.S. Application Data
(60) Provisional application No. 63/437,409, filed on Jan. 6, 2023.

(51) Int. Cl. C12N 15/74; C07K 11/02; C12N 1/20; C12N 9/10; C12N 9/16; C12P 7/42; C12R 1/01

(52) U.S. Cl. CPC C12N 15/74; C07K 11/02; C12N 1/20; C12N 9/1029; C12N 9/16; C12P 7/42; C12R 2001/01; C12Y 203/01; C12Y 203/0301; C12Y 301/02

(57) ABSTRACT

The present disclosure relates to recombinant cyanobacterial cells for the production of a chemical compound of interest. In particular, the present disclosure relates to genetic modifications that introduce one or more heterologous polyketide synthases (PKS) into a cyanobacterial cell. These cells can further comprise nucleic acid constructs that provide the cyanobacterial cells with the capability of producing chemicals of interest or compounds of interest, including Compound (I).

### CROSS-REFERENCE TO RELATED APPLICATIONS

This application claims the benefit of and priority under 35 U.S.C. § 119(e) to U.S. Provisional Application No. 63/437,409, filed Jan. 6, 2023, titled HETEROLOGOUS PRODUCTION OF THE C33-C45 POLYKETIDE FRAGMENT OF ANTICANCER APRATOXINS IN A CYANOBACTERIAL HOST, the contents of which are incorporated herewith by reference in their entirety.

### GOVERNMENT SUPPORT

This invention was made with government support under grants R01 CA172310, R35 GM128742, and RM1 GM145426 awarded by the National Institutes of Health. The government has certain rights in the invention.

### REFERENCE TO AN ELECTRONIC SEQUENCE LISTING

The contents of the electronic sequence listing (U119770220WO00-SEQ-WLC.xml; Size: 194,726 bytes; and Date of Creation: Jan. 2, 2024) is herein incorporated by reference in its entirety.

### BACKGROUND OF THE INVENTION

Polyketides (PKs) and PK-containing natural products (e.g., polyketide-peptide and -terpenoid hybrids) are large families of secondary metabolites of terrestrial and marine plants, animals, fungi and bacteria. These compounds are structurally complex, often with various stereoselective modifications, and possess extremely rich functional diversity. Indeed, 1% of known PKs possess drug activity, and tens of PK-related drugs (e.g., derivatives of erythromycin, epothilone and dolastatin 10) have been clinically used to treat diseases ranging from cancers and infections to hypocholesterolemia.¹ Continuous exploration of PKs and their hybrids would be expected to offer new therapeutic leads. However, the supply of these compounds for bioactivity studies remains challenging as total chemical synthesis, particularly PK (sub)structures, and isolation from biological sources have achieved limited successes in large-scale production, with notable exceptions where multigram synthesis and process development have been achieved.²

Depsipeptides are a class of PK-containing natural products with a rich diversity in structure and function. The generation of stereochemically rich PK fragments (e.g., unit A of cryptophycins) is often the most demanding task in depsipeptide synthesis.^{3, 4} Once PK substructures are available, solid phase peptide synthesis can be feasible to assemble natural and unnatural depsipeptides in drug research. The biosynthesis of PKs proceeds directly from simple acyl-CoAs catalyzed by polyketide synthases (PKSs).

### SUMMARY OF THE INVENTION

In one aspect, the present disclosure provides a recombinant cyanobacterial cell comprising a nucleic acid construct comprising a nucleic acid sequence encoding a heterologous polyketide synthase (PKS); and a nucleic acid sequence encoding a type-II thioesterase (TE-II).

In another aspect, the present disclosure provides a recombinant cyanobacterial cell comprising an expression construct comprising an inducible promoter operably linked to a nucleic acid sequence encoding, in the following order: Moorena bouillonii AprA, AprB, AprC, AprD, AprE, AprF, AprG; and a type-II thioesterase (TE-II) from an erythromycin cluster.

In another aspect, the present disclosure provides an expression construct comprising a promotor operably linked to a nucleic acid sequence encoding, in the following order: Moorena bouillonii AprA, AprB, AprC, AprD, AprE, AprF, AprG; and a type-II thioesterase (TE-II) from an erythromycin cluster.

In another aspect, the present disclosure provides a method of making a chemical compound comprising culturing a recombinant cyanobacterial cell under conditions permitting the production of the chemical compound; and isolating or recovering the chemical compound.

In another aspect, the present disclosure provides a recombinant cyanobacterial cell comprising at least one nucleic acid construct that provides for production of Compound (I):

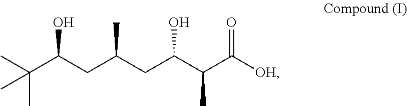 [SMILES: C[C@@H](C[C@H](O)[C@H](C)C(=O)O)C[C@H](O)C(C)(C)C]

or a salt thereof.

In another aspect, the present disclosure provides a method of making an apratoxin derivative compound comprising use of Compound (I):

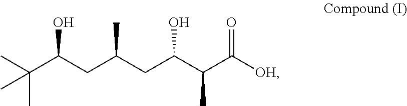 [SMILES: C[C@@H](C[C@H](O)[C@H](C)C(=O)O)C[C@H](O)C(C)(C)C]

or a salt thereof, in the synthesis of the apratoxin derivative.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE FIGURES

FIG. 1 shows structures of the natural product apratoxin A and synthetic analogs with improved properties.

FIG. 2 shows the biosynthetic scheme of the long polyketide fragment (APK) of cytotoxic cyanobacterial depsipeptide apratoxin A. The isolated 25-kb APK sub-cluster carries 7 genes encoding three PKSs and one complete set of HCS cassette for β-methylation. A type-II thioesterase domain (TEII) in the engineered sub-cluster releases the APK with a carboxylate terminus.

FIG. 3 shows a schematic representation of the construction of engineered APK sub-cluster for the heterologous expression in Anabaena.

FIGS. 4A-4C show the heterologous production of APK in engineered Anabaena. FIG. 4A shows the total-ion current (TIC) traces of the methanolic extracts of Anabaena cApra and rApra identified a new peak shaded in brown that showed the same retention time as the synthetic APK standard. This peak was missing in uninduced Anabaena rApra and the cells transformed with the pRL838-Ptrac vector. FIG. 4B shows the HRMS/MS spectrum of [M+H]⁺ of the new peak content. The m/z values of predicted APK fragments are provided. FIG. 4C shows the RT-qPCR analysis of AprA-G transcription in Anabaena rApra after being induced by 5 mM rhamnose for 0, 24, and 120 hours. The transcription level of RNA subunit of ribonuclease P (rnpB) was determined and used to normalize the signals of the biosynthetic genes in the same strain. Data represent mean±standard deviation (n=6).

FIG. 5 shows a schematic description of the research strategy for the identification of the apratoxin polyketide core genes from metagenomics DNA isolated from Moorena bouillonii sample. The library was screened with a pair of degenerate primers designed on conserved regions of HCS enzymes. The isolated fosmid bAprat4 carries a partial sequence of aprI.

FIG. 6 shows a comparison of the HMG-CoA synthase gene homologies (% identity) found in curacin A, jamaicamide A and mupirocin pathways for the catalysis of β-alkylation events. These genes were used to design a pair of degenerate primers for screening M. bouillonii fosmid library.

FIG. 7 shows an alignment of the HMG-CoA synthase genes from mupirocin (MupH, AF318063, SEQ ID NO: 60), jamaicamide A (JamH, AY522504, SEQ ID NO: 61) and curacin A (CurD, AY652953, SEQ ID NO: 62) gene clusters for the design of a pair of degenerate primers for screening M. bouillonii metagenomics DNA fosmid library. The boxed areas are the selected regions for the design of the forward and reverse primers. In the boxed areas, SEQ ID NO: 1 (GAACTCAAGCARGCYTGCTACTC) and SEQ ID NO: 63 (TTTCAYACKCCTTTGGTGGYATGGT) are shown. The expected size of the PCR product is 447 bp.

FIG. 8 shows PCR confirmation of the presence of HMG-CoA synthase gene in metagenomics DNA isolated from M. bouillonii sample using newly designed degenerate primers (M, 1 kb ladder).

FIG. 9 shows a DNA sequence (SEQ ID NO: 64) of codon-optimized TEII domain of the erythromycin cluster.

FIG. 10 shows the agarose gel analysis of five PCR-amplified fragments for assembling the APK sub-cluster with the engineered pRL838. All fragments showed the expected sizes.

FIG. 11 shows how the assembled constructs were isolated from 10 chloramphenicol resistant clones and digested by NcoI, EcoRI, and HindIII respectively. All digestion mixtures were analyzed by 0.6% agarose gel. Expected sizes (bp) of digested products for respective enzymes were NcoI: 14152, 11412, 6295, 2378, 98; EcoRI: 11888, 10093, 4095, 3804, 2176, 1409, 535, 298, 37; HindIII: 13895, 5002, 4066, 3995, 2800, 1212, 850, 708, 455, 417, 258, 249, 185, 75(2), 51, 42. M: Marker.

FIGS. 12A-12C show LC-MS analysis of shinorine produced by Anabaena cells transformed with pRL1383-shi.⁴ FIG. 12A shows MS analysis of methanolic extracts of wet biomasses of Anabaena cells transformed with pRL838-shino, vector control, and wild type. FIG. 12B shows the high-resolution MS (HR-MS) spectrum of expressed shinorine at 1.52 minutes. FIG. 12C shows the HR-MS/MS spectrum of shinorine. The expected fragment ions are 186.0987, 230.1258, 274.1155, and 318.1040 m/z.

FIGS. 13A-13B show (FIG. 13A) a schematic description of the promoter exchange using both in vitro Cas12a-based and in vivo Red/ET recombination approaches in E. coli BW25113/pKD46 cells, and (FIG. 13B) the PCR confirmation of promoter exchange. Colony 1-7: Co²⁺ inducible promoter (PcoaT), M: marker, 8-14: rhamnose inducible promoter (Prha). The expected sizes of PCR amplicons for PcoaT and Prha are around 1.2 kb and 1 kb, respectively.

FIGS. 14A-14B show PCR screening of positive transformant clones based on specific promoters after multiple rounds of segregation and selection of transformed Anabaena cells. FIG. 14A shows confirmation of pRL838-rApra in Anabaena clones. W: Wild type Anabaena, 1-4: four different Anabaena transformants, M: Marker. FIG. 14B shows confirmation of pRL838-cApra in Anabaena clones. W: Wild type Anabaena, 1-3: three different Anabaena transformants, M: Marker. The expected sizes of PCR amplicons were observed.

FIG. 15 shows the total-ion current (TIC) traces of the methanolic extracts of induced and uninduced Anabaena cApra and rApra. A new peak was identified from samples of Anabaena cApra and rApra and showed the same retention time as the synthetic APK standard. The peak was missing in uninduced Anabaena cApra and rApra and the cells transformed with the pRL838-Ptrac vector.

FIG. 16 shows HRMS analysis (ESI positive mode) of expressed (top) and synthetic APK (bottom). Both showed the expected [M+Na]⁺ ion, which is more abundant than the [M+H]⁺ ion. In addition, two ions corresponding to fragments with the loss of one and two waters are the top two abundant signals.

FIG. 17 shows a putative MS/MS fragmentation paths of APK [M+H]⁺ leading to observed fragment ions whose exact masses are shown in red.

FIG. 18 shows the preparation of authentic APK standard from compound 2.

FIG. 19 shows HRMS/MS analysis of expressed (top) and synthetic APK (bottom). The two showed the same fragmental pattern.

FIG. 20 shows MS-based quantitation of APK produced in Anabaena cApra and rApra induced by different concentrations of inducers.

FIG. 21 shows the gene table of the fosmid bAprat14.

FIG. 22 shows a comparison of ¹H NMR data of apratoxin A isolated from VPG14-77 and previous report³.

FIG. 23 shows the ¹H NMR spectrum of apratoxin A isolated from VPG14-77 in CDCl3 (600 MHz) at 25° C.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. The term “isomers” is intended to include diastereoisomers, enantiomers, regioisomers, structural isomers, rotational isomers, tautomers, and the like. All such isomers of such compounds herein are expressly included in the present invention.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen.

The term “carbonyl” refers to a group wherein the carbon directly attached to the parent molecule is sp² hybridized, and is substituted with an oxygen, nitrogen or sulfur atom, e.g., a group selected from ketones, carboxylic acids, aldehydes, esters, amides, and imines.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻. HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsFb⁻, SbF^{b}−, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁MesBr₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺ (C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “Cyanobacterium” refers to a member from the group of photoautotrophic prokaryotic microorganisms which can utilize solar energy and fix carbon dioxide. Cyanobacteria are also referred to as blue-green algae. Exemplary cyanobacteria include, but are not limited to, Anabaena sp. The cyanobacterial cell of the present invention can be selected from the group consisting of Anabaena, Synechocystis, Synechococcus, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geiderinema, Euhalothece, Calothrix, and Scytonema. Particularly preferred is Anabaena sp. PCC7120.

The terms “host cell” and “recombinant host cell” are intended to include a cell suitable for genetic manipulation, e.g., which can incorporate heterologous polynucleotide sequences, e.g., which can be transformed. The term is intended to include progeny of the cell originally transformed. In particular embodiments, the cell is a prokaryotic cell, e.g., a cyanobacterial cell. The term “recombinant host cell” is intended to include a cell that has already been selected or engineered to have certain desirable properties and to be suitable for further genetic enhancement.

“Competent to express” refers to a host cell that provides a sufficient cellular environment for expression of endogenous and/or heterologous polynucleotides.

The terms “polynucleotide” and “nucleic acid” also refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.”

The nucleic acids of this present invention may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, alkylators, intercalators, pendent moieties, modified linkages, and chelators. Also included are synthetic molecules that mimic polynucleotides in their ability to bind to a designated sequence via hydrogen bonding and other chemical interactions.

The term “nucleic acid” (also referred to as polynucleotide) is also intended to include nucleic acid molecules having an open reading frame encoding a polypeptide, and can further include non-coding regulatory sequences and introns. In addition, the terms are intended to include one or more genes that map to a functional locus. In addition, the terms are intended to include a specific gene for a selected purpose. The gene can be endogenous to the host cell or can be an endogenous or heterologous gene that is recombinantly introduced into the host cell.

In one aspect the invention also provides nucleic acids which are at least 60%, 70%, 80% 90%, 95%, 99%, or 99.5% identical to the nucleic acids disclosed herein.

The percentage of identity of two nucleic acid sequences or two amino acid sequences can be determined using the algorithm of Thompson et al. (CLUSTALW, 1994, Nucleic Acids Research 22: 4673-4680). A nucleotide sequence or an amino acid sequence can also be used as a so-called “query sequence” to perform a search against public nucleic acid or protein sequence databases in order, for example, to identify polyketide synthases (PKSs) within microorganisms, which can also be used in various embodiments of this invention for either transformation of a microorganism or inactivation of an endogenous PKS within a microorganism.

In addition, any nucleic acid sequences or protein sequences disclosed in this patent application can also be used as a “query sequence” in order to identify yet unknown sequences in public databases, which can encode PKSs which could be useful in this invention. Such searches can be performed using the algorithm of Karlin and Altschul (1990, Proceedings of the National Academy of Sciences U.S.A. 87: 2,264 to 2,268), modified as in Karlin and Altschul (1993, Proceedings of the National Academy of Sciences U.S.A. 90: 5,873 to 5,877). Such an algorithm is incorporated in the NBLAST and XBLAST programs of Altschul et al. (1990, Journal of Molecular Biology 215: 403 to 410). Suitable parameters for these database searches with these programs are, for example, a score of 100 and a word length of 12 for BLAST nucleotide searches as performed with the NBLAST program. BLAST protein searches are performed with the XBLAST program with a score of 50 and a word length of 3. Where gaps exist between two sequences, gapped BLAST is utilized as described in Altschul et al. (1997, Nucleic Acids Research, 25: 3,389 to 3,402).

As used herein, the term “genetically modified” refers to any change in the endogenous genome of a wild type cell or to the addition of non-endogenous (heterologous) genetic code to a wild type cell, e.g., the introduction of a heterologous gene. More specifically, such changes are made by the hand of man through the use of recombinant DNA technology or mutagenesis. The changes can involve protein coding sequences or non-protein coding sequences, including regulatory sequences such as promoters or enhancers.

The term “recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium” or a “recombinant cyanobacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well.

The term “homologous recombination” refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see Court et al., “Genetic engineering using homologous recombination,” Annual Review of Genetics 36:361-388; 2002.

The term “non-homologous or random integration” refers to any process by which DNA is integrated into the genome that does not involve homologous recombination. It appears to be a random process in which incorporation can occur at any of a large number of genomic locations.

The term “expressed endogenously” refers to polynucleotides that are native to the host cell and are naturally expressed in the host cell.

The term “operably linked” refers to a functional relationship between two parts in which the activity of one part (e.g., the ability to regulate transcription) results in an action on the other part (e.g., transcription of the sequence). Thus, a polynucleotide is “operably linked to a promoter” when there is a functional linkage between a polynucleotide expression control sequence (such as a promoter or other transcription regulation sequences) and a second polynucleotide sequence (e.g., a native or a heterologous polynucleotide), where the expression control sequence directs transcription of the polynucleotide.

The term “vector” as used herein is intended to refer to a nucleic acid molecule capable of transporting another nucleic acid to which it has been linked. One type of vector is a “plasmid,” which generally refers to a circular double stranded DNA molecule into which additional DNA segments may be ligated, but also includes linear double-stranded molecules such as those resulting from amplification by the polymerase chain reaction (PCR) or from treatment of a circular plasmid with a restriction enzyme.

Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”). Thus, transformable nucleic acid constructs can be transformed into a cyanobacterial cell and then integrated into the chromosomal to obtain the recombinant cyanobacterial cell of the present invention. Alternatively, the transformable nucleic acid constructs can be present within the recombinant cyanobacterial cell in the form of self-replicating plasmids or modules (see, for example, Taton, Arnaud et al. “Broad-Host-Range Vector System for Synthetic Biology and Biotechnology in Cyanobacteria.” Nucleic Acids Research 42.17 (2014): e136. PMC. Web. 26 Jul. 2017, the disclosure of which is hereby incorporated by reference in its entirety).

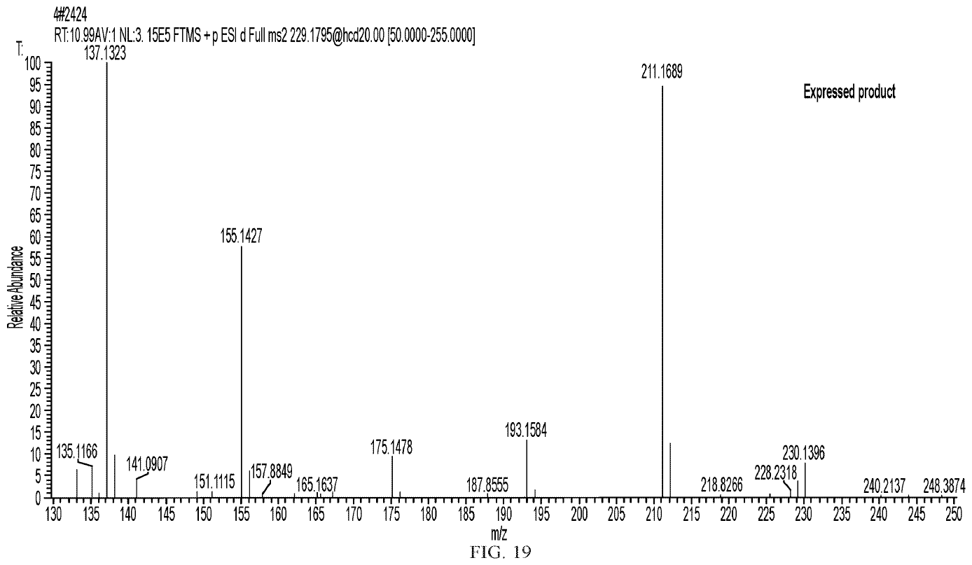

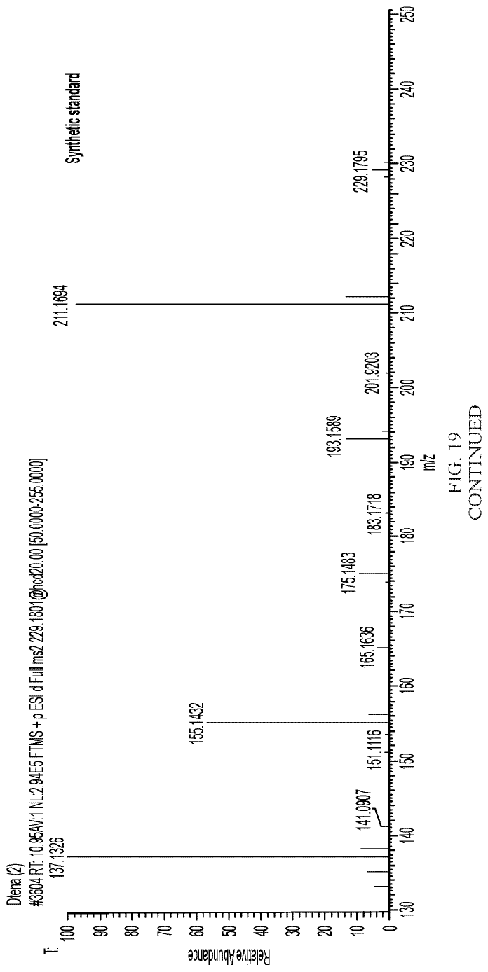

A “promoter” is an array of nucleic acid control sequences that direct transcription of an associated polynucleotide, which may be a heterologous or native polynucleotide. A promoter includes nucleic acid sequences near the start site of transcription, such as a polymerase binding site. The promoter also optionally includes distal enhancer or repressor elements which can be located as much as several thousand base pairs from the start site of transcription. The term “promoter” is intended to include a polynucleotide segment that can transcriptionally control a gene of interest, e.g., a PKS gene that it does or does not transcriptionally control in nature. In one embodiment, the transcriptional control of a promoter results in an increase in expression of the gene of interest. In an embodiment, a promoter is placed 5′ to the gene of interest. A heterologous promoter can be used to replace the natural promoter, or can be used in addition to the natural promoter. A promoter can be endogenous with regard to the host cell in which it is used or it can be a heterologous polynucleotide sequence introduced into the host cell, e.g., exogenous with regard to the host cell in which it is used. Promoters of the invention may also be inducible, meaning that certain exogenous stimuli (e.g., chemicals, nutrient starvation, heat shock, mechanical stress, metal ions, light exposure, etc.) will induce the promoter leading to the transcription of the gene. In certain embodiments, constitutive promoters, such as PcoaT or Prha, can be used to express heterologous PKSs within a recombinant cell. Other constitutive promoters that can be used in the context of this invention include Pcpc560, Ptrc, Ptrc2O-2, PA1lacO-1, L03, PnrsB, PpsbA2, PpsbA, the plastocyanin promoter and the promoters provided in the J23 library (a synthetic library of minimal and constitutive σ⁷⁰ promoters, examples of which are provided in FIG. 19). These and other promoters, such as inducible promoters, are disclosed in “Engineered transcriptional systems for cyanobacterial biotechnology”, Camsund and Lindblad, Frontiers in Bioengineering and Biotechnology, 2014, 2:40, which is hereby incorporated by reference in its entirety. Endogenous and exogenous promoters can also be identified using a bioinformatics algorithm, such as bTSSfinder: a novel tool for the prediction of promoters in cyanobacteria and Escherichia coli, Bioinformatics. 2017; 33(3): 334-340.

The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). The recombinant nucleic acid molecule (e.g., a recombinant DNA molecule) also includes an isolated nucleic acid molecule or gene of the present invention.

The term “gene” refers to an assembly of nucleotides that encode a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence. In particular embodiments, the subject invention provides genes encoding PKSs disclosed herein.

The term “endogenous gene” refers to a native gene in its natural location in the genome of an organism. The terms “endogenous” and “native” can be used interchangeably within this application. A “foreign” gene, “exogenous gene” or “heterologous” gene refers to a gene not normally found in the host organism, but that is introduced into the host organism by gene transfer or transformation of the microorganism. Foreign genes can comprise native genes inserted into a non-native organism, or chimeric genes. A “transgene” is a gene that has been introduced into the genome by a transformation procedure. A “foreign gene” can also comprise an endogenous gene that is introduced into another location in the genome of an organism (i.e., moved from its natural location within the genome of the organism) which is operably linked to its naturally occurring promoter or to a heterologous promoter. A heterologous gene can also include a native gene of a microorganism that is found in its native location but which has had its native promoter substituted with a heterologous (non-native) promoter, such as the constitutive or inducible promoters discussed within this application. The terms “heterologous”, “exogenous”, and foreign” can be used interchangeably within this application.

The term “fragment” refers to a nucleotide sequence of reduced length relative to the reference nucleic acid and comprising, over the common portion, a nucleotide sequence substantially identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least about 6, 50, 100, 200, 500, 1,000, to about 1,500 or more consecutive nucleotides of a polynucleotide according to the invention.

The term “open reading frame,” abbreviated as “ORF,” refers to a length of nucleic acid sequence, either DNA, cDNA or RNA that comprises a translation start signal or initiation codon, such as an ATG or AUG, and a termination codon and can be potentially translated into a polypeptide sequence.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments. The terms “homology” and “identity” can be used interchangeably within the subject application.

As used herein, “substantially similar” refers to nucleic acid fragments wherein changes in one or more nucleotide bases results in substitution of one or more amino acids, but do not affect the functional properties of the protein encoded by the DNA sequence.

The term “substantially similar” also refers to modifications of the nucleic acid fragments of the instant invention such as deletion or insertion of one or more nucleotide bases that do not substantially affect the functional properties of the resulting transcript.

The term “expression”, as used herein, refers to the transcription and stable accumulation mRNA derived from a nucleic acid or polynucleotide. Expression may also refer to translation of mRNA into a protein or polypeptide.

An “expression cassette” or “nucleic acid construct” or “genetic construct” refers to a series of polynucleotide elements that permit transcription of a gene in a host cell. Typically, the expression cassette or nucleic acid construct includes a promoter (native or heterologous) and a heterologous or native polynucleotide sequence that is transcribed. Expression cassettes or constructs may also include, e.g., transcription termination signals, polyadenylation signals, and enhancer elements.

The term “codon” refers to a triplet of nucleotides coding for a single amino acid.

The term “codon bias” refers to the fact that different organisms use different codon frequencies.

The term “codon optimization” refers to the modification of at least some of the codons present in a heterologous gene sequence from a triplet code that is not generally used in the host organism to a triplet code that is more common in the particular host organism. This can result in a higher expression level of the gene of interest.

The term “transformation” is used herein to mean the insertion of heterologous genetic material into the host cell. Typically, the genetic material is DNA on a plasmid vector, but other means can also be employed. General transformation methods and selectable markers for bacteria and cyanobacteria are known in the art (Wirth, Mol Gen Genet. 216:175-177 (1989); Koksharova, Appl Microbiol Biotechnol 58:123-137 (2002). Additionally, transformation methods and selectable markers for use in bacteria are well known (see, e.g., Sambrook et al, supra).

The term “knockout” generally refers to a partial or complete suppression of the expression of at least a portion of a protein encoded by an endogenous DNA sequence in a cell. As used herein, the “knockout” relates to the deletion of a target gene, such as an endogenous PKS.

The term “knockout construct” refers to a nucleic acid sequence that is designed to decrease or suppress expression of a protein encoded by endogenous DNA sequence in a cell. The knockout construct is inserted into a cell, and integrates with the genomic DNA of the cell in such a position so as to prevent or interrupt transcription of the native DNA sequence. Such insertion usually occurs by homologous recombination. The knockout construct nucleic acid sequence may comprise (1) a full or partial sequence of the gene to be suppressed, (2) a full or partial promoter sequence of the gene to be suppressed, or (3) combinations thereof. Typically, the knockout construct is inserted into a cyanobacterial host cell and is integrated into the cell genomic DNA to delete a target gene, such as an endogenous PKS, usually by the process of homologous recombination.

The phrases “disruption of the gene” and “gene disruption” refer to the deletion or insertion of a nucleic acid sequence into one region of the native DNA sequence and/or the promoter region of a gene so as to decrease or prevent expression of that gene in the cell as compared to the wild-type or naturally occurring sequence of the gene.

The term “plurality” means more than one.

The terms “chemical compound of interest” or “product of interest” refer to a product produced by the modified cyanobacteria. In one embodiment, the product is Compound (I):

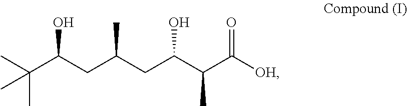 [SMILES: C[C@@H](C[C@H](O)[C@H](C)C(=O)O)C[C@H](O)C(C)(C)C]

or a salt thereof. In other embodiments, the product can be cyanobacterial secondary metabolites, polyketides, or non-ribosomal peptides and their hybrids. In other embodiments, the product can be analogs of cyanobacterial secondary metabolites, polyketides, or non-ribosomal peptides and their hybrids. In other embodiments, the product can be unnatural compounds of polyketides, or non-ribosomal peptides and their hybrids. In other embodiments, the product can be secondary metabolites, polyketides, or non-ribosomal peptides and their hybrids of bacterial species that do not belong to the cyanobacterium phylum.

In one embodiment of the subject invention, a recombinant host cell comprising inactivated endogenous PKSs and genetically modified to contain one or more exogenous PKS. Other embodiments provide for recombinant host cells that have been genetically modified to substitute a constitutive promoter for the endogenous promoter. For example, it is possible that the genetic modification causes a constitutive expression of the endogenous and/or exogenous PKS. These cells may further comprise additional nucleic acid constructs that permit the expression of a chemical or other compound of interest.

Certain embodiments of the invention demonstrate the ability of production of a compound of interest (Compound (I)) by the recombinant host cells disclosed herein.

As discussed above, the invention provides for the genetic modification of a recombinant host cell in a manner that decreases or eliminates the expression of endogenous PKSs. One possibility is that the genetic modification comprises a heterologous nucleic acid sequence encoding a knockdown component that reduces or eliminates the expression of the endogenous PKS. As used herein, the term “heterologous” refers to an element such as a gene, part of a gene or protein in a cyanobacterium which does not naturally have this element. For example, a “heterologous nucleic acid sequence” has been inserted into the host organism by recombinant DNA technology. The term “heterologous” also means a DNA sequence which appears endogenously in the cyanobacterium but is additionally present in a non-native form, for instance by forming part of a synthetic plasmid or by artificially controlling expression of the DNA sequence by a promoter which is not naturally controlling the sequence in the cyanobacterium. The knockdown component can comprise RNA transcribed from the heterologous nucleic acid that is at least partially complementary to mRNA transcribed from a PKS gene for binding to the mRNA and initiating degradation and/or inhibiting translation of at least part thereof. For example, the heterologous nucleic acid can encode a small RNA (sRNA) or an antisense RNA (asRNA) to silence the expression of the PKS gene.

The expression of the knockdown component is preferably controlled by a constitutive promoter or, as the case may be, a promoter that is at least constitutive under typical cyanobacterial culturing conditions. Suitable constitutive promoters for the various aspects of the present invention include, but are not limited to, PcoaT, Prha, Pcpc560, Ptrc, Ptrc2O-2, PA1lacO-1, L03, PnrsB, PpsbA2, PpsbA, the plastocyanin promoter and the promoters provided in the J23 library. Preferred constitutive promoters are PcoAT and Prha.

Alternatively, the genetic modification can comprise at least partial disruption or complete removal of an endogenous PKS gene. In this way, the gene may be translated into a protein which has an altered or reduced function or is non-functional. Preferably, the gene is not translated at all. It is possible that the genome of the cyanobacterial cell harbors more than one copy of the endogenous PKS gene. In such a case, it is further preferred that all copies of the gene comprise the at least partial disruption or, more preferably, have been completely removed in order to deprive the cyanobacterium of the possibility utilizing the endogenous PKS.

In the following, certain embodiments of the invention will be explained in more detail with reference to figures and experimental data. The figures and examples are not intended to be limiting with respect to specific details.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Recombinant Cyanobacterial Cells

In one aspect, the present disclosure provides a recombinant cyanobacterial cell comprising a nucleic acid construct comprising a nucleic acid sequence encoding a heterologous polyketide synthase (PKS); and a nucleic acid sequence encoding a type-II thioesterase (TE-II).

In certain embodiments, the first heterologous PKS is an apratoxin A PKS, optionally wherein the PKS is AprA. In certain embodiments, the nucleic acid construct further comprises a nucleic acid sequence encoding a second heterologous polyketide synthase (PKS). In certain embodiments, the second heterologous PKS is an apratoxin A PKS, optionally wherein the PKS is AprB. In certain embodiments, the nucleic acid construct further comprises a nucleic acid sequence encoding one or more hydroxymethylglutaryl (HMG)-CoA synthase (HCS)-like proteins. In certain embodiments, the one or more (HCS)-like proteins comprise AprC, AprD, AprE, and/or AprF. In certain embodiments, the nucleic acid construct further comprises a nucleic acid sequence encoding a third heterologous polyketide synthase (PKS). In certain embodiments, the third heterologous PKS is an apratoxin A PKS, optionally wherein the PKS is AprG. In certain embodiments, each of the heterologous PKS are Moorena bouillonii PKS.

In certain embodiments, the nucleic acid construct further comprises a promoter operably linked to the nucleic acid sequence encoding the first heterologous PKS. In certain embodiments, the promoter is an inducible promoter. In certain embodiments, the promoter comprises a PcoaT or Prha promoter. In certain embodiments, the nucleic acid construct is a plasmid.

In another aspect, the present disclosure provides a recombinant cyanobacterial cell comprising an expression construct comprising an inducible promoter operably linked to a nucleic acid sequence encoding, in the following order: Moorena bouillonii AprA, AprB, AprC, AprD, AprE, AprF, AprG; and a type-II thioesterase (TE-II) from an erythromycin cluster.

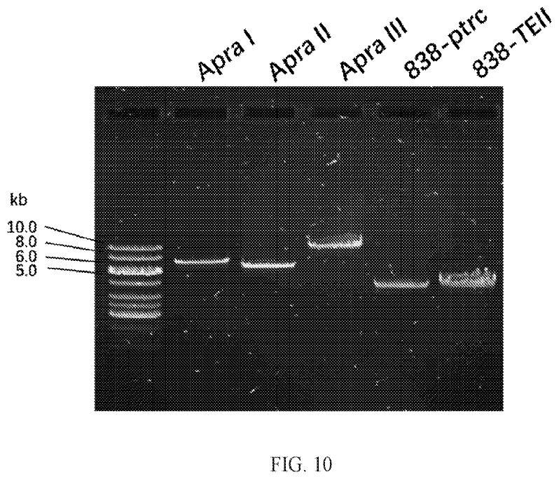

In certain embodiments, the nucleic acid sequence encoding TE-II comprises the sequence set forth in SEQ ID NO: 64 (FIG. 9). In certain embodiments, the cell is Anabaena sp. In certain embodiments, the recombinant cyanobacterial cell comprises pRL838-Apra. In certain embodiments, the recombinant cyanobacterial cell comprises pRL838-cApra. In certain embodiments, the recombinant cyanobacterial cell comprises pRL838-rApra.

In another aspect, the present disclosure provides a recombinant cyanobacterial cell comprising at least one nucleic acid construct that provides for production of Compound (I):

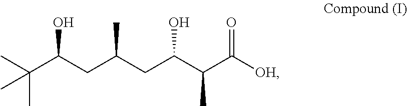 [SMILES: C[C@@H](C[C@H](O)[C@H](C)C(=O)O)C[C@H](O)C(C)(C)C]

or a salt thereof.

### Expression Constructs and Methods of Making Chemical Compounds

In another aspect, the present disclosure provides an expression construct comprising a promotor operably linked to a nucleic acid sequence encoding, in the following order: Moorena bouillonii AprA, AprB, AprC, AprD, AprE, AprF, AprG; and a type-II thioesterase (TE-II) from an erythromycin cluster.

In certain embodiments, the promoter is an inducible promoter. In certain embodiments, the promoter comprises a PcoaT or Prha promoter.

In another aspect, the present disclosure provides a method of making a chemical compound comprising culturing a recombinant cyanobacterial cell under conditions permitting the production of the chemical compound; and isolating or recovering the chemical compound.

In certain embodiments, the culturing of the recombinant cyanobacterial cell further comprises inducing with CoCl₂. In certain embodiments, the culturing of the recombinant cyanobacterial cell further comprises inducing with rhamnose. In certain embodiments, the compound is a polyketide. In certain embodiments, the compound is a polyketide fragment contained in a natural product. In certain embodiments, the compound is a polyketide fragment contained in an apratoxin natural product. In certain embodiments, the compound is Compound (I):

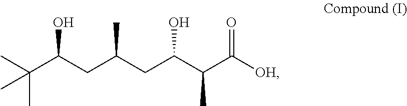 [SMILES: C[C@@H](C[C@H](O)[C@H](C)C(=O)O)C[C@H](O)C(C)(C)C]

or a salt thereof. In certain embodiments, the cyanobacterium is exposed to light and CO₂ during the method steps.

In another aspect, the present disclosure provides a method of making an apratoxin derivative compound comprising use of Compound (I):

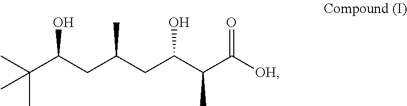 [SMILES: C[C@@H](C[C@H](O)[C@H](C)C(=O)O)C[C@H](O)C(C)(C)C]

or a salt thereof, in the synthesis of the apratoxin derivative.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Heterologous Production of the C33-C45 Polyketide Fragment of Anticancer Apratoxins in a Cyano-Bacterial Host

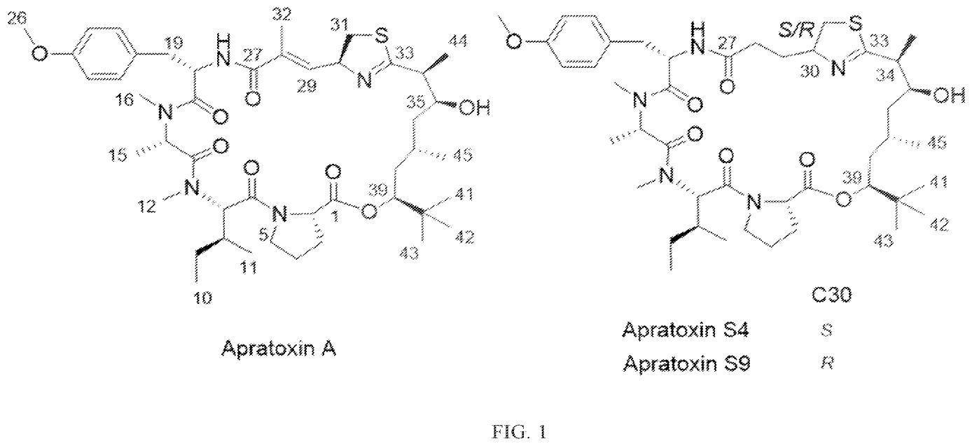

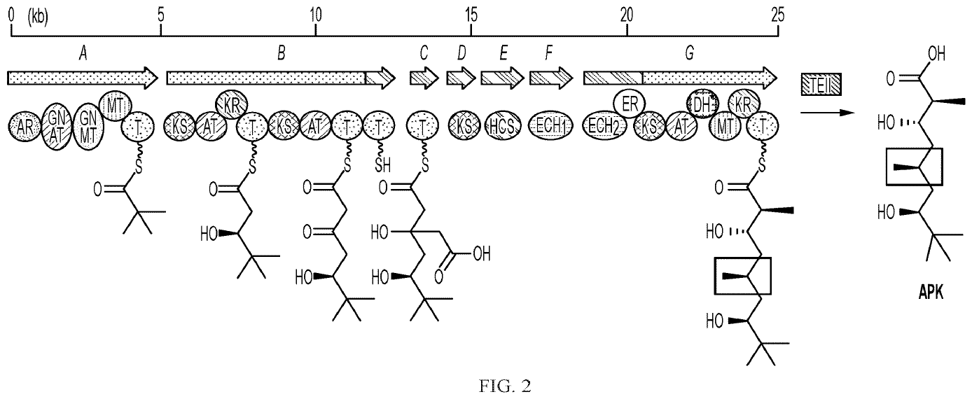

Apratoxin A (FIG. 1) is a cyclic PK-peptide hybrid produced by the marine filamentous cyanobacterium Moorena bouillonii⁵ and it possesses potent anticancer activity by inhibiting cotranslational translocation early in the secretory pathway, directly targeting Sec61α.^{6, 7} This compound serves as the starting point for preclinical investigations and medicinal chemistry campaigns to improve its activities. Apratoxin A carries two PK fragments, and the longer one (C33-C45, APK), (2R,3R,5R,7R)-3,7-dihydroxy-2,5,8,8-tetramethylnonanoic acid, has one characteristic terminal t-butyl group, two hydroxy and two methyl groups in stereochemically defined orientation (FIG. 1). Despite significant efforts in developing different synthetic strategies, available routes to the APK remain lengthy and laborious,^{4, 8} limiting their scalability and applicability for the preparation of apratoxins and analogs. Indeed, once in hand, APK could be used to generate improved synthetic analogs such as apratoxin S4 and S9 (FIG. 1) with differences in the C27-C31 unit, lacking the Michael acceptor and differing in configurations at C30 to modulate activity and selectivity.⁹ An economically feasible and simple route to APK would be a major advancement towards solving the supply problem for this promising class of anticancer, antiviral and antiangiogenic agents acting through a novel mechanism of action.^{7, 9-11}

Heterologous expression in surrogate hosts is an established strategy to produce microbial and plant natural products and analogs.¹² Escherichia coli, Streptomyces strains and yeast are common hosts in these studies. Recently, model cyanobacterial strains have received increased attention for the heterologous production of cyanobacterial natural products.¹³ For example, the unicellular cyanobacterium Synechocystis sp. PCC6803 was developed to produce the mycosporine-like amino acid shinorine,¹⁴ while the filamentous cyanobacterium Anabaena sp. PCC7120 (hereafter referred to as Anabaena) has been used to produce multiple types of cyanobacterial natural products (e.g., lyngbyatoxin A,¹⁵ cryptomaldamide¹⁶ and columbamides¹⁷). The present disclosure demonstrates the heterologous production of APK in Anabaena expressing an engineered PKS sub-cluster.

The biosynthesis of apratoxin A is deduced on the genetic basis of its 58-kb gene cluster that was previously identified through a combination of single-cell sequencing and genomic library screening.¹⁸ The cluster is comprised of 12 biosynthetic genes (aprA-L). The synthesis of APK presumably involves seven enzymes, AprA-G, whose corresponding genes span a 25-kb chromosomal region (FIG. 2). AprA is a loading PKS module for synthesizing the t-butyl terminus, functionally similar to the one in curacin biosynthesis.¹⁹ AprB is a bimodule PKS that elongates the intermediate by four carbons and stereospecifically introduces a —OH group. Subsequently, an HMG-CoA synthase (HCS)-like gene cassette (AprC-F), frequently associated with β-branching in PK biosynthesis, introduces the β-Me group (boxed, FIG. 2).²⁰ AprG then extends the intermediate chain with two carbons, introduces one α-Me group, and reduces the β-ketone to a chiral —OH group (FIG. 2). APK tethered on the thiolation (T) domain of AprG is then sequentially assembled with L-cysteine, malonyl-CoA, L-tyrosine, L-alanine, L-isoleucine, and L-proline by AprH-L.¹⁸ The elongated intermediates receive multiple in-line modifications (e.g., N-, O-, and C-methylations), and the macrolactonization via nucleophilic attack by the terminal —OH of APK generates the final structure of apratoxin A (FIG. 1).

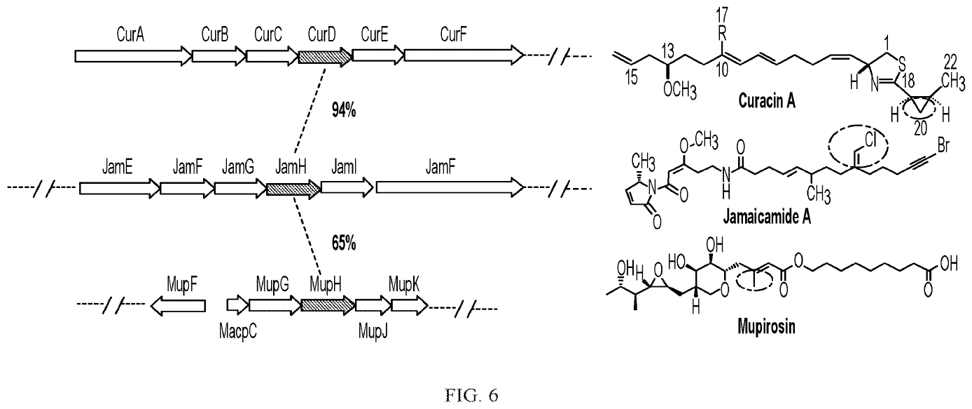

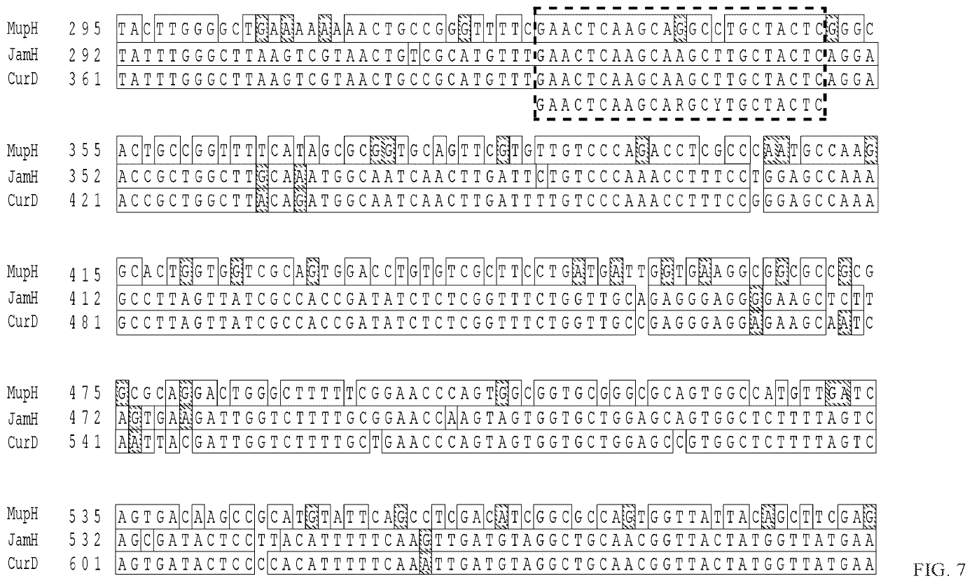

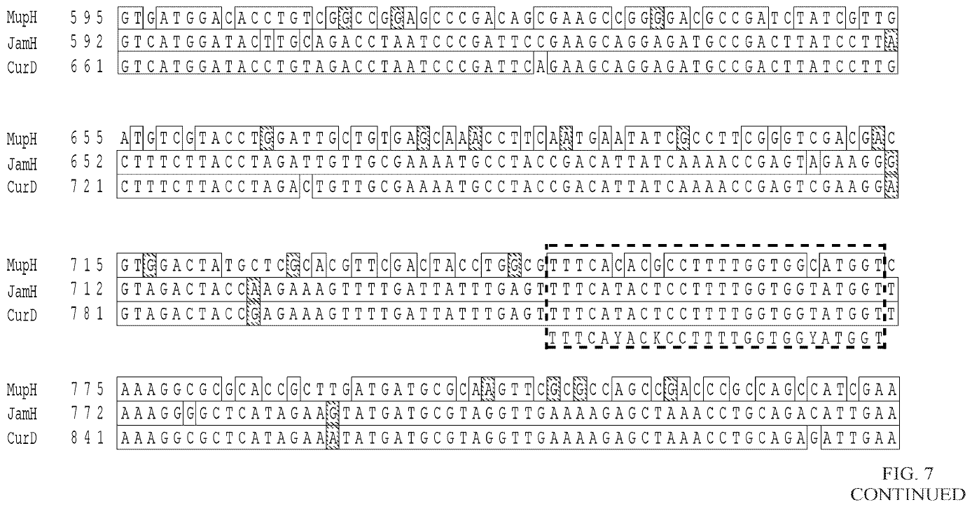

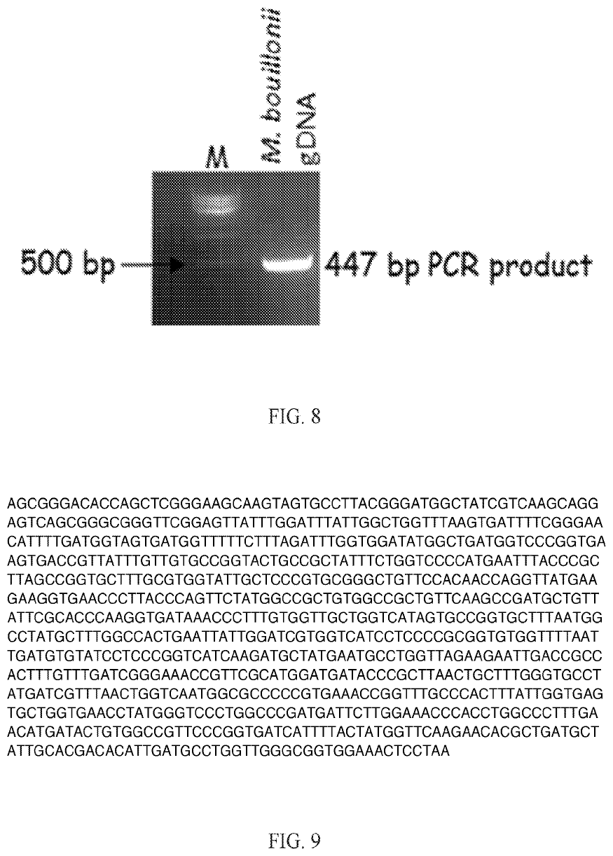

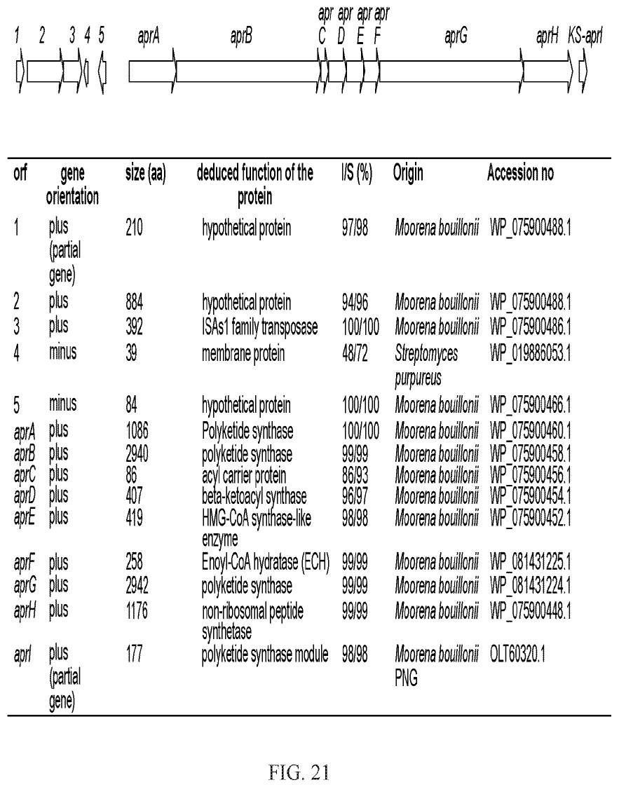

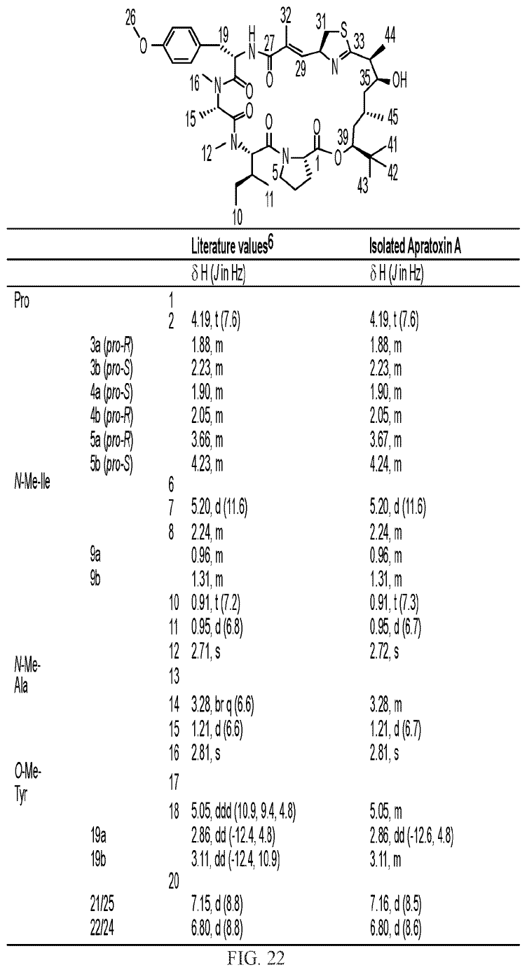

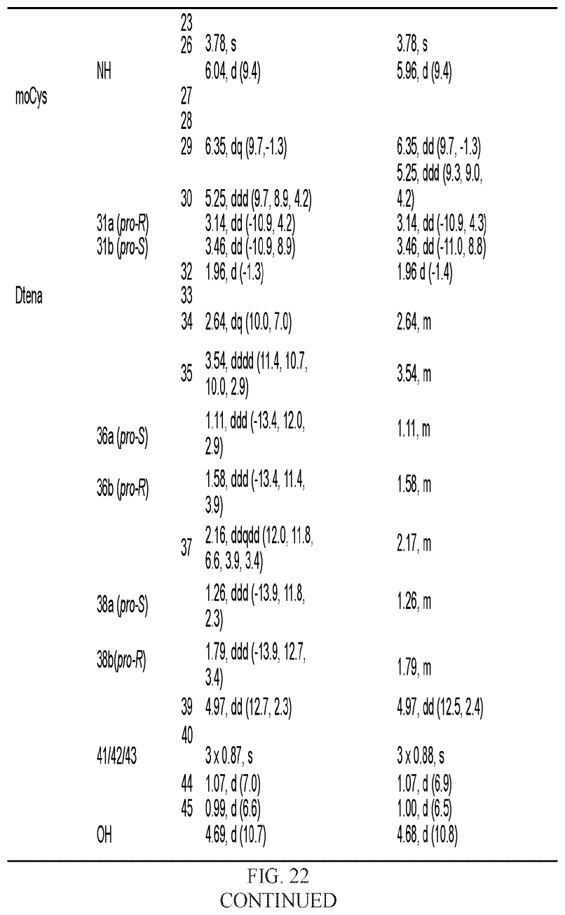

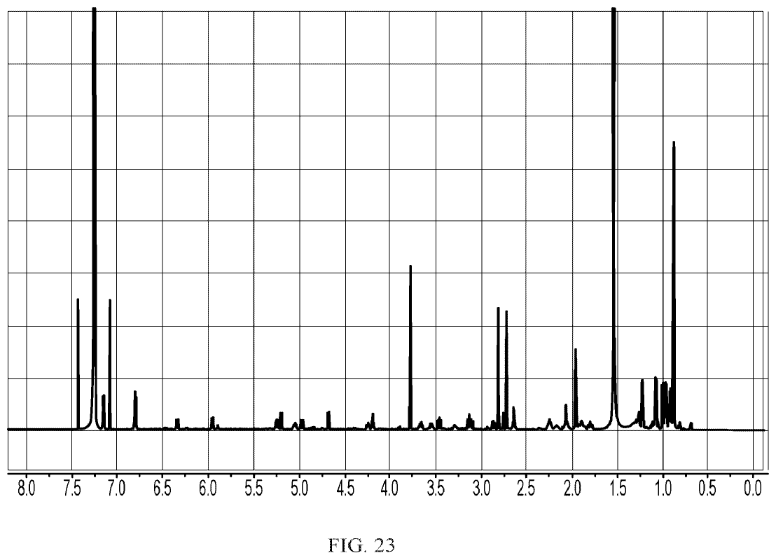

The genetic understanding of apratoxin biosynthesis sets the stage for the heterologous production of APK, which would be supported further by public access to the complete sequence information of the cluster. The APK sub-cluster (aprA-G) was identified by screening a metagenomic fosmid library prepared from the M. bouillonii sample VPG14-77 collected at Piti Bomb Holes in Guam (FIG. 5). VPG14-77 produced apratoxin A as confirmed by high-resolution mass spectroscopy (HRMS), ¹H NMR, and optical rotation analysis of the isolated compound (FIGS. 22-23). The 100,000-clone library represented >80-fold coverage of the entire M. bouillonii genome (estimated to be <10 Mb) and was archived in seven pools. One pair of degenerate primers was designed on conserved regions of HCS enzymes for the biosynthesis of curacin A, jamaicamide A and mupirocin (FIG. 6) and used to screen the metagenomic library (FIG. 7, Table 1). The primers successfully amplified the product of the expected size (447 bp) from the isolated metagenomic DNA (FIG. 8) and led to the recovery of three single HCS-positive clones. Two clones (bAprat1 and bAprat8) were identical and were not part of the apratoxin cluster, but the clone bAprat14 harbors modular PKSs, HCS-cassette, and NRPS genes (GenBank ID: MG890637.1) with gene order and domain organization highly similar to the reported APK sub-cluster (aprA-G), aprH, and the ketosynthase (KS) domain of aprI (FIGS. 2, 21). Compared with the published one,¹⁸ the APK sub-cluster reported herein carried 112-amino acid longer AprB and AprG with one extra dehydratase (DH) domain. The DH domain lacks a conserved active site motif HXXXGXXXXP, likely being nonfunctional. The notable differences between the two APK sub-clusters indicate divergence in the clusters within cyanobacterial species collected from different geographic locations. Moreover, apratoxin E was previously isolated from another Moorena sp.²¹ Compared with apratoxin A, the APK moiety of apratoxin E carries the C═C at C34-35 and lacks the C44 methyl, which suggested functional variations of AprG's DH and methyltransferase (MT) domains. The configuration at C30 in apratoxin E was 30R as in apratoxin S9 (FIG. 1).^{9, 22} Additionally, aprE-H of the cluster disclosed herein are translationally coupled as the stop codon of the preceding gene overlaps with the start codon of the succeeding gene, fixing corresponding biosynthetic enzymes with equal molarity at the translation level.

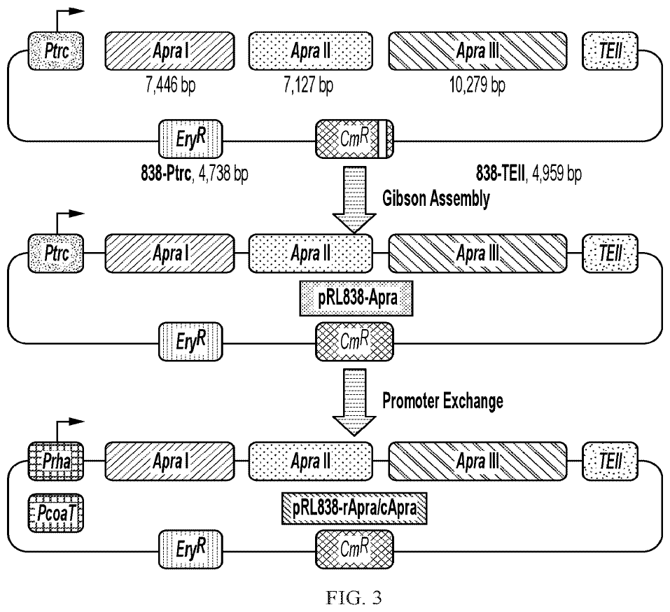

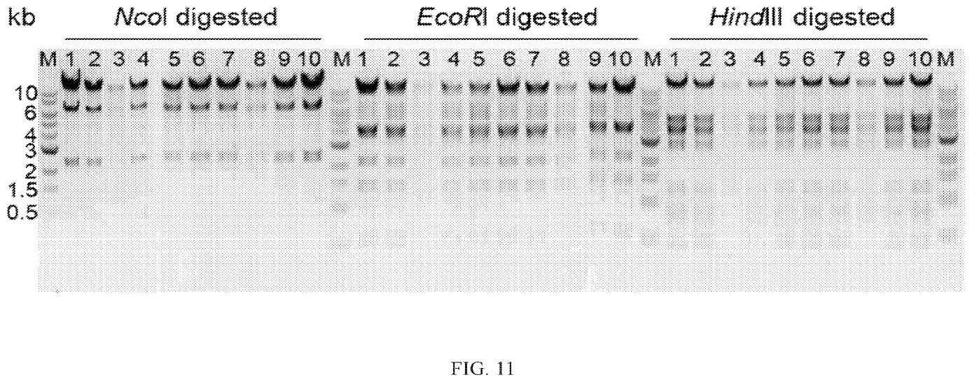

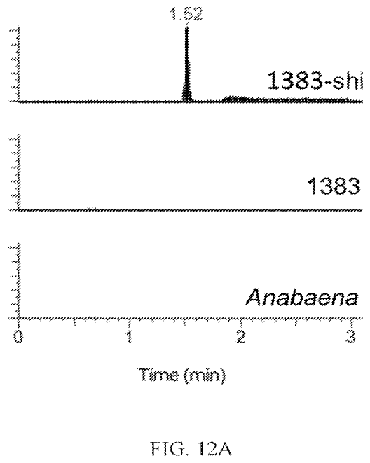

The aprA-G sub-cluster disclosed herein was engineered for the heterologous production of APK in Anabaena (FIG. 2). A replicative BAC vector pRL838 was selected with both erythromycin and chloramphenicol resistance markers (Ery^{R} and Cm^{R}) for cloning the sub-cluster,²³ and its expression was controlled by a proven strong promoter Ptrc (FIG. 3). To release the APK from the AprG T domain, the type-II thioesterase (TEII) domain of the erythromycin cluster (FIG. 9) was chosen.²⁴ Ptrc and codon-optimized TEII were first cloned into pRL838. The expression vector pRL838-Apra (-34 kb) was then constructed by the Gibson assembly using five fragments, including three fragments (Apra I, 7,446 bp; Apra II, 7,127 bp; Apra III, 10,279 bp) amplified from bAprat14 and two backbone fragments (838-ptrc, 4,738 bp; 838-TEII, 4,959 bp) amplified from the above engineered pRL838 vector (FIGS. 3 and 10). The stop codon of aprG was eliminated via polymerase chain reaction (PCR) amplification for functional co-transcription with TEII. The Cm^{R} was split between 838-ptrc and 838-TEII to facilitate the screening of clones carrying the properly assembled construct. Assembled constructs were further confirmed by digestion with three restriction enzymes (FIG. 11).

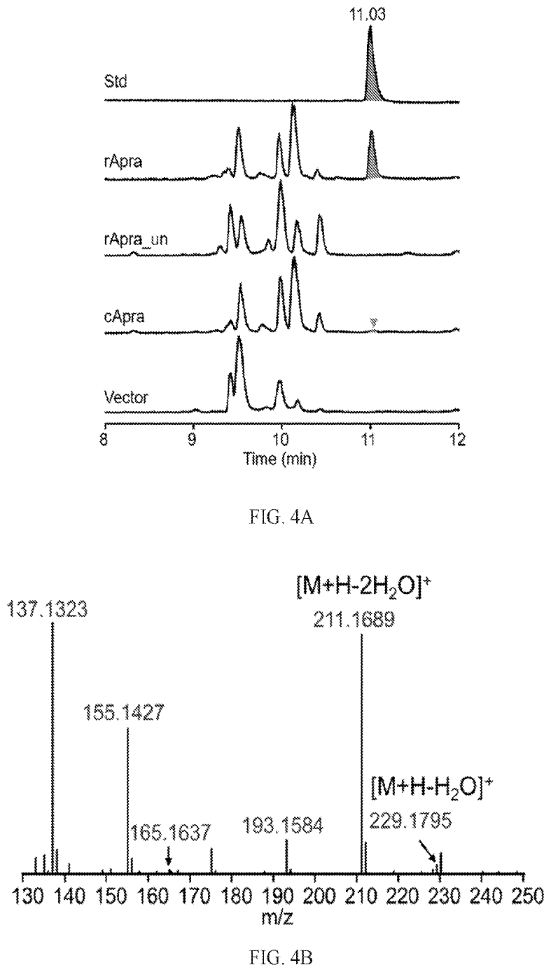

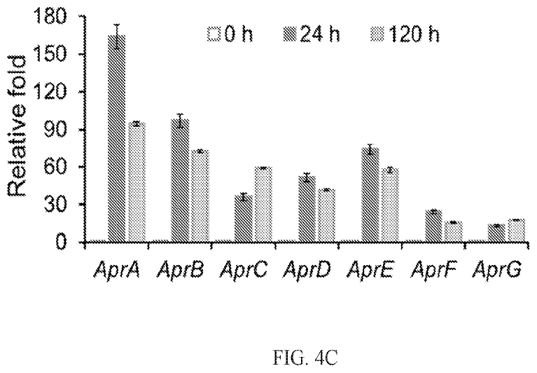

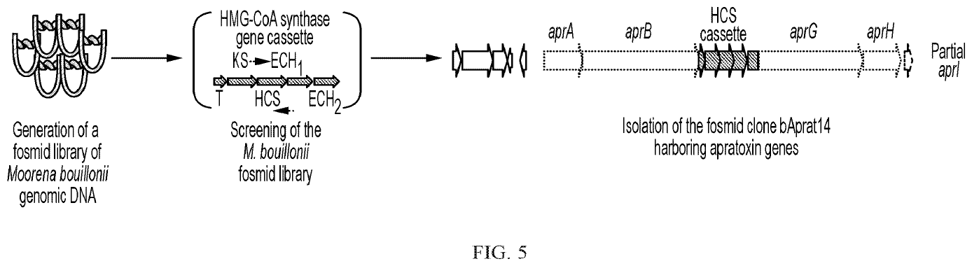

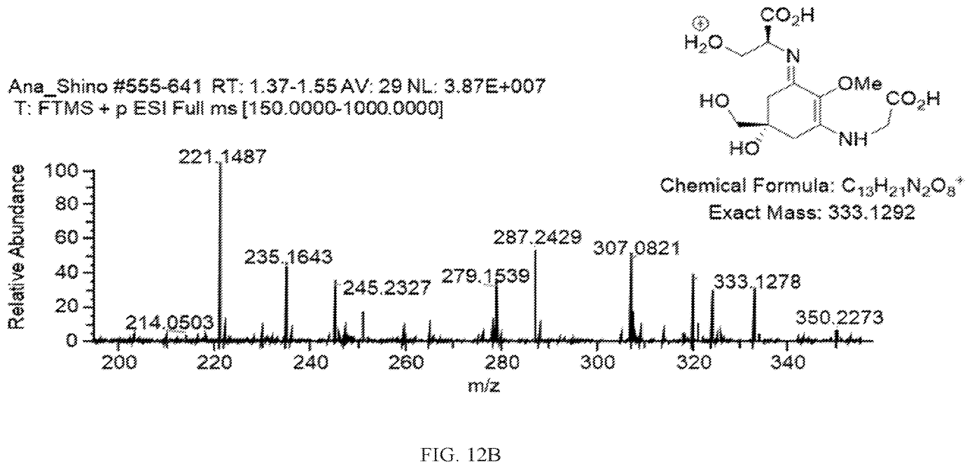

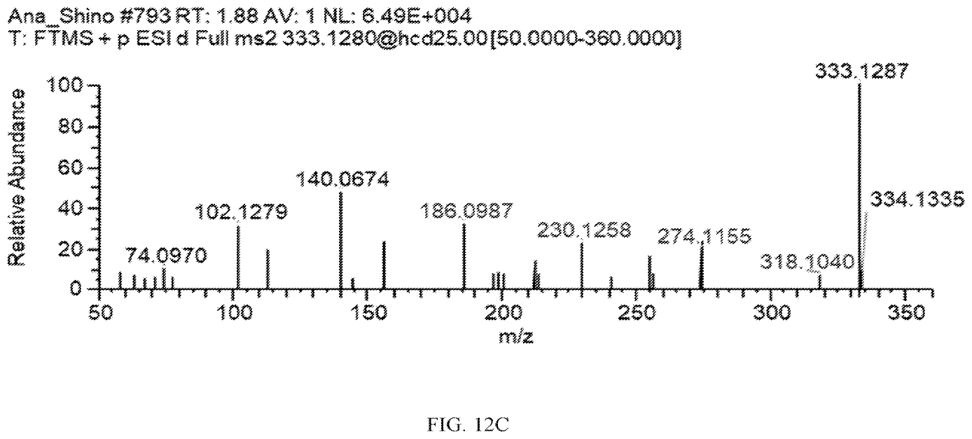

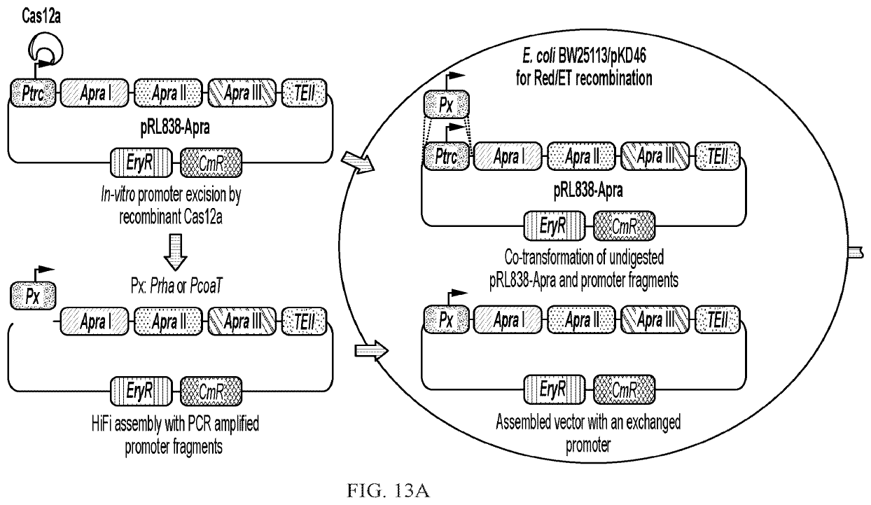

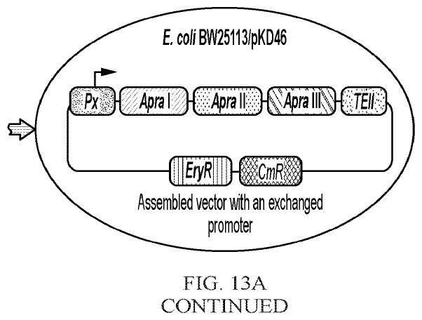

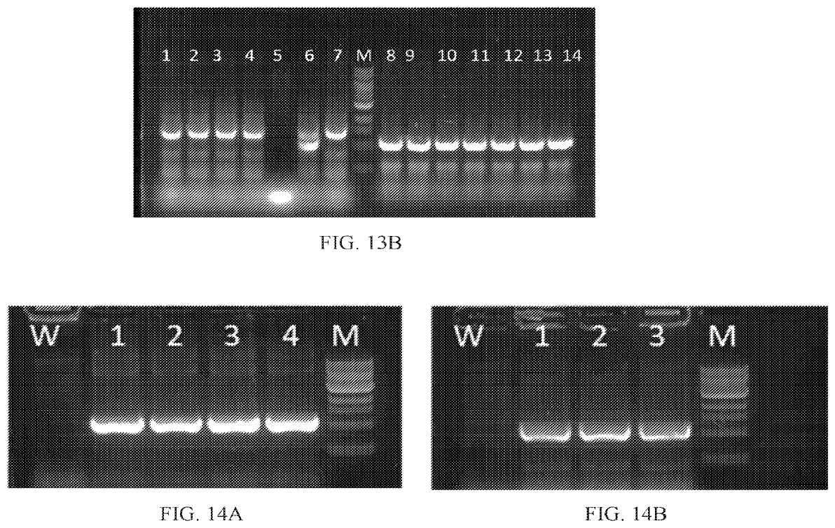

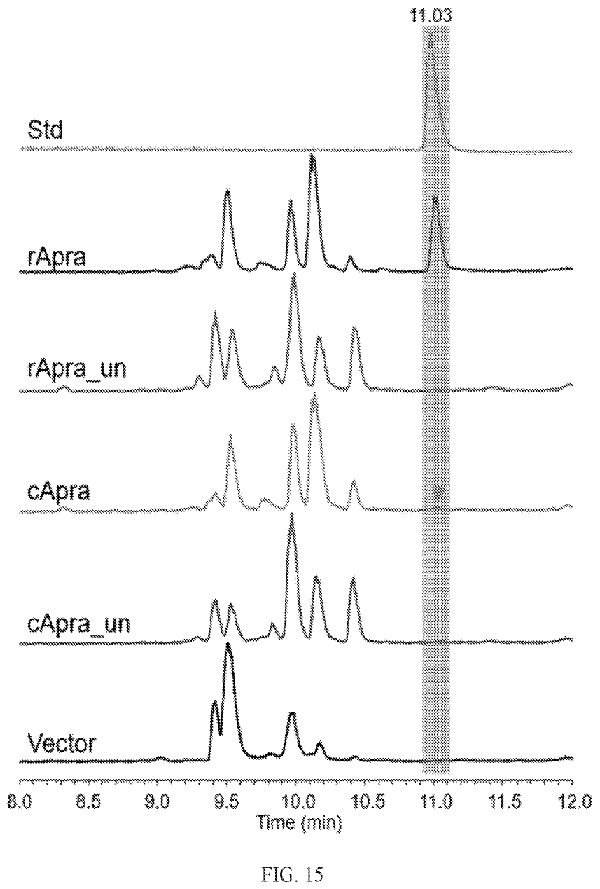

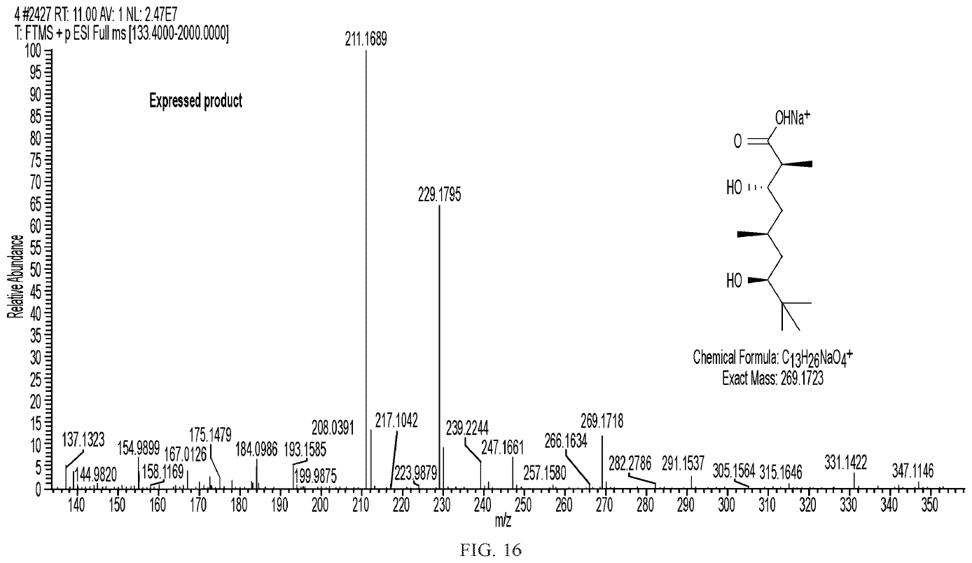

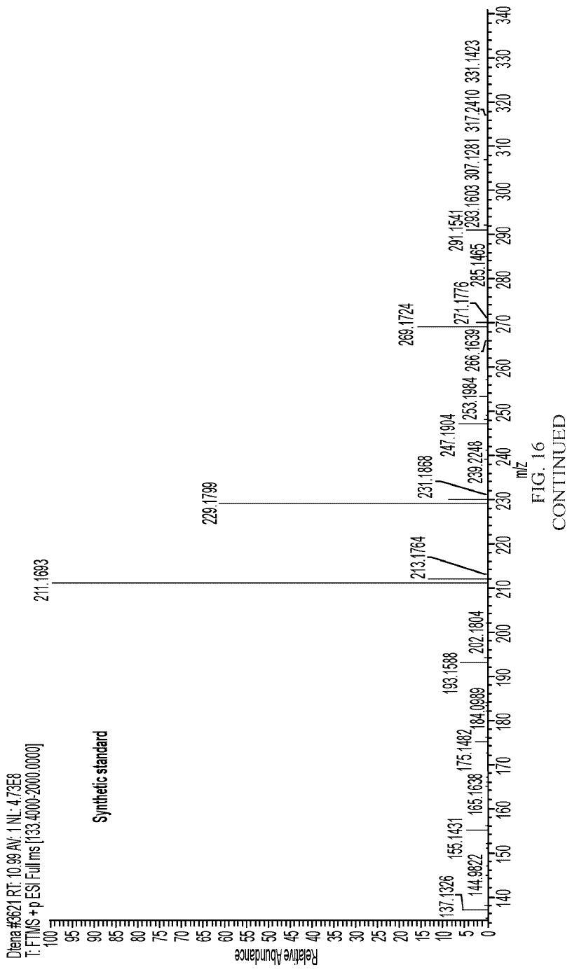

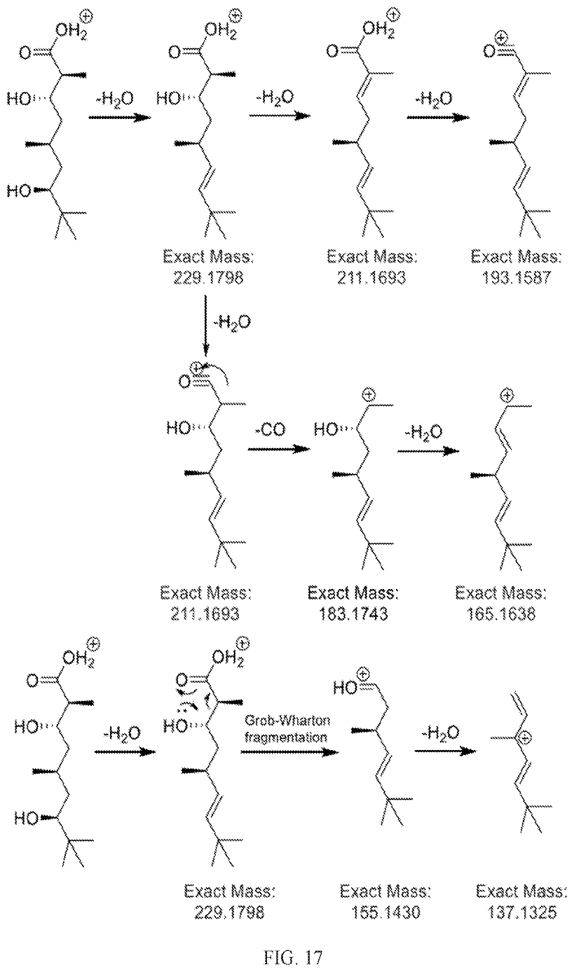

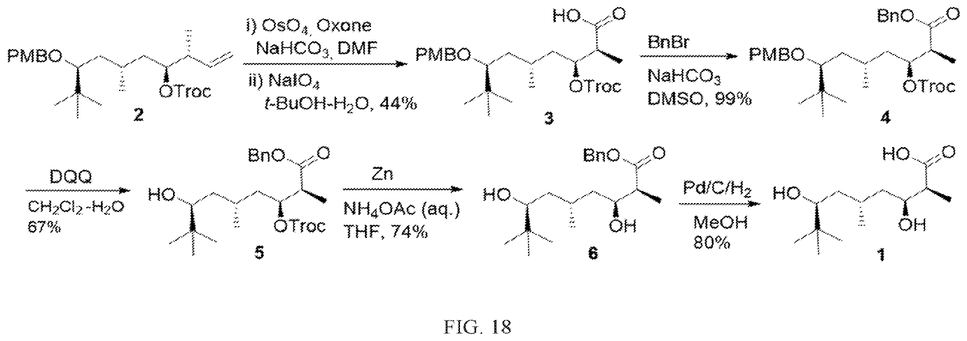

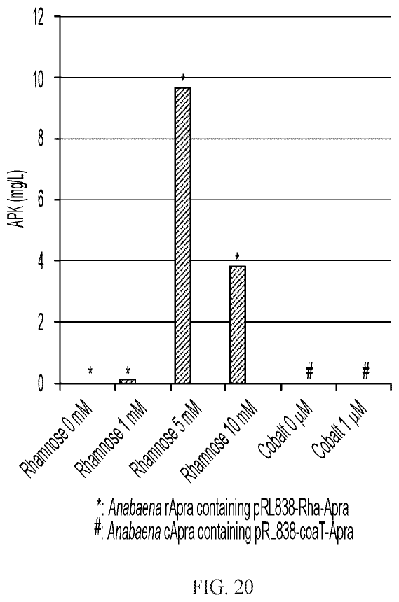

pRL838-Apra was introduced into Anabaena utilizing triparental conjugation.²⁵ However, multiple attempts yielded the same observation: transformed cyanobacterial cells gradually became less viable during segregation and selection. As a control, a construct was prepared for expressing the biosynthetic gene cluster of mycosporine-like amino acid shinorine under the Ptrc promoter¹³ in Anabaena. Positive Anabaena transformants were successfully identified, and produced shinorine in culture (FIGS. 12A-12C). These results suggested the potential toxicity of expressed APK on the host cells. Therefore, the constitutive Ptrc promoter in pRL838-Apra was replaced with a Co²⁺ or rhamnose inducible promoter (PcoaT or Prha) (FIG. 3). The promoter switch was achieved by combining in vitro Cas12a digestion²⁶ and HiFi assembly with in vivo Red-ET homologous recombination (FIG. 13A),27 and engineered constructs were identified by PCR screening (FIG. 13B). The resultant constructs carrying PcoaT or Prha were named as pRL838-cApra and pRL838-rApra, respectively, and were conjugated into Anabaena. After multiple rounds of segregation and selection, positive transformants of both constructs, named Anabaena cApra and rApra, were confirmed by PCR screening (FIGS. 14A-14B) and were then cultured in BG-11 medium. The growth of Anabaena wild type and rApra was similar but the cApra was significantly slower. After 21 days, the cultures were diluted to OD₇₃₀ of 0.1 and the production of APK was induced by 1 μM CoCl₂ or 5 mM rhamnose for 7 additional days. LC-high resolution MS (HRMS) analysis of the methanolic extract of wet biomass of Anabaena cApra and rApra identified a new peak (FIG. 4A), which was missing in those of the vector control and uninduced Anabaena rApra, as well as the uninduced cApra (FIG. 15). The peak content showed an expected [M+Na]⁺ value of APK at 269.1718 (C₁₃H₂₆NaO₄⁺; calculated [M+Na]⁺: 269.1723) in high resolution MS (HRMS) analysis (FIG. 16). Tandem HRMS analysis identified the ions of two fragments with loss of one and two water molecules, and multiple additional ions of predicted fragments (FIGS. 4B and 17). To further confirm the content identity of the new peak, the APK authentic standard was prepared from a previously synthesized intermediate⁹ in a 5-step route (FIG. 18). The structures of synthetic intermediates and the final product APK were confirmed by HRMS and ¹H and ¹³C NMR analyses. The new peak of the extracts of Anabaena cApra and rApra had the same retention time and MS/MS fragmentation pattern as the authentic standard (FIGS. 4A-4B and 19). These results indicated that APK was successfully produced in Anabaena expressing the engineered apratoxin PKS sub-cluster. This disclosure also confirmed the isolated cluster in the current and previous studies¹⁸ for the apratoxin biosynthesis. Compared with the PcoaT promoter, Prha demonstrated a better production of APK (FIGS. 4A and 20). Indeed, upregulated transcription of AprA-G was observed 24 hours and 120 hours after rhamnose induction (FIG. 4C). In addition, rhamnose at 5 mM gave rise to the highest yield (9.7 mg/L of culture), compared with 1 mM and 10 mM inducer (FIG. 20).

In conclusion, the present disclosure demonstrated the successful production of the C33-C45 PK fragment of the cyanobacterial depsipeptide apratoxin A using the filamentous cyanobacterium Anabaena as the host. The cyanobacterial platform provided a controllable means to supply APK and designed analogs for the modular synthesis of apratoxins for preclinical investigations. The present disclosure also provided the first functional characterization of the apratoxin gene cluster. The successful heterologous expression of APK depended on the use of multiple synthetic biology approaches to redesign the sub-cluster and address the toxicity issue. These approaches can find broader applications in studying entire or partial known and cryptic cyanobacterial biosynthetic gene clusters. This disclosure supports the development of cyanobacterial synthetic biology chassis for the environmentally friendly production of high-value chemicals.

### Example 2: Methods and Materials

Materials. All chemicals were purchased from Sigma Aldrich or Fisher Scientific unless noted otherwise. Q5® High-Fidelity DNA Polymerase, Gibson Assembly® or HIFI Master Mix were purchased from New England BioLabs, Inc. T4 DNA ligase, dNTP solutions, all restriction endonucleases, gel extraction and PCR purification kits were purchased from Thermo Fisher Scientific. Primers were ordered from Sigma-Aldrich. Polymerase chain reaction (PCR) amplifications were carried out using an automated thermocycler (Eppendorf 5344 Mastercycler ep 384). DNA sequencing and synthesis were performed at Eurofins. Plasmid miniprep kits were purchased from Zymo Research. A Shimadzu Prominence UHPLC system (Kyoto, Japan) fitted with an Agilent Eclipse Plus C18 column (3.5 μm, 2.1×100 mm, 90 Å), coupled with a PDA detector was used for HPLC analysis. High resolution LC-MS (HR-LC-MS) and LC-MS/MS (HR-LC-MS/MS) analyses were conducted using a Thermo Fisher Q Exactive Focus mass spectrometer equipped with UltiMateTM 3000 RSLCnano System and electrospray probe on Universal Ion Max API source. Thermo Xcalibur Roadmap™ was used to process HR-LC-MS data. Chemical structures were drawn using ChemBioDraw® Ultra. NMR spectra were recorded with a Bruker Avance II™ spectrometer.

Strains and culture conditions. Escherichia coli EPI300 (Epicentre) was used for fosmid DNA library construction and subcloning. Luria-Bertani broth (LB) was used for routine E. coli growth. Escherichia coli DH5a (Lucigen) and Escherichia coli 10 beta (New England BioLabs, Inc.) were used for routine molecular biology studies and pRL838-Apra construction respectively. When plasmid-containing clones were grown, the concentrations of antibiotics used to supplement the media were chloramphenicol 25 μg/mL, erythromycin 2 μg/mL, kanamycin 50 μg/mL, and spectinomycin 50 μg/mL when needed. Anabaena sp. PCC 7120 was provided by Dr. C. Peter Wolk at Michigan State University. All cyanobacteria strains were grown in media bottles containing 100-200 ml BG-11 medium. Chloramphenicol 25 mg/mL and erythromycin 25 mg/mL were added in a final concentration of 5 μg/ml and 2 μg/ml, respectively to the cultures of engineered Anabaena strains. All cultures were incubated at 26° C. with continuous air bubbling and under 16 hour/8 hour light/dark lighting cycle with illumination of 2000-2500 lux during the lighting period. For plate growth, BG-11 medium was supplemented with 1.0% (wt/vol) agar and 0.3% (wt/vol) sodium thiosulfate. To determine the growth curves, Anabaena strains were inoculated to 300 mL of BG-11 to reach an initial OD₇₃₀ of 0.1 and then grown under the above conditions. Measurements were taken by detecting the OD₇₃₀ on a Shimadzu UV-2700 UV-Vis spectrophotometer.

Moorena bouillonii genomic DNA isolation and fosmid DNA library construction. Moorena bouillonii environmental sample VPG 14-77 was collected at Piti Bomb Holes in Guam, USA, on Jun. 10, 2014. The sample was stored frozen in RNAlater™ Stabilization Solution. The sample was resuspended in 2× volume lysozyme solution (100 mM Tris-HCl, 50 mM EDTA and 2 mg/ml lysozyme). The mixture was kept at 37° C. for 30 minutes. EDTA and SDS were added to 100 mM and 2% final concentrations respectively. After thorough mixing, NaCl and CTAB (cetyl-trimethyl ammonium bromide) were added to a final concentration of 1.5 M and 1%, respectively. The mixture was incubated at 65° C. for 30 minutes with occasional inversion of the tube. The mixture was spun at 5,000 rpm for 5 minutes at 4° C. and the supernatant was mixed with 1 volume phenol/chloroform and kept on ice for 15 minutes. The mixture was then spun at 10,000 rpm for 5 minutes. DNA was precipitated from the aqueous phase with 0.7 vol isopropanol in the presence of 1/10 vol sodium acetate pH 5.5. The DNA pellet was washed with 70% ethanol, air-dried and resuspended in TE buffer. Fosmid DNA library was constructed using the CopyControl™ HTP Fosmid LibraryProduction Kit (Epicentre) following the manufacturer's instructions. The library was composed of 100,000 clones and archived in seven pools of ~14,000 clones.

PCR screening of M. bouillonii fosmid DNA library. M. bouillonii fosmid DNA library was screened by PCR using a pair of degenerate primers (HCS-F and —R, Table 1) targeting HMG-CoA synthase gene (HCS). PCR-positive library pools were screened further by serial dilution until single positive fosmid clones were detected. PCR reaction solutions contained 1×G buffer (Epicentre), 50 pmol of each primer, 2.5UTaq polymerase (New England BioLabs, Inc.) and 1 μl of the corresponding library pool. The PCR protocol was 1 cycle at 95° C. followed by 30 cycles consisting of 40 seconds at 95° C., 40 seconds at 58° C. and 40 seconds at 72° C., followed by a final extension at 72° C. for 5 minutes. The expected size of the PCR product was 447 bp as shown in the agarose gel analysis.

Sequencing of the fosmid bAprat14. The fosmid bAprat14 was sequenced by Illumina MiSeq platform (ICBR, UF sequencing services) and the short reads were assembled with SPAdes software.¹ The assembled SPAdes output file was additionally analyzed by SeqMan of DNASTAR software (dnastar.com/t-seqmanpro.aspx) for the second round of assembly to yield the full sequence of ~37 kb fosmid insert that carries the partial apratoxin biosynthetic gene cluster. GeneMark software was used for open reading frame predictions and gene annotation was carried out by BlastX analysis against the NCBI database. The partial apratoxin gene cluster identified from bAprat14 has been submitted to GenBank under the accession number of MG890637.1.

The construction of pRL838-Apra for heterologous expression of APK in Anabaena. The construction of the pRL838-Apra vector contained two stages: insertion of Ptrc promoter and subsequently codon-optimized TEII gene into pRL838 to yield vector pRL838-Ptrc-TEII and then assembly of DNA fragments encompassing Apratoxin PKS partial gene cluster and the backbone of pRL838-Ptrc-TEII to generate pRL838-Apra using Gibson assembly kit (NEB). The Ptrc promoter was amplified from plasmid pTrcHUM using primer pair Ptrc-FW and Ptrc-RV (Table 1) and was subsequently cloned into the multiple cloning site of pRL838 using BamHI and BsiWI to give the construct pRL838-Ptrc following standard molecular biology protocols. The insert in the construct was sequenced to exclude mutations introduced during PCR amplification and gene manipulation. Then pRL838-Ptrc-TEII vector was constructed using Gibson assembly method from PCR products generated in the following manner. DNA fragment encompassing pRL838-Ptrc backbone was amplified with primers pregb-838-FW and pregb-838-RV using pRL838-Ptrc as a template. DNA fragment encompassing TEII was amplified using primers pregb-TEII-FW and pregb-TEII-RV using codon-optimized TEII as a template. Gibson assembly and the subsequent transformation were performed following the manufacturer's protocol. Lastly, pRL838-Apra vector was constructed using Gibson assembly method from PCR products generated in the following manner. Apratoxin PKS sub-gene cluster was divided into three shorter fragments to facilitate the assembly. DNA fragments Apra-I, Apra-II and Apra-III encompassing aprA-G sub-cluster were amplified with primers gb-ApraI-FW, gb-ApraI-RV, gb-ApraII-FW, gb-ApraII-RV, gb-ApraII-FW and gb-ApraII-RV using bAprat14 as a template. pRL838-Ptrc-TEII vector was divided into two fragments to facilitate PCR amplification and to reduce the background related to self-ligation. DNA fragments encompassing 838-ptrc and 838-TEII were amplified using primers gb-838Ptrc-FW, gb-838Ptrc-RV, gb-838TEII-FW and gb-838TEII-RV from pRL838-Ptrc-TEII vector (Table 1). DNA fragments Apra-I, Apra-II, Apra-III, 838-ptrc and 838-TEII were treated by DpnI digestion to remove the intact vector before being subjected to Gibson assembly.

Promoter exchange of pRL838-Apra to generate pRL838-Rha-Apra and pRL838-coaT-Apra. The construction of pRL838-Rha-Apra and pRL838-coaT-Apra included two different approaches: in vitro fnCas12a cleavage and Gibson Assembly® and Red-ET mediated homologous recombination. In the first approach, the Ptrc promoter of pRL838-Apra was excised by in vitro cleavage using recombinant fn-Cas12a and guide RNA sets (Table 1). The FnCas12a expression plasmid pET28-FnCas12a-TEV was obtained from Jin Wang (Shanghai Institute of Plant Physiology & Ecology, Chinese Academy of Sciences, China) and transformed into E. coli KRX (Promega, WI) according to the manufacturer's protocol. The expression and purification of FnCas12a enzyme, DNA design for FnCas12a guide RNA, guide RNA transcription and in vitro cleavage of plasmid DNA were performed according to published protocols.² The cleaved plasmid was used for Gibson Assembly® with the PCR products containing rhamnose inducible promoter (Prha) or cobalt inducible promoter (PcoaT). The Prha was amplified from the pSHDY-Prha-mVenus_rhaS (Addgene Plasmid #137662) using the primers in Table 1. Similarly, the PcoaT was amplified from the chromosomal DNA of Synechocystis sp. PCC 6803 using primers in Table 1. After transformation in E. coli, the assembly of the promoter was confirmed by PCR amplification with primers in Table 1. The efficiency of this approach was ~25% based on the screening of 20 randomly picked colonies. In the second approach, the PCR products containing the Prha and PcoaT were used for direct promoter exchange by Red-ET recombination. For this purpose, the same PCR product generated previously was co-transformed with the construct pRL838-Apra in E. coli BW25113/pKD46. After transformation, the exchange of the promoter was screened by PCR. The efficiency of this approach was ~30% based on the screening of 30 randomly picked colonies. For optimal promoter exchange, the ligation mixture obtained after Gibson Assembly® following the Cas12a-mediated cleavage was transformed into E. coli BW25113/pKD46. This approach led the efficiency of promoter exchange to 60-80% based on the screening of 30 randomly picked colonies.

Genetic transformation of Anabaena. The three pRL838-Apra constructs and empty pRL838-Ptrac vector were introduced into Anabaena sp. PCC 7120 via triparental mating as previously described.³ The same procedure was applied to transform pRL1383 as the negative control and pRL1383-shi⁴ in Anabaena sp. 7120. Since pRL623 and pRL838 have the same Cm resistance, pRL623-SmR was generated by replacing its CmR with the Spectinomycin resistant marker (amplified from pRL1383a). The pRL838 and pRL1383 constructs were transformed into E. coli HB101 competent cells carrying pRL623-SmR to yield the cargo strains. The conjugal E. coli strain RP4 and cargo E. coli strains were grown in LB medium supplemented with the appropriate antibiotics overnight at 37° C. Each culture (0.75 ml) was then transferred to 1.5 ml Eppendorf tube and centrifuged to collect the cells. Cell pellets were washed with LB medium and resuspended in 0.75 ml fresh LB medium without antibiotics. Two tubes of cells were then mixed and centrifuged again to collect cells. Cell pellets were then resuspended into 60 μl of fresh LB medium without antibiotics. On the other hand, 1 ml of wild type Anabaena sp. PCC 7120 culture was centrifuged, and cell pellet was washed once with fresh BG-11 medium. The cell pellet was resuspended into 100 μl of BG-11 medium. Next, 5 μl of mixed E. coli culture and 5 μl of Anabaena sp. PCC 7120 culture were mixed gently and well and 2 μl of the mixture was transferred on the BG-11-agar plate containing no antibiotics. The conjugation was conducted under normal Anabaena sp. PCC 7120 growth conditions for 24 hours. Then, cells from the spot were spread on the BG-11 plate containing chloramphenicol 5 μg/mL and erythromycin 2 μg/mL. After single colonies appeared, colonies were selected and transferred to the liquid BG-11 medium containing chloramphenicol 5 μg/mL and erythromycin 2 μg/mL to culture under the above conditions for 10 to 14 days. The segregation and selection procedure was repeated at least three times. The final stable mutants were genotyped by the colony PCR using the primers q-ApraB-FW, q-ApraB-RV, q-ApraC-FW, q-ApraC-RV, q-ApraD-FW, q-ApraD-RV, q-ApraE-FW, q-ApraE-RV, q-TEII-FW, and q-TEII-RV (Table 1).

RT-qPCR analysis. Total RNA samples were isolated from engineered Anabaena strains using ZR Fungal/Bacterial RNA MiniPrepTM kit (Zymo Research). The quantity and quality of the isolated RNAs were determined using Nanodrop. Synthesis of cDNAs was performed with OneTaq® RT-PCR Kit (NEB) following the manufacturer's protocol (Thermo Scientific). The synthesized cDNAs were treated with DNase I (RNase-free NEB) to remove residue gDNA and were subsequently used as templates for qRT-PCR to detect the transcription levels of apratoxin biosynthetic genes using primers listed in Table 1. The rnpB gene was used as positive control and isolated RNA itself was used as a control to exclude genomic DNA contaminant.

Extraction and identification of APK and shinorine. Wild type and engineered Anabaena strains were grown in 100 ml of BG-11 medium at 26° C. with air bubbling for 21 days (OD₇₃₀ 0.5 to 3). For induced production of APK, the cultures were diluted to OD₇₃₀ of 0.1 and induced with L-rhamnose or CoCl₂ for 7 days. The culture media were then centrifuged (4° C., 3,000 rpm for 20 minutes) to collect cell pellets. The pellets were then resuspended in 10 ml cooled methanol and lysed by vertexing in presence of 0.1 mm glass beads. After centrifugation (4° C., 5,000 rpm for 30 minutes), clear supernatants were collected and evaporated under reduced pressure. The dry cyanobacterium (330 mg) was extracted using 1:1 MeOH/EtOAc to give 40 mg of crude extract for HPLC and LC-MS analysis. In case of shinorine, after 21 days of culture, the cell pellets were directly collected and processed as mentioned above.

### Example 3: Chemical Preparation of APK Standard

The synthesis of standard APK (1) is depicted in FIG. 18. The protected homoallyl diol 2 was synthesized with published protocols.⁵ The carboxylic acid 3 was obtained from the oxidation of 2 with the combination of oxidants, OsO₄/oxone and NaIO₄. To make the purification easier, the COOH group of 3 was protected with benzyl to give a fully protected compound 4. The successive cleavage of PMB, Troc and Bn with DDQ, Zn/NH₄OAc and hydrogenation separately to afford crude target 1, which was purified by preparative TLC plate to give pure product 1.

(2S,3S,5S,7S)-7-((4-methoxybenzyl)oxy)-2,5,8,8-tetramethyl-3-(((2,2,2-trichloroethoxy)carbonyl)-oxy)-nonanoic acid (3). To the solution of 2 (92.3 mg, 0.177 mmol) in DMF (1.5 mL) were added Oxone (439 mg, 0.714 mmol), NaHCO₃ (60.1 mg, 0.715 mmol), and OsO₄ (2.5% solution in t-BuOH) (22 μL, 1.8 μmol) at room temperature. After being stirred at the same temperature for 24 hours, the reaction mixture was diluted with water (1 mL) and t-BuOH (2 mL), and then NaIO₄(76.5 mg, 0.358 mmol) was added. The reaction mixture was stirred at room temperature for an additional 6 hours and poured into aqueous HCl (1M, 5 mL) and CH₂Cl₂(5 mL). The water layer was extracted with CH₂Cl₂ (10 mL×3). The combined CH₂Cl₂ layer was washed with 10 wt % Na₂S₂O₃ (10 mL×3) and brine (10 mL×1), dried over MgSO₄, and concentrated in vacuo. The residue was purified by silica gel column chromatography to give product 3 (42.1 mg, 44%) as a white solid. [α]²⁰_{D}-25.1 (c 0.28, MeOH). ¹H NMR (600 MHz, CDCl₃): δ 7.28 (d, J=8.6 Hz, 2H), 6.85 (d, J=8.7 Hz, 2H), 5.23 (dd, J=9.7, 6.6 1H), 4.78 (d, J=11.9 Hz, 1H), 4.58-4.46 (m, 3H), 3.79 (s, 3H), 3.07 (dd, J=9.4, 2.2 Hz, 1H), 2.94 (dt, J=7.1, 7.1 Hz, 1H), 1.96 (ddd, J=14.2, 10.5, 2.8 Hz, 1H), 1.82 (m, 1H), 1.50 (ddd, J=13.5, 9.4, 3.8 Hz, 1H), 1.36 (ddd, J=14.2, 9.6, 2.2 Hz, 1H), 1.26 (d, J=7.2 Hz, 3H), 1.21 (m, 1H), 1.03 (d, J=6.6 Hz, 3H), 0.93 (s, 9H) ppm. ¹³C NMR (150 MHz, CDCl₃): δ 178.6, 159.1, 153.9, 131.6, 129.0, 113.9, 94.6, 85.2, 77.9, 76.7, 74.6, 66.8, 55.4, 43.7, 39.8, 37.3, 36.3, 26.6, 26.3, 20.9, 12.2 ppm. HRMS (ESI) m/z calcd for C₂₄H₃₅Cl₃O₇ [M+Na]⁺ 563.1346, found 563.1340.

Benzyl (2S,3S,5S,7S)-7-((4-methoxybenzyl)oxy)-2,5,8,8-tetramethyl-3-(((2,2,2-trichloroethoxy)-carbonyl)oxy)nonanoate (4). BnBr (8.5 μL, 0.072 mmol) was added to the solution of compound 3 (9.7 mg, 0.018 mmol) and NaHCO₃ (6.03 mg, 0.072 mmol) in anhydrous DMF (0.5 mL) at 0° C. and was stirred at room temperature overnight. The resulting mixture was quenched with water (2 mL) and extracted with EtOAc (3 mL×4). The combined organic phase was washed with water (3 mL×4), dried with anhydrous MgSO₄, evaporated in vacuo and purified by preparative TLC plate of silica gel to give product 4 (11.2 mg, 99%). [α]²⁰_{D}: −20.8 (c 0.15, MeOH). ¹H NMR (600 MHz, CDCl₃): δ 7.37-7.31 (m, 5H), 7.27 (d, J=7.6 Hz, 2H), 6.85 (d, J=8.6 Hz, 2H), 5.24 (ddd, J=11.0, 6.4, 1.5, 1H), 5.14 (m, 2H), 4.69 (d, J=11.9 Hz, 1H), 4.55-4.46 (m, 3H), 3.79 (s, 3H), 3.02 (dd, J=9.3, 2.2 Hz, 1H), 2.96 (dt, J=7.1, 7.1 Hz, 1H), 1.94 (ddd, J=14.4, 10.8, 3.0 Hz, 1H), 1.78 (m, 1H), 1.46 (ddd, J=14.1, 9.6, 4.2 Hz, 1H), 1.30 (m, 1H), 1.24 (d, J=7.2 Hz, 3H), 1.12 (ddd, J=14.0, 11.2, 1.5 Hz, 1H), 0.95 (d, J=6.6 Hz, 3H), 0.91 (s, 9H) ppm. ¹³C NMR (150 MHz, CDCl₃): δ 172.9, 159.1, 153.9, 135.8, 131.6, 129.0, 128.8, 128.6, 128.5, 113.8, 94.6, 85.2, 78.1, 76.7, 74.6, 66.8, 55.4, 43.9, 39.8, 37.3, 36.2, 26.6, 26.3, 20.8, 12.2 ppm. HRMS (ESI) m/z calcd for C₃₁H₄₁Cl₃O₇[M+Na]⁺ 653.1816, found 653.1813.

Benzyl (2S,3S,5S,7S)-7-hydroxy-2,5,8,8-tetramethyl-3-(((2,2,2-trichloroethoxy)-carbonyl)oxy)nonanoate (5). To a solution of 4 (18.2 mg, 0.029 mmol) in a mixture of CH₂Cl₂ (0.5 mL) and H₂O (0.05 mL) was added 2,3-dichloro-5,6-dibenzoquinone (DDQ) (7.9 mg, 0.035 mmol) at 0° C. The reaction mixture was stirred at the same temperature for 1 hour, quenched with saturated aqueous NaHCO₃ (3 mL), and filtered. The organic layer was separated, and the water layer was extracted with CH₂Cl₂ (5 mL×4). The organic phase was combined and washed with brine (3×5 mL), dried with anhydrous MgSO₄, filtered, and concentrated in vacuo. This residue was purified by a preparative TLC plate of silica gel to give product 5 (9.9 mg, 67%). [α]²⁰_{D}: −18.7 (c 0.07, MeOH). ¹H NMR (600 MHz, CDCl₃): δ 7.36-7.31 (m, 5H), 5.21-5.09 (m, 3H), 4.72 (m, 2H), 3.22 (d, J=9.1 Hz, 1H), 2.94 (dt, J=7.1, 7.1 Hz, 1H), 1.84 (ddd, J=14.4, 10.8, 3.0 Hz, 1H), 1.76 (m, 1H), 1.37 (br, 1H), 1.31-1.25 (m, 2H), 1.22 (d, J=7.2 Hz, 3H), 1.15 (ddd, J=14.3, 10.6, 2.0 Hz, 1H), 0.95 (d, J=6.7 Hz, 3H), 0.86 (s, 9H) ppm. ¹³C NMR (150 MHz, CDCl₃): δ 173.0, 153.9, 135.8, 128.7, 128.5, 128.5, 94.7, 78.4, 77.1, 76.8, 66.8, 43.8, 39.5, 36.9, 35.0, 26.3, 25.7, 20.7, 12.4 ppm. HRMS (ESI) m/z calcd for C₂₃H₃₃Cl₃O₆[M+Na]⁺ 533.1240, found 533.1237.

Benzyl (2S,3S,5S,7S)-3,7-dihydroxy-2,5,8,8-tetramethylnonanoate (6). Compound 5 (9.2 mg, 0.018 mmol) was dissolved in THF (2.4 mL), and then aqueous NH₄OAc (1 M in water, 0.6 mL) and zinc powder (freshly activated with 1 M aqueous HCl) (40 mg) were added at room temperature. After being stirred at the same temperature for 1 hour, to the reaction were added ethyl acetate (3 mL) and brine (3 mL). The aqueous layer was extracted with ethyl acetate (5 mL×4). The combined organic layer was dried with MgSO₄, filtered, concentrated in vacuo, and purified by a preparative TLC plate of silica gel to give product 6 (4.5 mg, 74%). [α]²⁰_{D}: −47.0 (c 0.04, MeOH). ¹H NMR (600 MHz, CDCl₃): δ 7.38-7.31 (m, 5H), 5.16 (m, 2H), 3.80 (ddd, J=10.4, 6.9, 1.9 Hz, 1H), 3.29 (dd, J=10.1, 2.3 Hz, 1H), 2.88 (br, 1H), 2.55 (dt, J=7.1, 7.1 Hz, 1H), 1.94 (m, 2H), 1.54 (ddd, J=14.2, 10.7, 3.6 Hz, 1H), 1.33 (m, 2H), 1.21 (d, J=7.2 Hz, 3H), 1.15 (ddd, J=14.0, 9.1, 2.1 Hz, 1H), 0.96 (d, J=6.7 Hz, 3H), 0.87 (s, 9H) ppm. ¹³C NMR (150 MHz, CDCl₃): δ 175.8, 136.0, 128.8, 128.5, 128.3, 76.6, 72.0, 66.5, 46.5, 40.0, 39.0, 34.9, 26.9, 25.8, 21.5, 14.1 ppm. HRMS (ESI) m/z calcd for C₂₀H₃₂O₄[M+Na]⁺ 359.2198, found 359.2191.

(2S,3S,5S,7S)-3,7-dihydroxy-2,5,8,8-tetramethylnonanoic acid (1). MeOH (0.7 mL) was added to the mixture of compound 6 (4.0 mg, 0.012 mmol) and Pd/C (10% wt) (1.0 mg). The suspension was degassed with argon and hydrogen successively, then it was stirred under hydrogen atmosphere at room temperature for 30 min. The catalyst was removed by filtration through Celite and the filtrate cake was washed by MeOH. The combined filtrate was concentrated in vacuo and purified by a preparative TLC plate of silica gel to give product 1 (2.4 mg, 80%). [α]²⁰_{D}: −28.2 (c 0.38, MeOH). ¹H NMR (600 MHz, d₆ DMSO): δ 3.56 (br m, 1H), 3.07 (dd, J=10.0, 1.8 Hz, 1H), 2.16 (m, 1H), 1.87 (m, 1H), 1.47 (m, 1H), 1.20 (m, 1H), 1.12 (ddd, J=13.8, 10.0, 4.0 Hz, 1H), 0.98 (d, J=7.1 Hz, 3H), 0.94 (m, 1H), 0.87 (d, J=6.7 Hz, 3H), 0.79 (s, 9H) ppm. ¹³C NMR (150 MHz, d₆-DMSO): δ 176.6, 75.0, 70.0, 46.6, 40.2, 40.1, 39.8, 34.7, 26.0, 25.8, 20.9, ppm. HRMS (ESI) m/z calcd for C₁₃H₂₆O₄ [M+Na]⁺ 269.1729, found 269.1723.

LC-MS analysis. The extract was concentrated and filtered through a C18 column using ACN. All samples were prepared at standard concentrations in ACN and analyzed using a Kinetex C18 column (100×4.6 mm) with a gradient of H₂O/ACN/0.1% FA. The spectra were collected in positive and negative using a switching polarity. The synthetic standard was used as a reference for retention time and fragmentation pattern comparison. The standard was prepared at 5 concentrations (12.5, 2.5, 1.25, 0.25 and 0.125 pg/mL) to establish a calibration curve for quantification of expressed APK. All samples were injected at the same volume.

Isolation of apratoxin A from VPG14-77. The cyanobacterium was lyophilized to give 330 mg dry weight that was then extracted with EtOAc/MeOH (1:1) followed by EtOH:H₂O (1:1). The lipophilic extract was partitioned between hexanes and 80% aq. MeOH. The aq. MeOH fraction was concentrated and subjected to a silica column using DCM and increasing amounts of i-PrOH, followed by MeOH. The SiO₂ fraction eluting with 6% i-PrOH was further purified using a H₂O/ACN gradient (35% for 5 min, 35-100% for 25 min, followed by 100% over 10 min) by HPLC (gemini C18 column 150×3.0 mm, 1.0 mL/min; UV detection at 220 and 240 nm), which yielded apratoxin A (t_{R} 18.7 min, 0.3 mg). Apratoxin A: colorless, amorphous solid [α]²⁰_{D}=−99.6 (c 0.015, MeOH), reported [α]²⁵_{D}=−161 (c 1.33, MeOH)³³, ¹H NMR see FIGS. 22-23, HRESIMS m/z ([M+H]⁺ 840.4929 (calcd. for C₄₅H₇₀N₅O₈S⁺, 840.4945).

TABLE 1
Oligonucleotides Used in the Present Disclosure.

| Primer names | Sequence (5′ to 3′) | SEQ ID NO: |
|---|---|---|
| HCS-F | GAACTCAAGCARGCYTGCTACTC | 1 |
| HCS-R | ACCATRCCACCAAAAGGMGTRTGAAA | 2 |
| Ptrc-FW | AAAGGATCCCGACATCATAACGGTTCTGGC | 3 |
| Ptrc-RV | AAACGTACGGGTCTGTTTCCTGTGTGAAATTGTTATC | 4 |
| pregb-TEII-FW | GGATAACAATTTCACACAGGAAACAGACCAGCGGGACACCAGCTCG | 5 |
| pregb-TEII-RV | GGCCGCTAATACGACTCACTATAGGGAGTTAGGAGTTTCCACCGCCCAAC | 6 |
| pregb-838-FW | GTTGGGCGGTGGAAACTCCTAACTCCCTATAGTGAGTCGTATTAGCGGCC | 7 |
| Pregb-838-RV | CGAGCTGGTGTCCCGCTGGTCTGTTTCCTGTGTGAAATTGTTATCC | 8 |
| gb-ApraI-FW | GGATAACAATTTCACACAGGAAACAGACCATGCTAGATAAAATAAATCGTTATGCTCATGG | 9 |
| gb-ApraI-RV | CACTCCTGCTGTATGAATTATGCCTTTGAGTGTTGGCATTGATATCTGAATTTGTTTGAG | 10 |
| gb-ApraII-FW | CTCAAACAAATTCAGATATCAATGCCAACACTCAAAGGCATAATTCATACAGCAGGAGTG | 11 |
| gb-ApraII-RV | CTCCTGCTTCTGAGTCTGGGTTAGGTCTGCAAGTATCCATGACTTCATAGCCATAG | 12 |
| gb-ApraIII-FW | CTATGGCTATGAAGTCATGGATACTTGCAGACCTAACCCAGACTCAGAAGCAGGAG | 13 |
| gb-ApraIII-RV | CGAGCTGGTGTCCCGCTTAGAGTAATCTCCTCCCTATCTTCATTGATTTC | 14 |
| gb-838TEII-FW | GAAATCAATGAAGATAGGGAGGAGATTACTCTAAGCGGGACACCAGCTCG | 15 |
| gb-838TEII-RV | CGGCGCTGGAGAATAGGTG | 16 |
| gb-838Ptrc-FW | ATCCGGGTGCGGTAGTCG | 17 |
| gb-838Ptrc-RV | CCATGAGCATAACGATTTATTTTATCTAGCATGGTCTGTTTCCTGTGTGAAATTGTTATCC | 18 |
| Prha-F1 | TTGACAGCTAGCTCAGTCCTAGG | 19 |
| Prha-R1 | CACAATTTGCTGAATTGTGGCTTATTGCAGAAAGCCATCCCGTC | 20 |
| Prha-F2 | GACGGGATGGCTTTCTGCAATAAGCCACAATTCAGCAAATTGTG | 21 |
| Prha-R2 | TTCATTACGACCAGTCTAAAA | 22 |
| PcoaT-F | GCTTTTTAACTTGGATTTTTA | 23 |
| PcoaT-R | CTAAAGACAAGTGAGATAGCA | 24 |
| gb-Prha-F | CGAGCGGCCGCATACGATTTATTGACAGCTAGCTCAGTCCTAGG | 25 |
| gb-Prha-R | TTATCTAGCATGGTCTGTTTCTTCATTACGACCAGTCTAAAAA | 26 |
| gb-PcoaT-F | CGAGCGGCCGCATACGATTTAGCTTTTTAACTTGGATT | 27 |
| gb-PcoaT-R | TTATCTAGCATGGTCTGTTTCCTAAAGACAAGTGAGATAGC | 28 |
| apra-TC-F | TAATTATTTAGGGGATATTTGCT | 29 |
| apra-TC-R | TGAACGTAAACTGGGCAACCATAG | 30 |
| q-ApraA-F | GGGTGTATTTCTCAGAGAGGGT | 31 |
| q-ApraA-R | AGTCTCTGGTAACGTGGTGA | 32 |
| q-ApraB-F | GGAGCGCTCAAGAAGTGAAG | 33 |
| q-ApraB-R | AAGCTTTCTGGTGTGTTGGC | 34 |
| q-ApraC-F | AATGATGGTAATGGAAGAGTTATCA | 35 |
| q-ApraC-R | TGCAAATATATCAGCCAGTTCTCC | 36 |
| q-ApraD-F | AGGCCATTTGACCAAGACCA | 37 |
| q-ApraD-R | GCGCGGTCAGTTCTCTCTA | 38 |
| q-ApraE-F | GATAGCCTCAGCGTTCTGGA | 39 |
| q-ApraE-R | AGTGCTCATAGACTTGCCA | 40 |
| q-ApraF-F | ACCCTCATCAGGGGTGAAAG | 41 |
| q-ApraF-R | CTCGTTGACGGCTAAACCTT | 42 |
| q-rnp-F | GAGAGGTACTGGCTCGGTAA | 43 |
| q-rnp-R | GGCTCTCTGATAGCGGAACT | 44 |
| q-16S-F | TAAGCATCGGCTAACTCCGT | 45 |
| q-16S-R | GACGCTTTACGCCCAATCAT | 46 |

Non-limiting examples of polyketide synthases (PKSs) that can be used in the context of this invention for transformation into recombinant cells are identified below.

AprA
(SEQ ID NO: 47)
ATGCTAGATAAAATAAATCGTTATGCTCATGGGTTTGTAGCTGTACCAGTGATTTGTGCTTGTTCAGAA
GCAGGGGTATTCGAGTTACTATCACAGAAAAAATCACTGAAATTAGAAGAAATAGTAGAGCATTTAGC
AGCAAATAGTGGACATCTAATGGTAGCTATGCGACTCCTAGAGTCATTATCATTCCTATATCGCTCTCA
AGCAGAAGAATACATATTGACAGAACAAAGTCAACAACATCAAATCATCCCTAAAGCTCTGATGAGCT
TATATAAATATCCATTTGAGTTATATTTAAAAGGAGAAGTAGAAACAGGAATCAGCAATTGGATTAAT
TGTTCATCTCGAAGATGGGATACAGAAAACTCATTACTAAGTGATTTATTAGATGGAGTATTACTCATC
CCCCTGCTACTAGAACTGAAAAAACAAAACTTACTGGATGAATCAAAAAAAATATTTAACACATTAAC
AAATTCCCTTAAACAAGAACTATCAACATTATTCATCAATTTAGGATGGGCAGAAGAAAAAACAGAAG
GGCTATATTTAACAGATATAGGTAGATTTATGCGTGACAGATCCTTAAACTTAGGAACAACAGCGTCTT
ATGCTCCTATGTTGTTACAGATGAAAGAATTACTATTTGGAAATCCTCAGAGAGTATTTCAAAGAAATA
AGACTGAAAAAGAAAGACACGTAAATAGAACATTAAATGTAGTAGCAAGTGGCTTTCAACACGAAAA
GTTTTTTGCCGATACAGATAAAATCATCATATCTATATTTAACCAACAGCCAATAGAAGAGCAACCAA
GCTATATAGTAGACATGGGTTGTGGGGATGGAACGCTACTGAAACGAATATATAAAATCATCAAACAA
TTCTCTGCTAGGGGAAAAGTATTAACAGAGTATCCTATCATCATGGTAGGAGTAGACTACAATCAAGA
AGCATTAGATGTCACAGATAAAAATTTAGTAGATATTCCCCATCTAGTCATTCCAGGAGATATAGGAG
CACCAGAAAAATTATTAGAGCAATTGAAAGCACAAGGAATAGAACCAGAAAAAGTATTACATATTCG
TTCATTCCTAGATCATGACCGACCATTTATAGCTCCAAAAAATACAGAAATAGCTCAAGCACGTTCTCA
GTTAGATTATCAAGTAGTAGATGTAGATCGAGAAGGAAAGCTGATACCACCTCACATAGCAGTACAAA
GTTTAGTAGAACACTTAGAAAGATGGTCATCAATAATAACTAGACATGGATTATTGCTCCTAGAAGTA
CATAGTTTAACGCCAGCAGTAGTCAAAAAATATATAGACGAAAGTGAATCTCTGCATTTTGATGCCTA
TCATGCCTTCTCAATGCAACATTTAGTAGAAGCAGATGTGTTCTTGATGGCAGCAGCAGAGGTGGGTTT
ATTTTCACGGAAAGAAGCTTTTCGTAAGTATCCGAAAACGTTGCCATTGACTCGAATCACAGTTAATCA
TTTTGAAAAGCGTAAATATCAGATACGGTATGCAACTGTAAACGATATACCAAATCTGTTAAAGTGTG
CGACATTTAATCAACCTGTAAACGAACCCTTCTTTCAAGTTTTATTGAAACAAACTCCAACAGCACATT
TATTATTAGAATATCAAGGTGAATTGGTAGCTGCAATCTTCACAGAAACTAAAAATTCTAATGAGGTG
CTAGGAATTCGTGAGTTTTTAGTAAGGACTTCTGTAGAAAATTGGCAAGTATTAGCAAAAGATTTACTA
GAATTTGTGGAACAGTGGGGAGTAGTCAAACCAGGAATAAAAGAAATAGAAGGATTGTTAAAATACC
ATGAAGCCATCTCAAACTTTCAAAAATCAAAGTGGTATCAATCTTCAGTTTTAAACAAAAAGCTTATA
GAAAAAATAACTCTACACGAATTAGCTACTTTAGAACTATGTAATTTAATGGCTCCAGAATATGAGCT
AGAAGCCTTTGCCGCACGTTGGCTTTTGCGTGTTTTTCAAGATATGGGTGTATTTCTCAGAGAGGGTGA
ATCTTATCAGGAGTCTGAGTTGGTTTCCCAGCTAAACATCTCACCACGTTACCAGAGACTTTTAGGTGC
TTTGTTACAAATTTTGCATAAGCGTGGTATTCTAAAAATTGAGAAAGATAGAGTGTTCACATTAGCAAG
ATGTAAGACCTTTGCCTTAGAAAATATTTCGTCTGAAGTTTCTGCTTTTTATGATTATTTTTCTGAAAAA
TATCCTGCTCATTTATCCTGGTTAACAGTAGTCAAAAGGTGTCTAGAGAAATATCCTTTGATTCTACGC
GGTGAAGTTGATGTCAACGAAGTTGTTTTTACAGATGGGGATATGGAGCTATTTGCTGGACTATTTCTA
GGACATCGTGTTGCTGACTACTTTAATGAGTTGCTAGCAGATGGGGTTTGCTGGGAAGTAGAACAGCG
GTTGTTAGAAGAAAAGAGGGCACAACCTATTCGGATCTTGGAGATTGGAGCAGGAACAGGAGGTGTT
ACAGGAATATTGCTAGAAAAGCTAGCTTCTCATGCAGAGCAAATTGAATTTTGGTTTACTGATATTTCT
AGCGTTTTTACACGTTATGGTGAGAGTAAGTTCAAGCAGTTTCCTTGGGTGAAATATCAAACCTTTGAC
ATAGAAAAATCTCTTGATGCTCAGGGGATAAAGTCTGAAAGTTTTGATGTGGTAATTGCTAATAACGT
ACTCCATAACACAAAATTAATTCATCAAACCTTAAATAACAGTAACTCACTATTAAATACTGGGGGGT
TATTGGCATTACTAGAGTTTACTCAACCAATTGATATTCTTTTATACTTTGGAGGGTTGCTTCAAGGATT
TTGGTTGTTTGAAGATCCAGAATACCGACTAGAAGTTGGTTGTTTACTGAGTATACCACTGTGGCAAAA
AGTTCTCAGTGATTGTGGGTTTGATGAAATAATACCATTGGGATTACCCTGTGAGATGCACGCTCTTTC
TAAGGCAAGAGAATCTGTTATATTTGCTCGAAAGCATCAAGTACAGGAGAAAACATTTTCTGAAAAAA
TTAAACAAAATTTGACAGAAAATGGTAAGCATGGGCAGGCAGAATTCGATTTTATTAGTATTAATAAT
TCACAAGAAAGTTCATCCAAATTAGAAATTTTTGAACAGGAATGTCGAAAATTATTAAAATCTCTACT
GGGTGTTCAACGTATGGAGAGATTGCCTGGTGACACACCACTAATGGAGTCAGGAATGGATTCACTGG
AGTTGTTAGAATTTCGTGCTCTTATAGAAAGAAAGTTTGGGATTAAGTTAAAGTCTACCTTCTTTTTTA
GTTACAAAACTCTTATAGCGGTAGCAGAGTATCTTTCAGAACGGGAAGATATTAATTTTAGTTAG
AprB
(SEQ ID NO: 48)
ATGCATTCTAACCAGAGCAGTTCACTAGCAAGAATAGCCATTGTAGGTGTGGCTTGCCGTTTTCCTGGA
GCAGATACTAAAGAAGAATTTTGGCAGCTACTTAAGGAAGGTCGGGACTCAATGCAGAATCTTCCTTC
AGAGCGTTGGGGAAATCTATTTAACTCCATGAGTTCAGAAATAGATTTATCTATTCAACGCGGTGGTTT
TTTGAAAGATATAGATTTGTTTGACTCATCCTTTTTTAGGATTACTCCTAGGGAAGCGCAGTTAATGGA
CCCCCAGCAAAGGCTGTTGTTAGAATTGTCCTGGGAAGCGATGGAAGATGCAGGATATGCAAGGGATA
CATTAAAGGGTAAATCAGTAGGGGTTTATGTAGGAGTTTGTCACTATGACTACAGAAGTTTATTAGAA
AAAGGTTTGGAGACAGCCGAAATAGCTCAAATTGCTACTGGTACAGCTCCAGCAACTTTTGCCAATCG
CCTTTCCTATTTTTATAATTTTCACGGCCCTAGCTTAACTGTTGATACGGCTTGTTCCAGCTCTTTGGTA
GCAATGTATGAAGCAGTAAATGCAATACGTCGTGGGCAGTGTCAAACAGCATTGGTAGGAGGCGTTAA
TTTAATGTGTTCTCCTGTGAATAACCAAGTTTATAGTGCTGCGGGTATGTTATCTCCTGATGGAGTCTGC
CGGGTGTTTGATGCGGGTGCTAATGGGTTTGTCAGGGGAGAAGGAGGGGCAGTAGTTGTACTTAAAGA
TTATCAAAAAGCGTTGACGGATGGTGATTCTATTTATGGGGTTGTTCGCAGTGTAGCAGTGAATCATGG
AGGGCAAGCAAGTTCTTTTACAGCACCAAATCCCCAAGCTCAGGCAAAATTGTTGGAACAGGCTTATA
GGGAAGCTAATATTGATATAGAGTCGGTAGGTTATATTGAAGCACATGGTACGGGTACATCTCTAGGA
GACCCAATTGAGGTCGAGGCTTTAAATGAAGCGTTTAAAGGGTTGAGTTCAAGTGGTAAGTTACCTGC
TAATAGTTGTGGTTTGGGCTCAGTTAAAACAAACATTGGTCATTTGGAAGGGGCAGCAGGTTTGGCTG
GACTAATCAAAGTTTTGCTGTGCATGAGGTATGCAACTCTTCCATGTTCATTAAATTATCAGCAACTTA
ATCCTGATATTGAATTAGAAGAAGGTCCGTTTTTTGTGGTGGACAAGCTTCAATCTTGGGAAATAAAA
GTTGATAGGGTAGGAAAACCTTATCCACTTAGAGCAGGGTTGAGTAGCTTTGGTTTTGGCGGTACAAA
TGCTCATGTAGTTCTTGAGGAAGGAGAAAATAAGAAGGAAGGAAGAAGAGGGGATAAGGAACCTTCA
GTTCATCTATTAACCCTTTCAGCTAAAACTGAAACAGCTCTTTCAGAGTTAGTCAGTCGTTATCAAAAA
TATTTAAACACTAATGCAGAATCAGAGTTAGCCGATATTTGTTATACCGCTAATACTGGGCGAGTCCAT
TTTAACCATCGACTAGCAGCGATCGCCTCAAACAAACAGGAGTTAGTAGAAAAACTTAAGGAATACCA
AGCTGGGGAGGCAACAGCGGGAGTATTAATAGGGGAACTTGCAGAACGCATAAGAACACCGAAATTA
GGTTTCTTATTCACAGGTCAAGGTTCCCAGTATGTCAATATGGGAAAACAACTGTATAAAACACAGCC
TGTATTCCGTGAAGTATTGGATAAGTGTGATATAATATTGGAAACGGAAATAGAATGTTCTCTATTAGA
TGTTCTATATAACAAAACTACAGATTCTCAAGATTCATCTTTAATAAACCAAACAGCTTACACTCAACC
AGTTCTGTTTGCAATAGAGTATGCGCTATTTAAGTTATGGGAATCTTGGGGAATTAAACCCAGCATAGT
AATGGGTCACAGTGTAGGAGAATATGTGGCTGCTTGTGTAGCAGGAGTCTTCAATCTTGAAGATGGTT
TGAGATTAATAGCTGCTAGGGGCAGGCTGATGCAACAGTTACCTTCAGGTGGAGAAATGGTTTCTGTG
ATGGCTTCAGAATCTACAGTTAGCAGACTCCTAGAACCTCACAAAAAAGAAATAGCGTTGGCGAAGCC
CGCCGTAGGCATCGCAGCAATAAATGGACCAGAAAGCACAGTTATTTCTGGAGACTCTGTAGCAGTAA
CAGGTGTAGTAAATGACCTGGAAGCAAAGGGAATAAAAACTAAAAAACTAGAGGTATCTCATGCTTTC
CATTCACCATTAATGGAACCAATGTTAGGAGAGTTTGAAGCTATAGCTAATCAACTAACCTACAATCA
ACCTAAAATACCTATTATATCTAACGTTACAGGTACAAAAGCAGATAATACTATTGCTTCCCCTCAATA
CTGGGTTAATCATGTCGGTAAACCTGTAAGATTTGCCCAAGGAATGGAAACGCTACATCAACAAGGGT
ATGAAACCTTTATAGAAGTTGGACCGAAACCAGTATTGTTAGGAATGGGCAGGCAATGTCTACCAGGA
AATGTAGGTGTCTGGTTGCCATCATTACGTCCGGGAGTGGATGAGTGGCAACAAATGCTTTATAGCTTG
GGAAAATTGTATGTAACAGGAGTAAAAATAGATTGGTCAGGATTTGAGTCTGACTCTAGTCGCCAGAA
AGTAGCATTGCCAACTTATCCATTCCAGCGAGAAAGATATTGGATAGAAATAACAGAAAACAAACATA
AGGAACATCAAAAGTCAGAAAATATAAGTGACACTTCAATTGTTAAACTACTCACTCAAGGAAAAACA
GAAGCTCTCACTCAACAACTAGAAACAGAAGCCAAATTTTCACCAGAAGAGCTTAAACTTTTACCAGA
AATATTAGAGACATTAGCCAAACAACATCAAGAACAATTAACAGCAGTAACCATCAAAAACTGGTTCT
ACGAAATCCAGTGGAAACCCTTAGCTCAAAACAACCCCAATGCAAACATTGAACCTAGTCATTGGTTA
ATTTTAGCCGATACCACAGGAGTAGCAGAAAAATTAGCTCAAAAATTACAACAACAGGGTCATAAATA
CAGCTTAGTTTATCGAGGAGAGAGCTATCAAAGACAAGCAACAGGTACTTATCAACTTAATCCTCAGA
TTCCCGAAGCATTTGAAAAGCTGTATCAAGAAATTCAACAAAGTAGTGAAACTGCCATTACGAAGTTA
ATTCACTTGTGGAGTTTAGATGCTCCCCAATCAAAAGACTTAACCCTGGAAACCCTAGAAGAAGCTCA
ATTATGGGGATGTGGCAGCGTAGTACACCTGTTACAGACCTTAGTCAAAAACTCTAGCATTCCTGAACT
ATGGTTAGTAACCCGTGGGTCTCAATCAGTATTATCCCAAACAGAAAAAAATCTAACAGGACTAGCAG
CGTCACCCTTGTGGGGATTAGGTAGAGTAGTGTCTAATGAACATCCCCAATTATGGGGAGGATTAGTA
GATTTAGACCCACAAGCTGCAGCAGGAGACGAAGTAGAAATGCTGTGGCAATTATTAGTTAATGAACA
AGAAGAAGATAATCTAGCTGTACGGGGAGAAAATACCTATGTAGCTCGTCTGGCCAGGCAAGAACCTC
AAGAATTTCCTGAATCCCTATCCTTATCATCAGATGGTAGTTACCTAATAACAGGAGGGTTAGGAGCTT
TAGGGTTACATACTGCCCAATGGTTAGTATCCAAGGGAGCGAAAAATATTGTCTTAACTGGGCGTCGC
CCTCCCTCAGAAAAAGTAAGTGAATCTATAAAAAAATTAGAAGAAACAGGATGTCAAGTGAAGGTGA
TGTTGGGGGATGTTTCTGTTGAAGAAGACATAGGCAAAATTCTCAAACAAATTCAGATATCAATGCCA
ACACTCAAAGGCATAATTCATACAGCAGGAGTGTTAGATGATGGAACCATACAACAAATGAATTGGG
AGCGTTTTGCCAAAGTCATGTCGCCCAAGGTAAAAGGAAGTTGGCATTTACATAAATTAACTGAAAAT
CAGCCATTAGATTTCTTTGTGTGTTTCTCCTCAATAGCTTCGATGTTGGGGACTCTTGGCCAAGGAAAC
TATGCTGCAGCCAATGCCTTTATGGATGCTTTAGCTAGTTATCGTTGCAGCAGAGGATTATCAGGATTG
GCGATTAATTGGGGAGCATGGGCATCAGGGGGAATGGCTGCTCGTTTAGCAGTGGAGCATCAAAATAG
GATGCACAGCAGTGGCATAACTGAGATGGCGACCAAAGAAGGAATGTATGCCTTAGATTTACTATTAA
CAAATGAATCTGCTACAGCTCAGGTAGGTGTAGCAAGTATAGAGTGGCAGGTACTCTCAGAAAGTTGG
AGTGGTATCAAAAAAAATTCATTGCTACGAGAATTATTAGAGAAGGAGGAATGGGCAGCAAAAGATA
CACGACACCAAAAAGTTAAAGCCGAATTTTTAGCAAAATTAGAGGAGGCATCACTAGAGAAACGTCA
GGAAATTTTAACTGAACACATTCGAGTGCAAGTATCTCAGGTACTAGGTTTAAGTTCATCTAAATTACC
AGAAGTAAATGTAGGTTTTGCCTCTATGGGAATGGACTCTTTGATGACAATAGAATTAAAAAATCGAC
TGCAAAATCAACTAGGAACCAAGTTGCCTGAGACAATAGCTATCGAATATCCAACTATTGCAAAATTG
TCCTTAGGTATCAAAAAAAATTCATTGCTACGAGAATTATTAGAGAAGGAGGAATGGGCAGCAAAAG
ATACACGACACCAAAAAGTTAAAGCCGAATTTTTAGCAAAATTAGAGGAGGCATCACTAGAGAAACG
TCAGGAAATTTTAACTGAACACATTCGAGTGCAAGTATCTCAGGTACTAGGTTTAAGTTCATCTAAATT
ACCAGAAGTAAATGTAGGTTTTGCCTCTATGGGAATGGACTCTTTGATGACAATAGAATTAAAAAATC
GACTGCAAAATCAACTAGGAACCAAGTTGCCTGAGACAATAGCTATCGAATATCCAACTATTGCAAAA
TTGTCCTTATGTATAGAGGAATTAATGGGATGGAAGACAACAGAAATTGACCCATTATCTGAAGAAAC
AAGATTAGAGATGATGGAGCGCTCAAGAAGTGAAGCAATAGCCATCATCGGCATAGGCTGTAGATTTC
CCGGAAATGCCAACACACCAGAAAGCTTCTGGCAATTGTTATCCAATGGCAAAGACTCCATTACAGAG
ATTCCCCTAGAACGTTGGGATCTAGATTCCTACTACGACCCCAACCCTGATACTCCAGGGAAAATGTAT
ATCCGTCACGCAGCATTAGTAGAAAAAGTAGATCAGTTCGACCCACGATTTTTTGGAATCTCTAACCG
AGAAGCTTATAGTCTTGATCCACAGCAACGCTTCATTTTGGAAGTAACTTGGGAAGCTTTAGAAAGAG
CTGGTATTAACCCTCAACAATTAGAAAACACTCAGACAGGGGTGTTCCTGGGTATCGGTCAAAATGAT
TACGCGAATTTAGGTTTTCACCAAGCAGCCGAAGATATAAGCCCTTACGATGCTACAGGAAATTTGTTT
TGTTTTGTAGCAGGTAGGTTATCTTACTTTTTAGGAACGCAAGGTCCATCAATGGCAATAGATACAGCT
TGTTCATCATCTCTAGTAGCTATCCATGAAGCTTGTGAGAGTCTGCGTCAGGGTGAGTCCAACTTGGCT
TTGGCTGGAGGAGTTCAACTAATTCTCTCTCCCGAAGTAACAACCGCACTATCAAGATTAAAGGCTCTA
GCACCTGATGGTAAGTGTAAAACTTTTGATGCTGCTGCTGATGGTTTTGGTAGGGGAGAGGGATGTGG
CATTGTGGTACTGAAGCGTTTGTCGGATGCGCTCAAGGATGGGGACAGGATATCAGCAGTAATTCCTG
GTTCGGCTGTTAACCATGATGGACCAAGTAGCGGAATGACAGTACCGAATAAACTGGCTCAGGAAAA
ACTGATTCAGAAAGCTCTCAAAGCAGCCAAGGTAAAACCGTTACAAGTGAGTTATGTGGAAGCTCATG
GTACAGGAACTTCTCTAGGAGATCCTATGGAAGTGAGAGCTTTAGCTAGGGTGTTTGAGGAGGGACGT
GATCAGGAAAATCCATTGAACATCGGTTCAGTTAAAACTAATATCGGTCATCTGGAAGCAGCAGCTGG
AATAGCAGGTATGATTAAGGTGATTTTGCAATTGCAACATCAGGAAATTGTGCCCCATCTGCATTTTGC
TAATCCTAACCCCTATGTTGATTGGGAGAATATGCCTCTACAAGTACCGACTCAACTGACTCCTTGGTT
GTCGAAAGGGGAGAAAAGGGTGGCAGGAGTTAGTTCTTTTGGTATGAGTGGTACGAATGCTCATATAG
TTTTAGAAGAGGCTCCTATTGAAGTCAGAAGGCAGAAGTCAGAAGTCAGAAGTGAAGAATATCTAGA
ACGTCCGGTTCATATACTAACTCTGTCGGCCAAGACTGAAAAAGCACTAGAAGATTTAGTTAATAGTT
ATGAAAGTTATTTAGAAGCCGAAAATAATGATAATTATTTAGGGGATATTTGCTACACAGCCAACATC
GGGAGAGCAAAATTTGACCACAAATTAGCAGTGGTTACTTCTGATAAACAAGAGTTATTAGAGAAACT
CAAACAATATAAACAAGGTGAGAATGTTGCTGGAATCTTTTCAGGAAAACAAATAAGTGAAACTAGA
ACAAAAATAGCCTTTATATTTACTGGTCAAGGTTCTCAATATCTGCAAATGGGAAGGCAGTTATACGA
AACTCAACCCACTTTTCATAAAATTATTGACCAGTGTAGTGAAATGCTGGTAAAATATTTAGATGTTTC
TTTATTAGATATACTTTATCCAGTTGAGGTTAAAGATGAAAGTTCGACTTTGATAGACCAAACAGCTTA
TACTCAACCCGCTATATTTGCCCTTGAATATGCCCTAGCTAAATTATGGGAATCATGGGGTATAAAGCC
AGATGTAGTCATGGGTCACAGTGTAGGAGAATATGTGGCAGCAACAGTAGCAGGGGTATTTAGTTTAG
AAGATGGTTTAAAATTAATAGCCATGCGGGGACAGTTGATGCAAAAGTTACCCTCCGGAGGTCAGATG
GCATCTATAATGGCATCAGAATCTCAGGTAATAGAGGCGATAAAAGAATATAGTTCCCAAGTAACAAT
AGCAGCAGTTAATGGACCAGAAATTATAGTAATTTCAGGTGAGAGTCCAGCCATTTCAAAAATTTGTA
GTAAATTTGAATCAGAAGGAGTCAAGACCAAGGCGCTACAAGTATCTCATGCTTTCCATTCCCCATTG
ATGGAACCAATGTTAACAGAATTTGAAGCAGTAGCCAAAGAAATATCCTATAATCATCCCCAAATACC
ACTAATATCAAATGTTACTGGTCAAGAAGTAAATGGAGAAATAACCACTGCTGAATATTGGGTGCGTC
ATGTGCGTCAACCAGTAAGATTTGCGGAAGGGATGGAAACTTTACACAAACAAGGTGCTGAAATTTTC
CTAGAAATAGGCTCAAAACCGATACTTTTAGGTATGGGTCGTGAGTGTCTCATGGGAGAAAAAAAACT
ATGGTTGCCCAGTTTACGTTCAGGAAAACCAGACTGGTTACAAATGCTACAAAGTCTGGGGCAATTGT
ATGTACGAGCAATAAAAATTGATTGGTTAGGATTTGATCGGGATTATTTCCGTAATAAGGTAGAGTTA
CCAACATATCCTTGGCAACGAAAAAGGTATTGGATAACAGATATTAGACAACGGAAAAGTCAAGACA
AAAAAAGCATAACATCTGAAAAAAAAGTACAACTTGATGGAGTAAATATTCAACTAAAGGAAATACA
AATGAACGACAAAATCTTACAGCAACCAAAACTAAAATTATCAGATCCAGAATCACTATATTTATCGA
ATGCTGAATCAACTATAGAGGTTCCAACAAAAGCAGTTCAAGTAAAACCTGCTGCTGATATTGAATCT
GAAAATATAACTCAATTGAATAGCCTTGATAGAGATGTGACTCAAATCATAGAAACCCTCAAAGAAAG
TTTGGCAGATGCTTTATATGCAGATATAAGTGAAATTGAAGAAGACAAAAAGTTTGTTGATTTAGGTTT
AGATTCTATTGTGGGAGTAGAATGGATAACTAACATCAATAAAATCTACAATTTAAATATAAAAGCTA
CTAAACTATACGATTACCCTACTTTGCCAGATTTGGCTAAATATGTCGCTCACACCCTATCAGCTCAGG
GTAGAAATATTGATGTAGAGCGATCGCCATCTGACTCAAGTCAAGCAAGCATTAGCAAGCAATCTCAA
CTAACTGATACCCAGAGCAATTTCTCACAAGTCAAGGAAATTTTGAAACAACAATTAGCAGATGCTCT
GTATGCGGATATTAGTGAAATTGAAGTAAACAAGAAGTTTGTTGATTTAGGTTTAGATTCTATTGTTGG
AGTAGAATGGATAACTAATATCAATAAAACCTACAATTTGAATATCAAAGCCACTAAAATATACGACT
ACCCTACCTTATTAGATTTCGCAAAATATATTAACCAGGAAATTTATTCTACAGGAGTAAGTAGATTTT
CAACGGAGCATAAAGAATTTAATCAAAAAGACTATTCATCTGGAGGTTCGCAAGAGGAAATGACACA
AAAATTACGATCAATATTGAAGAAAGTGGCCAATAAAGAATTAACAGTTCAGGAAGGAAATAAAATG
ATTCAACAAATAAAAAATCAATTAAAGTAA
AprC
(SEQ ID NO: 49)
ATGAACAAAGAGCAAATATTTCAAATAATAAAAAAATATACCTACGAAATAGCACCAGAATTAGAAG
AAGTACCTATATCACCTACTGATAGTCTCAAAAACTTGGGGATTGATTCAGTAAATAGGGCAGAAATT
ATAATGATGGTAATGGAAGAGTTATCATTAAATATCCCGCGTATTGAATTAGCTGGATCCAAAAATAT
AGGAGAACTGGCTGATATATTTGCATTCAAATTAGAAGCAATAAATTCACAAAGCTAG
AprD
(SEQ ID NO: 50)
ATGAGTAACATAGAAATAACAGGTATGGGCATTGTTACTTCCATTGGTCAAGGAGTTGCTACTTTTAAA
GAAGCTCTGTTATCAGGGAAAACTCAATTTGCTTATCTAAAACAGCCAGGACGTGAAAGCATCAAACC
ATTTATTGGTGCCGAAATTCCCGATATTGACGCCAAAACCCTATTTCCTGAGTACAGTGGACTGTTACG
TACTGCTACTAAGAGCGCCCAAGTTGCAATAGTAGCTGTCGCTGAAGCCTGGCAAGATGCTCAACTCA
CCTCCAGCCAAGTTAACCCAGAACGAGTAGGATTAGTTGTAGGTGGTTCAAATTTACAACAACGTTAT
CAGCAGCAAACCTGGCAACGTTATCACTCACGTCAGGAGTTTATACGACCAACTTATGGTCTGACCTTT
TGGGATACAGATATATTAGGTCTGATTTCCCAGTGTTTTCAGATTCAAGGTGAGGGCTATAGTGTAGGT
GGAGCTTCGGCCAGCGGAGCAGTTGCTATAATTCATGCTGCTCGTCAAATTTTAATGGGGAACAGTGA
TGTTTGCATTGCTCTGGGAGCTTTATCCGATCTATCAGGGTATGAATTCCAAGCACTAATGAATTTAGG
TGCAATGGGGAGCGAACGTTTTGCCGATCGCCCAAACCTTGCTTGTAGGCCATTTGACCAAGACCATG
ATGGCTTTATTTATGGAGAGGGTTGTGGAGCAGTGATTTTAGAGAGAACTGACCGCGCTCAACAACGG
GGCGCTCAATCCCATGGCCAACTTAAGGGTTGGGGATTAACTTTAGATGGGAACCGTAGTCCCGAACC
TTCCCAAAAAGGGGAAGAACGGGCTATAAATACTGCTTTAGCCATGGCAGACCTTCAGCCAGAGAGTA
TTGATTATGTGAATACCCACGGTACTGGTTCCCCCCTTGGCGACAAAACAGAAGTGGCAGCTTTCAAAT
CAGTAGGACTTCAGCATTGCCTATTTAATTCAACAAAATCTTTAATAGGCCACTGCTTAACTGCTGCTG
GTGTAGTGGAAGCGATCGCCACCATATTACAAATGAAGTTTGGTTTTTGTCATCCAACTAAAAATTTAG
TTAATCCTATTGATACCAGTCTCAATTGGGTGAAAGAAACTTCTGTTCAAGCTGAGATTAAATATGCTA
TCAGCAACAGTTTTGGTTTTGGGGGTATCAATACAGCTTTATTAATTGGACAGGAGTAA
AprE
(SEQ ID NO: 51)
ATGCAACAAGTTGGAATTGAAGCACTAAATGTATATGGGGGTTCAGCTAAACTAGATGTGCGAATGCT
AGGCGAAGCACGTCACTTAGACATGACTCGCTTCGATAATCTTCTGATGAAAGAGAAGACAGTTGCTA
TGCCCTATGAAGATCCAGTTTCTTATGCTATTAATGCAGCAAAACCTATTATTGATAGCCTCAGCGTTC
TGGAAAAGCAACAAATCAAAATGGTGATAGCTTGTAGCGAATCTGGCATTGATTTTGGCAAGTCTATG
AGCACTTATATCCAGGATTATTTGGGACTAAATCGTAACTGTCGAATGTTTGAAATAAAGCAAGCTTGC
TACTCAGGTACAGCAGGTTTACAAATGGCATTCAATTTAATTTTGTCCCAAACCTGTCCAGGGGCTAAA
GCTTTGGTTATAGGAACTGATATATTTCGGCCTGTTGTTGTAGAGGGAGGAGAAGCTCTCAGCGAAGA
TTGGTCTTTTGTGGAACCAAGCAGTGGTGCTGGAGCAGTAGCTATTTTAGTCAGTGATGTTCCTAAAAT
TTTCCAAGCTGATGTAGGATGTAATGGTTACTATGGCTATGAAGTCATGGATACTTGCAGACCTAACCC
AGACTCAGAAGCAGGAGATGCAGATTTATCATTGCTATCTTACCTAGATTGTTGTGAAAATGCTTACCG
GGAATATCAAGATCGAGTAGAAGGAGTAGATTACCAAAAAACTTTTGATTACTTGAGCTTTCATACTC
CTTTTGGGGGTATGGTGAAAGGGGCTCACAGAAGTATGATGCGTAAGTTTAAAAGGGCAAAACCTGTG
GAGATTGAAGAGGACTTTCAGAAACGGGTGATGCCGGGATTAGTCTATTGTCAACAGGTGGGTAATAT
TATGGGAGCTACAGTATTTTTATCTTTAGCTAGCATGATTGATAATGCAGATTTTAGCAAAGCCCGACG
AATTGGTGTATTCTCTTATGGCTCTGGTTGTTGTTCGGAATTTTATAGTGGAGTCGTCACCCCGCAAGG
AAAGGAAATTCAAGCTCAACAAAAAATTTCATCACAGTTAGCAATGCGCTATTCCTTAAGTATGGAAG
AATATGAGCAGTTACTAAGTCATAGTTCGGCAGTTGCCTTTGGCACTAGAAATGTTACCCTAGATTACA
AACTATTTCCTGGTGTGTGGGAACAAATTGAAGGTAAAGGTCGCTTAGTGCTGAAAAGAATCAAGGAA
TTTCACCGAGAATATGAATGGGTATAG
AprF
(SEQ ID NO: 52)
ATGGGTATAGCCATGAGTTATCAAACCCTGAAAATCAGTTATCAAGATGTTGTACAAAGGATTCAGAT
ATATCGACCTGAATCTAACAATAGCATCAATAGTCAATTAACGATGGAATTGTTGTCAGCTTTGCAAGC
TGCTGAAGCAGAGGAAGTTGTTAAAGTAGTGATATTAGAAGGACTACCTGATGTATTTTGTACAGGGA
TGGATTTTGAAGAAGTGGCAACAGCAAAACAATTTGATCCAAAAGCTAGTGCTAATGGTTACTACAAT
ATTTTGAAACAAATGTCTCAAAGTAGCAAAGTAATTTTGTCACTTGTGCGTGGTAAAGTACAGGCAGG
GGGAGTTGGTTTAGTAGCAGCAAGCGATCTAGTTATTGCTGATGAAACGGCAACTTTTGTTTTATCAGA
ATTATTATTTGGATTATTACCAGCTTGTGTATTGCCTTTTTTGATTCGTCGAGTGGGATTTCAAAAAGCC
TACCGTTTAGCACTAACGACTCAAGCTATTTCAGTATCAGAGGCTGATAAGTGGGGATTAATAGATGA
ATATGGCAGTAATATTAATCAGTTAATAAGTAAATATATTCGACGTTTAAAGTATTTACCCTCATCAGG
GGTGAAAGAGTTAAAAAATTATATTAATCAGTTATGGATTATTCAGGCAGAAACTCAAGGTTTAGCCG
TCAACGAGATTTCTAGCTTAATAGCAGAACCTACTGTTCAAGAAAAAATTAAACGTTTTCAAAAAGAA
GGATTATTTCCATGGCAAACCTAA
AprG + type-II thioesterase (TEII) domain of the erythromycin cluster (TEII)
(SEQ ID NO: 53)
ATGGCAAACCTAAATCTTAATTTGGACTTAGTAGAGGGCAACTCTGATGTAGTACAACTGGTGGAGTT
GGGTAATGGTGTCGTGCAAATCACGATGAAAGATGAAGAAAGCTGCAATGGCTTTTCTCCTGGAATAA
TTGAAGGATTATACAAATGTTTTGGTGCAGTTGCTCAAAACCAAAGTTATAAAGTAGTAATTTTGACAG
GCTATGGAAATTATTTCTGTTCGGGAGGGACAAAAGAACGGTTAATCAGTATTTGGAAAGGAGACAGC
AAATGTAATGATTTAGATTTTTTTAGAATAGCATTAGATTGTGAAATACCAGTAATTGCAGCTATGCAA
GGTCATAGTATTGGCGGTGGTTTGGTTTTGGGATTGTATGCAGATTTAGTAGTGTTAAGTCAAGAAAGT
ATTTACACTACTAATTTTATGAAGTATGGTTTTACTCCAGGTGTCGGATGCACGTTAATTCTCCCTGAG
AAATTCGGTGCTTTAGGGTTTGAAATGATGTATACTGCCCAAAATTATCGAGGGAAAGAATTAGCTGA
ACGGGGTGTTTCTTTTCCAGTTGTACCGAGAAAAGATGTGCTAGAAGTGGCTAAAAATATAGCCTATG
AAATGTCGGAAAAACCCAGACTATCTTTAATAACTTTAAAGGAACACTTAACTTCAAAAATCCGCAAA
ACGCTACCAGGATTTATAGATAAAGAAGTAGCTATGCACGAAATAACCTTTCACCAACCAGAGGTAGC
AAGTAGAATAGAGGAAAATTTTGACAAAAGGACAACGGCAAGTAATAACCCTCAAAATTTTCCTCAA
GAGGCTGTAAGAAAAGAAATAAGAACAGATTCATTGAATTGTCAACCATTTCAGTTGAAGACATTTAG
TTATGGTTCGTTAAACAATTTAACTTTGGTACCTCTAGAACGTAGAGTTCCAAGCCCAAGTGAAGTTGA
GGTTCAAATCAAAACTGTACCAGTTAATTTTCGGGATATACTCAATGCACTTGGTATGCTCCAAGAGTA
TTACGAAAAAACATTTGGCATTGCTAATGCTGAAGATCTCACTTTTGGTTTTGAAGGTGCAGGTACTAT
CGTAGCTGTTGGGGCAGAAGTATCGCAGTGGCAAGTCGGCGATGAAGTAATGGTAATGAGAATTCACG
ATGCATTTAGTAGCTTTATTATCTGCTCGCCGGACAAACTGGTGCGTAAAAATTTTAATCTGAATATGG
AAGATGGAGCTAGTATTTGGGGGCCGTTTTCGACTGCATATCACGGGTTGATTAACTTAGCCAAAATTC
AGCCAGGAGATAGAGTATTAATTCACGCTGCTTCAGGTGGGATTGGACAAGCAGCAATTCAGTTGGCT
CAACTTTTTGGGGCGGAAGTATTTGCTACTACTAGTCCAGGTAAGATGAATTATCTCCGGGAACAGGG
AATTAAGTATGTGATGAATTCTCGAACGATGGAGTTTGTAAATGATGTGATGGAATTCACTCAGGGCC
GTGGGGTAGATGTTATTCTCAATAGCCTGACTCATGGAGAATACATCCAGAAAAATCTAGAGATCCTT
GCCGATAGAGGGCGATATGTTGAACTTGGTAAGTTGGGCATTTGGAGTCACGAGCAAGTCTATCAGAA
ACGCCCAGATATCAAATATTTTACTTTTGATTTGTTAGAAGAATTTGCCAAAGATAATCAATTGTTTTCT
CAGATATGGGATAATTTGGCACTTGAATTTGACCGCGATCGCTTGAAGCCACTACCTTACAAAACATTT
CCAATAGAAGATGTTCTTAAAGCCTTTGATTATATGCGACGCGGGAAGCATTTTGGCAGGGTAGTGGT
AGTTATGCCTGACTCCTATTCTAGACAGGAACAAGAGTTGGATGCTCGGTTATCCATTGAAAACAAAA
TGACTAAAGAGGAACAGATATTATTTCAATTACAATCTGGTGAAGTTTCCTTAGAAAATGCGGAACAA
CTATTGTTAGGAAATACGGAAACAGAAACACAAGATAAAGCTATAGCAGAAAATCAAATAGATAATA
TTCAAAACAAGTTAATTAACATGGATAGCTCAGAGAAAATCTTATCTTTGATTAGTTCAGTAGAAATAT
CTTTAGAAACAGCAGAAAAATTATTATTAGAAGTAGTAGAACCAGAAGTTAAAACAGAGGTTAATGAT
GAGGTTAATCCTAGTCAAAATCATATACCAACTACAGATATAGCGATTATTGGTATTTCATGTAGATAT
CCAGGAGCGAATAACTGGAAAGAATTTTGGGAAAATTTAAAGAATGGAATTGACAGTGTAACGGAAG
CTCCTCCTGGAAGATGGGAAGAAAAAAATTGGTATCATCCAGATCCAGATAATCCAGGTACTTCCTAT
TCAAAATGTGCCGGTTTTTTAGATGAAATTGATAAATTTGACCCTTTATTTTTTCATATTTCTCCGGAAG
AAGCTTGGTTTATGGAGCCTGAGCAAAGAATATTTTTAGAAGAAGCTTACCACGCTATAGAAGATGCT
GGATATGCTACAGACTCTCTTAGAGGTAAACAATATGGAGTATTTGTGGGAGTTACGGTAAATGGTGG
TTATCTTAAGTTGTTGTCAATTTCAGGATTAGATGTTCATAGGATGGCGGCTACAGGAAATGGTCCGTC
AATGATACCAGCAAGAATTGCCTATATGCTTGACCTTCAAGGGCCAGTAGTAGCTATTGATACTGCCTG
CTCGTCGTCATTGGTAGCTGTTCATCAAGCTTGCCAAAGCATACAACGAGGAGAGAGCGAAATAGCGA
TCGCTGGAGGTATTACTCTAATGCCAACATCAGACTTCCAAATAATGTCAAGTCAGTTTCAAGTTGTAT
CTCCTGATGGACGTTGTAAGACTTTTGATGCTTCAGCATCGGGTACAGCCTGGAGCGAAGGTTGTGGTG
TTCTCTTATTAAAAAGCTACAGTCAAGCAATTCAAGACAACGACCATATTTATGGAGTAATCAAAGGA
ACAGGAGTTAATTATGATGGTAATACTAACGGGATTAGTGCCCCTAGTAGTCAATCTCAAGCCAGTTT
AGAAGAAGCAGTTTATCAGAAATTTGGAATTAACCCAGAAACTATTAGTTATGTAGAAGCTCATGGTA
CAGCAACACCTCTGGGAGACCCGATAGAAGTAGAAGCTTTAACAGAAGCCTTTTCTAAATGGACGAAC
AAAAAACAGTTTTGTGCGATTGGCTCGGTAAAAACTAATATTGGTCATTCAGCAGCGGCTGCCGGAGT
TTCTGGTTTGATCAAGACAATTTTGTGCCTCAAAAATCAAAAATTAGTTCCATCCTTACATTTTAATCA
ACCAAACCCACATATTGACTTTGAAAATAGCCCTTTTTATGTCAATACAAAATTAAAAGATTGGGAAG
TGCTTGAAGGCCAACCAAGGCAAGCTACAGTTAGTTCTTTTGGTTTTAGTGGCACTAATGCTCATATAG
TTATAGAAGAGGCTCCTTCTCAAGTTAAAAGTCAGAATATTGTTGAACGTCCGATCCATCTGTTAACTC
TATCTGCGAAAACAGAAAAGGCTCTAGAAGATTTAGTCAGTAATTATCAAAATTATTTAGAAACTAAT
CCCGAGTTACCACTAGCAGATGTATGTTATACAGCCTCTACAGGTAGAGCACATTTTAATTATCGATTA
GGAGTTATTGCTTCTGAACCAAAAGCATTAATAGAGAAACTACTTGGGTGGAAAGCTCAGGAAGAATT
AGTAGGACTATTTTCAGGAAAACGAAATAGCGAAGGTCAGAAAATAGCATTCCTGTTCACAGGTCAAG
GTTCCCAGTATGCGAATATGGGAAGGCAACTTTATGAAAAAGCACCAACTTTCCGTCAAGCTTTAGAG
GAATGTGACCAAATTTTACAACCCTATCTAGAAGTACCTCTATTAGAGGTCATATACTCTGAGGATGCA
CAAAAGTCAAGTGATAATCTATTAGACCAAACAGCTTACACCCAACCAGCTGTGTTTGCTGTTGAATAT
GCTTTGGCTAAATTATGGTCTTCATGGGGAATCAAACCATGTGTAGTCATGGGTCACAGCGTAGGAGA
ATATGTAGCAGCAACAGTAGCCGGAGTATTCAGTTTAGAAGATGGTCTGAAACTAATAGCCATGCGAG
GAAAGTTGATGCAAAAGTTACCCTCCGGTGGTGAGATGGTATCCGTAATGGCATCAGAGTCTCAGGTA
ACAGAGGCTATAAAAGAATATAGTTCCCAAGTAACAATAGCAGCGATAAATGGACCAGAAAGTATAG
TAATTTCTGGTGAGAGTGTAGCCATAAAAAATATTTGTAGTTTATTTGAATCAGAGGGAATAAAGACC
AAGCAGCTACAAGTATCCCACGCTTTCCATTCTCCAATGATGGAACCGATGTTAGCAGAGTTTGAATCA
GTAGCTAAACAAGTCACCTATAATCAACCCCAAATACCACTAATATCAAACGTCACAGGTACTGAAGT
AGATGGAGAAATAACAAATGCTGAATATTGGGTAGATCATGTGCGTCAACCAGTAAGATTTGCCCAGA
GTATGAAAACTCTAGAGTCGGAAGGATATGAAACCTTCCTAGAAATAGGACCGAGACCAATATTGCTA
GGAATGGGAAGACAATGTGTAACAGAAGATGTAGGAGAATGGCTGCCATCATTACGTCCAGGGGTGG
ATGAATGGGAACAAATGCTATCAAGCTTAGGAAAATTGTATGTAAAAGGAGCCAAAATAGACTGGTC
AGGGTTTGACTCTGATTATACTCGCCAGAAAGTAGTATTACCCACATATCCATTCCAGGGAGAACGTTA
TTGGGTAGAAACCAACAACAACTTCTGGCCTCAACGGCAATTTTCCCAAGGGGAAAACCTCCATCCCC
TATTAGGTCAAAAGCTAAATTGTGCAGGGGAACAACAAATATTTGCATCACAAATAGGAGAAAACTCA
CCCAACTATCTGAGGGACCACCGAGTATTTAATCAAGCACTATTTCCCATAACAGGCTACCTAGAAAT
AGCAATAGCAGCAGGAAATCACCAATTAAAAACATCCCAGATAGTAATAGAAGACCTAACCATAACT
AGAGGATGGATACTACCAACAGGAGAATTAACCAATGCCCAAACCATACTCACCCCAATAGATAACC
AAAGCTATAAGTTTCAAATATTTTCTCAACCAGAACAACAGGAGTGGAGACTCCACACGACAGGAAAA
ATCAGAAAAGAGTCAACCCCCCCTACTCAGACAAAAGTTGACCTAGAAAAATACAAGAGTGAATGTA
ATCAAACAATAGAAGTCAAACAACATTATCAAAAATGTCAACAAGTAGGGATAGACTACGGGAATAC
CTTCCAAGGCATCCAAGAATTGTGGTCAGGTTCAAACCAAGCATTAGGTTATATCAAACTGCCCGAAG
AATTGATAACACAAACAAGCGACTATCATTTCCATCCAGCACTATTAGATGCAGCCTTGCAAGTAATG
TTTTATGCACTGCCAGCAACGGATAATGACAAAACTTATCTGTCAGCAGGAATAGAAGAATTCAGACT
ATATAAGACTCCCGGGCTGAGTATATGGGCATATGTATCAGTAACCAGTCAAGAAGTGGAAACTCCAG
AAAGTTTGACGGCTATTGTCACCATAGTAACTCCAGAAGGAGAAATAATTGCCAACATCAAAGGTTTA
CAAGTCAAACTAGCAACAAAACAGACCCTACTGGGAACAGAAACCGAATCAATAGAAAATTGGTTAT
ATGAAGTAGAGTGGAGAAACAAAGGTATTTTAGGTAAACTACTCCCACCAGATTTCCTCATACCTCCT
ATACAAATCAACCAAAAATTAACTCCAACTCTGACAGAATTAGTAACTCAAGTAGATAATGAAACAAC
AGCGTCTTTTGAAACAAGCTTAGAAGAATTAAGCAGAGATTATATAGTACAAGCATTACAGTCAATGA
GTTGGTCATACAAACCAACAGAAAGCTTTGCATTTGATGTAGCAGCCCAAAAATTAGGTATAGTTCCT
ACCCATCGACCACTGTTTAAGCGTTTGCTGCAAATATTAACAGAGTCAGGAATACTCAACTCAAAGAA
TCAGCAGTGGGAAGTAGCACAAACCTTACCTGAAGTCAAGCCTACGGAAAAAATCAGCAGTTTACAG
AAGAAATATCCAGAAGAAACAGCAGCATTGACACTACTCTCTCGTTGTGGGTCTAAACTAAGTGGGGT
ATTACGAGGAGCAATAGACCCAGGAGAGTTAGTGTTCCCCCAAGGAGATTTGACAGCAGCGACTCAAC
TTTATGAAGACTCAACAGTAGCGAAAGTGATGAACACAATAGTAGAAAAATCCATCACCAAAGCTATA
GAAAAATCCCCGAAAAGCCGGGGGCTCAGGTTGTTGGAAATAGGAGGGGGAACAGGAGGGACTACAA
GCTATATCCTACCTCATCTAACTCCTCAGCAAACCGAATATACATTCACGGATATAGGGGCATTATTTA
CAGCCAAAGCTCAAGAGAAATTCCGGGATTATAAGTTCATAAAGTATCAAACTTTAGACATAGAAGTA
GACCCGACAACTCAAGGATTTGAGGCTCATCAATATGATGTAATTATTGCAGCTAATGTACTTCATGCA
ACGACAGATATGAAGCAGACATTATCTCATGTGCGAGAACTGTTAGCAGATGGGGGAATGTTGGTGTT
ATCTGAAGCAACAGCTAAAACACTATGGGTAGATTTAGTATTTGGGTTGTTAGAAGGATGGTGGAAAT
TCAGGGATTATGAATTACGACCAGATTATCCTTTGTTGAGTCGTGAAAAATGGCATCATGTCTTGAGAG
AAACGGGTTTTACTGAAGTAGTTACTATGCCAGAAGTGGAGGGAATGGCAGAAACATTGTCAGCACAA
ACGGTAATTGTAGCTAAAAGCTCTCAAACGAAGTTAGAACAAAGGAATGATGATTCTAAGAGTTGGTT
GATACTGGCAGACTCTGAGGGAGTAGGGCAACAGTTAGCGACCTTACTCCGCTCAGTAGGAGAGGTTT
GTACTTTAGTATTTGCCGGAGAAAGGTATCAACAGATAGCTCCGGGAGAATTTAGTATTAATCCTAATC
AAGCTAAAGATTTTGAGGAGGTAATAGAGACAGTGGCAGGGAAGTCACCATCATTGTATGGAGTGGT
ACAATGTTGGACGACTGAAGCCGGAGTGGGGAATGGGATTAATTCTGAGGAGTTAGGAAGTTTATCGA
AGTTAGGGTGTGGCACAACTCTATCTTTAGTACAAGCATTGGTGAAAGGCGGGTTATCAACTGTCCCTC
GATTATGGTTGGTGACAAATGGTGCTCAGGCGGTGCCGAATAATCATCCGGTGATACCAGGAGTAGCT
CAATCTTCGGTATGGGGAATGGGGAAAGTGATTAGCTGGGAACATCCAGAGTTGAACTGTACTCGTAT
AGATTTGGACCCAGAGGAGACTTTAGAGGGTAAAGTTGATGCCCTATTTAAAGAAATTTGGTCGGAGG
ATAGGGAAGACCAGGTAGCATGGCGTGGGGATAGTCGTTATGTAGCTCGGTTGGTGGGTAGTCATCAT
CGGCAATTAGTGGCACAACAAGCTGATGGTAAAACTCAAAAGCCCTTAAGTTTCCGTTCTGAAGCAAG
CTATTTGATTACAGGAGGTATGGGAGGTTTGGGTTTGCTGGTAGCTAGTTGGATGGTGTCAAAGGGAG
CTAAACATTTGATATTGTTAGGACGCCGTTCACCGGATGATGCTACGAGGAAAAAAATAACCGAGTTA
GAAATGGCAGGAGCGTCCGTGGTAGTGGAAAAAGCCGATGTGACTGATTTGGAGTCGATGAAAGGGG
TGTTGCAGAGGATTGAGGAGTCAAAGCGACCGTTAGTAGGAGTGATTCATTCTGTGATGGTGCTATCA
AATGGAGTGCTACGAAATCAGACTTGGTCTAGTTTTGAACAGGTGATGGAACCGAAAGTTCAAGGTGC
TTGGCACTTGCATCAATTGACTCAAAGTCAACCATTAGACTTCTTTGTGCTGTTTTCTTCAGCAACATCT
CTGTTGGGTTCACTGGGTCAGGGCATGGCAAATTATTCTGCTGCTAATGGGTTTCTTGATGGTTTAGCT
CATTATCGTCGAACTATGGGATTACCGGGATTGAGTATCCATTGGGGAGCAGTTTCTCAAGTGGGACG
AGCGCTCGGGCGAGATACAGAGACAGCCGCTATGCTCAGTAAGAATGGGATGGGTTTAATATCTCCGG
CTCAGGTATTGGAGTCTTTGGAACTATTGATGAGTAGTTCGGATGTGGAAGTAGGGGTGATGCCTATTG
AGTGGTCAGGGTGGCAGGAGAGAGTGGCACAGTGGCCGTTTTTAGTGGATTGGCAGGAAACTATATTG
GAGGTAGCCCAACCATCGAAGTCAGATTTTCTGTTAAAGTTGGAGGCTACACCACCTAATGAGCTTCG
CTTGTTGTTGGTGGCTCATGTGCGTCGTCAGGTGGCTCAAGTGTTGAGAATTAGTCATCCTGAATCAAT
TGAAATGGATACAGGGTTTTTTGATTTGGGTATAGACTCTTTGACTTCTGTGGAGTTGAGGAATAAGTT
GCAAGGTAGTTTAAAGTGTTCAGTACCTTCTACTGTAACTTTGGACTACCCTACCATTAAGGCATTGGT
AGAATATTTATATCAACAGCTATTGTTAGAACAAGTTAGTTACTCAAATACTGTACCAACTGAAGAAA
TCAATGAAGATAGGGAGGAGATTACTCTAAGCGGGACACCAGCTCGGGAAGCAAGTAGTGCCTTACG
GGATGGCTATCGTCAAGCAGGAGTCAGCGGGCGGGTTCGGAGTTATTTGGATTTATTGGCTGGTTTAA
GTGATTTTCGGGAACATTTTGATGGTAGTGATGGTTTTTCTTTAGATTTGGTGGATATGGCTGATGGTCC
CGGTGAAGTGACCGTTATTTGTTGTGCCGGTACTGCCGCTATTTCTGGTCCCCATGAATTTACCCGCTTA
GCCGGTGCTTTGCGTGGTATTGCTCCCGTGCGGGCTGTTCCACAACCAGGTTATGAAGAAGGTGAACC
CTTACCCAGTTCTATGGCCGCTGTGGCCGCTGTTCAAGCCGATGCTGTTATTCGCACCCAAGGTGATAA
ACCCTTTGTGGTTGCTGGTCATAGTGCCGGTGCTTTAATGGCCTATGCTTTGGCCACTGAATTATTGGAT
CGTGGTCATCCTCCCCGCGGTGTGGTTTTAATTGATGTGTATCCTCCCGGTCATCAAGATGCTATGAAT
GCCTGGTTAGAAGAATTGACCGCCACTTTGTTTGATCGGGAAACCGTTCGCATGGATGATACCCGCTTA
ACTGCTTTGGGTGCCTATGATCGTTTAACTGGTCAATGGCGCCCCCGTGAAACCGGTTTGCCCACTTTA
TTGGTGAGTGCTGGTGAACCTATGGGTCCCTGGCCCGATGATTCTTGGAAACCCACCTGGCCCTTTGAA
CATGATACTGTGGCCGTTCCCGGTGATCATTTTACTATGGTTCAAGAACACGCTGATGCTATTGCACGA
CACATTGATGCCTGGTTGGGCGGTGGAAACTCCTAA
AprG
(SEQ ID NO: 54)
ATGGCAAACCTAAATCTTAATTTGGACTTAGTAGAGGGCAACTCTGATGTAGTACAACTGGTGGAGTT
GGGTAATGGTGTCGTGCAAATCACGATGAAAGATGAAGAAAGCTGCAATGGCTTTTCTCCTGGAATAA
TTGAAGGATTATACAAATGTTTTGGTGCAGTTGCTCAAAACCAAAGTTATAAAGTAGTAATTTTGACAG
GCTATGGAAATTATTTCTGTTCGGGAGGGACAAAAGAACGGTTAATCAGTATTTGGAAAGGAGACAGC
AAATGTAATGATTTAGATTTTTTTAGAATAGCATTAGATTGTGAAATACCAGTAATTGCAGCTATGCAA
GGTCATAGTATTGGCGGTGGTTTGGTTTTGGGATTGTATGCAGATTTAGTAGTGTTAAGTCAAGAAAGT
ATTTACACTACTAATTTTATGAAGTATGGTTTTACTCCAGGTGTCGGATGCACGTTAATTCTCCCTGAG
AAATTCGGTGCTTTAGGGTTTGAAATGATGTATACTGCCCAAAATTATCGAGGGAAAGAATTAGCTGA
ACGGGGTGTTTCTTTTCCAGTTGTACCGAGAAAAGATGTGCTAGAAGTGGCTAAAAATATAGCCTATG
AAATGTCGGAAAAACCCAGACTATCTTTAATAACTTTAAAGGAACACTTAACTTCAAAAATCCGCAAA
ACGCTACCAGGATTTATAGATAAAGAAGTAGCTATGCACGAAATAACCTTTCACCAACCAGAGGTAGC
AAGTAGAATAGAGGAAAATTTTGACAAAAGGACAACGGCAAGTAATAACCCTCAAAATTTTCCTCAA
GAGGCTGTAAGAAAAGAAATAAGAACAGATTCATTGAATTGTCAACCATTTCAGTTGAAGACATTTAG
TTATGGTTCGTTAAACAATTTAACTTTGGTACCTCTAGAACGTAGAGTTCCAAGCCCAAGTGAAGTTGA
GGTTCAAATCAAAACTGTACCAGTTAATTTTCGGGATATACTCAATGCACTTGGTATGCTCCAAGAGTA
TTACGAAAAAACATTTGGCATTGCTAATGCTGAAGATCTCACTTTTGGTTTTGAAGGTGCAGGTACTAT
CGTAGCTGTTGGGGCAGAAGTATCGCAGTGGCAAGTCGGCGATGAAGTAATGGTAATGAGAATTCACG
ATGCATTTAGTAGCTTTATTATCTGCTCGCCGGACAAACTGGTGCGTAAAAATTTTAATCTGAATATGG
AAGATGGAGCTAGTATTTGGGGGCCGTTTTCGACTGCATATCACGGGTTGATTAACTTAGCCAAAATTC
AGCCAGGAGATAGAGTATTAATTCACGCTGCTTCAGGTGGGATTGGACAAGCAGCAATTCAGTTGGCT
CAACTTTTTGGGGCGGAAGTATTTGCTACTACTAGTCCAGGTAAGATGAATTATCTCCGGGAACAGGG
AATTAAGTATGTGATGAATTCTCGAACGATGGAGTTTGTAAATGATGTGATGGAATTCACTCAGGGCC
GTGGGGTAGATGTTATTCTCAATAGCCTGACTCATGGAGAATACATCCAGAAAAATCTAGAGATCCTT
GCCGATAGAGGGCGATATGTTGAACTTGGTAAGTTGGGCATTTGGAGTCACGAGCAAGTCTATCAGAA
ACGCCCAGATATCAAATATTTTACTTTTGATTTGTTAGAAGAATTTGCCAAAGATAATCAATTGTTTTCT
CAGATATGGGATAATTTGGCACTTGAATTTGACCGCGATCGCTTGAAGCCACTACCTTACAAAACATTT
CCAATAGAAGATGTTCTTAAAGCCTTTGATTATATGCGACGCGGGAAGCATTTTGGCAGGGTAGTGGT
AGTTATGCCTGACTCCTATTCTAGACAGGAACAAGAGTTGGATGCTCGGTTATCCATTGAAAAAAAA
TGACTAAAGAGGAACAGATATTATTTCAATTACAATCTGGTGAAGTTTCCTTAGAAAATGCGGAACAA
CTATTGTTAGGAAATACGGAAACAGAAACACAAGATAAAGCTATAGCAGAAAATCAAATAGATAATA
TTCAAAACAAGTTAATTAACATGGATAGCTCAGAGAAAATCTTATCTTTGATTAGTTCAGTAGAAATAT
CTTTAGAAACAGCAGAAAAATTATTATTAGAAGTAGTAGAACCAGAAGTTAAAACAGAGGTTAATGAT
GAGGTTAATCCTAGTCAAAATCATATACCAACTACAGATATAGCGATTATTGGTATTTCATGTAGATAT
CCAGGAGCGAATAACTGGAAAGAATTTTGGGAAAATTTAAAGAATGGAATTGACAGTGTAACGGAAG
CTCCTCCTGGAAGATGGGAAGAAAAAAATTGGTATCATCCAGATCCAGATAATCCAGGTACTTCCTAT
TCAAAATGTGCCGGTTTTTTAGATGAAATTGATAAATTTGACCCTTTATTTTTTCATATTTCTCCGGAAG
AAGCTTGGTTTATGGAGCCTGAGCAAAGAATATTTTTAGAAGAAGCTTACCACGCTATAGAAGATGCT
GGATATGCTACAGACTCTCTTAGAGGTAAACAATATGGAGTATTTGTGGGAGTTACGGTAAATGGTGG
TTATCTTAAGTTGTTGTCAATTTCAGGATTAGATGTTCATAGGATGGCGGCTACAGGAAATGGTCCGTC
AATGATACCAGCAAGAATTGCCTATATGCTTGACCTTCAAGGGCCAGTAGTAGCTATTGATACTGCCTG
CTCGTCGTCATTGGTAGCTGTTCATCAAGCTTGCCAAAGCATACAACGAGGAGAGAGCGAAATAGCGA
TCGCTGGAGGTATTACTCTAATGCCAACATCAGACTTCCAAATAATGTCAAGTCAGTTTCAAGTTGTAT
CTCCTGATGGACGTTGTAAGACTTTTGATGCTTCAGCATCGGGTACAGCCTGGAGCGAAGGTTGTGGTG
TTCTCTTATTAAAAAGCTACAGTCAAGCAATTCAAGACAACGACCATATTTATGGAGTAATCAAAGGA
ACAGGAGTTAATTATGATGGTAATACTAACGGGATTAGTGCCCCTAGTAGTCAATCTCAAGCCAGTTT
AGAAGAAGCAGTTTATCAGAAATTTGGAATTAACCCAGAAACTATTAGTTATGTAGAAGCTCATGGTA
CAGCAACACCTCTGGGAGACCCGATAGAAGTAGAAGCTTTAACAGAAGCCTTTTCTAAATGGACGAAC
AAAAAACAGTTTTGTGCGATTGGCTCGGTAAAAACTAATATTGGTCATTCAGCAGCGGCTGCCGGAGT
TTCTGGTTTGATCAAGACAATTTTGTGCCTCAAAAATCAAAAATTAGTTCCATCCTTACATTTTAATCA
ACCAAACCCACATATTGACTTTGAAAATAGCCCTTTTTATGTCAATACAAAATTAAAAGATTGGGAAG
TGCTTGAAGGCCAACCAAGGCAAGCTACAGTTAGTTCTTTTGGTTTTAGTGGCACTAATGCTCATATAG
TTATAGAAGAGGCTCCTTCTCAAGTTAAAAGTCAGAATATTGTTGAACGTCCGATCCATCTGTTAACTC
TATCTGCGAAAACAGAAAAGGCTCTAGAAGATTTAGTCAGTAATTATCAAAATTATTTAGAAACTAAT
CCCGAGTTACCACTAGCAGATGTATGTTATACAGCCTCTACAGGTAGAGCACATTTTAATTATCGATTA
GGAGTTATTGCTTCTGAACCAAAAGCATTAATAGAGAAACTACTTGGGTGGAAAGCTCAGGAAGAATT
AGTAGGACTATTTTCAGGAAAACGAAATAGCGAAGGTCAGAAAATAGCATTCCTGTTCACAGGTCAAG
GTTCCCAGTATGCGAATATGGGAAGGCAACTTTATGAAAAAGCACCAACTTTCCGTCAAGCTTTAGAG
GAATGTGACCAAATTTTACAACCCTATCTAGAAGTACCTCTATTAGAGGTCATATACTCTGAGGATGCA
CAAAAGTCAAGTGATAATCTATTAGACCAAACAGCTTACACCCAACCAGCTGTGTTTGCTGTTGAATAT
GCTTTGGCTAAATTATGGTCTTCATGGGGAATCAAACCATGTGTAGTCATGGGTCACAGCGTAGGAGA
ATATGTAGCAGCAACAGTAGCCGGAGTATTCAGTTTAGAAGATGGTCTGAAACTAATAGCCATGCGAG
GAAAGTTGATGCAAAAGTTACCCTCCGGTGGTGAGATGGTATCCGTAATGGCATCAGAGTCTCAGGTA
ACAGAGGCTATAAAAGAATATAGTTCCCAAGTAACAATAGCAGCGATAAATGGACCAGAAAGTATAG
TAATTTCTGGTGAGAGTGTAGCCATAAAAAATATTTGTAGTTTATTTGAATCAGAGGGAATAAAGACC
AAGCAGCTACAAGTATCCCACGCTTTCCATTCTCCAATGATGGAACCGATGTTAGCAGAGTTTGAATCA
GTAGCTAAACAAGTCACCTATAATCAACCCCAAATACCACTAATATCAAACGTCACAGGTACTGAAGT
AGATGGAGAAATAACAAATGCTGAATATTGGGTAGATCATGTGCGTCAACCAGTAAGATTTGCCCAGA
GTATGAAAACTCTAGAGTCGGAAGGATATGAAACCTTCCTAGAAATAGGACCGAGACCAATATTGCTA
GGAATGGGAAGACAATGTGTAACAGAAGATGTAGGAGAATGGCTGCCATCATTACGTCCAGGGGTGG
ATGAATGGGAACAAATGCTATCAAGCTTAGGAAAATTGTATGTAAAAGGAGCCAAAATAGACTGGTC
AGGGTTTGACTCTGATTATACTCGCCAGAAAGTAGTATTACCCACATATCCATTCCAGGGAGAACGTTA
TTGGGTAGAAACCAACAACAACTTCTGGCCTCAACGGCAATTTTCCCAAGGGGAAAACCTCCATCCCC
TATTAGGTCAAAAGCTAAATTGTGCAGGGGAACAACAAATATTTGCATCACAAATAGGAGAAAACTCA
CCCAACTATCTGAGGGACCACCGAGTATTTAATCAAGCACTATTTCCCATAACAGGCTACCTAGAAAT
AGCAATAGCAGCAGGAAATCACCAATTAAAAACATCCCAGATAGTAATAGAAGACCTAACCATAACT
AGAGGATGGATACTACCAACAGGAGAATTAACCAATGCCCAAACCATACTCACCCCAATAGATAACC
AAAGCTATAAGTTTCAAATATTTTCTCAACCAGAACAACAGGAGTGGAGACTCCACACGACAGGAAAA
ATCAGAAAAGAGTCAACCCCCCCTACTCAGACAAAAGTTGACCTAGAAAAATACAAGAGTGAATGTA
ATCAAACAATAGAAGTCAAACAACATTATCAAAAATGTCAACAAGTAGGGATAGACTACGGGAATAC
CTTCCAAGGCATCCAAGAATTGTGGTCAGGTTCAAACCAAGCATTAGGTTATATCAAACTGCCCGAAG
AATTGATAACACAAACAAGCGACTATCATTTCCATCCAGCACTATTAGATGCAGCCTTGCAAGTAATG
TTTTATGCACTGCCAGCAACGGATAATGACAAAACTTATCTGTCAGCAGGAATAGAAGAATTCAGACT
ATATAAGACTCCCGGGCTGAGTATATGGGCATATGTATCAGTAACCAGTCAAGAAGTGGAAACTCCAG
AAAGTTTGACGGCTATTGTCACCATAGTAACTCCAGAAGGAGAAATAATTGCCAACATCAAAGGTTTA
CAAGTCAAACTAGCAACAAAACAGACCCTACTGGGAACAGAAACCGAATCAATAGAAAATTGGTTAT
ATGAAGTAGAGTGGAGAAACAAAGGTATTTTAGGTAAACTACTCCCACCAGATTTCCTCATACCTCCT
ATACAAATCAACCAAAAATTAACTCCAACTCTGACAGAATTAGTAACTCAAGTAGATAATGAAACAAC
AGCGTCTTTTGAAACAAGCTTAGAAGAATTAAGCAGAGATTATATAGTACAAGCATTACAGTCAATGA
GTTGGTCATACAAACCAACAGAAAGCTTTGCATTTGATGTAGCAGCCCAAAAATTAGGTATAGTTCCT
ACCCATCGACCACTGTTTAAGCGTTTGCTGCAAATATTAACAGAGTCAGGAATACTCAACTCAAAGAA
TCAGCAGTGGGAAGTAGCACAAACCTTACCTGAAGTCAAGCCTACGGAAAAAATCAGCAGTTTACAG
AAGAAATATCCAGAAGAAACAGCAGCATTGACACTACTCTCTCGTTGTGGGTCTAAACTAAGTGGGGT
ATTACGAGGAGCAATAGACCCAGGAGAGTTAGTGTTCCCCCAAGGAGATTTGACAGCAGCGACTCAAC
TTTATGAAGACTCAACAGTAGCGAAAGTGATGAACACAATAGTAGAAAAATCCATCACCAAAGCTATA
GAAAAATCCCCGAAAAGCCGGGGGCTCAGGTTGTTGGAAATAGGAGGGGGAACAGGAGGGACTACAA
GCTATATCCTACCTCATCTAACTCCTCAGCAAACCGAATATACATTCACGGATATAGGGGCATTATTTA
CAGCCAAAGCTCAAGAGAAATTCCGGGATTATAAGTTCATAAAGTATCAAACTTTAGACATAGAAGTA
GACCCGACAACTCAAGGATTTGAGGCTCATCAATATGATGTAATTATTGCAGCTAATGTACTTCATGCA
ACGACAGATATGAAGCAGACATTATCTCATGTGCGAGAACTGTTAGCAGATGGGGGAATGTTGGTGTT
ATCTGAAGCAACAGCTAAAACACTATGGGTAGATTTAGTATTTGGGTTGTTAGAAGGATGGTGGAAAT
TCAGGGATTATGAATTACGACCAGATTATCCTTTGTTGAGTCGTGAAAAATGGCATCATGTCTTGAGAG
AAACGGGTTTTACTGAAGTAGTTACTATGCCAGAAGTGGAGGGAATGGCAGAAACATTGTCAGCACAA
ACGGTAATTGTAGCTAAAAGCTCTCAAACGAAGTTAGAACAAAGGAATGATGATTCTAAGAGTTGGTT
GATACTGGCAGACTCTGAGGGAGTAGGGCAACAGTTAGCGACCTTACTCCGCTCAGTAGGAGAGGTTT
GTACTTTAGTATTTGCCGGAGAAAGGTATCAACAGATAGCTCCGGGAGAATTTAGTATTAATCCTAATC
AAGCTAAAGATTTTGAGGAGGTAATAGAGACAGTGGCAGGGAAGTCACCATCATTGTATGGAGTGGT
ACAATGTTGGACGACTGAAGCCGGAGTGGGGAATGGGATTAATTCTGAGGAGTTAGGAAGTTTATCGA
AGTTAGGGTGTGGCACAACTCTATCTTTAGTACAAGCATTGGTGAAAGGCGGGTTATCAACTGTCCCTC
GATTATGGTTGGTGACAAATGGTGCTCAGGCGGTGCCGAATAATCATCCGGTGATACCAGGAGTAGCT
CAATCTTCGGTATGGGGAATGGGGAAAGTGATTAGCTGGGAACATCCAGAGTTGAACTGTACTCGTAT
AGATTTGGACCCAGAGGAGACTTTAGAGGGTAAAGTTGATGCCCTATTTAAAGAAATTTGGTCGGAGG
ATAGGGAAGACCAGGTAGCATGGCGTGGGGATAGTCGTTATGTAGCTCGGTTGGTGGGTAGTCATCAT
CGGCAATTAGTGGCACAACAAGCTGATGGTAAAACTCAAAAGCCCTTAAGTTTCCGTTCTGAAGCAAG
CTATTTGATTACAGGAGGTATGGGAGGTTTGGGTTTGCTGGTAGCTAGTTGGATGGTGTCAAAGGGAG
CTAAACATTTGATATTGTTAGGACGCCGTTCACCGGATGATGCTACGAGGAAAAAAATAACCGAGTTA
GAAATGGCAGGAGCGTCCGTGGTAGTGGAAAAAGCCGATGTGACTGATTTGGAGTCGATGAAAGGGG
TGTTGCAGAGGATTGAGGAGTCAAAGCGACCGTTAGTAGGAGTGATTCATTCTGTGATGGTGCTATCA
AATGGAGTGCTACGAAATCAGACTTGGTCTAGTTTTGAACAGGTGATGGAACCGAAAGTTCAAGGTGC
TTGGCACTTGCATCAATTGACTCAAAGTCAACCATTAGACTTCTTTGTGCTGTTTTCTTCAGCAACATCT
CTGTTGGGTTCACTGGGTCAGGGCATGGCAAATTATTCTGCTGCTAATGGGTTTCTTGATGGTTTAGCT
CATTATCGTCGAACTATGGGATTACCGGGATTGAGTATCCATTGGGGAGCAGTTTCTCAAGTGGGACG
AGCGCTCGGGCGAGATACAGAGACAGCCGCTATGCTCAGTAAGAATGGGATGGGTTTAATATCTCCGG
CTCAGGTATTGGAGTCTTTGGAACTATTGATGAGTAGTTCGGATGTGGAAGTAGGGGTGATGCCTATTG
AGTGGTCAGGGTGGCAGGAGAGAGTGGCACAGTGGCCGTTTTTAGTGGATTGGCAGGAAACTATATTG
GAGGTAGCCCAACCATCGAAGTCAGATTTTCTGTTAAAGTTGGAGGCTACACCACCTAATGAGCTTCG
CTTGTTGTTGGTGGCTCATGTGCGTCGTCAGGTGGCTCAAGTGTTGAGAATTAGTCATCCTGAATCAAT
TGAAATGGATACAGGGTTTTTTGATTTGGGTATAGACTCTTTGACTTCTGTGGAGTTGAGGAATAAGTT
GCAAGGTAGTTTAAAGTGTTCAGTACCTTCTACTGTAACTTTGGACTACCCTACCATTAAGGCATTGGT
AGAATATTTATATCAACAGCTATTGTTAGAACAAGTTAGTTACTCAAATACTGTACCAACTGAAGAAA
TCAATGAAGATAGGGAGGAGATTACTCTA

Non-limiting examples of promoters that can be used in the context of this invention are identified below.

PRham promoter
(SEQ ID NO: 55)
TTGACAGCTAGCTCAGTCCTAGGTATAATGCTAGCATGACCGTATTACATAGTGTGGATTTTTTTCCGT
CTGGTAACGCGTCCGTGGCGATAGAACCCCGGCTCCCGCAGGCGGATTTTCCTGAACATCATCATGATT
TTCATGAAATTGTGATTGTCGAACATGGCACGGGTATTCATGTGTTTAATGGGCAGCCCTATACCATCA
CCGGTGGCACGGTCTGTTTCGTACGCGATCATGATCGGCATCTGTATGAACATACCGATAATCTGTGTC
TGACCAATGTGCTGTATCGCTCGCCGGATCGATTTCAGTTTCTCGCCGGGCTGAATCAGTTGCTGCCAC
AAGAGCTGGATGGGCAGTATCCGTCTCACTGGCGCGTTAACCACAGCGTATTGCAGCAGGTGCGACAG
CTGGTTGCACAGATGGAACAGCAGGAAGGGGAAAATGATTTACCCTCGACCGCCAGTCGCGAGATCTT
GTTTATGCAATTACTGCTCTTGCTGCGTAAAAGCAGTTTGCAGGAGAACCTGGAAAACAGCGCATCAC
GTCTCAACTTGCTTCTGGCCTGGCTGGAGGACCATTTTGCCGATGAGGTGAATTGGGATGCCGTGGCGG
ATCAATTTTCTCTTTCACTGCGTACGCTACATCGGCAGCTTAAGCAGCAAACGGGACTGACGCCTCAGC
GATACCTGAACCGCCTGCGACTGATGAAAGCCCGACATCTGCTACGCCACAGCGAGGCCAGCGTTACT
GACATCGCCTATCGCTGTGGATTCAGCGACAGTAACCACTTTTCGACGCTTTTTCGCCGAGAGTTTAAC
TGGTCACCGCGTGATATTCGCCAGGGACGGGATGGCTTTCTGCAATAAGCCACAATTCAGCAAATTGT
GAACATCATCACGTTCATCTTTCCCTGGTTGCCAATGGCCCATTTTCCTGTCAGTAACGAGAAGGTCGC
GAATTCAGGCGCTTTTTAGACTGGTCGTAATGAA
PCoAT promoter
(SEQ ID NO: 56)
GCTTTTTAACTTGGATTTTTACCTTCTCAGCCTAAACCTTAACATTAGTGTCAATGTCAAGGTTTTGAGT
AAACTAAAGGGATGAAGACTAATCACTTAACGATTAAAGAACTCACAGATGCAGTGGGAGGTGGCGT
TACGCCTCGCATGGTGCGCCATTACCACACCCTGGGATTGCTTCCCCCCGTTCAACGCTCAGAGGGCAA
CTACCGCCTTTATACTCAGCAGGACGTACAACGGCTCCAACGAGTCATTGCCCTCAAACAGCAGGGCT
TTCAGTTGTCTCATATTCGGCAACTGCTGGATAGCCATTCTGAAGAGAGCCTTGATCCCACCCTAATGG
TGCAGTTGCAACAGCAATATCAGGCTGTGATTCAGCAGATTACTCGACTCCGCCAAACCGCATCTGCTT
TAGAAGGATTACTGGGACGCGACCAGAGTTGCCAGATTACCCAAGCGGAAGCTCTGGCCCAACTGAA
ACAGCTTGATGTGGATGTCCAGGAGGGGTTAGGAAAACTCGATCAGTTGTGGACGAACCTAGATGCGG
AGACAACAACTCATCCAGAAGCCTTTCAGGAATCCCTCAAACACCTGCTACCGGATTTATCGGCTTACT
CTGAAATTACCATTCACTTACTACATCAATTAGTGCTGGCCTGTGGTGATGTTAGCTTGGTAAACGCCG
TTCGATTGAGTCAGGGAGCGATCGCCTCGGCACGAGATGCACTGAAAGCAGGGTGTCCAGTCGTCACC
GATGTTCCGGTTGTGGCTGCGGCTCTTGATCAAACTCGGTTAGCTCATTTAGGATGTACGGTTAAAACG
CTGATTGACGACCCTCACATCACAGGGCTTAGGGAAGCCGAGCAAGCTTTTTGGCACCATGACCATTG
GCAACAGCGGTTACAACAGATTCCCCAAGGATGTGTGCTGGCGATCGGCTATGCCCCTTCTGTTTTACT
CACTGCCTGTAAGCTGATAGAGCAACAACATATTCAGCCGGCTCTTGTGATCGGAATGCCGATCGGTTT
TAGTCATGCTCCGGGGGCAAAACGACGACTGATGACCAGTCCCATTCCCCATATCACCATTCAGGGGA
GCCTCGGTGGAGGACTTCTAGCCGCAGTAACGCTAAACGCTTTAGTGGAAACATTGATTGCAAAGCCA
GATTGCCACTGCTATCTCACTTGTCTTTAG
Non-limiting examples of constructs that can be used in the context of this invention are
identified below.
pRL838Apra
(SEQ ID NO: 57)
ATCGCGAGAATTAATTCAGATAAAAAAAATCCTTAGCTTTCGCTAAGGATGATTTCTAGCGATGACCCT
GCTGATTGGTTCGCTGACCATTTCCGGGTGCGGGACGGCGTTACCAGAAACTCAGAAGGTTCGTCCAA
CCAAACCGACTCTGACGGCAGTTTACGAGAGAGATGATAGGGTCTGCTTCAGTAAGCCAGATGCTACA
CAATTAGGCTTGTACGGGTACTCGACCTGCATCCCTTAACTTACTTATTAAATAATTTATAGCTATTGA
AAAGAGATAAGAATTGTTCAAAGCTAATATTGTTTAAATCGTCAATTCCTGCATGTTTTAAGGAATTGT
TAAATTGATTTTTTGTAAATATTTTCTTGTATTCTTTGTTAACCCATTTCATAACGAAATAATTATACTTT
TGTTTATCTTTGTGTGATATTCTTGATTTTTTTCTACTTAATCTGATAAGTGAGCTATTCACTTTAGGTTT
AGGATGAAAATATTCTCTTGGAACCATACTTAATATAGAAATATCAACTTCTGCCATTAAAAGTAATGC
CAATGAGCGTTTTGTATTTAATAATCTTTTAGCAAACCCGTATTCCACGATTAAATAAATCTCATTAGC
TATACTATCAAAAACAATTTTGCGTATTATATCCGTACTTATGTTATAAGGTATATTACCATATATTTTA
TAGGATTGGTTTTTAGGAAATTTAAACTGCAATATATCCTTGTTTAAAACTTGGAAATTATCGTGATCA
ACAAGTTTATTTTCTGTAGTTTTGCATAATTTATGGTCTATTTCAATGGCAGTTACGAAATTACACCTCT
TTACTAATTCAAGGGTAAAATGGCCTTTTCCTGAGCCGATTTCAAAGATATTATCATGTTCATTTAATCT
TATATTTGTCATTATTTTATCTATATTATGTTTTGAAGTAATAAAGTTTTGACTGTGTTTTATATTTTTCT
CGTTCATTATAACCCTCTTTAATTTGGTTATATGAATTTTGCTTATTAACGATTCATTATAACCACTTAT
TTTTTGTTTGGTTGATAATGAACTGTGCTGATTACAAAAATACTAAAAATGCCCATATTTTTTCCTCCTT
ATAAAATTAGTATAATTATAGCACGCGAATTCATCGAATTTCTGCCATTCATCCGCTTATTATCACTTAT
TCAGGCGTAGCACCAGGCGTTTAAGGGCACCAATAACTGCCTTAAAAAAATTACGCCCCGCCCTGCCA
CTCATCGCAGTACTGTTGTAATTCATTAAGCATTCTGCCGACATGGAAGCCATCACAAACGGCATGATG
AACCTGAATCGCCAGCGGCATCAGCACCTTGTCGCCTTGCGTATAATATTTGCCCATGGTGAAAACGG
GGGCGAAGAAGTTGTCCATATTGGCCACGTTTAAATCAAAACTGGTGAAACTCACCCAGGGATTGGCT
GAGACGAAAAACATATTCTCAATAAACCCTTTAGGGAAATAGGCCAGGTTTTCACCGTAACACGCCAC
ATCTTGCGAATATATGTGTAGAAACTGCCGGAAATCGTCGTGGTATTCACTCCAGAGCGATGAAAACG
TTTCAGTTTGCTCATGGAAAACGGTGTAACAAGGGTGAACACTATCCCATATCACCAGCTCACCGTCTT
TCATTGCCATACGGAATTCCGGATGAGCATTCATCAGGCGGGCAAGAATGTGAATAAAGGCCGGATAA
AACTTGTGCTTATTTTTCTTTACGGTCTTTAAAAAGGCCGTAATATCCAGCTGAACGGTCTGGTTATAG
GTACATTGAGCAACTGACTGAAATGCCTCAAAATGTTCTTTACGATGCCATTGGGATATATCAACGGTG
GTATATCCAGTGATTTTTTTCTCCATTTTAGCTTCCTTAGCTCCTGAAAATCTCGATAACTCAAAAAATA
CGCCCGGTAGTGATCTTATTTCATTATGGTGAAAGTTGGAACCTCTTACGAGCGGCCGCATACGATTTA
GGTGACACTATAGGATCCCGACATCATAACGGTTCTGGCAAATATTCTGAAATGAGCTGTTGACAATT
AATCATCCGGCTCGTATAATGTGTGGAATTGTGAGCGGATAACAATTTCACACAGGAAACAGACCATG
CTAGATAAAATAAATCGTTATGCTCATGGGTTTGTAGCTGTACCAGTGATTTGTGCTTGTTCAGAAGCA
GGGGTATTCGAGTTACTATCACAGAAAAAATCACTGAAATTAGAAGAAATAGTAGAGCATTTAGCAGC
AAATAGTGGACATCTAATGGTAGCTATGCGACTCCTAGAGTCATTATCATTCCTATATCGCTCTCAAGC
AGAAGAATACATATTGACAGAACAAAGTCAACAACATCAAATCATCCCTAAAGCTCTGATGAGCTTAT
ATAAATATCCATTTGAGTTATATTTAAAAGGAGAAGTAGAAACAGGAATCAGCAATTGGATTAATTGT
TCATCTCGAAGATGGGATACAGAAAACTCATTACTAAGTGATTTATTAGATGGAGTATTACTCATCCCC
CTGCTACTAGAACTGAAAAAACAAAACTTACTGGATGAATCAAAAAAAATATTTAACACATTAACAAA
TTCCCTTAAACAAGAACTATCAACATTATTCATCAATTTAGGATGGGCAGAAGAAAAAACAGAAGGGC
TATATTTAACAGATATAGGTAGATTTATGCGTGACAGATCCTTAAACTTAGGAACAACAGCGTCTTATG
CTCCTATGTTGTTACAGATGAAAGAATTACTATTTGGAAATCCTCAGAGAGTATTTCAAAGAAATAAG
ACTGAAAAAGAAAGACACGTAAATAGAACATTAAATGTAGTAGCAAGTGGCTTTCAACACGAAAAGT
TTTTTGCCGATACAGATAAAATCATCATATCTATATTTAACCAACAGCCAATAGAAGAGCAACCAAGC
TATATAGTAGACATGGGTTGTGGGGATGGAACGCTACTGAAACGAATATATAAAATCATCAAACAATT
CTCTGCTAGGGGAAAAGTATTAACAGAGTATCCTATCATCATGGTAGGAGTAGACTACAATCAAGAAG
CATTAGATGTCACAGATAAAAATTTAGTAGATATTCCCCATCTAGTCATTCCAGGAGATATAGGAGCA
CCAGAAAAATTATTAGAGCAATTGAAAGCACAAGGAATAGAACCAGAAAAAGTATTACATATTCGTTC
ATTCCTAGATCATGACCGACCATTTATAGCTCCAAAAAATACAGAAATAGCTCAAGCACGTTCTCAGTT
AGATTATCAAGTAGTAGATGTAGATCGAGAAGGAAAGCTGATACCACCTCACATAGCAGTACAAAGTT
TAGTAGAACACTTAGAAAGATGGTCATCAATAATAACTAGACATGGATTATTGCTCCTAGAAGTACAT
AGTTTAACGCCAGCAGTAGTCAAAAAATATATAGACGAAAGTGAATCTCTGCATTTTGATGCCTATCA
TGCCTTCTCAATGCAACATTTAGTAGAAGCAGATGTGTTCTTGATGGCAGCAGCAGAGGTGGGTTTATT
TTCACGGAAAGAAGCTTTTCGTAAGTATCCGAAAACGTTGCCATTGACTCGAATCACAGTTAATCATTT
TGAAAAGCGTAAATATCAGATACGGTATGCAACTGTAAACGATATACCAAATCTGTTAAAGTGTGCGA
CATTTAATCAACCTGTAAACGAACCCTTCTTTCAAGTTTTATTGAAACAAACTCCAACAGCACATTTAT
TATTAGAATATCAAGGTGAATTGGTAGCTGCAATCTTCACAGAAACTAAAAATTCTAATGAGGTGCTA
GGAATTCGTGAGTTTTTAGTAAGGACTTCTGTAGAAAATTGGCAAGTATTAGCAAAAGATTTACTAGA
ATTTGTGGAACAGTGGGGAGTAGTCAAACCAGGAATAAAAGAAATAGAAGGATTGTTAAAATACCAT
GAAGCCATCTCAAACTTTCAAAAATCAAAGTGGTATCAATCTTCAGTTTTAAACAAAAAGCTTATAGA
AAAAATAACTCTACACGAATTAGCTACTTTAGAACTATGTAATTTAATGGCTCCAGAATATGAGCTAG
AAGCCTTTGCCGCACGTTGGCTTTTGCGTGTTTTTCAAGATATGGGTGTATTTCTCAGAGAGGGTGAAT
CTTATCAGGAGTCTGAGTTGGTTTCCCAGCTAAACATCTCACCACGTTACCAGAGACTTTTAGGTGCTT
TGTTACAAATTTTGCATAAGCGTGGTATTCTAAAAATTGAGAAAGATAGAGTGTTCACATTAGCAAGA
TGTAAGACCTTTGCCTTAGAAAATATTTCGTCTGAAGTTTCTGCTTTTTATGATTATTTTTCTGAAAAAT
ATCCTGCTCATTTATCCTGGTTAACAGTAGTCAAAAGGTGTCTAGAGAAATATCCTTTGATTCTACGCG
GTGAAGTTGATGTCAACGAAGTTGTTTTTACAGATGGGGATATGGAGCTATTTGCTGGACTATTTCTAG
GACATCGTGTTGCTGACTACTTTAATGAGTTGCTAGCAGATGGGGTTTGCTGGGAAGTAGAACAGCGG
TTGTTAGAAGAAAAGAGGGCACAACCTATTCGGATCTTGGAGATTGGAGCAGGAACAGGAGGTGTTA
CAGGAATATTGCTAGAAAAGCTAGCTTCTCATGCAGAGCAAATTGAATTTTGGTTTACTGATATTTCTA
GCGTTTTTACACGTTATGGTGAGAGTAAGTTCAAGCAGTTTCCTTGGGTGAAATATCAAACCTTTGACA
TAGAAAAATCTCTTGATGCTCAGGGGATAAAGTCTGAAAGTTTTGATGTGGTAATTGCTAATAACGTA
CTCCATAACACAAAATTAATTCATCAAACCTTAAATAACAGTAACTCACTATTAAATACTGGGGGGTT
ATTGGCATTACTAGAGTTTACTCAACCAATTGATATTCTTTTATACTTTGGAGGGTTGCTTCAAGGATTT
TGGTTGTTTGAAGATCCAGAATACCGACTAGAAGTTGGTTGTTTACTGAGTATACCACTGTGGCAAAA
AGTTCTCAGTGATTGTGGGTTTGATGAAATAATACCATTGGGATTACCCTGTGAGATGCACGCTCTTTC
TAAGGCAAGAGAATCTGTTATATTTGCTCGAAAGCATCAAGTACAGGAGAAAACATTTTCTGAAAAAA
TTAAACAAAATTTGACAGAAAATGGTAAGCATGGGCAGGCAGAATTCGATTTTATTAGTATTAATAAT
TCACAAGAAAGTTCATCCAAATTAGAAATTTTTGAACAGGAATGTCGAAAATTATTAAAATCTCTACT
GGGTGTTCAACGTATGGAGAGATTGCCTGGTGACACACCACTAATGGAGTCAGGAATGGATTCACTGG
AGTTGTTAGAATTTCGTGCTCTTATAGAAAGAAAGTTTGGGATTAAGTTAAAGTCTACCTTCTTTTTTA
GTTACAAAACTCTTATAGCGGTAGCAGAGTATCTTTCAGAACGGGAAGATATTAATTTTAGTTAGTAG
GACTATGACTGAAAAAAAAACAACGTTTTCACAGGTCCACAGGGGGAAATTTTGAATCAGGGGGAAA
ATTTGAATGCATTCTAACCAGAGCAGTTCACTAGCAAGAATAGCCATTGTAGGTGTGGCTTGCCGTTTT
CCTGGAGCAGATACTAAAGAAGAATTTTGGCAGCTACTTAAGGAAGGTCGGGACTCAATGCAGAATCT
TCCTTCAGAGCGTTGGGGAAATCTATTTAACTCCATGAGTTCAGAAATAGATTTATCTATTCAACGCGG
TGGTTTTTTGAAAGATATAGATTTGTTTGACTCATCCTTTTTTAGGATTACTCCTAGGGAAGCGCAGTTA
ATGGACCCCCAGCAAAGGCTGTTGTTAGAATTGTCCTGGGAAGCGATGGAAGATGCAGGATATGCAAG
GGATACATTAAAGGGTAAATCAGTAGGGGTTTATGTAGGAGTTTGTCACTATGACTACAGAAGTTTAT
TAGAAAAAGGTTTGGAGACAGCCGAAATAGCTCAAATTGCTACTGGTACAGCTCCAGCAACTTTTGCC
AATCGCCTTTCCTATTTTTATAATTTTCACGGCCCTAGCTTAACTGTTGATACGGCTTGTTCCAGCTCTT
TGGTAGCAATGTATGAAGCAGTAAATGCAATACGTCGTGGGCAGTGTCAAACAGCATTGGTAGGAGGC
GTTAATTTAATGTGTTCTCCTGTGAATAACCAAGTTTATAGTGCTGCGGGTATGTTATCTCCTGATGGA
GTCTGCCGGGTGTTTGATGCGGGTGCTAATGGGTTTGTCAGGGGAGAAGGAGGGGCAGTAGTTGTACT
TAAAGATTATCAAAAAGCGTTGACGGATGGTGATTCTATTTATGGGGTTGTTCGCAGTGTAGCAGTGA
ATCATGGAGGGCAAGCAAGTTCTTTTACAGCACCAAATCCCCAAGCTCAGGCAAAATTGTTGGAACAG
GCTTATAGGGAAGCTAATATTGATATAGAGTCGGTAGGTTATATTGAAGCACATGGTACGGGTACATC
TCTAGGAGACCCAATTGAGGTCGAGGCTTTAAATGAAGCGTTTAAAGGGTTGAGTTCAAGTGGTAAGT
TACCTGCTAATAGTTGTGGTTTGGGCTCAGTTAAAACAAACATTGGTCATTTGGAAGGGGCAGCAGGT
TTGGCTGGACTAATCAAAGTTTTGCTGTGCATGAGGTATGCAACTCTTCCATGTTCATTAAATTATCAG
CAACTTAATCCTGATATTGAATTAGAAGAAGGTCCGTTTTTTGTGGTGGACAAGCTTCAATCTTGGGAA
ATAAAAGTTGATAGGGTAGGAAAACCTTATCCACTTAGAGCAGGGTTGAGTAGCTTTGGTTTTGGCGG
TACAAATGCTCATGTAGTTCTTGAGGAAGGAGAAAATAAGAAGGAAGGAAGAAGAGGGGATAAGGA
ACCTTCAGTTCATCTATTAACCCTTTCAGCTAAAACTGAAACAGCTCTTTCAGAGTTAGTCAGTCGTTA
TCAAAAATATTTAAACACTAATGCAGAATCAGAGTTAGCCGATATTTGTTATACCGCTAATACTGGGC
GAGTCCATTTTAACCATCGACTAGCAGCGATCGCCTCAAACAAACAGGAGTTAGTAGAAAAACTTAAG
GAATACCAAGCTGGGGAGGCAACAGCGGGAGTATTAATAGGGGAACTTGCAGAACGCATAAGAACAC
CGAAATTAGGTTTCTTATTCACAGGTCAAGGTTCCCAGTATGTCAATATGGGAAAACAACTGTATAAA
ACACAGCCTGTATTCCGTGAAGTATTGGATAAGTGTGATATAATATTGGAAACGGAAATAGAATGTTC
TCTATTAGATGTTCTATATAACAAAACTACAGATTCTCAAGATTCATCTTTAATAAACCAAACAGCTTA
CACTCAACCAGTTCTGTTTGCAATAGAGTATGCGCTATTTAAGTTATGGGAATCTTGGGGAATTAAACC
CAGCATAGTAATGGGTCACAGTGTAGGAGAATATGTGGCTGCTTGTGTAGCAGGAGTCTTCAATCTTG
AAGATGGTTTGAGATTAATAGCTGCTAGGGGCAGGCTGATGCAACAGTTACCTTCAGGTGGAGAAATG
GTTTCTGTGATGGCTTCAGAATCTACAGTTAGCAGACTCCTAGAACCTCACAAAAAAGAAATAGCGTT
GGCGAAGCCCGCCGTAGGCATCGCAGCAATAAATGGACCAGAAAGCACAGTTATTTCTGGAGACTCTG
TAGCAGTAACAGGTGTAGTAAATGACCTGGAAGCAAAGGGAATAAAAACTAAAAAACTAGAGGTATC
TCATGCTTTCCATTCACCATTAATGGAACCAATGTTAGGAGAGTTTGAAGCTATAGCTAATCAACTAAC
CTACAATCAACCTAAAATACCTATTATATCTAACGTTACAGGTACAAAAGCAGATAATACTATTGCTTC
CCCTCAATACTGGGTTAATCATGTCGGTAAACCTGTAAGATTTGCCCAAGGAATGGAAACGCTACATC
AACAAGGGTATGAAACCTTTATAGAAGTTGGACCGAAACCAGTATTGTTAGGAATGGGCAGGCAATGT
CTACCAGGAAATGTAGGTGTCTGGTTGCCATCATTACGTCCGGGAGTGGATGAGTGGCAACAAATGCT
TTATAGCTTGGGAAAATTGTATGTAACAGGAGTAAAAATAGATTGGTCAGGATTTGAGTCTGACTCTA
GTCGCCAGAAAGTAGCATTGCCAACTTATCCATTCCAGCGAGAAAGATATTGGATAGAAATAACAGAA
AACAAACATAAGGAACATCAAAAGTCAGAAAATATAAGTGACACTTCAATTGTTAAACTACTCACTCA
AGGAAAAACAGAAGCTCTCACTCAACAACTAGAAACAGAAGCCAAATTTTCACCAGAAGAGCTTAAA
CTTTTACCAGAAATATTAGAGACATTAGCCAAACAACATCAAGAACAATTAACAGCAGTAACCATCAA
AAACTGGTTCTACGAAATCCAGTGGAAACCCTTAGCTCAAAACAACCCCAATGCAAACATTGAACCTA
GTCATTGGTTAATTTTAGCCGATACCACAGGAGTAGCAGAAAAATTAGCTCAAAAATTACAACAACAG
GGTCATAAATACAGCTTAGTTTATCGAGGAGAGAGCTATCAAAGACAAGCAACAGGTACTTATCAACT
TAATCCTCAGATTCCCGAAGCATTTGAAAAGCTGTATCAAGAAATTCAACAAAGTAGTGAAACTGCCA
TTACGAAGTTAATTCACTTGTGGAGTTTAGATGCTCCCCAATCAAAAGACTTAACCCTGGAAACCCTAG
AAGAAGCTCAATTATGGGGATGTGGCAGCGTAGTACACCTGTTACAGACCTTAGTCAAAAACTCTAGC
ATTCCTGAACTATGGTTAGTAACCCGTGGGTCTCAATCAGTATTATCCCAAACAGAAAAAAATCTAAC
AGGACTAGCAGCGTCACCCTTGTGGGGATTAGGTAGAGTAGTGTCTAATGAACATCCCCAATTATGGG
GAGGATTAGTAGATTTAGACCCACAAGCTGCAGCAGGAGACGAAGTAGAAATGCTGTGGCAATTATTA
GTTAATGAACAAGAAGAAGATAATCTAGCTGTACGGGGAGAAAATACCTATGTAGCTCGTCTGGCCAG
GCAAGAACCTCAAGAATTTCCTGAATCCCTATCCTTATCATCAGATGGTAGTTACCTAATAACAGGAG
GGTTAGGAGCTTTAGGGTTACATACTGCCCAATGGTTAGTATCCAAGGGAGCGAAAAATATTGTCTTA
ACTGGGCGTCGCCCTCCCTCAGAAAAAGTAAGTGAATCTATAAAAAAATTAGAAGAAACAGGATGTC
AAGTGAAGGTGATGTTGGGGGATGTTTCTGTTGAAGAAGACATAGGCAAAATTCTCAAACAAATTCAG
ATATCAATGCCAACACTCAAAGGCATAATTCATACAGCAGGAGTGTTAGATGATGGAACCATACAACA
AATGAATTGGGAGCGTTTTGCCAAAGTCATGTCGCCCAAGGTAAAAGGAAGTTGGCATTTACATAAAT
TAACTGAAAATCAGCCATTAGATTTCTTTGTGTGTTTCTCCTCAATAGCTTCGATGTTGGGGACTCTTGG
CCAAGGAAACTATGCTGCAGCCAATGCCTTTATGGATGCTTTAGCTAGTTATCGTTGCAGCAGAGGATT
ATCAGGATTGGCGATTAATTGGGGAGCATGGGCATCAGGGGGAATGGCTGCTCGTTTAGCAGTGGAGC
ATCAAAATAGGATGCACAGCAGTGGCATAACTGAGATGGCGACCAAAGAAGGAATGTATGCCTTAGA
TTTACTATTAACAAATGAATCTGCTACAGCTCAGGTAGGTGTAGCAAGTATAGAGTGGCAGGTACTCT
CAGAAAGTTGGAGTGGTATCAAAAAAAATTCATTGCTACGAGAATTATTAGAGAAGGAGGAATGGGC
AGCAAAAGATACACGACACCAAAAAGTTAAAGCCGAATTTTTAGCAAAATTAGAGGAGGCATCACTA
GAGAAACGTCAGGAAATTTTAACTGAACACATTCGAGTGCAAGTATCTCAGGTACTAGGTTTAAGTTC
ATCTAAATTACCAGAAGTAAATGTAGGTTTTGCCTCTATGGGAATGGACTCTTTGATGACAATAGAATT
AAAAAATCGACTGCAAAATCAACTAGGAACCAAGTTGCCTGAGACAATAGCTATCGAATATCCAACTA
TTGCAAAATTGTCCTTAGGTATCAAAAAAAATTCATTGCTACGAGAATTATTAGAGAAGGAGGAATGG
GCAGCAAAAGATACACGACACCAAAAAGTTAAAGCCGAATTTTTAGCAAAATTAGAGGAGGCATCAC
TAGAGAAACGTCAGGAAATTTTAACTGAACACATTCGAGTGCAAGTATCTCAGGTACTAGGTTTAAGT
TCATCTAAATTACCAGAAGTAAATGTAGGTTTTGCCTCTATGGGAATGGACTCTTTGATGACAATAGAA
TTAAAAAATCGACTGCAAAATCAACTAGGAACCAAGTTGCCTGAGACAATAGCTATCGAATATCCAAC
TATTGCAAAATTGTCCTTATGTATAGAGGAATTAATGGGATGGAAGACAACAGAAATTGACCCATTAT
CTGAAGAAACAAGATTAGAGATGATGGAGCGCTCAAGAAGTGAAGCAATAGCCATCATCGGCATAGG
CTGTAGATTTCCCGGAAATGCCAACACACCAGAAAGCTTCTGGCAATTGTTATCCAATGGCAAAGACT
CCATTACAGAGATTCCCCTAGAACGTTGGGATCTAGATTCCTACTACGACCCCAACCCTGATACTCCAG
GGAAAATGTATATCCGTCACGCAGCATTAGTAGAAAAAGTAGATCAGTTCGACCCACGATTTTTTGGA
ATCTCTAACCGAGAAGCTTATAGTCTTGATCCACAGCAACGCTTCATTTTGGAAGTAACTTGGGAAGCT
TTAGAAAGAGCTGGTATTAACCCTCAACAATTAGAAAACACTCAGACAGGGGTGTTCCTGGGTATCGG
TCAAAATGATTACGCGAATTTAGGTTTTCACCAAGCAGCCGAAGATATAAGCCCTTACGATGCTACAG
GAAATTTGTTTTGTTTTGTAGCAGGTAGGTTATCTTACTTTTTAGGAACGCAAGGTCCATCAATGGCAA
TAGATACAGCTTGTTCATCATCTCTAGTAGCTATCCATGAAGCTTGTGAGAGTCTGCGTCAGGGTGAGT
CCAACTTGGCTTTGGCTGGAGGAGTTCAACTAATTCTCTCTCCCGAAGTAACAACCGCACTATCAAGAT
TAAAGGCTCTAGCACCTGATGGTAAGTGTAAAACTTTTGATGCTGCTGCTGATGGTTTTGGTAGGGGAG
AGGGATGTGGCATTGTGGTACTGAAGCGTTTGTCGGATGCGCTCAAGGATGGGGACAGGATATCAGCA
GTAATTCCTGGTTCGGCTGTTAACCATGATGGACCAAGTAGCGGAATGACAGTACCGAATAAACTGGC
TCAGGAAAAACTGATTCAGAAAGCTCTCAAAGCAGCCAAGGTAAAACCGTTACAAGTGAGTTATGTGG
AAGCTCATGGTACAGGAACTTCTCTAGGAGATCCTATGGAAGTGAGAGCTTTAGCTAGGGTGTTTGAG
GAGGGACGTGATCAGGAAAATCCATTGAACATCGGTTCAGTTAAAACTAATATCGGTCATCTGGAAGC
AGCAGCTGGAATAGCAGGTATGATTAAGGTGATTTTGCAATTGCAACATCAGGAAATTGTGCCCCATC
TGCATTTTGCTAATCCTAACCCCTATGTTGATTGGGAGAATATGCCTCTACAAGTACCGACTCAACTGA
CTCCTTGGTTGTCGAAAGGGGAGAAAAGGGTGGCAGGAGTTAGTTCTTTTGGTATGAGTGGTACGAAT
GCTCATATAGTTTTAGAAGAGGCTCCTATTGAAGTCAGAAGGCAGAAGTCAGAAGTCAGAAGTGAAG
AATATCTAGAACGTCCGGTTCATATACTAACTCTGTCGGCCAAGACTGAAAAAGCACTAGAAGATTTA
GTTAATAGTTATGAAAGTTATTTAGAAGCCGAAAATAATGATAATTATTTAGGGGATATTTGCTACACA
GCCAACATCGGGAGAGCAAAATTTGACCACAAATTAGCAGTGGTTACTTCTGATAAACAAGAGTTATT
AGAGAAACTCAAACAATATAAACAAGGTGAGAATGTTGCTGGAATCTTTTCAGGAAAACAAATAAGT
GAAACTAGAACAAAAATAGCCTTTATATTTACTGGTCAAGGTTCTCAATATCTGCAAATGGGAAGGCA
GTTATACGAAACTCAACCCACTTTTCATAAAATTATTGACCAGTGTAGTGAAATGCTGGTAAAATATTT
AGATGTTTCTTTATTAGATATACTTTATCCAGTTGAGGTTAAAGATGAAAGTTCGACTTTGATAGACCA
AACAGCTTATACTCAACCCGCTATATTTGCCCTTGAATATGCCCTAGCTAAATTATGGGAATCATGGGG
TATAAAGCCAGATGTAGTCATGGGTCACAGTGTAGGAGAATATGTGGCAGCAACAGTAGCAGGGGTA
TTTAGTTTAGAAGATGGTTTAAAATTAATAGCCATGCGGGGACAGTTGATGCAAAAGTTACCCTCCGG
AGGTCAGATGGCATCTATAATGGCATCAGAATCTCAGGTAATAGAGGCGATAAAAGAATATAGTTCCC
AAGTAACAATAGCAGCAGTTAATGGACCAGAAATTATAGTAATTTCAGGTGAGAGTCCAGCCATTTCA
AAAATTTGTAGTAAATTTGAATCAGAAGGAGTCAAGACCAAGGCGCTACAAGTATCTCATGCTTTCCA
TTCCCCATTGATGGAACCAATGTTAACAGAATTTGAAGCAGTAGCCAAAGAAATATCCTATAATCATC
CCCAAATACCACTAATATCAAATGTTACTGGTCAAGAAGTAAATGGAGAAATAACCACTGCTGAATAT
TGGGTGCGTCATGTGCGTCAACCAGTAAGATTTGCGGAAGGGATGGAAACTTTACACAAACAAGGTGC
TGAAATTTTCCTAGAAATAGGCTCAAAACCGATACTTTTAGGTATGGGTCGTGAGTGTCTCATGGGAG
AAAAAAAACTATGGTTGCCCAGTTTACGTTCAGGAAAACCAGACTGGTTACAAATGCTACAAAGTCTG
GGGCAATTGTATGTACGAGCAATAAAAATTGATTGGTTAGGATTTGATCGGGATTATTTCCGTAATAA
GGTAGAGTTACCAACATATCCTTGGCAACGAAAAAGGTATTGGATAACAGATATTAGACAACGGAAA
AGTCAAGACAAAAAAAGCATAACATCTGAAAAAAAAGTACAACTTGATGGAGTAAATATTCAACTAA
AGGAAATACAAATGAACGACAAAATCTTACAGCAACCAAAACTAAAATTATCAGATCCAGAATCACT
ATATTTATCGAATGCTGAATCAACTATAGAGGTTCCAACAAAAGCAGTTCAAGTAAAACCTGCTGCTG
ATATTGAATCTGAAAATATAACTCAATTGAATAGCCTTGATAGAGATGTGACTCAAATCATAGAAACC
CTCAAAGAAAGTTTGGCAGATGCTTTATATGCAGATATAAGTGAAATTGAAGAAGACAAAAAGTTTGT
TGATTTAGGTTTAGATTCTATTGTGGGAGTAGAATGGATAACTAACATCAATAAAATCTACAATTTAAA
TATAAAAGCTACTAAACTATACGATTACCCTACTTTGCCAGATTTGGCTAAATATGTCGCTCACACCCT
ATCAGCTCAGGGTAGAAATATTGATGTAGAGCGATCGCCATCTGACTCAAGTCAAGCAAGCATTAGCA
AGCAATCTCAACTAACTGATACCCAGAGCAATTTCTCACAAGTCAAGGAAATTTTGAAACAACAATTA
GCAGATGCTCTGTATGCGGATATTAGTGAAATTGAAGTAAACAAGAAGTTTGTTGATTTAGGTTTAGAT
TCTATTGTTGGAGTAGAATGGATAACTAATATCAATAAAACCTACAATTTGAATATCAAAGCCACTAA
AATATACGACTACCCTACCTTATTAGATTTCGCAAAATATATTAACCAGGAAATTTATTCTACAGGAGT
AAGTAGATTTTCAACGGAGCATAAAGAATTTAATCAAAAAGACTATTCATCTGGAGGTTCGCAAGAGG
AAATGACACAAAAATTACGATCAATATTGAAGAAAGTGGCCAATAAAGAATTAACAGTTCAGGAAGG
AAATAAAATGATTCAACAAATAAAAAATCAATTAAAGTAAAAATATAGTATAATTTTTAAGGAAATAA
AATTTTATGAACAAAGAGCAAATATTTCAAATAATAAAAAAATATACCTACGAAATAGCACCAGAATT
AGAAGAAGTACCTATATCACCTACTGATAGTCTCAAAAACTTGGGGATTGATTCAGTAAATAGGGCAG
AAATTATAATGATGGTAATGGAAGAGTTATCATTAAATATCCCGCGTATTGAATTAGCTGGATCCAAA
AATATAGGAGAACTGGCTGATATATTTGCATTCAAATTAGAAGCAATAAATTCACAAAGCTAGAAGGC
CTATCAATGAGTAACATAGAAATAACAGGTATGGGCATTGTTACTTCCATTGGTCAAGGAGTTGCTACT
TTTAAAGAAGCTCTGTTATCAGGGAAAACTCAATTTGCTTATCTAAAACAGCCAGGACGTGAAAGCAT
CAAACCATTTATTGGTGCCGAAATTCCCGATATTGACGCCAAAACCCTATTTCCTGAGTACAGTGGACT
GTTACGTACTGCTACTAAGAGCGCCCAAGTTGCAATAGTAGCTGTCGCTGAAGCCTGGCAAGATGCTC
AACTCACCTCCAGCCAAGTTAACCCAGAACGAGTAGGATTAGTTGTAGGTGGTTCAAATTTACAACAA
CGTTATCAGCAGCAAACCTGGCAACGTTATCACTCACGTCAGGAGTTTATACGACCAACTTATGGTCTG
ACCTTTTGGGATACAGATATATTAGGTCTGATTTCCCAGTGTTTTCAGATTCAAGGTGAGGGCTATAGT
GTAGGTGGAGCTTCGGCCAGCGGAGCAGTTGCTATAATTCATGCTGCTCGTCAAATTTTAATGGGGAA
CAGTGATGTTTGCATTGCTCTGGGAGCTTTATCCGATCTATCAGGGTATGAATTCCAAGCACTAATGAA
TTTAGGTGCAATGGGGAGCGAACGTTTTGCCGATCGCCCAAACCTTGCTTGTAGGCCATTTGACCAAG
ACCATGATGGCTTTATTTATGGAGAGGGTTGTGGAGCAGTGATTTTAGAGAGAACTGACCGCGCTCAA
CAACGGGGCGCTCAATCCCATGGCCAACTTAAGGGTTGGGGATTAACTTTAGATGGGAACCGTAGTCC
CGAACCTTCCCAAAAAGGGGAAGAACGGGCTATAAATACTGCTTTAGCCATGGCAGACCTTCAGCCAG
AGAGTATTGATTATGTGAATACCCACGGTACTGGTTCCCCCCTTGGCGACAAAACAGAAGTGGCAGCT
TTCAAATCAGTAGGACTTCAGCATTGCCTATTTAATTCAACAAAATCTTTAATAGGCCACTGCTTAACT
GCTGCTGGTGTAGTGGAAGCGATCGCCACCATATTACAAATGAAGTTTGGTTTTTGTCATCCAACTAAA
AATTTAGTTAATCCTATTGATACCAGTCTCAATTGGGTGAAAGAAACTTCTGTTCAAGCTGAGATTAAA
TATGCTATCAGCAACAGTTTTGGTTTTGGGGGTATCAATACAGCTTTATTAATTGGACAGGAGTAAACT
CATGCAACAAGTTGGAATTGAAGCACTAAATGTATATGGGGGTTCAGCTAAACTAGATGTGCGAATGC
TAGGCGAAGCACGTCACTTAGACATGACTCGCTTCGATAATCTTCTGATGAAAGAGAAGACAGTTGCT
ATGCCCTATGAAGATCCAGTTTCTTATGCTATTAATGCAGCAAAACCTATTATTGATAGCCTCAGCGTT
CTGGAAAAGCAACAAATCAAAATGGTGATAGCTTGTAGCGAATCTGGCATTGATTTTGGCAAGTCTAT
GAGCACTTATATCCAGGATTATTTGGGACTAAATCGTAACTGTCGAATGTTTGAAATAAAGCAAGCTT
GCTACTCAGGTACAGCAGGTTTACAAATGGCATTCAATTTAATTTTGTCCCAAACCTGTCCAGGGGCTA
AAGCTTTGGTTATAGGAACTGATATATTTCGGCCTGTTGTTGTAGAGGGAGGAGAAGCTCTCAGCGAA
GATTGGTCTTTTGTGGAACCAAGCAGTGGTGCTGGAGCAGTAGCTATTTTAGTCAGTGATGTTCCTAAA
ATTTTCCAAGCTGATGTAGGATGTAATGGTTACTATGGCTATGAAGTCATGGATACTTGCAGACCTAAC
CCAGACTCAGAAGCAGGAGATGCAGATTTATCATTGCTATCTTACCTAGATTGTTGTGAAAATGCTTAC
CGGGAATATCAAGATCGAGTAGAAGGAGTAGATTACCAAAAAACTTTTGATTACTTGAGCTTTCATAC
TCCTTTTGGGGGTATGGTGAAAGGGGCTCACAGAAGTATGATGCGTAAGTTTAAAAGGGCAAAACCTG
TGGAGATTGAAGAGGACTTTCAGAAACGGGTGATGCCGGGATTAGTCTATTGTCAACAGGTGGGTAAT
ATTATGGGAGCTACAGTATTTTTATCTTTAGCTAGCATGATTGATAATGCAGATTTTAGCAAAGCCCGA
CGAATTGGTGTATTCTCTTATGGCTCTGGTTGTTGTTCGGAATTTTATAGTGGAGTCGTCACCCCGCAA
GGAAAGGAAATTCAAGCTCAACAAAAAATTTCATCACAGTTAGCAATGCGCTATTCCTTAAGTATGGA
AGAATATGAGCAGTTACTAAGTCATAGTTCGGCAGTTGCCTTTGGCACTAGAAATGTTACCCTAGATTA
CAAACTATTTCCTGGTGTGTGGGAACAAATTGAAGGTAAAGGTCGCTTAGTGCTGAAAAGAATCAAGG
AATTTCACCGAGAATATGAATGGGTATAGCCATGAGTTATCAAACCCTGAAAATCAGTTATCAAGATG
TTGTACAAAGGATTCAGATATATCGACCTGAATCTAACAATAGCATCAATAGTCAATTAACGATGGAA
TTGTTGTCAGCTTTGCAAGCTGCTGAAGCAGAGGAAGTTGTTAAAGTAGTGATATTAGAAGGACTACC
TGATGTATTTTGTACAGGGATGGATTTTGAAGAAGTGGCAACAGCAAAACAATTTGATCCAAAAGCTA
GTGCTAATGGTTACTACAATATTTTGAAACAAATGTCTCAAAGTAGCAAAGTAATTTTGTCACTTGTGC
GTGGTAAAGTACAGGCAGGGGGAGTTGGTTTAGTAGCAGCAAGCGATCTAGTTATTGCTGATGAAACG
GCAACTTTTGTTTTATCAGAATTATTATTTGGATTATTACCAGCTTGTGTATTGCCTTTTTTGATTCGTCG
AGTGGGATTTCAAAAAGCCTACCGTTTAGCACTAACGACTCAAGCTATTTCAGTATCAGAGGCTGATA
AGTGGGGATTAATAGATGAATATGGCAGTAATATTAATCAGTTAATAAGTAAATATATTCGACGTTTA
AAGTATTTACCCTCATCAGGGGTGAAAGAGTTAAAAAATTATATTAATCAGTTATGGATTATTCAGGC
AGAAACTCAAGGTTTAGCCGTCAACGAGATTTCTAGCTTAATAGCAGAACCTACTGTTCAAGAAAAAA
TTAAACGTTTTCAAAAAGAAGGATTATTTCCATGGCAAACCTAAATCTTAATTTGGACTTAGTAGAGGG
CAACTCTGATGTAGTACAACTGGTGGAGTTGGGTAATGGTGTCGTGCAAATCACGATGAAAGATGAAG
AAAGCTGCAATGGCTTTTCTCCTGGAATAATTGAAGGATTATACAAATGTTTTGGTGCAGTTGCTCAAA
ACCAAAGTTATAAAGTAGTAATTTTGACAGGCTATGGAAATTATTTCTGTTCGGGAGGGACAAAAGAA
CGGTTAATCAGTATTTGGAAAGGAGACAGCAAATGTAATGATTTAGATTTTTTTAGAATAGCATTAGAT
TGTGAAATACCAGTAATTGCAGCTATGCAAGGTCATAGTATTGGCGGTGGTTTGGTTTTGGGATTGTAT
GCAGATTTAGTAGTGTTAAGTCAAGAAAGTATTTACACTACTAATTTTATGAAGTATGGTTTTACTCCA
GGTGTCGGATGCACGTTAATTCTCCCTGAGAAATTCGGTGCTTTAGGGTTTGAAATGATGTATACTGCC
CAAAATTATCGAGGGAAAGAATTAGCTGAACGGGGTGTTTCTTTTCCAGTTGTACCGAGAAAAGATGT
GCTAGAAGTGGCTAAAAATATAGCCTATGAAATGTCGGAAAAACCCAGACTATCTTTAATAACTTTAA
AGGAACACTTAACTTCAAAAATCCGCAAAACGCTACCAGGATTTATAGATAAAGAAGTAGCTATGCAC
GAAATAACCTTTCACCAACCAGAGGTAGCAAGTAGAATAGAGGAAAATTTTGACAAAAGGACAACGG
CAAGTAATAACCCTCAAAATTTTCCTCAAGAGGCTGTAAGAAAAGAAATAAGAACAGATTCATTGAAT
TGTCAACCATTTCAGTTGAAGACATTTAGTTATGGTTCGTTAAACAATTTAACTTTGGTACCTCTAGAA
CGTAGAGTTCCAAGCCCAAGTGAAGTTGAGGTTCAAATCAAAACTGTACCAGTTAATTTTCGGGATAT
ACTCAATGCACTTGGTATGCTCCAAGAGTATTACGAAAAAACATTTGGCATTGCTAATGCTGAAGATCT
CACTTTTGGTTTTGAAGGTGCAGGTACTATCGTAGCTGTTGGGGCAGAAGTATCGCAGTGGCAAGTCG
GCGATGAAGTAATGGTAATGAGAATTCACGATGCATTTAGTAGCTTTATTATCTGCTCGCCGGACAAA
CTGGTGCGTAAAAATTTTAATCTGAATATGGAAGATGGAGCTAGTATTTGGGGGCCGTTTTCGACTGCA
TATCACGGGTTGATTAACTTAGCCAAAATTCAGCCAGGAGATAGAGTATTAATTCACGCTGCTTCAGGT
GGGATTGGACAAGCAGCAATTCAGTTGGCTCAACTTTTTGGGGCGGAAGTATTTGCTACTACTAGTCCA
GGTAAGATGAATTATCTCCGGGAACAGGGAATTAAGTATGTGATGAATTCTCGAACGATGGAGTTTGT
AAATGATGTGATGGAATTCACTCAGGGCCGTGGGGTAGATGTTATTCTCAATAGCCTGACTCATGGAG
AATACATCCAGAAAAATCTAGAGATCCTTGCCGATAGAGGGCGATATGTTGAACTTGGTAAGTTGGGC
ATTTGGAGTCACGAGCAAGTCTATCAGAAACGCCCAGATATCAAATATTTTACTTTTGATTTGTTAGAA
GAATTTGCCAAAGATAATCAATTGTTTTCTCAGATATGGGATAATTTGGCACTTGAATTTGACCGCGAT
CGCTTGAAGCCACTACCTTACAAAACATTTCCAATAGAAGATGTTCTTAAAGCCTTTGATTATATGCGA
CGCGGGAAGCATTTTGGCAGGGTAGTGGTAGTTATGCCTGACTCCTATTCTAGACAGGAACAAGAGTT
GGATGCTCGGTTATCCATTGAAAACAAAATGACTAAAGAGGAACAGATATTATTTCAATTACAATCTG
GTGAAGTTTCCTTAGAAAATGCGGAACAACTATTGTTAGGAAATACGGAAACAGAAACACAAGATAA
AGCTATAGCAGAAAATCAAATAGATAATATTCAAAACAAGTTAATTAACATGGATAGCTCAGAGAAA
ATCTTATCTTTGATTAGTTCAGTAGAAATATCTTTAGAAACAGCAGAAAAATTATTATTAGAAGTAGTA
GAACCAGAAGTTAAAACAGAGGTTAATGATGAGGTTAATCCTAGTCAAAATCATATACCAACTACAGA
TATAGCGATTATTGGTATTTCATGTAGATATCCAGGAGCGAATAACTGGAAAGAATTTTGGGAAAATT
TAAAGAATGGAATTGACAGTGTAACGGAAGCTCCTCCTGGAAGATGGGAAGAAAAAAATTGGTATCA
TCCAGATCCAGATAATCCAGGTACTTCCTATTCAAAATGTGCCGGTTTTTTAGATGAAATTGATAAATT
TGACCCTTTATTTTTTCATATTTCTCCGGAAGAAGCTTGGTTTATGGAGCCTGAGCAAAGAATATTTTTA
GAAGAAGCTTACCACGCTATAGAAGATGCTGGATATGCTACAGACTCTCTTAGAGGTAAACAATATGG
AGTATTTGTGGGAGTTACGGTAAATGGTGGTTATCTTAAGTTGTTGTCAATTTCAGGATTAGATGTTCA
TAGGATGGCGGCTACAGGAAATGGTCCGTCAATGATACCAGCAAGAATTGCCTATATGCTTGACCTTC
AAGGGCCAGTAGTAGCTATTGATACTGCCTGCTCGTCGTCATTGGTAGCTGTTCATCAAGCTTGCCAAA
GCATACAACGAGGAGAGAGCGAAATAGCGATCGCTGGAGGTATTACTCTAATGCCAACATCAGACTTC
CAAATAATGTCAAGTCAGTTTCAAGTTGTATCTCCTGATGGACGTTGTAAGACTTTTGATGCTTCAGCA
TCGGGTACAGCCTGGAGCGAAGGTTGTGGTGTTCTCTTATTAAAAAGCTACAGTCAAGCAATTCAAGA
CAACGACCATATTTATGGAGTAATCAAAGGAACAGGAGTTAATTATGATGGTAATACTAACGGGATTA
GTGCCCCTAGTAGTCAATCTCAAGCCAGTTTAGAAGAAGCAGTTTATCAGAAATTTGGAATTAACCCA
GAAACTATTAGTTATGTAGAAGCTCATGGTACAGCAACACCTCTGGGAGACCCGATAGAAGTAGAAGC
TTTAACAGAAGCCTTTTCTAAATGGACGAACAAAAAACAGTTTTGTGCGATTGGCTCGGTAAAAACTA
ATATTGGTCATTCAGCAGCGGCTGCCGGAGTTTCTGGTTTGATCAAGACAATTTTGTGCCTCAAAAATC
AAAAATTAGTTCCATCCTTACATTTTAATCAACCAAACCCACATATTGACTTTGAAAATAGCCCTTTTT
ATGTCAATACAAAATTAAAAGATTGGGAAGTGCTTGAAGGCCAACCAAGGCAAGCTACAGTTAGTTCT
TTTGGTTTTAGTGGCACTAATGCTCATATAGTTATAGAAGAGGCTCCTTCTCAAGTTAAAAGTCAGAAT
ATTGTTGAACGTCCGATCCATCTGTTAACTCTATCTGCGAAAACAGAAAAGGCTCTAGAAGATTTAGTC
AGTAATTATCAAAATTATTTAGAAACTAATCCCGAGTTACCACTAGCAGATGTATGTTATACAGCCTCT
ACAGGTAGAGCACATTTTAATTATCGATTAGGAGTTATTGCTTCTGAACCAAAAGCATTAATAGAGAA
ACTACTTGGGTGGAAAGCTCAGGAAGAATTAGTAGGACTATTTTCAGGAAAACGAAATAGCGAAGGT
CAGAAAATAGCATTCCTGTTCACAGGTCAAGGTTCCCAGTATGCGAATATGGGAAGGCAACTTTATGA
AAAAGCACCAACTTTCCGTCAAGCTTTAGAGGAATGTGACCAAATTTTACAACCCTATCTAGAAGTAC
CTCTATTAGAGGTCATATACTCTGAGGATGCACAAAAGTCAAGTGATAATCTATTAGACCAAACAGCT
TACACCCAACCAGCTGTGTTTGCTGTTGAATATGCTTTGGCTAAATTATGGTCTTCATGGGGAATCAAA
CCATGTGTAGTCATGGGTCACAGCGTAGGAGAATATGTAGCAGCAACAGTAGCCGGAGTATTCAGTTT
AGAAGATGGTCTGAAACTAATAGCCATGCGAGGAAAGTTGATGCAAAAGTTACCCTCCGGTGGTGAG
ATGGTATCCGTAATGGCATCAGAGTCTCAGGTAACAGAGGCTATAAAAGAATATAGTTCCCAAGTAAC
AATAGCAGCGATAAATGGACCAGAAAGTATAGTAATTTCTGGTGAGAGTGTAGCCATAAAAAATATTT
GTAGTTTATTTGAATCAGAGGGAATAAAGACCAAGCAGCTACAAGTATCCCACGCTTTCCATTCTCCA
ATGATGGAACCGATGTTAGCAGAGTTTGAATCAGTAGCTAAACAAGTCACCTATAATCAACCCCAAAT
ACCACTAATATCAAACGTCACAGGTACTGAAGTAGATGGAGAAATAACAAATGCTGAATATTGGGTAG
ATCATGTGCGTCAACCAGTAAGATTTGCCCAGAGTATGAAAACTCTAGAGTCGGAAGGATATGAAACC
TTCCTAGAAATAGGACCGAGACCAATATTGCTAGGAATGGGAAGACAATGTGTAACAGAAGATGTAG
GAGAATGGCTGCCATCATTACGTCCAGGGGTGGATGAATGGGAACAAATGCTATCAAGCTTAGGAAA
ATTGTATGTAAAAGGAGCCAAAATAGACTGGTCAGGGTTTGACTCTGATTATACTCGCCAGAAAGTAG
TATTACCCACATATCCATTCCAGGGAGAACGTTATTGGGTAGAAACCAACAACAACTTCTGGCCTCAA
CGGCAATTTTCCCAAGGGGAAAACCTCCATCCCCTATTAGGTCAAAAGCTAAATTGTGCAGGGGAACA
ACAAATATTTGCATCACAAATAGGAGAAAACTCACCCAACTATCTGAGGGACCACCGAGTATTTAATC
AAGCACTATTTCCCATAACAGGCTACCTAGAAATAGCAATAGCAGCAGGAAATCACCAATTAAAAACA
TCCCAGATAGTAATAGAAGACCTAACCATAACTAGAGGATGGATACTACCAACAGGAGAATTAACCA
ATGCCCAAACCATACTCACCCCAATAGATAACCAAAGCTATAAGTTTCAAATATTTTCTCAACCAGAA
CAACAGGAGTGGAGACTCCACACGACAGGAAAAATCAGAAAAGAGTCAACCCCCCCTACTCAGACAA
AAGTTGACCTAGAAAAATACAAGAGTGAATGTAATCAAACAATAGAAGTCAAACAACATTATCAAAA
ATGTCAACAAGTAGGGATAGACTACGGGAATACCTTCCAAGGCATCCAAGAATTGTGGTCAGGTTCAA
ACCAAGCATTAGGTTATATCAAACTGCCCGAAGAATTGATAACACAAACAAGCGACTATCATTTCCAT
CCAGCACTATTAGATGCAGCCTTGCAAGTAATGTTTTATGCACTGCCAGCAACGGATAATGACAAAAC
TTATCTGTCAGCAGGAATAGAAGAATTCAGACTATATAAGACTCCCGGGCTGAGTATATGGGCATATG
TATCAGTAACCAGTCAAGAAGTGGAAACTCCAGAAAGTTTGACGGCTATTGTCACCATAGTAACTCCA
GAAGGAGAAATAATTGCCAACATCAAAGGTTTACAAGTCAAACTAGCAACAAAACAGACCCTACTGG
GAACAGAAACCGAATCAATAGAAAATTGGTTATATGAAGTAGAGTGGAGAAACAAAGGTATTTTAGG
TAAACTACTCCCACCAGATTTCCTCATACCTCCTATACAAATCAACCAAAAATTAACTCCAACTCTGAC
AGAATTAGTAACTCAAGTAGATAATGAAACAACAGCGTCTTTTGAAACAAGCTTAGAAGAATTAAGCA
GAGATTATATAGTACAAGCATTACAGTCAATGAGTTGGTCATACAAACCAACAGAAAGCTTTGCATTT
GATGTAGCAGCCCAAAAATTAGGTATAGTTCCTACCCATCGACCACTGTTTAAGCGTTTGCTGCAAATA
TTAACAGAGTCAGGAATACTCAACTCAAAGAATCAGCAGTGGGAAGTAGCACAAACCTTACCTGAAGT
CAAGCCTACGGAAAAAATCAGCAGTTTACAGAAGAAATATCCAGAAGAAACAGCAGCATTGACACTA
CTCTCTCGTTGTGGGTCTAAACTAAGTGGGGTATTACGAGGAGCAATAGACCCAGGAGAGTTAGTGTT
CCCCCAAGGAGATTTGACAGCAGCGACTCAACTTTATGAAGACTCAACAGTAGCGAAAGTGATGAACA
CAATAGTAGAAAAATCCATCACCAAAGCTATAGAAAAATCCCCGAAAAGCCGGGGGCTCAGGTTGTT
GGAAATAGGAGGGGGAACAGGAGGGACTACAAGCTATATCCTACCTCATCTAACTCCTCAGCAAACC
GAATATACATTCACGGATATAGGGGCATTATTTACAGCCAAAGCTCAAGAGAAATTCCGGGATTATAA
GTTCATAAAGTATCAAACTTTAGACATAGAAGTAGACCCGACAACTCAAGGATTTGAGGCTCATCAAT
ATGATGTAATTATTGCAGCTAATGTACTTCATGCAACGACAGATATGAAGCAGACATTATCTCATGTGC
GAGAACTGTTAGCAGATGGGGGAATGTTGGTGTTATCTGAAGCAACAGCTAAAACACTATGGGTAGAT
TTAGTATTTGGGTTGTTAGAAGGATGGTGGAAATTCAGGGATTATGAATTACGACCAGATTATCCTTTG
TTGAGTCGTGAAAAATGGCATCATGTCTTGAGAGAAACGGGTTTTACTGAAGTAGTTACTATGCCAGA
AGTGGAGGGAATGGCAGAAACATTGTCAGCACAAACGGTAATTGTAGCTAAAAGCTCTCAAACGAAG
TTAGAACAAAGGAATGATGATTCTAAGAGTTGGTTGATACTGGCAGACTCTGAGGGAGTAGGGCAACA
GTTAGCGACCTTACTCCGCTCAGTAGGAGAGGTTTGTACTTTAGTATTTGCCGGAGAAAGGTATCAACA
GATAGCTCCGGGAGAATTTAGTATTAATCCTAATCAAGCTAAAGATTTTGAGGAGGTAATAGAGACAG
TGGCAGGGAAGTCACCATCATTGTATGGAGTGGTACAATGTTGGACGACTGAAGCCGGAGTGGGGAAT
GGGATTAATTCTGAGGAGTTAGGAAGTTTATCGAAGTTAGGGTGTGGCACAACTCTATCTTTAGTACA
AGCATTGGTGAAAGGCGGGTTATCAACTGTCCCTCGATTATGGTTGGTGACAAATGGTGCTCAGGCGG
TGCCGAATAATCATCCGGTGATACCAGGAGTAGCTCAATCTTCGGTATGGGGAATGGGGAAAGTGATT
AGCTGGGAACATCCAGAGTTGAACTGTACTCGTATAGATTTGGACCCAGAGGAGACTTTAGAGGGTAA
AGTTGATGCCCTATTTAAAGAAATTTGGTCGGAGGATAGGGAAGACCAGGTAGCATGGCGTGGGGATA
GTCGTTATGTAGCTCGGTTGGTGGGTAGTCATCATCGGCAATTAGTGGCACAACAAGCTGATGGTAAA
ACTCAAAAGCCCTTAAGTTTCCGTTCTGAAGCAAGCTATTTGATTACAGGAGGTATGGGAGGTTTGGGT
TTGCTGGTAGCTAGTTGGATGGTGTCAAAGGGAGCTAAACATTTGATATTGTTAGGACGCCGTTCACCG
GATGATGCTACGAGGAAAAAAATAACCGAGTTAGAAATGGCAGGAGCGTCCGTGGTAGTGGAAAAAG
CCGATGTGACTGATTTGGAGTCGATGAAAGGGGTGTTGCAGAGGATTGAGGAGTCAAAGCGACCGTTA
GTAGGAGTGATTCATTCTGTGATGGTGCTATCAAATGGAGTGCTACGAAATCAGACTTGGTCTAGTTTT
GAACAGGTGATGGAACCGAAAGTTCAAGGTGCTTGGCACTTGCATCAATTGACTCAAAGTCAACCATT
AGACTTCTTTGTGCTGTTTTCTTCAGCAACATCTCTGTTGGGTTCACTGGGTCAGGGCATGGCAAATTAT
TCTGCTGCTAATGGGTTTCTTGATGGTTTAGCTCATTATCGTCGAACTATGGGATTACCGGGATTGAGT
ATCCATTGGGGAGCAGTTTCTCAAGTGGGACGAGCGCTCGGGCGAGATACAGAGACAGCCGCTATGCT
CAGTAAGAATGGGATGGGTTTAATATCTCCGGCTCAGGTATTGGAGTCTTTGGAACTATTGATGAGTA
GTTCGGATGTGGAAGTAGGGGTGATGCCTATTGAGTGGTCAGGGTGGCAGGAGAGAGTGGCACAGTG
GCCGTTTTTAGTGGATTGGCAGGAAACTATATTGGAGGTAGCCCAACCATCGAAGTCAGATTTTCTGTT
AAAGTTGGAGGCTACACCACCTAATGAGCTTCGCTTGTTGTTGGTGGCTCATGTGCGTCGTCAGGTGGC
TCAAGTGTTGAGAATTAGTCATCCTGAATCAATTGAAATGGATACAGGGTTTTTTGATTTGGGTATAGA
CTCTTTGACTTCTGTGGAGTTGAGGAATAAGTTGCAAGGTAGTTTAAAGTGTTCAGTACCTTCTACTGT
AACTTTGGACTACCCTACCATTAAGGCATTGGTAGAATATTTATATCAACAGCTATTGTTAGAACAAGT
TAGTTACTCAAATACTGTACCAACTGAAGAAATCAATGAAGATAGGGAGGAGATTACTCTAAGCGGGA
CACCAGCTCGGGAAGCAAGTAGTGCCTTACGGGATGGCTATCGTCAAGCAGGAGTCAGCGGGCGGGTT
CGGAGTTATTTGGATTTATTGGCTGGTTTAAGTGATTTTCGGGAACATTTTGATGGTAGTGATGGTTTTT
CTTTAGATTTGGTGGATATGGCTGATGGTCCCGGTGAAGTGACCGTTATTTGTTGTGCCGGTACTGCCG
CTATTTCTGGTCCCCATGAATTTACCCGCTTAGCCGGTGCTTTGCGTGGTATTGCTCCCGTGCGGGCTGT
TCCACAACCAGGTTATGAAGAAGGTGAACCCTTACCCAGTTCTATGGCCGCTGTGGCCGCTGTTCAAG
CCGATGCTGTTATTCGCACCCAAGGTGATAAACCCTTTGTGGTTGCTGGTCATAGTGCCGGTGCTTTAA
TGGCCTATGCTTTGGCCACTGAATTATTGGATCGTGGTCATCCTCCCCGCGGTGTGGTTTTAATTGATGT
GTATCCTCCCGGTCATCAAGATGCTATGAATGCCTGGTTAGAAGAATTGACCGCCACTTTGTTTGATCG
GGAAACCGTTCGCATGGATGATACCCGCTTAACTGCTTTGGGTGCCTATGATCGTTTAACTGGTCAATG
GCGCCCCCGTGAAACCGGTTTGCCCACTTTATTGGTGAGTGCTGGTGAACCTATGGGTCCCTGGCCCGA
TGATTCTTGGAAACCCACCTGGCCCTTTGAACATGATACTGTGGCCGTTCCCGGTGATCATTTTACTAT
GGTTCAAGAACACGCTGATGCTATTGCACGACACATTGATGCCTGGTTGGGCGGTGGAAACTCCTAA
CTCCCTATAGTGAGTCGTATTAGCGGCCGCATCGAATATAACTTCGTATAATGTATGCTATACGAAGTT
ATTAGCGATGAGGACATGAGGTTGCCCCGTATTCAGTGTCGCTGATTTGTATTGTCTGAAGTTGTTTTT
ACGTTAAGTTGATGCAGATCAATTAATACGATACCTGCGTCATAATTGATTATTTGACGTGGTTTGATG
GCCTCCACGCACGTTGTGATATGTAGATGATAATCATTATCACTTTACGGGTCCTTTCCGGTGATCCGA
CAGGTTACGGGGCGGCGACCTCGCGGGTTTTCGCTATTTATGAAAATTTTCCGGTTTAAGGCGTTTCCG
TTCTTCTTCGTCATAACTTAATGTTTTTATTTAAAATACCCTCTGAAAAGAAAGGAAACGACAGGTGCT
GAAAGCGAGGCTTTTTGGCCTCTGTCGTTTCCTTTCTCTGTTTTTGTCCGTGGAATGAACAATGGAAGTC
CTCGTCGAGGACGATCTTCCGCTGCATAACCCTGCTTCGGGGTCATTATAGCGATTTTTTCGGTATATC
CATCCTTTTTCGCACGATATACAGGATTTTGCCAAAGGGTTCGTGTAGACTTTCCTTGGTGTATCCAAC
GGCGTCAGCCGGGCAGGATAGGTGAAGTAGGCCCACCCGCGAGCGGGTGTTCCTTCTTCACTGTCCCT
TATTCGCACCTGGCGGTGCTCAACGGGAATCCTGCTCTGCGAGGCTGGCCGGCTACCGCCGGCGTAAC
AGATGAGGGCAAGCGGATGGCTGATGAAACCAAGCCAACCAGGAAGGGCAGCCCACCTATCAAGGTG
TACTGCCTTCCAGACGAACGAAGAGCGATTGAGGAAAAGGCGGCGGCGGCCGGCATGAGCCTGTCGG
CCTACCTGCTGGCCGTCGGCCAGGGCTACAAAATCACGGGCGTCGTGGACTATGAGCACGTCCGCGAG
CTGGCCCGCATCAATGGCGACCTGGGCCGCCTGGGCGGCCTGCTGAAACTCTGGCTCACCGACGACCC
GCGCACGGCGCGGTTCGGTGATGCCACGATCCTCGCCCTGCTGGCGAAGATCGAAGAGAAGCAGGAC
GAGCTTGGCAAGGTCATGATGGGCGTGGTCCGCCCGAGGGCAGAGCCATGACTTTTTTAGCCGCTAAA
ACGGCCGGGGGGTGCGCGTGATTGCCAAGCACGTCCCCATGCGCTCCATCAAGAAGAGCGACTTCGCG
GAGCTGGTGAAGTACATCACCGACGAGCAAGGCAAGACGATCCCCTCGACAGCGACACACTTGCATC
GGATGCAGCCCGGTTAACGTGCCGGCACGGCCTGGGTAACCAGGTATTTTGTCCACATAACCGTGCGC
AAAATGTTGTGGATAAGCAGGACACAGCAGCAATCCACAGCAGGCATACAACCGCACACCGAGGTTA
CTCCGTTCTACAGGTTACGACGACATGTCAATACTTGCCCTTGACAGGCATTGATGGAATCGTAGTCTC
ACGCTGATAGTCTGATCGACAATACAAGTGGGACCGTGGTCCCAGACCGATAATCAGACCGACAACAC
GAGTGGGATCGTGGTCCCAGACTAATAATCAGACCGACGATACGAGTGGGACCGTGGTCCCAGACTAA
TAATCAGACCGACGATACGAGTGGGACCGTGGTTCCAGACTAATAATCAGACCGACGATACGAGTGG
GACCGTGGTCCCAGACTAATAATCAGACCGACGATACGAGTGGGACCATGGTCCCAGACTAATAATCA
GACCGACGATACGAGTGGGACCGTGGTCCCAGTCTGATTATCAGACCGACGATACGAGTGGGACCGTG
GTCCCAGACTAATAATCAGACCGACGATACGAGTGGGACCGTGGTCCCAGACTAATAATCAGACCGAC
GATACGAGTGGGACCGTGGTCCCAGTCTGATTATCAGACCGACGATACAAGTGGAACAGTGGGCCCAG
AGAGAATATTCAGGCCAGTTATGCTTTCTGGCCTGTAACAAAGGACATTAAGTAAAGACAGATAAACG
TAGACTAAAACGTGGTCGCATCAGGGTGCTGGCTTTTCAAGTTCCTTAAGAATGGCCTCAATTTTCTCT
ATACACTCAGTTGGAACACGAGACCTGTCCAGGTTAAGCACCATTTTATCGCCCTTATACAATACTGTC
GCTCCAGGAGCAAACTGATGTCGTGAGCTTAAACTAGTTCTTGATGCAGATGACGTTTTAAGCACAGA
AGTTAAAAGAGTGATAACTTCTTCAGCTTCAAATATCACCCCAGCTTTTTTCTGCTCATGAAGGTTAGA
TGCCTGCTGCTTAAGTAATTCCTCTTTATCTGTAAAGGCTTTTTGAAGTGCATCACCTGACCGGGCAGA
TAGTTCACCGGGGTGAGAAAAAAGAGCAACAACTGATTTAGGCAATTTGGCGGTGTTGATACAGCGGG
TAATAATCTTACGTGAAATATTTTCCGCATCAGCCAGCGCAGAAATATTTCCAGCAAATTCATTCTGCA
ATCGGCTTGCATAACGCTGACCACGTTCATAAGCACTTGTTGGGCGATAATCGTTACCCAATCTGGATA
ATGCAGCCATCTGCTCATCATCCAGCTCGCCAACCAGAACACGATAATCACTTTCGGTAAGTGCAGCA
GCTTTACGACGGCGACTCCCATCGGCAATTTCTATGACACCAGATACTCTTCGACCGAACGCCGGTGTC
TGTTGACCAGTCAGTAGAAAAGAAGGGATGAGATCATCCAGTGCGTCCTCAGTAAGCAGCTCCTGGTC
ACGTTCATTACCTGACCATACCCGAGAGGTCTTCTCAACACTATCACCCCGGAGCACTTCAAGAGTAA
ACTTCACATCCCGACCACATACAGGCAAAGTAATGGCATTACCGCGAGCCATTACTCCTACGCGCGCA
ATTAACGAATCCACCATCGGGGCAGCTGGTGTCGATAACGAAGTATCTTCAACCGGTTGAGTATTGAG
CGTATGTTTTGGAATAACAGGCGCACGCTTCATTATCTAATCTCCCAGCGTGGTTTAATCAGACGATCG
AAAATTTCATTGCAGACAGGTTCCCAAATAGAAAGAGCATTTCTCCAGGCACCAGTTGAAGAGCGTTG
ATCAATGGCCTGTTCAAAAACAGTTCTCATCCGGATCTGACCTTTACCAACTTCATCCGTTTCACGTAC
AACATTTTTTAGAACCATGCTTCCCCAGGCATCCCGAATTTGCTCCTCCATCCACGGGGACTGAGAGCC
ATTACTATTGCTGTATTTGGTAAGCAAAATACGTACATCAGGCTCGAACCCTTTAAGATCAACGTTCTT
GAGCAGATCACGAAGCATATCGAAAAACTGCAGTGCGGAGGTGTAGTCAAACAACTCAGCAGGCGTG
GGAACAATCAGCACATCAGCAGCACATACGACATTAATCGTGCCGATACCCAGGTTAGGCGCGCTGTC
AATAACTATGACATCATAGTCATGAGCAACAGTTTCAATGGCCAGTCGGAGCATCAGGTGTGGATCGG
TGGGCAGTTTACCTTCATCAAATTTGCCCATTAACTCAGTTTCAATACGGTGCAGAGCCAGACAGGAA
GGAATAATGTCAAGCCCCGGCCAGCAAGTGGGCTTTATTGCATAAGTGACATCGTCCTTTTCCCCAAG
ATAGAAAGGCAGGAGAGTGTCTTCTGCATGAATATGAAGATCTGGTACCCATCCGTGATACATTGAGG
CTGTTCCCTGGGGGTCGTTACCTTCCACGAGCAAAACACGTAGCCCCTTCAGAGCCAGATCCTGAGCA
AGATGAACAGAAACTGAGGTTTTGTAAACGCCACCTTTATGGGCAGCAACCCCGATCACCGGTGGAAA
TACGTCTTCAGCACGTCGCAATCGCGTACCAAACACATCACGCATATGATTAATTTGTTCAATTGTATA
ACCAACACGTTGCTCAACCCGTCCTCGAATTTCCATATCCGGGTGCGGTAGTCGCCCTGCTTTCTCGGC
ATCTCTGATAGCCTGAGAAGAAACCCCAACTAAATCCGCTGCTTCACCTATTCTCCAGCGCCGGGTTAT
TTTCCTCGCTTCCGGGCTGTCATCATTAAACTGTGCAATGGCGATAGCCTTCGTCATTTCATGACCAGC
GTTTATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAGTTG
TTTAAAATAAGAGCAACACTACAAAAGGAGATAAGAAGAGCACATACCTCAGTCACTTATTATCACTA
GCGCTCGCCGCAGCCGTGTAACCGAGCATAGCGAGCGAACTGGCGAGGAAGCAAAGAAGAACTGTTC
TGTCAGATAGCTCTTACGCTCAGCGCAAGAAGAAATATCCACCGTGGGAAAAACTCCAGGTAGAGGTA
CACACGCGGATAGCCAATTCAGAGTAATAAACTGTGATAATCAACCCTCATCAATGATGACGAACTAA
CCCCCGATATCAGGTCACATGACGAAGGGAAAGAGAAGGAAATCAACTGTGACAAACTGCCCTCAAA
TTTGGCTTCCTTAAAAATTACAGTTCAAAAAGTATGAGAAAATCCATGCAGGCTGAAGGAAACAGCAA
AACTGTGACAAATTACCCTCAGTAGGTCAGAACAAATGTGACGAACCACCCTCAAATCTGTGACAGAT
AACCCTCAGACTATCCTGTCGTCATGGAAGTGATATCGCGGAAGGAAAATACGATATGAGTCGTCTGG
CGGCCTTTCTTTTTCTCAATGTATGAGAGGCGCATTGGAGTTCTGCTGTTGATCTCATTAACACAGACCT
GCAGGAAGCGGCGGCGGAAGTCAGGCATACGCTGGTAACTTTGAGGCAGCTGGTAACGCTCTATGATC
CAGTCGATTTTCAGAGAGACGATGCCTGAGCCATCCGGCTTACGATACTGACACAGGGATTCGTATAA
ACGCATGGCATACGGATTGGTGATTTCTTTTGTTTCACTAAGCCGAAACTGCGTAAACCGGTTCTGTAA
CCCGATAAAGAAGGGAATGAGATATGGGTTGATATGTACACTGTAAAGCCCTCTGGATGGACTGTGCG
CACGTTTGATAAACCAAGGAAAAGATTCATAGCCTTTTTCATCGCCGGCATCCTCTTCAGGGCGATAAA
AAACCACTTCCTTCCCCGCGAAACTCTTCAATGCCTGCCGTATATCCTTACTGGCTTCCGCAGAGGTCA
ATCCGAATATTTCAGCATATTTAGCAACATGGATCTCGCAGATACCGTCATGTTCCTGTAGGGTGCCAT
CAGATTTTCTGATCTGGTCAACGAACAGATACAGCATACGTTTTTGATCCCGGGAGAGACTATATGCCG
CCTCAGTGAGGTCGTTTGACTGGACGATTCGCGGGCTATTTTTACGTTTCTTGTGATTGATAACCGCTGT
TTCCGCCATGACAGATCCATGTGAAGTGTGACAAGTTTTTAGATTGTCACACTAAATAAAAAAGAGTC
AATAAGCAGGGATAACTTTGTGAAAAAACAGCTTCTTCTGAGGGCAATTTGTCACAGGGTTAAGGGCA
ATTTGTCACAGACAGGACTGTCATTTGAGGGTGATTTGTCACACTGAAAGGGCAATTTGTCACAACAC
CTTCTCTAGAACCAGCATGGATAAAGGCCTACAAGGCGCTCTAAAAAAGAAGATCTAAAAACTATAAA
AAAAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGGGCTTCGCCCTGTCGC
TCGACTGCGGCGAGCCTACTGGCTGTAAAAGGACAGACCACATCATGGTTCTGTGTTCATTAGGTTGTT
CTGTCCATTGCTGACATAATCCGCTCCACTTCAACGTAACACCGCACGAAGATTTCTATTGTTCCTGAA
GGCATATTCAAATCGTTTTCGTTACCGCTTGCAGGCATCATGACAGAACACTACTTCCTATAAACGCTA
CACAGGCTCCTGAGATTAATAATGCGGATCTCTACGATAATGGGAGATTTTCCCGACTGTTTCGTTCGC
TTCTCAGTGGATAACAGCCAGCTTCTCTGTTTAACAGACAAAAACAGCATATCCACTCAGTTCCACATT
TCCATATAAAGGCCAAGGCATTTATTCTCAGGATAATTGTTTCAGCATCGCAACCGCATCAGACTCCGG
CATCGCAAACTGCACCCGGTGCCGGGCAGCCACATCCAGCGCAAAAACCTTCGTGTAGACTTCCGTTG
AACTGATGGACTTATGTCCCATCAGGCTTTGCAGAACTTTCAGCGGTATACCGGCATACAGCATGTGCA
TCGCATAGGAATGGCGGAACGTATGTGGTGTGACCGGAACAGAGAACGTCACACCGTCAGCAGCAGC
GGCGGCAACCGCCTCCCCAATCCAGGTCCTGACCGTTCTGTCCGTCACTTCCCAGATCCGCGCTTTCTC
TGTCCTTCCTGTGCGACGGTTACGCCGCTCCATGAGCTT
pRL838-cApra
(SEQ ID NO: 58)
ATCGCGAGAATTAATTCAGATAAAAAAAATCCTTAGCTTTCGCTAAGGATGATTTCTAGCGATGACCCT
GCTGATTGGTTCGCTGACCATTTCCGGGTGCGGGACGGCGTTACCAGAAACTCAGAAGGTTCGTCCAA
CCAAACCGACTCTGACGGCAGTTTACGAGAGAGATGATAGGGTCTGCTTCAGTAAGCCAGATGCTACA
CAATTAGGCTTGTACGGGTACTCGACCTGCATCCCTTAACTTACTTATTAAATAATTTATAGCTATTGA
AAAGAGATAAGAATTGTTCAAAGCTAATATTGTTTAAATCGTCAATTCCTGCATGTTTTAAGGAATTGT
TAAATTGATTTTTTGTAAATATTTTCTTGTATTCTTTGTTAACCCATTTCATAACGAAATAATTATACTTT
TGTTTATCTTTGTGTGATATTCTTGATTTTTTTCTACTTAATCTGATAAGTGAGCTATTCACTTTAGGTTT
AGGATGAAAATATTCTCTTGGAACCATACTTAATATAGAAATATCAACTTCTGCCATTAAAAGTAATGC
CAATGAGCGTTTTGTATTTAATAATCTTTTAGCAAACCCGTATTCCACGATTAAATAAATCTCATTAGC
TATACTATCAAAAACAATTTTGCGTATTATATCCGTACTTATGTTATAAGGTATATTACCATATATTTTA
TAGGATTGGTTTTTAGGAAATTTAAACTGCAATATATCCTTGTTTAAAACTTGGAAATTATCGTGATCA
ACAAGTTTATTTTCTGTAGTTTTGCATAATTTATGGTCTATTTCAATGGCAGTTACGAAATTACACCTCT
TTACTAATTCAAGGGTAAAATGGCCTTTTCCTGAGCCGATTTCAAAGATATTATCATGTTCATTTAATCT
TATATTTGTCATTATTTTATCTATATTATGTTTTGAAGTAATAAAGTTTTGACTGTGTTTTATATTTTTCT
CGTTCATTATAACCCTCTTTAATTTGGTTATATGAATTTTGCTTATTAACGATTCATTATAACCACTTAT
TTTTTGTTTGGTTGATAATGAACTGTGCTGATTACAAAAATACTAAAAATGCCCATATTTTTTCCTCCTT
ATAAAATTAGTATAATTATAGCACGCGAATTCATCGAATTTCTGCCATTCATCCGCTTATTATCACTTAT
TCAGGCGTAGCACCAGGCGTTTAAGGGCACCAATAACTGCCTTAAAAAAATTACGCCCCGCCCTGCCA
CTCATCGCAGTACTGTTGTAATTCATTAAGCATTCTGCCGACATGGAAGCCATCACAAACGGCATGATG
AACCTGAATCGCCAGCGGCATCAGCACCTTGTCGCCTTGCGTATAATATTTGCCCATGGTGAAAACGG
GGGCGAAGAAGTTGTCCATATTGGCCACGTTTAAATCAAAACTGGTGAAACTCACCCAGGGATTGGCT
GAGACGAAAAACATATTCTCAATAAACCCTTTAGGGAAATAGGCCAGGTTTTCACCGTAACACGCCAC
ATCTTGCGAATATATGTGTAGAAACTGCCGGAAATCGTCGTGGTATTCACTCCAGAGCGATGAAAACG
TTTCAGTTTGCTCATGGAAAACGGTGTAACAAGGGTGAACACTATCCCATATCACCAGCTCACCGTCTT
TCATTGCCATACGGAATTCCGGATGAGCATTCATCAGGCGGGCAAGAATGTGAATAAAGGCCGGATAA
AACTTGTGCTTATTTTTCTTTACGGTCTTTAAAAAGGCCGTAATATCCAGCTGAACGGTCTGGTTATAG
GTACATTGAGCAACTGACTGAAATGCCTCAAAATGTTCTTTACGATGCCATTGGGATATATCAACGGTG
GTATATCCAGTGATTTTTTTCTCCATTTTAGCTTCCTTAGCTCCTGAAAATCTCGATAACTCAAAAAATA
CGCCCGGTAGTGATCTTATTTCATTATGGTGAAAGTTGGAACCTCTTACGAGCGGCCGCATACGATTTA
GCTTTTTAACTTGGATTTTTACCTTCTCAGCCTAAACCTTAACATTAGTGTCAATGTCAAGGTTTTGAGT
AAACTAAAGGGATGAAGACTAATCACTTAACGATTAAAGAACTCACAGATGCAGTGGGAGGTGGCGT
TACGCCTCGCATGGTGCGCCATTACCACACCCTGGGATTGCTTCCCCCCGTTCAACGCTCAGAGGGCAA
CTACCGCCTTTATACTCAGCAGGACGTACAACGGCTCCAACGAGTCATTGCCCTCAAACAGCAGGGCT
TTCAGTTGTCTCATATTCGGCAACTGCTGGATAGCCATTCTGAAGAGAGCCTTGATCCCACCCTAATGG
TGCAGTTGCAACAGCAATATCAGGCTGTGATTCAGCAGATTACTCGACTCCGCCAAACCGCATCTGCTT
TAGAAGGATTACTGGGACGCGACCAGAGTTGCCAGATTACCCAAGCGGAAGCTCTGGCCCAACTGAA
ACAGCTTGATGTGGATGTCCAGGAGGGGTTAGGAAAACTCGATCAGTTGTGGACGAACCTAGATGCGG
AGACAACAACTCATCCAGAAGCCTTTCAGGAATCCCTCAAACACCTGCTACCGGATTTATCGGCTTACT
CTGAAATTACCATTCACTTACTACATCAATTAGTGCTGGCCTGTGGTGATGTTAGCTTGGTAAACGCCG
TTCGATTGAGTCAGGGAGCGATCGCCTCGGCACGAGATGCACTGAAAGCAGGGTGTCCAGTCGTCACC
GATGTTCCGGTTGTGGCTGCGGCTCTTGATCAAACTCGGTTAGCTCATTTAGGATGTACGGTTAAAACG
CTGATTGACGACCCTCACATCACAGGGCTTAGGGAAGCCGAGCAAGCTTTTTGGCACCATGACCATTG
GCAACAGCGGTTACAACAGATTCCCCAAGGATGTGTGCTGGCGATCGGCTATGCCCCTTCTGTTTTACT
CACTGCCTGTAAGCTGATAGAGCAACAACATATTCAGCCGGCTCTTGTGATCGGAATGCCGATCGGTTT
TAGTCATGCTCCGGGGGCAAAACGACGACTGATGACCAGTCCCATTCCCCATATCACCATTCAGGGGA
GCCTCGGTGGAGGACTTCTAGCCGCAGTAACGCTAAACGCTTTAGTGGAAACATTGATTGCAAAGCCA
GATTGCCACTGCTATCTCACTTGTCTTTAGGAAACAGACCATGCTAGATAAAATAAATCGTTATGCTCA
TGGGTTTGTAGCTGTACCAGTGATTTGTGCTTGTTCAGAAGCAGGGGTATTCGAGTTACTATCACAGAA
AAAATCACTGAAATTAGAAGAAATAGTAGAGCATTTAGCAGCAAATAGTGGACATCTAATGGTAGCTA
TGCGACTCCTAGAGTCATTATCATTCCTATATCGCTCTCAAGCAGAAGAATACATATTGACAGAACAA
AGTCAACAACATCAAATCATCCCTAAAGCTCTGATGAGCTTATATAAATATCCATTTGAGTTATATTTA
AAAGGAGAAGTAGAAACAGGAATCAGCAATTGGATTAATTGTTCATCTCGAAGATGGGATACAGAAA
ACTCATTACTAAGTGATTTATTAGATGGAGTATTACTCATCCCCCTGCTACTAGAACTGAAAAAACAAA
ACTTACTGGATGAATCAAAAAAAATATTTAACACATTAACAAATTCCCTTAAACAAGAACTATCAACA
TTATTCATCAATTTAGGATGGGCAGAAGAAAAAACAGAAGGGCTATATTTAACAGATATAGGTAGATT
TATGCGTGACAGATCCTTAAACTTAGGAACAACAGCGTCTTATGCTCCTATGTTGTTACAGATGAAAGA
ATTACTATTTGGAAATCCTCAGAGAGTATTTCAAAGAAATAAGACTGAAAAAGAAAGACACGTAAATA
GAACATTAAATGTAGTAGCAAGTGGCTTTCAACACGAAAAGTTTTTTGCCGATACAGATAAAATCATC
ATATCTATATTTAACCAACAGCCAATAGAAGAGCAACCAAGCTATATAGTAGACATGGGTTGTGGGGA
TGGAACGCTACTGAAACGAATATATAAAATCATCAAACAATTCTCTGCTAGGGGAAAAGTATTAACAG
AGTATCCTATCATCATGGTAGGAGTAGACTACAATCAAGAAGCATTAGATGTCACAGATAAAAATTTA
GTAGATATTCCCCATCTAGTCATTCCAGGAGATATAGGAGCACCAGAAAAATTATTAGAGCAATTGAA
AGCACAAGGAATAGAACCAGAAAAAGTATTACATATTCGTTCATTCCTAGATCATGACCGACCATTTA
TAGCTCCAAAAAATACAGAAATAGCTCAAGCACGTTCTCAGTTAGATTATCAAGTAGTAGATGTAGAT
CGAGAAGGAAAGCTGATACCACCTCACATAGCAGTACAAAGTTTAGTAGAACACTTAGAAAGATGGT
CATCAATAATAACTAGACATGGATTATTGCTCCTAGAAGTACATAGTTTAACGCCAGCAGTAGTCAAA
AAATATATAGACGAAAGTGAATCTCTGCATTTTGATGCCTATCATGCCTTCTCAATGCAACATTTAGTA
GAAGCAGATGTGTTCTTGATGGCAGCAGCAGAGGTGGGTTTATTTTCACGGAAAGAAGCTTTTCGTAA
GTATCCGAAAACGTTGCCATTGACTCGAATCACAGTTAATCATTTTGAAAAGCGTAAATATCAGATAC
GGTATGCAACTGTAAACGATATACCAAATCTGTTAAAGTGTGCGACATTTAATCAACCTGTAAACGAA
CCCTTCTTTCAAGTTTTATTGAAACAAACTCCAACAGCACATTTATTATTAGAATATCAAGGTGAATTG
GTAGCTGCAATCTTCACAGAAACTAAAAATTCTAATGAGGTGCTAGGAATTCGTGAGTTTTTAGTAAG
GACTTCTGTAGAAAATTGGCAAGTATTAGCAAAAGATTTACTAGAATTTGTGGAACAGTGGGGAGTAG
TCAAACCAGGAATAAAAGAAATAGAAGGATTGTTAAAATACCATGAAGCCATCTCAAACTTTCAAAA
ATCAAAGTGGTATCAATCTTCAGTTTTAAACAAAAAGCTTATAGAAAAAATAACTCTACACGAATTAG
CTACTTTAGAACTATGTAATTTAATGGCTCCAGAATATGAGCTAGAAGCCTTTGCCGCACGTTGGCTTT
TGCGTGTTTTTCAAGATATGGGTGTATTTCTCAGAGAGGGTGAATCTTATCAGGAGTCTGAGTTGGTTT
CCCAGCTAAACATCTCACCACGTTACCAGAGACTTTTAGGTGCTTTGTTACAAATTTTGCATAAGCGTG
GTATTCTAAAAATTGAGAAAGATAGAGTGTTCACATTAGCAAGATGTAAGACCTTTGCCTTAGAAAAT
ATTTCGTCTGAAGTTTCTGCTTTTTATGATTATTTTTCTGAAAAATATCCTGCTCATTTATCCTGGTTAAC
AGTAGTCAAAAGGTGTCTAGAGAAATATCCTTTGATTCTACGCGGTGAAGTTGATGTCAACGAAGTTG
TTTTTACAGATGGGGATATGGAGCTATTTGCTGGACTATTTCTAGGACATCGTGTTGCTGACTACTTTA
ATGAGTTGCTAGCAGATGGGGTTTGCTGGGAAGTAGAACAGCGGTTGTTAGAAGAAAAGAGGGCACA
ACCTATTCGGATCTTGGAGATTGGAGCAGGAACAGGAGGTGTTACAGGAATATTGCTAGAAAAGCTAG
CTTCTCATGCAGAGCAAATTGAATTTTGGTTTACTGATATTTCTAGCGTTTTTACACGTTATGGTGAGAG
TAAGTTCAAGCAGTTTCCTTGGGTGAAATATCAAACCTTTGACATAGAAAAATCTCTTGATGCTCAGGG
GATAAAGTCTGAAAGTTTTGATGTGGTAATTGCTAATAACGTACTCCATAACACAAAATTAATTCATCA
AACCTTAAATAACAGTAACTCACTATTAAATACTGGGGGGTTATTGGCATTACTAGAGTTTACTCAACC
AATTGATATTCTTTTATACTTTGGAGGGTTGCTTCAAGGATTTTGGTTGTTTGAAGATCCAGAATACCG
ACTAGAAGTTGGTTGTTTACTGAGTATACCACTGTGGCAAAAAGTTCTCAGTGATTGTGGGTTTGATGA
AATAATACCATTGGGATTACCCTGTGAGATGCACGCTCTTTCTAAGGCAAGAGAATCTGTTATATTTGC
TCGAAAGCATCAAGTACAGGAGAAAACATTTTCTGAAAAAATTAAACAAAATTTGACAGAAAATGGT
AAGCATGGGCAGGCAGAATTCGATTTTATTAGTATTAATAATTCACAAGAAAGTTCATCCAAATTAGA
AATTTTTGAACAGGAATGTCGAAAATTATTAAAATCTCTACTGGGTGTTCAACGTATGGAGAGATTGCC
TGGTGACACACCACTAATGGAGTCAGGAATGGATTCACTGGAGTTGTTAGAATTTCGTGCTCTTATAGA
AAGAAAGTTTGGGATTAAGTTAAAGTCTACCTTCTTTTTTAGTTACAAAACTCTTATAGCGGTAGCAGA
GTATCTTTCAGAACGGGAAGATATTAATTTTAGTTAGTAGGACTATGACTGAAAAAAAAACAACGTTT
TCACAGGTCCACAGGGGGAAATTTTGAATCAGGGGGAAAATTTGAATGCATTCTAACCAGAGCAGTTC
ACTAGCAAGAATAGCCATTGTAGGTGTGGCTTGCCGTTTTCCTGGAGCAGATACTAAAGAAGAATTTT
GGCAGCTACTTAAGGAAGGTCGGGACTCAATGCAGAATCTTCCTTCAGAGCGTTGGGGAAATCTATTT
AACTCCATGAGTTCAGAAATAGATTTATCTATTCAACGCGGTGGTTTTTTGAAAGATATAGATTTGTTT
GACTCATCCTTTTTTAGGATTACTCCTAGGGAAGCGCAGTTAATGGACCCCCAGCAAAGGCTGTTGTTA
GAATTGTCCTGGGAAGCGATGGAAGATGCAGGATATGCAAGGGATACATTAAAGGGTAAATCAGTAG
GGGTTTATGTAGGAGTTTGTCACTATGACTACAGAAGTTTATTAGAAAAAGGTTTGGAGACAGCCGAA
ATAGCTCAAATTGCTACTGGTACAGCTCCAGCAACTTTTGCCAATCGCCTTTCCTATTTTTATAATTTTC
ACGGCCCTAGCTTAACTGTTGATACGGCTTGTTCCAGCTCTTTGGTAGCAATGTATGAAGCAGTAAATG
CAATACGTCGTGGGCAGTGTCAAACAGCATTGGTAGGAGGCGTTAATTTAATGTGTTCTCCTGTGAATA
ACCAAGTTTATAGTGCTGCGGGTATGTTATCTCCTGATGGAGTCTGCCGGGTGTTTGATGCGGGTGCTA
ATGGGTTTGTCAGGGGAGAAGGAGGGGCAGTAGTTGTACTTAAAGATTATCAAAAAGCGTTGACGGAT
GGTGATTCTATTTATGGGGTTGTTCGCAGTGTAGCAGTGAATCATGGAGGGCAAGCAAGTTCTTTTACA
GCACCAAATCCCCAAGCTCAGGCAAAATTGTTGGAACAGGCTTATAGGGAAGCTAATATTGATATAGA
GTCGGTAGGTTATATTGAAGCACATGGTACGGGTACATCTCTAGGAGACCCAATTGAGGTCGAGGCTT
TAAATGAAGCGTTTAAAGGGTTGAGTTCAAGTGGTAAGTTACCTGCTAATAGTTGTGGTTTGGGCTCAG
TTAAAACAAACATTGGTCATTTGGAAGGGGCAGCAGGTTTGGCTGGACTAATCAAAGTTTTGCTGTGC
ATGAGGTATGCAACTCTTCCATGTTCATTAAATTATCAGCAACTTAATCCTGATATTGAATTAGAAGAA
GGTCCGTTTTTTGTGGTGGACAAGCTTCAATCTTGGGAAATAAAAGTTGATAGGGTAGGAAAACCTTA
TCCACTTAGAGCAGGGTTGAGTAGCTTTGGTTTTGGCGGTACAAATGCTCATGTAGTTCTTGAGGAAGG
AGAAAATAAGAAGGAAGGAAGAAGAGGGGATAAGGAACCTTCAGTTCATCTATTAACCCTTTCAGCT
AAAACTGAAACAGCTCTTTCAGAGTTAGTCAGTCGTTATCAAAAATATTTAAACACTAATGCAGAATC
AGAGTTAGCCGATATTTGTTATACCGCTAATACTGGGCGAGTCCATTTTAACCATCGACTAGCAGCGAT
CGCCTCAAACAAACAGGAGTTAGTAGAAAAACTTAAGGAATACCAAGCTGGGGAGGCAACAGCGGGA
GTATTAATAGGGGAACTTGCAGAACGCATAAGAACACCGAAATTAGGTTTCTTATTCACAGGTCAAGG
TTCCCAGTATGTCAATATGGGAAAACAACTGTATAAAACACAGCCTGTATTCCGTGAAGTATTGGATA
AGTGTGATATAATATTGGAAACGGAAATAGAATGTTCTCTATTAGATGTTCTATATAACAAAACTACA
GATTCTCAAGATTCATCTTTAATAAACCAAACAGCTTACACTCAACCAGTTCTGTTTGCAATAGAGTAT
GCGCTATTTAAGTTATGGGAATCTTGGGGAATTAAACCCAGCATAGTAATGGGTCACAGTGTAGGAGA
ATATGTGGCTGCTTGTGTAGCAGGAGTCTTCAATCTTGAAGATGGTTTGAGATTAATAGCTGCTAGGGG
CAGGCTGATGCAACAGTTACCTTCAGGTGGAGAAATGGTTTCTGTGATGGCTTCAGAATCTACAGTTA
GCAGACTCCTAGAACCTCACAAAAAAGAAATAGCGTTGGCGAAGCCCGCCGTAGGCATCGCAGCAAT
AAATGGACCAGAAAGCACAGTTATTTCTGGAGACTCTGTAGCAGTAACAGGTGTAGTAAATGACCTGG
AAGCAAAGGGAATAAAAACTAAAAAACTAGAGGTATCTCATGCTTTCCATTCACCATTAATGGAACCA
ATGTTAGGAGAGTTTGAAGCTATAGCTAATCAACTAACCTACAATCAACCTAAAATACCTATTATATCT
AACGTTACAGGTACAAAAGCAGATAATACTATTGCTTCCCCTCAATACTGGGTTAATCATGTCGGTAA
ACCTGTAAGATTTGCCCAAGGAATGGAAACGCTACATCAACAAGGGTATGAAACCTTTATAGAAGTTG
GACCGAAACCAGTATTGTTAGGAATGGGCAGGCAATGTCTACCAGGAAATGTAGGTGTCTGGTTGCCA
TCATTACGTCCGGGAGTGGATGAGTGGCAACAAATGCTTTATAGCTTGGGAAAATTGTATGTAACAGG
AGTAAAAATAGATTGGTCAGGATTTGAGTCTGACTCTAGTCGCCAGAAAGTAGCATTGCCAACTTATC
CATTCCAGCGAGAAAGATATTGGATAGAAATAACAGAAAACAAACATAAGGAACATCAAAAGTCAGA
AAATATAAGTGACACTTCAATTGTTAAACTACTCACTCAAGGAAAAACAGAAGCTCTCACTCAACAAC
TAGAAACAGAAGCCAAATTTTCACCAGAAGAGCTTAAACTTTTACCAGAAATATTAGAGACATTAGCC
AAACAACATCAAGAACAATTAACAGCAGTAACCATCAAAAACTGGTTCTACGAAATCCAGTGGAAAC
CCTTAGCTCAAAACAACCCCAATGCAAACATTGAACCTAGTCATTGGTTAATTTTAGCCGATACCACAG
GAGTAGCAGAAAAATTAGCTCAAAAATTACAACAACAGGGTCATAAATACAGCTTAGTTTATCGAGGA
GAGAGCTATCAAAGACAAGCAACAGGTACTTATCAACTTAATCCTCAGATTCCCGAAGCATTTGAAAA
GCTGTATCAAGAAATTCAACAAAGTAGTGAAACTGCCATTACGAAGTTAATTCACTTGTGGAGTTTAG
ATGCTCCCCAATCAAAAGACTTAACCCTGGAAACCCTAGAAGAAGCTCAATTATGGGGATGTGGCAGC
GTAGTACACCTGTTACAGACCTTAGTCAAAAACTCTAGCATTCCTGAACTATGGTTAGTAACCCGTGGG
TCTCAATCAGTATTATCCCAAACAGAAAAAAATCTAACAGGACTAGCAGCGTCACCCTTGTGGGGATT
AGGTAGAGTAGTGTCTAATGAACATCCCCAATTATGGGGAGGATTAGTAGATTTAGACCCACAAGCTG
CAGCAGGAGACGAAGTAGAAATGCTGTGGCAATTATTAGTTAATGAACAAGAAGAAGATAATCTAGC
TGTACGGGGAGAAAATACCTATGTAGCTCGTCTGGCCAGGCAAGAACCTCAAGAATTTCCTGAATCCC
TATCCTTATCATCAGATGGTAGTTACCTAATAACAGGAGGGTTAGGAGCTTTAGGGTTACATACTGCCC
AATGGTTAGTATCCAAGGGAGCGAAAAATATTGTCTTAACTGGGCGTCGCCCTCCCTCAGAAAAAGTA
AGTGAATCTATAAAAAAATTAGAAGAAACAGGATGTCAAGTGAAGGTGATGTTGGGGGATGTTTCTGT
TGAAGAAGACATAGGCAAAATTCTCAAACAAATTCAGATATCAATGCCAACACTCAAAGGCATAATTC
ATACAGCAGGAGTGTTAGATGATGGAACCATACAACAAATGAATTGGGAGCGTTTTGCCAAAGTCATG
TCGCCCAAGGTAAAAGGAAGTTGGCATTTACATAAATTAACTGAAAATCAGCCATTAGATTTCTTTGTG
TGTTTCTCCTCAATAGCTTCGATGTTGGGGACTCTTGGCCAAGGAAACTATGCTGCAGCCAATGCCTTT
ATGGATGCTTTAGCTAGTTATCGTTGCAGCAGAGGATTATCAGGATTGGCGATTAATTGGGGAGCATG
GGCATCAGGGGGAATGGCTGCTCGTTTAGCAGTGGAGCATCAAAATAGGATGCACAGCAGTGGCATA
ACTGAGATGGCGACCAAAGAAGGAATGTATGCCTTAGATTTACTATTAACAAATGAATCTGCTACAGC
TCAGGTAGGTGTAGCAAGTATAGAGTGGCAGGTACTCTCAGAAAGTTGGAGTGGTATCAAAAAAAATT
CATTGCTACGAGAATTATTAGAGAAGGAGGAATGGGCAGCAAAAGATACACGACACCAAAAAGTTAA
AGCCGAATTTTTAGCAAAATTAGAGGAGGCATCACTAGAGAAACGTCAGGAAATTTTAACTGAACACA
TTCGAGTGCAAGTATCTCAGGTACTAGGTTTAAGTTCATCTAAATTACCAGAAGTAAATGTAGGTTTTG
CCTCTATGGGAATGGACTCTTTGATGACAATAGAATTAAAAAATCGACTGCAAAATCAACTAGGAACC
AAGTTGCCTGAGACAATAGCTATCGAATATCCAACTATTGCAAAATTGTCCTTAGGTATCAAAAAAAA
TTCATTGCTACGAGAATTATTAGAGAAGGAGGAATGGGCAGCAAAAGATACACGACACCAAAAAGTT
AAAGCCGAATTTTTAGCAAAATTAGAGGAGGCATCACTAGAGAAACGTCAGGAAATTTTAACTGAACA
CATTCGAGTGCAAGTATCTCAGGTACTAGGTTTAAGTTCATCTAAATTACCAGAAGTAAATGTAGGTTT
TGCCTCTATGGGAATGGACTCTTTGATGACAATAGAATTAAAAAATCGACTGCAAAATCAACTAGGAA
CCAAGTTGCCTGAGACAATAGCTATCGAATATCCAACTATTGCAAAATTGTCCTTATGTATAGAGGAAT
TAATGGGATGGAAGACAACAGAAATTGACCCATTATCTGAAGAAACAAGATTAGAGATGATGGAGCG
CTCAAGAAGTGAAGCAATAGCCATCATCGGCATAGGCTGTAGATTTCCCGGAAATGCCAACACACCAG
AAAGCTTCTGGCAATTGTTATCCAATGGCAAAGACTCCATTACAGAGATTCCCCTAGAACGTTGGGAT
CTAGATTCCTACTACGACCCCAACCCTGATACTCCAGGGAAAATGTATATCCGTCACGCAGCATTAGTA
GAAAAAGTAGATCAGTTCGACCCACGATTTTTTGGAATCTCTAACCGAGAAGCTTATAGTCTTGATCCA
CAGCAACGCTTCATTTTGGAAGTAACTTGGGAAGCTTTAGAAAGAGCTGGTATTAACCCTCAACAATT
AGAAAACACTCAGACAGGGGTGTTCCTGGGTATCGGTCAAAATGATTACGCGAATTTAGGTTTTCACC
AAGCAGCCGAAGATATAAGCCCTTACGATGCTACAGGAAATTTGTTTTGTTTTGTAGCAGGTAGGTTAT
CTTACTTTTTAGGAACGCAAGGTCCATCAATGGCAATAGATACAGCTTGTTCATCATCTCTAGTAGCTA
TCCATGAAGCTTGTGAGAGTCTGCGTCAGGGTGAGTCCAACTTGGCTTTGGCTGGAGGAGTTCAACTA
ATTCTCTCTCCCGAAGTAACAACCGCACTATCAAGATTAAAGGCTCTAGCACCTGATGGTAAGTGTAA
AACTTTTGATGCTGCTGCTGATGGTTTTGGTAGGGGAGAGGGATGTGGCATTGTGGTACTGAAGCGTTT
GTCGGATGCGCTCAAGGATGGGGACAGGATATCAGCAGTAATTCCTGGTTCGGCTGTTAACCATGATG
GACCAAGTAGCGGAATGACAGTACCGAATAAACTGGCTCAGGAAAAACTGATTCAGAAAGCTCTCAA
AGCAGCCAAGGTAAAACCGTTACAAGTGAGTTATGTGGAAGCTCATGGTACAGGAACTTCTCTAGGAG
ATCCTATGGAAGTGAGAGCTTTAGCTAGGGTGTTTGAGGAGGGACGTGATCAGGAAAATCCATTGAAC
ATCGGTTCAGTTAAAACTAATATCGGTCATCTGGAAGCAGCAGCTGGAATAGCAGGTATGATTAAGGT
GATTTTGCAATTGCAACATCAGGAAATTGTGCCCCATCTGCATTTTGCTAATCCTAACCCCTATGTTGA
TTGGGAGAATATGCCTCTACAAGTACCGACTCAACTGACTCCTTGGTTGTCGAAAGGGGAGAAAAGGG
TGGCAGGAGTTAGTTCTTTTGGTATGAGTGGTACGAATGCTCATATAGTTTTAGAAGAGGCTCCTATTG
AAGTCAGAAGGCAGAAGTCAGAAGTCAGAAGTGAAGAATATCTAGAACGTCCGGTTCATATACTAAC
TCTGTCGGCCAAGACTGAAAAAGCACTAGAAGATTTAGTTAATAGTTATGAAAGTTATTTAGAAGCCG
AAAATAATGATAATTATTTAGGGGATATTTGCTACACAGCCAACATCGGGAGAGCAAAATTTGACCAC
AAATTAGCAGTGGTTACTTCTGATAAACAAGAGTTATTAGAGAAACTCAAACAATATAAACAAGGTGA
GAATGTTGCTGGAATCTTTTCAGGAAAACAAATAAGTGAAACTAGAACAAAAATAGCCTTTATATTTA
CTGGTCAAGGTTCTCAATATCTGCAAATGGGAAGGCAGTTATACGAAACTCAACCCACTTTTCATAAA
ATTATTGACCAGTGTAGTGAAATGCTGGTAAAATATTTAGATGTTTCTTTATTAGATATACTTTATCCA
GTTGAGGTTAAAGATGAAAGTTCGACTTTGATAGACCAAACAGCTTATACTCAACCCGCTATATTTGCC
CTTGAATATGCCCTAGCTAAATTATGGGAATCATGGGGTATAAAGCCAGATGTAGTCATGGGTCACAG
TGTAGGAGAATATGTGGCAGCAACAGTAGCAGGGGTATTTAGTTTAGAAGATGGTTTAAAATTAATAG
CCATGCGGGGACAGTTGATGCAAAAGTTACCCTCCGGAGGTCAGATGGCATCTATAATGGCATCAGAA
TCTCAGGTAATAGAGGCGATAAAAGAATATAGTTCCCAAGTAACAATAGCAGCAGTTAATGGACCAG
AAATTATAGTAATTTCAGGTGAGAGTCCAGCCATTTCAAAAATTTGTAGTAAATTTGAATCAGAAGGA
GTCAAGACCAAGGCGCTACAAGTATCTCATGCTTTCCATTCCCCATTGATGGAACCAATGTTAACAGA
ATTTGAAGCAGTAGCCAAAGAAATATCCTATAATCATCCCCAAATACCACTAATATCAAATGTTACTG
GTCAAGAAGTAAATGGAGAAATAACCACTGCTGAATATTGGGTGCGTCATGTGCGTCAACCAGTAAGA
TTTGCGGAAGGGATGGAAACTTTACACAAACAAGGTGCTGAAATTTTCCTAGAAATAGGCTCAAAACC
GATACTTTTAGGTATGGGTCGTGAGTGTCTCATGGGAGAAAAAAAACTATGGTTGCCCAGTTTACGTTC
AGGAAAACCAGACTGGTTACAAATGCTACAAAGTCTGGGGCAATTGTATGTACGAGCAATAAAAATTG
ATTGGTTAGGATTTGATCGGGATTATTTCCGTAATAAGGTAGAGTTACCAACATATCCTTGGCAACGAA
AAAGGTATTGGATAACAGATATTAGACAACGGAAAAGTCAAGACAAAAAAAGCATAACATCTGAAAA
AAAAGTACAACTTGATGGAGTAAATATTCAACTAAAGGAAATACAAATGAACGACAAAATCTTACAG
CAACCAAAACTAAAATTATCAGATCCAGAATCACTATATTTATCGAATGCTGAATCAACTATAGAGGT
TCCAACAAAAGCAGTTCAAGTAAAACCTGCTGCTGATATTGAATCTGAAAATATAACTCAATTGAATA
GCCTTGATAGAGATGTGACTCAAATCATAGAAACCCTCAAAGAAAGTTTGGCAGATGCTTTATATGCA
GATATAAGTGAAATTGAAGAAGACAAAAAGTTTGTTGATTTAGGTTTAGATTCTATTGTGGGAGTAGA
ATGGATAACTAACATCAATAAAATCTACAATTTAAATATAAAAGCTACTAAACTATACGATTACCCTA
CTTTGCCAGATTTGGCTAAATATGTCGCTCACACCCTATCAGCTCAGGGTAGAAATATTGATGTAGAGC
GATCGCCATCTGACTCAAGTCAAGCAAGCATTAGCAAGCAATCTCAACTAACTGATACCCAGAGCAAT
TTCTCACAAGTCAAGGAAATTTTGAAACAACAATTAGCAGATGCTCTGTATGCGGATATTAGTGAAAT
TGAAGTAAACAAGAAGTTTGTTGATTTAGGTTTAGATTCTATTGTTGGAGTAGAATGGATAACTAATAT
CAATAAAACCTACAATTTGAATATCAAAGCCACTAAAATATACGACTACCCTACCTTATTAGATTTCGC
AAAATATATTAACCAGGAAATTTATTCTACAGGAGTAAGTAGATTTTCAACGGAGCATAAAGAATTTA
ATCAAAAAGACTATTCATCTGGAGGTTCGCAAGAGGAAATGACACAAAAATTACGATCAATATTGAAG
AAAGTGGCCAATAAAGAATTAACAGTTCAGGAAGGAAATAAAATGATTCAACAAATAAAAAATCAAT
TAAAGTAAAAATATAGTATAATTTTTAAGGAAATAAAATTTTATGAACAAAGAGCAAATATTTCAAAT
AATAAAAAAATATACCTACGAAATAGCACCAGAATTAGAAGAAGTACCTATATCACCTACTGATAGTC
TCAAAAACTTGGGGATTGATTCAGTAAATAGGGCAGAAATTATAATGATGGTAATGGAAGAGTTATCA
TTAAATATCCCGCGTATTGAATTAGCTGGATCCAAAAATATAGGAGAACTGGCTGATATATTTGCATTC
AAATTAGAAGCAATAAATTCACAAAGCTAGAAGGCCTATCAATGAGTAACATAGAAATAACAGGTAT
GGGCATTGTTACTTCCATTGGTCAAGGAGTTGCTACTTTTAAAGAAGCTCTGTTATCAGGGAAAACTCA
ATTTGCTTATCTAAAACAGCCAGGACGTGAAAGCATCAAACCATTTATTGGTGCCGAAATTCCCGATAT
TGACGCCAAAACCCTATTTCCTGAGTACAGTGGACTGTTACGTACTGCTACTAAGAGCGCCCAAGTTGC
AATAGTAGCTGTCGCTGAAGCCTGGCAAGATGCTCAACTCACCTCCAGCCAAGTTAACCCAGAACGAG
TAGGATTAGTTGTAGGTGGTTCAAATTTACAACAACGTTATCAGCAGCAAACCTGGCAACGTTATCACT
CACGTCAGGAGTTTATACGACCAACTTATGGTCTGACCTTTTGGGATACAGATATATTAGGTCTGATTT
CCCAGTGTTTTCAGATTCAAGGTGAGGGCTATAGTGTAGGTGGAGCTTCGGCCAGCGGAGCAGTTGCT
ATAATTCATGCTGCTCGTCAAATTTTAATGGGGAACAGTGATGTTTGCATTGCTCTGGGAGCTTTATCC
GATCTATCAGGGTATGAATTCCAAGCACTAATGAATTTAGGTGCAATGGGGAGCGAACGTTTTGCCGA
TCGCCCAAACCTTGCTTGTAGGCCATTTGACCAAGACCATGATGGCTTTATTTATGGAGAGGGTTGTGG
AGCAGTGATTTTAGAGAGAACTGACCGCGCTCAACAACGGGGCGCTCAATCCCATGGCCAACTTAAGG
GTTGGGGATTAACTTTAGATGGGAACCGTAGTCCCGAACCTTCCCAAAAAGGGGAAGAACGGGCTATA
AATACTGCTTTAGCCATGGCAGACCTTCAGCCAGAGAGTATTGATTATGTGAATACCCACGGTACTGGT
TCCCCCCTTGGCGACAAAACAGAAGTGGCAGCTTTCAAATCAGTAGGACTTCAGCATTGCCTATTTAAT
TCAACAAAATCTTTAATAGGCCACTGCTTAACTGCTGCTGGTGTAGTGGAAGCGATCGCCACCATATTA
CAAATGAAGTTTGGTTTTTGTCATCCAACTAAAAATTTAGTTAATCCTATTGATACCAGTCTCAATTGG
GTGAAAGAAACTTCTGTTCAAGCTGAGATTAAATATGCTATCAGCAACAGTTTTGGTTTTGGGGGTATC
AATACAGCTTTATTAATTGGACAGGAGTAAACTCATGCAACAAGTTGGAATTGAAGCACTAAATGTAT
ATGGGGGTTCAGCTAAACTAGATGTGCGAATGCTAGGCGAAGCACGTCACTTAGACATGACTCGCTTC
GATAATCTTCTGATGAAAGAGAAGACAGTTGCTATGCCCTATGAAGATCCAGTTTCTTATGCTATTAAT
GCAGCAAAACCTATTATTGATAGCCTCAGCGTTCTGGAAAAGCAACAAATCAAAATGGTGATAGCTTG
TAGCGAATCTGGCATTGATTTTGGCAAGTCTATGAGCACTTATATCCAGGATTATTTGGGACTAAATCG
TAACTGTCGAATGTTTGAAATAAAGCAAGCTTGCTACTCAGGTACAGCAGGTTTACAAATGGCATTCA
ATTTAATTTTGTCCCAAACCTGTCCAGGGGCTAAAGCTTTGGTTATAGGAACTGATATATTTCGGCCTG
TTGTTGTAGAGGGAGGAGAAGCTCTCAGCGAAGATTGGTCTTTTGTGGAACCAAGCAGTGGTGCTGGA
GCAGTAGCTATTTTAGTCAGTGATGTTCCTAAAATTTTCCAAGCTGATGTAGGATGTAATGGTTACTAT
GGCTATGAAGTCATGGATACTTGCAGACCTAACCCAGACTCAGAAGCAGGAGATGCAGATTTATCATT
GCTATCTTACCTAGATTGTTGTGAAAATGCTTACCGGGAATATCAAGATCGAGTAGAAGGAGTAGATT
ACCAAAAAACTTTTGATTACTTGAGCTTTCATACTCCTTTTGGGGGTATGGTGAAAGGGGCTCACAGAA
GTATGATGCGTAAGTTTAAAAGGGCAAAACCTGTGGAGATTGAAGAGGACTTTCAGAAACGGGTGAT
GCCGGGATTAGTCTATTGTCAACAGGTGGGTAATATTATGGGAGCTACAGTATTTTTATCTTTAGCTAG
CATGATTGATAATGCAGATTTTAGCAAAGCCCGACGAATTGGTGTATTCTCTTATGGCTCTGGTTGTTG
TTCGGAATTTTATAGTGGAGTCGTCACCCCGCAAGGAAAGGAAATTCAAGCTCAACAAAAAATTTCAT
CACAGTTAGCAATGCGCTATTCCTTAAGTATGGAAGAATATGAGCAGTTACTAAGTCATAGTTCGGCA
GTTGCCTTTGGCACTAGAAATGTTACCCTAGATTACAAACTATTTCCTGGTGTGTGGGAACAAATTGAA
GGTAAAGGTCGCTTAGTGCTGAAAAGAATCAAGGAATTTCACCGAGAATATGAATGGGTATAGCCATG
AGTTATCAAACCCTGAAAATCAGTTATCAAGATGTTGTACAAAGGATTCAGATATATCGACCTGAATC
TAACAATAGCATCAATAGTCAATTAACGATGGAATTGTTGTCAGCTTTGCAAGCTGCTGAAGCAGAGG
AAGTTGTTAAAGTAGTGATATTAGAAGGACTACCTGATGTATTTTGTACAGGGATGGATTTTGAAGAA
GTGGCAACAGCAAAACAATTTGATCCAAAAGCTAGTGCTAATGGTTACTACAATATTTTGAAACAAAT
GTCTCAAAGTAGCAAAGTAATTTTGTCACTTGTGCGTGGTAAAGTACAGGCAGGGGGAGTTGGTTTAG
TAGCAGCAAGCGATCTAGTTATTGCTGATGAAACGGCAACTTTTGTTTTATCAGAATTATTATTTGGAT
TATTACCAGCTTGTGTATTGCCTTTTTTGATTCGTCGAGTGGGATTTCAAAAAGCCTACCGTTTAGCACT
AACGACTCAAGCTATTTCAGTATCAGAGGCTGATAAGTGGGGATTAATAGATGAATATGGCAGTAATA
TTAATCAGTTAATAAGTAAATATATTCGACGTTTAAAGTATTTACCCTCATCAGGGGTGAAAGAGTTAA
AAAATTATATTAATCAGTTATGGATTATTCAGGCAGAAACTCAAGGTTTAGCCGTCAACGAGATTTCTA
GCTTAATAGCAGAACCTACTGTTCAAGAAAAAATTAAACGTTTTCAAAAAGAAGGATTATTTCCATGG
CAAACCTAAATCTTAATTTGGACTTAGTAGAGGGCAACTCTGATGTAGTACAACTGGTGGAGTTGGGT
AATGGTGTCGTGCAAATCACGATGAAAGATGAAGAAAGCTGCAATGGCTTTTCTCCTGGAATAATTGA
AGGATTATACAAATGTTTTGGTGCAGTTGCTCAAAACCAAAGTTATAAAGTAGTAATTTTGACAGGCT
ATGGAAATTATTTCTGTTCGGGAGGGACAAAAGAACGGTTAATCAGTATTTGGAAAGGAGACAGCAA
ATGTAATGATTTAGATTTTTTTAGAATAGCATTAGATTGTGAAATACCAGTAATTGCAGCTATGCAAGG
TCATAGTATTGGCGGTGGTTTGGTTTTGGGATTGTATGCAGATTTAGTAGTGTTAAGTCAAGAAAGTAT
TTACACTACTAATTTTATGAAGTATGGTTTTACTCCAGGTGTCGGATGCACGTTAATTCTCCCTGAGAA
ATTCGGTGCTTTAGGGTTTGAAATGATGTATACTGCCCAAAATTATCGAGGGAAAGAATTAGCTGAAC
GGGGTGTTTCTTTTCCAGTTGTACCGAGAAAAGATGTGCTAGAAGTGGCTAAAAATATAGCCTATGAA
ATGTCGGAAAAACCCAGACTATCTTTAATAACTTTAAAGGAACACTTAACTTCAAAAATCCGCAAAAC
GCTACCAGGATTTATAGATAAAGAAGTAGCTATGCACGAAATAACCTTTCACCAACCAGAGGTAGCAA
GTAGAATAGAGGAAAATTTTGACAAAAGGACAACGGCAAGTAATAACCCTCAAAATTTTCCTCAAGA
GGCTGTAAGAAAAGAAATAAGAACAGATTCATTGAATTGTCAACCATTTCAGTTGAAGACATTTAGTT
ATGGTTCGTTAAACAATTTAACTTTGGTACCTCTAGAACGTAGAGTTCCAAGCCCAAGTGAAGTTGAG
GTTCAAATCAAAACTGTACCAGTTAATTTTCGGGATATACTCAATGCACTTGGTATGCTCCAAGAGTAT
TACGAAAAAACATTTGGCATTGCTAATGCTGAAGATCTCACTTTTGGTTTTGAAGGTGCAGGTACTATC
GTAGCTGTTGGGGCAGAAGTATCGCAGTGGCAAGTCGGCGATGAAGTAATGGTAATGAGAATTCACG
ATGCATTTAGTAGCTTTATTATCTGCTCGCCGGACAAACTGGTGCGTAAAAATTTTAATCTGAATATGG
AAGATGGAGCTAGTATTTGGGGGCCGTTTTCGACTGCATATCACGGGTTGATTAACTTAGCCAAAATTC
AGCCAGGAGATAGAGTATTAATTCACGCTGCTTCAGGTGGGATTGGACAAGCAGCAATTCAGTTGGCT
CAACTTTTTGGGGCGGAAGTATTTGCTACTACTAGTCCAGGTAAGATGAATTATCTCCGGGAACAGGG
AATTAAGTATGTGATGAATTCTCGAACGATGGAGTTTGTAAATGATGTGATGGAATTCACTCAGGGCC
GTGGGGTAGATGTTATTCTCAATAGCCTGACTCATGGAGAATACATCCAGAAAAATCTAGAGATCCTT
GCCGATAGAGGGCGATATGTTGAACTTGGTAAGTTGGGCATTTGGAGTCACGAGCAAGTCTATCAGAA
ACGCCCAGATATCAAATATTTTACTTTTGATTTGTTAGAAGAATTTGCCAAAGATAATCAATTGTTTTCT
CAGATATGGGATAATTTGGCACTTGAATTTGACCGCGATCGCTTGAAGCCACTACCTTACAAAACATTT
CCAATAGAAGATGTTCTTAAAGCCTTTGATTATATGCGACGCGGGAAGCATTTTGGCAGGGTAGTGGT
AGTTATGCCTGACTCCTATTCTAGACAGGAACAAGAGTTGGATGCTCGGTTATCCATTGAAAACAAAA
TGACTAAAGAGGAACAGATATTATTTCAATTACAATCTGGTGAAGTTTCCTTAGAAAATGCGGAACAA
CTATTGTTAGGAAATACGGAAACAGAAACACAAGATAAAGCTATAGCAGAAAATCAAATAGATAATA
TTCAAAACAAGTTAATTAACATGGATAGCTCAGAGAAAATCTTATCTTTGATTAGTTCAGTAGAAATAT
CTTTAGAAACAGCAGAAAAATTATTATTAGAAGTAGTAGAACCAGAAGTTAAAACAGAGGTTAATGAT
GAGGTTAATCCTAGTCAAAATCATATACCAACTACAGATATAGCGATTATTGGTATTTCATGTAGATAT
CCAGGAGCGAATAACTGGAAAGAATTTTGGGAAAATTTAAAGAATGGAATTGACAGTGTAACGGAAG
CTCCTCCTGGAAGATGGGAAGAAAAAAATTGGTATCATCCAGATCCAGATAATCCAGGTACTTCCTAT
TCAAAATGTGCCGGTTTTTTAGATGAAATTGATAAATTTGACCCTTTATTTTTTCATATTTCTCCGGAAG
AAGCTTGGTTTATGGAGCCTGAGCAAAGAATATTTTTAGAAGAAGCTTACCACGCTATAGAAGATGCT
GGATATGCTACAGACTCTCTTAGAGGTAAACAATATGGAGTATTTGTGGGAGTTACGGTAAATGGTGG
TTATCTTAAGTTGTTGTCAATTTCAGGATTAGATGTTCATAGGATGGCGGCTACAGGAAATGGTCCGTC
AATGATACCAGCAAGAATTGCCTATATGCTTGACCTTCAAGGGCCAGTAGTAGCTATTGATACTGCCTG
CTCGTCGTCATTGGTAGCTGTTCATCAAGCTTGCCAAAGCATACAACGAGGAGAGAGCGAAATAGCGA
TCGCTGGAGGTATTACTCTAATGCCAACATCAGACTTCCAAATAATGTCAAGTCAGTTTCAAGTTGTAT
CTCCTGATGGACGTTGTAAGACTTTTGATGCTTCAGCATCGGGTACAGCCTGGAGCGAAGGTTGTGGTG
TTCTCTTATTAAAAAGCTACAGTCAAGCAATTCAAGACAACGACCATATTTATGGAGTAATCAAAGGA
ACAGGAGTTAATTATGATGGTAATACTAACGGGATTAGTGCCCCTAGTAGTCAATCTCAAGCCAGTTT
AGAAGAAGCAGTTTATCAGAAATTTGGAATTAACCCAGAAACTATTAGTTATGTAGAAGCTCATGGTA
CAGCAACACCTCTGGGAGACCCGATAGAAGTAGAAGCTTTAACAGAAGCCTTTTCTAAATGGACGAAC
AAAAAACAGTTTTGTGCGATTGGCTCGGTAAAAACTAATATTGGTCATTCAGCAGCGGCTGCCGGAGT
TTCTGGTTTGATCAAGACAATTTTGTGCCTCAAAAATCAAAAATTAGTTCCATCCTTACATTTTAATCA
ACCAAACCCACATATTGACTTTGAAAATAGCCCTTTTTATGTCAATACAAAATTAAAAGATTGGGAAG
TGCTTGAAGGCCAACCAAGGCAAGCTACAGTTAGTTCTTTTGGTTTTAGTGGCACTAATGCTCATATAG
TTATAGAAGAGGCTCCTTCTCAAGTTAAAAGTCAGAATATTGTTGAACGTCCGATCCATCTGTTAACTC
TATCTGCGAAAACAGAAAAGGCTCTAGAAGATTTAGTCAGTAATTATCAAAATTATTTAGAAACTAAT
CCCGAGTTACCACTAGCAGATGTATGTTATACAGCCTCTACAGGTAGAGCACATTTTAATTATCGATTA
GGAGTTATTGCTTCTGAACCAAAAGCATTAATAGAGAAACTACTTGGGTGGAAAGCTCAGGAAGAATT
AGTAGGACTATTTTCAGGAAAACGAAATAGCGAAGGTCAGAAAATAGCATTCCTGTTCACAGGTCAAG
GTTCCCAGTATGCGAATATGGGAAGGCAACTTTATGAAAAAGCACCAACTTTCCGTCAAGCTTTAGAG
GAATGTGACCAAATTTTACAACCCTATCTAGAAGTACCTCTATTAGAGGTCATATACTCTGAGGATGCA
CAAAAGTCAAGTGATAATCTATTAGACCAAACAGCTTACACCCAACCAGCTGTGTTTGCTGTTGAATAT
GCTTTGGCTAAATTATGGTCTTCATGGGGAATCAAACCATGTGTAGTCATGGGTCACAGCGTAGGAGA
ATATGTAGCAGCAACAGTAGCCGGAGTATTCAGTTTAGAAGATGGTCTGAAACTAATAGCCATGCGAG
GAAAGTTGATGCAAAAGTTACCCTCCGGTGGTGAGATGGTATCCGTAATGGCATCAGAGTCTCAGGTA
ACAGAGGCTATAAAAGAATATAGTTCCCAAGTAACAATAGCAGCGATAAATGGACCAGAAAGTATAG
TAATTTCTGGTGAGAGTGTAGCCATAAAAAATATTTGTAGTTTATTTGAATCAGAGGGAATAAAGACC
AAGCAGCTACAAGTATCCCACGCTTTCCATTCTCCAATGATGGAACCGATGTTAGCAGAGTTTGAATCA
GTAGCTAAACAAGTCACCTATAATCAACCCCAAATACCACTAATATCAAACGTCACAGGTACTGAAGT
AGATGGAGAAATAACAAATGCTGAATATTGGGTAGATCATGTGCGTCAACCAGTAAGATTTGCCCAGA
GTATGAAAACTCTAGAGTCGGAAGGATATGAAACCTTCCTAGAAATAGGACCGAGACCAATATTGCTA
GGAATGGGAAGACAATGTGTAACAGAAGATGTAGGAGAATGGCTGCCATCATTACGTCCAGGGGTGG
ATGAATGGGAACAAATGCTATCAAGCTTAGGAAAATTGTATGTAAAAGGAGCCAAAATAGACTGGTC
AGGGTTTGACTCTGATTATACTCGCCAGAAAGTAGTATTACCCACATATCCATTCCAGGGAGAACGTTA
TTGGGTAGAAACCAACAACAACTTCTGGCCTCAACGGCAATTTTCCCAAGGGGAAAACCTCCATCCCC
TATTAGGTCAAAAGCTAAATTGTGCAGGGGAACAACAAATATTTGCATCACAAATAGGAGAAAACTCA
CCCAACTATCTGAGGGACCACCGAGTATTTAATCAAGCACTATTTCCCATAACAGGCTACCTAGAAAT
AGCAATAGCAGCAGGAAATCACCAATTAAAAACATCCCAGATAGTAATAGAAGACCTAACCATAACT
AGAGGATGGATACTACCAACAGGAGAATTAACCAATGCCCAAACCATACTCACCCCAATAGATAACC
AAAGCTATAAGTTTCAAATATTTTCTCAACCAGAACAACAGGAGTGGAGACTCCACACGACAGGAAAA
ATCAGAAAAGAGTCAACCCCCCCTACTCAGACAAAAGTTGACCTAGAAAAATACAAGAGTGAATGTA
ATCAAACAATAGAAGTCAAACAACATTATCAAAAATGTCAACAAGTAGGGATAGACTACGGGAATAC
CTTCCAAGGCATCCAAGAATTGTGGTCAGGTTCAAACCAAGCATTAGGTTATATCAAACTGCCCGAAG
AATTGATAACACAAACAAGCGACTATCATTTCCATCCAGCACTATTAGATGCAGCCTTGCAAGTAATG
TTTTATGCACTGCCAGCAACGGATAATGACAAAACTTATCTGTCAGCAGGAATAGAAGAATTCAGACT
ATATAAGACTCCCGGGCTGAGTATATGGGCATATGTATCAGTAACCAGTCAAGAAGTGGAAACTCCAG
AAAGTTTGACGGCTATTGTCACCATAGTAACTCCAGAAGGAGAAATAATTGCCAACATCAAAGGTTTA
CAAGTCAAACTAGCAACAAAACAGACCCTACTGGGAACAGAAACCGAATCAATAGAAAATTGGTTAT
ATGAAGTAGAGTGGAGAAACAAAGGTATTTTAGGTAAACTACTCCCACCAGATTTCCTCATACCTCCT
ATACAAATCAACCAAAAATTAACTCCAACTCTGACAGAATTAGTAACTCAAGTAGATAATGAAACAAC
AGCGTCTTTTGAAACAAGCTTAGAAGAATTAAGCAGAGATTATATAGTACAAGCATTACAGTCAATGA
GTTGGTCATACAAACCAACAGAAAGCTTTGCATTTGATGTAGCAGCCCAAAAATTAGGTATAGTTCCT
ACCCATCGACCACTGTTTAAGCGTTTGCTGCAAATATTAACAGAGTCAGGAATACTCAACTCAAAGAA
TCAGCAGTGGGAAGTAGCACAAACCTTACCTGAAGTCAAGCCTACGGAAAAAATCAGCAGTTTACAG
AAGAAATATCCAGAAGAAACAGCAGCATTGACACTACTCTCTCGTTGTGGGTCTAAACTAAGTGGGGT
ATTACGAGGAGCAATAGACCCAGGAGAGTTAGTGTTCCCCCAAGGAGATTTGACAGCAGCGACTCAAC
TTTATGAAGACTCAACAGTAGCGAAAGTGATGAACACAATAGTAGAAAAATCCATCACCAAAGCTATA
GAAAAATCCCCGAAAAGCCGGGGGCTCAGGTTGTTGGAAATAGGAGGGGGAACAGGAGGGACTACAA
GCTATATCCTACCTCATCTAACTCCTCAGCAAACCGAATATACATTCACGGATATAGGGGCATTATTTA
CAGCCAAAGCTCAAGAGAAATTCCGGGATTATAAGTTCATAAAGTATCAAACTTTAGACATAGAAGTA
GACCCGACAACTCAAGGATTTGAGGCTCATCAATATGATGTAATTATTGCAGCTAATGTACTTCATGCA
ACGACAGATATGAAGCAGACATTATCTCATGTGCGAGAACTGTTAGCAGATGGGGGAATGTTGGTGTT
ATCTGAAGCAACAGCTAAAACACTATGGGTAGATTTAGTATTTGGGTTGTTAGAAGGATGGTGGAAAT
TCAGGGATTATGAATTACGACCAGATTATCCTTTGTTGAGTCGTGAAAAATGGCATCATGTCTTGAGAG
AAACGGGTTTTACTGAAGTAGTTACTATGCCAGAAGTGGAGGGAATGGCAGAAACATTGTCAGCACAA
ACGGTAATTGTAGCTAAAAGCTCTCAAACGAAGTTAGAACAAAGGAATGATGATTCTAAGAGTTGGTT
GATACTGGCAGACTCTGAGGGAGTAGGGCAACAGTTAGCGACCTTACTCCGCTCAGTAGGAGAGGTTT
GTACTTTAGTATTTGCCGGAGAAAGGTATCAACAGATAGCTCCGGGAGAATTTAGTATTAATCCTAATC
AAGCTAAAGATTTTGAGGAGGTAATAGAGACAGTGGCAGGGAAGTCACCATCATTGTATGGAGTGGT
ACAATGTTGGACGACTGAAGCCGGAGTGGGGAATGGGATTAATTCTGAGGAGTTAGGAAGTTTATCGA
AGTTAGGGTGTGGCACAACTCTATCTTTAGTACAAGCATTGGTGAAAGGCGGGTTATCAACTGTCCCTC
GATTATGGTTGGTGACAAATGGTGCTCAGGCGGTGCCGAATAATCATCCGGTGATACCAGGAGTAGCT
CAATCTTCGGTATGGGGAATGGGGAAAGTGATTAGCTGGGAACATCCAGAGTTGAACTGTACTCGTAT
AGATTTGGACCCAGAGGAGACTTTAGAGGGTAAAGTTGATGCCCTATTTAAAGAAATTTGGTCGGAGG
ATAGGGAAGACCAGGTAGCATGGCGTGGGGATAGTCGTTATGTAGCTCGGTTGGTGGGTAGTCATCAT
CGGCAATTAGTGGCACAACAAGCTGATGGTAAAACTCAAAAGCCCTTAAGTTTCCGTTCTGAAGCAAG
CTATTTGATTACAGGAGGTATGGGAGGTTTGGGTTTGCTGGTAGCTAGTTGGATGGTGTCAAAGGGAG
CTAAACATTTGATATTGTTAGGACGCCGTTCACCGGATGATGCTACGAGGAAAAAAATAACCGAGTTA
GAAATGGCAGGAGCGTCCGTGGTAGTGGAAAAAGCCGATGTGACTGATTTGGAGTCGATGAAAGGGG
TGTTGCAGAGGATTGAGGAGTCAAAGCGACCGTTAGTAGGAGTGATTCATTCTGTGATGGTGCTATCA
AATGGAGTGCTACGAAATCAGACTTGGTCTAGTTTTGAACAGGTGATGGAACCGAAAGTTCAAGGTGC
TTGGCACTTGCATCAATTGACTCAAAGTCAACCATTAGACTTCTTTGTGCTGTTTTCTTCAGCAACATCT
CTGTTGGGTTCACTGGGTCAGGGCATGGCAAATTATTCTGCTGCTAATGGGTTTCTTGATGGTTTAGCT
CATTATCGTCGAACTATGGGATTACCGGGATTGAGTATCCATTGGGGAGCAGTTTCTCAAGTGGGACG
AGCGCTCGGGCGAGATACAGAGACAGCCGCTATGCTCAGTAAGAATGGGATGGGTTTAATATCTCCGG
CTCAGGTATTGGAGTCTTTGGAACTATTGATGAGTAGTTCGGATGTGGAAGTAGGGGTGATGCCTATTG
AGTGGTCAGGGTGGCAGGAGAGAGTGGCACAGTGGCCGTTTTTAGTGGATTGGCAGGAAACTATATTG
GAGGTAGCCCAACCATCGAAGTCAGATTTTCTGTTAAAGTTGGAGGCTACACCACCTAATGAGCTTCG
CTTGTTGTTGGTGGCTCATGTGCGTCGTCAGGTGGCTCAAGTGTTGAGAATTAGTCATCCTGAATCAAT
TGAAATGGATACAGGGTTTTTTGATTTGGGTATAGACTCTTTGACTTCTGTGGAGTTGAGGAATAAGTT
GCAAGGTAGTTTAAAGTGTTCAGTACCTTCTACTGTAACTTTGGACTACCCTACCATTAAGGCATTGGT
AGAATATTTATATCAACAGCTATTGTTAGAACAAGTTAGTTACTCAAATACTGTACCAACTGAAGAAA
TCAATGAAGATAGGGAGGAGATTACTCTAAGCGGGACACCAGCTCGGGAAGCAAGTAGTGCCTTACG
GGATGGCTATCGTCAAGCAGGAGTCAGCGGGCGGGTTCGGAGTTATTTGGATTTATTGGCTGGTTTAA
GTGATTTTCGGGAACATTTTGATGGTAGTGATGGTTTTTCTTTAGATTTGGTGGATATGGCTGATGGTCC
CGGTGAAGTGACCGTTATTTGTTGTGCCGGTACTGCCGCTATTTCTGGTCCCCATGAATTTACCCGCTTA
GCCGGTGCTTTGCGTGGTATTGCTCCCGTGCGGGCTGTTCCACAACCAGGTTATGAAGAAGGTGAACC
CTTACCCAGTTCTATGGCCGCTGTGGCCGCTGTTCAAGCCGATGCTGTTATTCGCACCCAAGGTGATAA
ACCCTTTGTGGTTGCTGGTCATAGTGCCGGTGCTTTAATGGCCTATGCTTTGGCCACTGAATTATTGGAT
CGTGGTCATCCTCCCCGCGGTGTGGTTTTAATTGATGTGTATCCTCCCGGTCATCAAGATGCTATGAAT
GCCTGGTTAGAAGAATTGACCGCCACTTTGTTTGATCGGGAAACCGTTCGCATGGATGATACCCGCTTA
ACTGCTTTGGGTGCCTATGATCGTTTAACTGGTCAATGGCGCCCCCGTGAAACCGGTTTGCCCACTTTA
TTGGTGAGTGCTGGTGAACCTATGGGTCCCTGGCCCGATGATTCTTGGAAACCCACCTGGCCCTTTGAA
CATGATACTGTGGCCGTTCCCGGTGATCATTTTACTATGGTTCAAGAACACGCTGATGCTATTGCACGA
CACATTGATGCCTGGTTGGGCGGTGGAAACTCCTAA
CTCCCTATAGTGAGTCGTATTAGCGGCCGCATCGAATATAACTTCGTATAATGTATGCTATACGAAGTT
ATTAGCGATGAGGACATGAGGTTGCCCCGTATTCAGTGTCGCTGATTTGTATTGTCTGAAGTTGTTTTT
ACGTTAAGTTGATGCAGATCAATTAATACGATACCTGCGTCATAATTGATTATTTGACGTGGTTTGATG
GCCTCCACGCACGTTGTGATATGTAGATGATAATCATTATCACTTTACGGGTCCTTTCCGGTGATCCGA
CAGGTTACGGGGCGGCGACCTCGCGGGTTTTCGCTATTTATGAAAATTTTCCGGTTTAAGGCGTTTCCG
TTCTTCTTCGTCATAACTTAATGTTTTTATTTAAAATACCCTCTGAAAAGAAAGGAAACGACAGGTGCT
GAAAGCGAGGCTTTTTGGCCTCTGTCGTTTCCTTTCTCTGTTTTTGTCCGTGGAATGAACAATGGAAGTC
CTCGTCGAGGACGATCTTCCGCTGCATAACCCTGCTTCGGGGTCATTATAGCGATTTTTTCGGTATATC
CATCCTTTTTCGCACGATATACAGGATTTTGCCAAAGGGTTCGTGTAGACTTTCCTTGGTGTATCCAAC
GGCGTCAGCCGGGCAGGATAGGTGAAGTAGGCCCACCCGCGAGCGGGTGTTCCTTCTTCACTGTCCCT
TATTCGCACCTGGCGGTGCTCAACGGGAATCCTGCTCTGCGAGGCTGGCCGGCTACCGCCGGCGTAAC
AGATGAGGGCAAGCGGATGGCTGATGAAACCAAGCCAACCAGGAAGGGCAGCCCACCTATCAAGGTG
TACTGCCTTCCAGACGAACGAAGAGCGATTGAGGAAAAGGCGGCGGCGGCCGGCATGAGCCTGTCGG
CCTACCTGCTGGCCGTCGGCCAGGGCTACAAAATCACGGGCGTCGTGGACTATGAGCACGTCCGCGAG
CTGGCCCGCATCAATGGCGACCTGGGCCGCCTGGGCGGCCTGCTGAAACTCTGGCTCACCGACGACCC
GCGCACGGCGCGGTTCGGTGATGCCACGATCCTCGCCCTGCTGGCGAAGATCGAAGAGAAGCAGGAC
GAGCTTGGCAAGGTCATGATGGGCGTGGTCCGCCCGAGGGCAGAGCCATGACTTTTTTAGCCGCTAAA
ACGGCCGGGGGGTGCGCGTGATTGCCAAGCACGTCCCCATGCGCTCCATCAAGAAGAGCGACTTCGCG
GAGCTGGTGAAGTACATCACCGACGAGCAAGGCAAGACGATCCCCTCGACAGCGACACACTTGCATC
GGATGCAGCCCGGTTAACGTGCCGGCACGGCCTGGGTAACCAGGTATTTTGTCCACATAACCGTGCGC
AAAATGTTGTGGATAAGCAGGACACAGCAGCAATCCACAGCAGGCATACAACCGCACACCGAGGTTA
CTCCGTTCTACAGGTTACGACGACATGTCAATACTTGCCCTTGACAGGCATTGATGGAATCGTAGTCTC
ACGCTGATAGTCTGATCGACAATACAAGTGGGACCGTGGTCCCAGACCGATAATCAGACCGACAACAC
GAGTGGGATCGTGGTCCCAGACTAATAATCAGACCGACGATACGAGTGGGACCGTGGTCCCAGACTAA
TAATCAGACCGACGATACGAGTGGGACCGTGGTTCCAGACTAATAATCAGACCGACGATACGAGTGG
GACCGTGGTCCCAGACTAATAATCAGACCGACGATACGAGTGGGACCATGGTCCCAGACTAATAATCA
GACCGACGATACGAGTGGGACCGTGGTCCCAGTCTGATTATCAGACCGACGATACGAGTGGGACCGTG
GTCCCAGACTAATAATCAGACCGACGATACGAGTGGGACCGTGGTCCCAGACTAATAATCAGACCGAC
GATACGAGTGGGACCGTGGTCCCAGTCTGATTATCAGACCGACGATACAAGTGGAACAGTGGGCCCAG
AGAGAATATTCAGGCCAGTTATGCTTTCTGGCCTGTAACAAAGGACATTAAGTAAAGACAGATAAACG
TAGACTAAAACGTGGTCGCATCAGGGTGCTGGCTTTTCAAGTTCCTTAAGAATGGCCTCAATTTTCTCT
ATACACTCAGTTGGAACACGAGACCTGTCCAGGTTAAGCACCATTTTATCGCCCTTATACAATACTGTC
GCTCCAGGAGCAAACTGATGTCGTGAGCTTAAACTAGTTCTTGATGCAGATGACGTTTTAAGCACAGA
AGTTAAAAGAGTGATAACTTCTTCAGCTTCAAATATCACCCCAGCTTTTTTCTGCTCATGAAGGTTAGA
TGCCTGCTGCTTAAGTAATTCCTCTTTATCTGTAAAGGCTTTTTGAAGTGCATCACCTGACCGGGCAGA
TAGTTCACCGGGGTGAGAAAAAAGAGCAACAACTGATTTAGGCAATTTGGCGGTGTTGATACAGCGGG
TAATAATCTTACGTGAAATATTTTCCGCATCAGCCAGCGCAGAAATATTTCCAGCAAATTCATTCTGCA
ATCGGCTTGCATAACGCTGACCACGTTCATAAGCACTTGTTGGGCGATAATCGTTACCCAATCTGGATA
ATGCAGCCATCTGCTCATCATCCAGCTCGCCAACCAGAACACGATAATCACTTTCGGTAAGTGCAGCA
GCTTTACGACGGCGACTCCCATCGGCAATTTCTATGACACCAGATACTCTTCGACCGAACGCCGGTGTC
TGTTGACCAGTCAGTAGAAAAGAAGGGATGAGATCATCCAGTGCGTCCTCAGTAAGCAGCTCCTGGTC
ACGTTCATTACCTGACCATACCCGAGAGGTCTTCTCAACACTATCACCCCGGAGCACTTCAAGAGTAA
ACTTCACATCCCGACCACATACAGGCAAAGTAATGGCATTACCGCGAGCCATTACTCCTACGCGCGCA
ATTAACGAATCCACCATCGGGGCAGCTGGTGTCGATAACGAAGTATCTTCAACCGGTTGAGTATTGAG
CGTATGTTTTGGAATAACAGGCGCACGCTTCATTATCTAATCTCCCAGCGTGGTTTAATCAGACGATCG
AAAATTTCATTGCAGACAGGTTCCCAAATAGAAAGAGCATTTCTCCAGGCACCAGTTGAAGAGCGTTG
ATCAATGGCCTGTTCAAAAACAGTTCTCATCCGGATCTGACCTTTACCAACTTCATCCGTTTCACGTAC
AACATTTTTTAGAACCATGCTTCCCCAGGCATCCCGAATTTGCTCCTCCATCCACGGGGACTGAGAGCC
ATTACTATTGCTGTATTTGGTAAGCAAAATACGTACATCAGGCTCGAACCCTTTAAGATCAACGTTCTT
GAGCAGATCACGAAGCATATCGAAAAACTGCAGTGCGGAGGTGTAGTCAAACAACTCAGCAGGCGTG
GGAACAATCAGCACATCAGCAGCACATACGACATTAATCGTGCCGATACCCAGGTTAGGCGCGCTGTC
AATAACTATGACATCATAGTCATGAGCAACAGTTTCAATGGCCAGTCGGAGCATCAGGTGTGGATCGG
TGGGCAGTTTACCTTCATCAAATTTGCCCATTAACTCAGTTTCAATACGGTGCAGAGCCAGACAGGAA
GGAATAATGTCAAGCCCCGGCCAGCAAGTGGGCTTTATTGCATAAGTGACATCGTCCTTTTCCCCAAG
ATAGAAAGGCAGGAGAGTGTCTTCTGCATGAATATGAAGATCTGGTACCCATCCGTGATACATTGAGG
CTGTTCCCTGGGGGTCGTTACCTTCCACGAGCAAAACACGTAGCCCCTTCAGAGCCAGATCCTGAGCA
AGATGAACAGAAACTGAGGTTTTGTAAACGCCACCTTTATGGGCAGCAACCCCGATCACCGGTGGAAA
TACGTCTTCAGCACGTCGCAATCGCGTACCAAACACATCACGCATATGATTAATTTGTTCAATTGTATA
ACCAACACGTTGCTCAACCCGTCCTCGAATTTCCATATCCGGGTGCGGTAGTCGCCCTGCTTTCTCGGC
ATCTCTGATAGCCTGAGAAGAAACCCCAACTAAATCCGCTGCTTCACCTATTCTCCAGCGCCGGGTTAT
TTTCCTCGCTTCCGGGCTGTCATCATTAAACTGTGCAATGGCGATAGCCTTCGTCATTTCATGACCAGC
GTTTATGCACTGGTTAAGTGTTTCCATGAGTTTCATTCTGAACATCCTTTAATCATTGCTTTGCGTTTTTT
TATTAAATCTTGCAATTTACTGCAAAGCAACAACAAAATCGCAAAGTCATCAAAAAACCGCAAAGTTG
TTTAAAATAAGAGCAACACTACAAAAGGAGATAAGAAGAGCACATACCTCAGTCACTTATTATCACTA
GCGCTCGCCGCAGCCGTGTAACCGAGCATAGCGAGCGAACTGGCGAGGAAGCAAAGAAGAACTGTTC
TGTCAGATAGCTCTTACGCTCAGCGCAAGAAGAAATATCCACCGTGGGAAAAACTCCAGGTAGAGGTA
CACACGCGGATAGCCAATTCAGAGTAATAAACTGTGATAATCAACCCTCATCAATGATGACGAACTAA
CCCCCGATATCAGGTCACATGACGAAGGGAAAGAGAAGGAAATCAACTGTGACAAACTGCCCTCAAA
TTTGGCTTCCTTAAAAATTACAGTTCAAAAAGTATGAGAAAATCCATGCAGGCTGAAGGAAACAGCAA
AACTGTGACAAATTACCCTCAGTAGGTCAGAACAAATGTGACGAACCACCCTCAAATCTGTGACAGAT
AACCCTCAGACTATCCTGTCGTCATGGAAGTGATATCGCGGAAGGAAAATACGATATGAGTCGTCTGG
CGGCCTTTCTTTTTCTCAATGTATGAGAGGCGCATTGGAGTTCTGCTGTTGATCTCATTAACACAGACCT
GCAGGAAGCGGCGGCGGAAGTCAGGCATACGCTGGTAACTTTGAGGCAGCTGGTAACGCTCTATGATC
CAGTCGATTTTCAGAGAGACGATGCCTGAGCCATCCGGCTTACGATACTGACACAGGGATTCGTATAA
ACGCATGGCATACGGATTGGTGATTTCTTTTGTTTCACTAAGCCGAAACTGCGTAAACCGGTTCTGTAA
CCCGATAAAGAAGGGAATGAGATATGGGTTGATATGTACACTGTAAAGCCCTCTGGATGGACTGTGCG
CACGTTTGATAAACCAAGGAAAAGATTCATAGCCTTTTTCATCGCCGGCATCCTCTTCAGGGCGATAAA
AAACCACTTCCTTCCCCGCGAAACTCTTCAATGCCTGCCGTATATCCTTACTGGCTTCCGCAGAGGTCA
ATCCGAATATTTCAGCATATTTAGCAACATGGATCTCGCAGATACCGTCATGTTCCTGTAGGGTGCCAT
CAGATTTTCTGATCTGGTCAACGAACAGATACAGCATACGTTTTTGATCCCGGGAGAGACTATATGCCG
CCTCAGTGAGGTCGTTTGACTGGACGATTCGCGGGCTATTTTTACGTTTCTTGTGATTGATAACCGCTGT
TTCCGCCATGACAGATCCATGTGAAGTGTGACAAGTTTTTAGATTGTCACACTAAATAAAAAAGAGTC
AATAAGCAGGGATAACTTTGTGAAAAAACAGCTTCTTCTGAGGGCAATTTGTCACAGGGTTAAGGGCA
ATTTGTCACAGACAGGACTGTCATTTGAGGGTGATTTGTCACACTGAAAGGGCAATTTGTCACAACAC
CTTCTCTAGAACCAGCATGGATAAAGGCCTACAAGGCGCTCTAAAAAAGAAGATCTAAAAACTATAAA
AAAAATAATTATAAAAATATCCCCGTGGATAAGTGGATAACCCCAAGGGAAGTTTTTTCAGGCATCGT
GTGTAAGCAGAATATATAAGTGCTGTTCCCTGGTGCTTCCTCGCTCACTCGAGGGCTTCGCCCTGTCGC
TCGACTGCGGCGAGCCTACTGGCTGTAAAAGGACAGACCACATCATGGTTCTGTGTTCATTAGGTTGTT
CTGTCCATTGCTGACATAATCCGCTCCACTTCAACGTAACACCGCACGAAGATTTCTATTGTTCCTGAA
GGCATATTCAAATCGTTTTCGTTACCGCTTGCAGGCATCATGACAGAACACTACTTCCTATAAACGCTA
CACAGGCTCCTGAGATTAATAATGCGGATCTCTACGATAATGGGAGATTTTCCCGACTGTTTCGTTCGC
TTCTCAGTGGATAACAGCCAGCTTCTCTGTTTAACAGACAAAAACAGCATATCCACTCAGTTCCACATT
TCCATATAAAGGCCAAGGCATTTATTCTCAGGATAATTGTTTCAGCATCGCAACCGCATCAGACTCCGG
CATCGCAAACTGCACCCGGTGCCGGGCAGCCACATCCAGCGCAAAAACCTTCGTGTAGACTTCCGTTG
AACTGATGGACTTATGTCCCATCAGGCTTTGCAGAACTTTCAGCGGTATACCGGCATACAGCATGTGCA
TCGCATAGGAATGGCGGAACGTATGTGGTGTGACCGGAACAGAGAACGTCACACCGTCAGCAGCAGC
GGCGGCAACCGCCTCCCCAATCCAGGTCCTGACCGTTCTGTCCGTCACTTCCCAGATCCGCGCTTTCTC
TGTCCTTCCTGTGCGACGGTTACGCCGCTCCATGAGCTT
pRL838-rApra
(SEQ ID NO: 59)
ATCGCGAGAATTAATTCAGATAAAAAAAATCCTTAGCTTTCGCTAAGGATGATTTCTAGCGATGACCCT
GCTGATTGGTTCGCTGACCATTTCCGGGTGCGGGACGGCGTTACCAGAAACTCAGAAGGTTCGTCCAA
CCAAACCGACTCTGACGGCAGTTTACGAGAGAGATGATAGGGTCTGCTTCAGTAAGCCAGATGCTACA
CAATTAGGCTTGTACGGGTACTCGACCTGCATCCCTTAACTTACTTATTAAATAATTTATAGCTATTGA
AAAGAGATAAGAATTGTTCAAAGCTAATATTGTTTAAATCGTCAATTCCTGCATGTTTTAAGGAATTGT
TAAATTGATTTTTTGTAAATATTTTCTTGTATTCTTTGTTAACCCATTTCATAACGAAATAATTATACTTT
TGTTTATCTTTGTGTGATATTCTTGATTTTTTTCTACTTAATCTGATAAGTGAGCTATTCACTTTAGGTTT
AGGATGAAAATATTCTCTTGGAACCATACTTAATATAGAAATATCAACTTCTGCCATTAAAAGTAATGC
CAATGAGCGTTTTGTATTTAATAATCTTTTAGCAAACCCGTATTCCACGATTAAATAAATCTCATTAGC
TATACTATCAAAAACAATTTTGCGTATTATATCCGTACTTATGTTATAAGGTATATTACCATATATTTTA
TAGGATTGGTTTTTAGGAAATTTAAACTGCAATATATCCTTGTTTAAAACTTGGAAATTATCGTGATCA
ACAAGTTTATTTTCTGTAGTTTTGCATAATTTATGGTCTATTTCAATGGCAGTTACGAAATTACACCTCT
TTACTAATTCAAGGGTAAAATGGCCTTTTCCTGAGCCGATTTCAAAGATATTATCATGTTCATTTAATCT
TATATTTGTCATTATTTTATCTATATTATGTTTTGAAGTAATAAAGTTTTGACTGTGTTTTATATTTTTCT
CGTTCATTATAACCCTCTTTAATTTGGTTATATGAATTTTGCTTATTAACGATTCATTATAACCACTTAT
TTTTTGTTTGGTTGATAATGAACTGTGCTGATTACAAAAATACTAAAAATGCCCATATTTTTTCCTCCTT
ATAAAATTAGTATAATTATAGCACGCGAATTCATCGAATTTCTGCCATTCATCCGCTTATTATCACTTAT
TCAGGCGTAGCACCAGGCGTTTAAGGGCACCAATAACTGCCTTAAAAAAATTACGCCCCGCCCTGCCA
CTCATCGCAGTACTGTTGTAATTCATTAAGCATTCTGCCGACATGGAAGCCATCACAAACGGCATGATG
AACCTGAATCGCCAGCGGCATCAGCACCTTGTCGCCTTGCGTATAATATTTGCCCATGGTGAAAACGG
GGGCGAAGAAGTTGTCCATATTGGCCACGTTTAAATCAAAACTGGTGAAACTCACCCAGGGATTGGCT
GAGACGAAAAACATATTCTCAATAAACCCTTTAGGGAAATAGGCCAGGTTTTCACCGTAACACGCCAC
ATCTTGCGAATATATGTGTAGAAACTGCCGGAAATCGTCGTGGTATTCACTCCAGAGCGATGAAAACG
TTTCAGTTTGCTCATGGAAAACGGTGTAACAAGGGTGAACACTATCCCATATCACCAGCTCACCGTCTT
TCATTGCCATACGGAATTCCGGATGAGCATTCATCAGGCGGGCAAGAATGTGAATAAAGGCCGGATAA
AACTTGTGCTTATTTTTCTTTACGGTCTTTAAAAAGGCCGTAATATCCAGCTGAACGGTCTGGTTATAG
GTACATTGAGCAACTGACTGAAATGCCTCAAAATGTTCTTTACGATGCCATTGGGATATATCAACGGTG
GTATATCCAGTGATTTTTTTCTCCATTTTAGCTTCCTTAGCTCCTGAAAATCTCGATAACTCAAAAAATA
CGCCCGGTAGTGATCTTATTTCATTATGGTGAAAGTTGGAACCTCTTACGAGCGGCCGCATACGATTTA
TTGACAGCTAGCTCAGTCCTAGGTATAATGCTAGCATGACCGTATTACATAGTGTGGATTTTTTTCCGT
CTGGTAACGCGTCCGTGGCGATAGAACCCCGGCTCCCGCAGGCGGATTTTCCTGAACATCATCATGATT
TTCATGAAATTGTGATTGTCGAACATGGCACGGGTATTCATGTGTTTAATGGGCAGCCCTATACCATCA
CCGGTGGCACGGTCTGTTTCGTACGCGATCATGATCGGCATCTGTATGAACATACCGATAATCTGTGTC
TGACCAATGTGCTGTATCGCTCGCCGGATCGATTTCAGTTTCTCGCCGGGCTGAATCAGTTGCTGCCAC
AAGAGCTGGATGGGCAGTATCCGTCTCACTGGCGCGTTAACCACAGCGTATTGCAGCAGGTGCGACAG
CTGGTTGCACAGATGGAACAGCAGGAAGGGGAAAATGATTTACCCTCGACCGCCAGTCGCGAGATCTT
GTTTATGCAATTACTGCTCTTGCTGCGTAAAAGCAGTTTGCAGGAGAACCTGGAAAACAGCGCATCAC
GTCTCAACTTGCTTCTGGCCTGGCTGGAGGACCATTTTGCCGATGAGGTGAATTGGGATGCCGTGGCGG
ATCAATTTTCTCTTTCACTGCGTACGCTACATCGGCAGCTTAAGCAGCAAACGGGACTGACGCCTCAGC
GATACCTGAACCGCCTGCGACTGATGAAAGCCCGACATCTGCTACGCCACAGCGAGGCCAGCGTTACT
GACATCGCCTATCGCTGTGGATTCAGCGACAGTAACCACTTTTCGACGCTTTTTCGCCGAGAGTTTAAC
TGGTCACCGCGTGATATTCGCCAGGGACGGGATGGCTTTCTGCAATAAGCCACAATTCAGCAAATTGT
GAACATCATCACGTTCATCTTTCCCTGGTTGCCAATGGCCCATTTTCCTGTCAGTAACGAGAAGGTCGC
GAATTCAGGCGCTTTTTAGACTGGTCGTAATGAAGAAACAGACCATGCTAGATAAAATAAATCGTTAT
GCTCATGGGTTTGTAGCTGTACCAGTGATTTGTGCTTGTTCAGAAGCAGGGGTATTCGAGTTACTATCA
CAGAAAAAATCACTGAAATTAGAAGAAATAGTAGAGCATTTAGCAGCAAATAGTGGACATCTAATGG
TAGCTATGCGACTCCTAGAGTCATTATCATTCCTATATCGCTCTCAAGCAGAAGAATACATATTGACAG
AACAAAGTCAACAACATCAAATCATCCCTAAAGCTCTGATGAGCTTATATAAATATCCATTTGAGTTAT
ATTTAAAAGGAGAAGTAGAAACAGGAATCAGCAATTGGATTAATTGTTCATCTCGAAGATGGGATACA
GAAAACTCATTACTAAGTGATTTATTAGATGGAGTATTACTCATCCCCCTGCTACTAGAACTGAAAAAA
CAAAACTTACTGGATGAATCAAAAAAAATATTTAACACATTAACAAATTCCCTTAAACAAGAACTATC
AACATTATTCATCAATTTAGGATGGGCAGAAGAAAAAACAGAAGGGCTATATTTAACAGATATAGGTA
GATTTATGCGTGACAGATCCTTAAACTTAGGAACAACAGCGTCTTATGCTCCTATGTTGTTACAGATGA
AAGAATTACTATTTGGAAATCCTCAGAGAGTATTTCAAAGAAATAAGACTGAAAAAGAAAGACACGT
AAATAGAACATTAAATGTAGTAGCAAGTGGCTTTCAACACGAAAAGTTTTTTGCCGATACAGATAAAA
TCATCATATCTATATTTAACCAACAGCCAATAGAAGAGCAACCAAGCTATATAGTAGACATGGGTTGT
GGGGATGGAACGCTACTGAAACGAATATATAAAATCATCAAACAATTCTCTGCTAGGGGAAAAGTATT
AACAGAGTATCCTATCATCATGGTAGGAGTAGACTACAATCAAGAAGCATTAGATGTCACAGATAAAA
ATTTAGTAGATATTCCCCATCTAGTCATTCCAGGAGATATAGGAGCACCAGAAAAATTATTAGAGCAA
TTGAAAGCACAAGGAATAGAACCAGAAAAAGTATTACATATTCGTTCATTCCTAGATCATGACCGACC
ATTTATAGCTCCAAAAAATACAGAAATAGCTCAAGCACGTTCTCAGTTAGATTATCAAGTAGTAGATG
TAGATCGAGAAGGAAAGCTGATACCACCTCACATAGCAGTACAAAGTTTAGTAGAACACTTAGAAAG
ATGGTCATCAATAATAACTAGACATGGATTATTGCTCCTAGAAGTACATAGTTTAACGCCAGCAGTAG
TCAAAAAATATATAGACGAAAGTGAATCTCTGCATTTTGATGCCTATCATGCCTTCTCAATGCAACATT
TAGTAGAAGCAGATGTGTTCTTGATGGCAGCAGCAGAGGTGGGTTTATTTTCACGGAAAGAAGCTTTT
CGTAAGTATCCGAAAACGTTGCCATTGACTCGAATCACAGTTAATCATTTTGAAAAGCGTAAATATCA
GATACGGTATGCAACTGTAAACGATATACCAAATCTGTTAAAGTGTGCGACATTTAATCAACCTGTAA
ACGAACCCTTCTTTCAAGTTTTATTGAAACAAACTCCAACAGCACATTTATTATTAGAATATCAAGGTG
AATTGGTAGCTGCAATCTTCACAGAAACTAAAAATTCTAATGAGGTGCTAGGAATTCGTGAGTTTTTAG
TAAGGACTTCTGTAGAAAATTGGCAAGTATTAGCAAAAGATTTACTAGAATTTGTGGAACAGTGGGGA
GTAGTCAAACCAGGAATAAAAGAAATAGAAGGATTGTTAAAATACCATGAAGCCATCTCAAACTTTCA
AAAATCAAAGTGGTATCAATCTTCAGTTTTAAACAAAAAGCTTATAGAAAAAATAACTCTACACGAAT
TAGCTACTTTAGAACTATGTAATTTAATGGCTCCAGAATATGAGCTAGAAGCCTTTGCCGCACGTTGGC
TTTTGCGTGTTTTTCAAGATATGGGTGTATTTCTCAGAGAGGGTGAATCTTATCAGGAGTCTGAGTTGG
TTTCCCAGCTAAACATCTCACCACGTTACCAGAGACTTTTAGGTGCTTTGTTACAAATTTTGCATAAGC
GTGGTATTCTAAAAATTGAGAAAGATAGAGTGTTCACATTAGCAAGATGTAAGACCTTTGCCTTAGAA
AATATTTCGTCTGAAGTTTCTGCTTTTTATGATTATTTTTCTGAAAAATATCCTGCTCATTTATCCTGGTT
AACAGTAGTCAAAAGGTGTCTAGAGAAATATCCTTTGATTCTACGCGGTGAAGTTGATGTCAACGAAG
TTGTTTTTACAGATGGGGATATGGAGCTATTTGCTGGACTATTTCTAGGACATCGTGTTGCTGACTACTT
TAATGAGTTGCTAGCAGATGGGGTTTGCTGGGAAGTAGAACAGCGGTTGTTAGAAGAAAAGAGGGCA
CAACCTATTCGGATCTTGGAGATTGGAGCAGGAACAGGAGGTGTTACAGGAATATTGCTAGAAAAGCT
AGCTTCTCATGCAGAGCAAATTGAATTTTGGTTTACTGATATTTCTAGCGTTTTTACACGTTATGGTGAG
AGTAAGTTCAAGCAGTTTCCTTGGGTGAAATATCAAACCTTTGACATAGAAAAATCTCTTGATGCTCAG
GGGATAAAGTCTGAAAGTTTTGATGTGGTAATTGCTAATAACGTACTCCATAACACAAAATTAATTCAT
CAAACCTTAAATAACAGTAACTCACTATTAAATACTGGGGGGTTATTGGCATTACTAGAGTTTACTCAA
CCAATTGATATTCTTTTATACTTTGGAGGGTTGCTTCAAGGATTTTGGTTGTTTGAAGATCCAGAATACC
GACTAGAAGTTGGTTGTTTACTGAGTATACCACTGTGGCAAAAAGTTCTCAGTGATTGTGGGTTTGATG
AAATAATACCATTGGGATTACCCTGTGAGATGCACGCTCTTTCTAAGGCAAGAGAATCTGTTATATTTG
CTCGAAAGCATCAAGTACAGGAGAAAACATTTTCTGAAAAAATTAAACAAAATTTGACAGAAAATGG
TAAGCATGGGCAGGCAGAATTCGATTTTATTAGTATTAATAATTCACAAGAAAGTTCATCCAAATTAG
AAATTTTTGAACAGGAATGTCGAAAATTATTAAAATCTCTACTGGGTGTTCAACGTATGGAGAGATTG
CCTGGTGACACACCACTAATGGAGTCAGGAATGGATTCACTGGAGTTGTTAGAATTTCGTGCTCTTATA
GAAAGAAAGTTTGGGATTAAGTTAAAGTCTACCTTCTTTTTTAGTTACAAAACTCTTATAGCGGTAGCA
GAGTATCTTTCAGAACGGGAAGATATTAATTTTAGTTAGTAGGACTATGACTGAAAAAAAAACAACGT
TTTCACAGGTCCACAGGGGGAAATTTTGAATCAGGGGGAAAATTTGAATGCATTCTAACCAGAGCAGT
TCACTAGCAAGAATAGCCATTGTAGGTGTGGCTTGCCGTTTTCCTGGAGCAGATACTAAAGAAGAATT
TTGGCAGCTACTTAAGGAAGGTCGGGACTCAATGCAGAATCTTCCTTCAGAGCGTTGGGGAAATCTAT
TTAACTCCATGAGTTCAGAAATAGATTTATCTATTCAACGCGGTGGTTTTTTGAAAGATATAGATTTGT
TTGACTCATCCTTTTTTAGGATTACTCCTAGGGAAGCGCAGTTAATGGACCCCCAGCAAAGGCTGTTGT
TAGAATTGTCCTGGGAAGCGATGGAAGATGCAGGATATGCAAGGGATACATTAAAGGGTAAATCAGT
AGGGGTTTATGTAGGAGTTTGTCACTATGACTACAGAAGTTTATTAGAAAAAGGTTTGGAGACAGCCG
AAATAGCTCAAATTGCTACTGGTACAGCTCCAGCAACTTTTGCCAATCGCCTTTCCTATTTTTATAATTT
TCACGGCCCTAGCTTAACTGTTGATACGGCTTGTTCCAGCTCTTTGGTAGCAATGTATGAAGCAGTAAA
TGCAATACGTCGTGGGCAGTGTCAAACAGCATTGGTAGGAGGCGTTAATTTAATGTGTTCTCCTGTGAA
TAACCAAGTTTATAGTGCTGCGGGTATGTTATCTCCTGATGGAGTCTGCCGGGTGTTTGATGCGGGTGC
TAATGGGTTTGTCAGGGGAGAAGGAGGGGCAGTAGTTGTACTTAAAGATTATCAAAAAGCGTTGACGG
ATGGTGATTCTATTTATGGGGTTGTTCGCAGTGTAGCAGTGAATCATGGAGGGCAAGCAAGTTCTTTTA
CAGCACCAAATCCCCAAGCTCAGGCAAAATTGTTGGAACAGGCTTATAGGGAAGCTAATATTGATATA
GAGTCGGTAGGTTATATTGAAGCACATGGTACGGGTACATCTCTAGGAGACCCAATTGAGGTCGAGGC
TTTAAATGAAGCGTTTAAAGGGTTGAGTTCAAGTGGTAAGTTACCTGCTAATAGTTGTGGTTTGGGCTC
AGTTAAAACAAACATTGGTCATTTGGAAGGGGCAGCAGGTTTGGCTGGACTAATCAAAGTTTTGCTGT
GCATGAGGTATGCAACTCTTCCATGTTCATTAAATTATCAGCAACTTAATCCTGATATTGAATTAGAAG
AAGGTCCGTTTTTTGTGGTGGACAAGCTTCAATCTTGGGAAATAAAAGTTGATAGGGTAGGAAAACCT
TATCCACTTAGAGCAGGGTTGAGTAGCTTTGGTTTTGGCGGTACAAATGCTCATGTAGTTCTTGAGGAA
GGAGAAAATAAGAAGGAAGGAAGAAGAGGGGATAAGGAACCTTCAGTTCATCTATTAACCCTTTCAG
CTAAAACTGAAACAGCTCTTTCAGAGTTAGTCAGTCGTTATCAAAAATATTTAAACACTAATGCAGAA
TCAGAGTTAGCCGATATTTGTTATACCGCTAATACTGGGCGAGTCCATTTTAACCATCGACTAGCAGCG
ATCGCCTCAAACAAACAGGAGTTAGTAGAAAAACTTAAGGAATACCAAGCTGGGGAGGCAACAGCGG
GAGTATTAATAGGGGAACTTGCAGAACGCATAAGAACACCGAAATTAGGTTTCTTATTCACAGGTCAA
GGTTCCCAGTATGTCAATATGGGAAAACAACTGTATAAAACACAGCCTGTATTCCGTGAAGTATTGGA
TAAGTGTGATATAATATTGGAAACGGAAATAGAATGTTCTCTATTAGATGTTCTATATAACAAAACTAC
AGATTCTCAAGATTCATCTTTAATAAACCAAACAGCTTACACTCAACCAGTTCTGTTTGCAATAGAGTA
TGCGCTATTTAAGTTATGGGAATCTTGGGGAATTAAACCCAGCATAGTAATGGGTCACAGTGTAGGAG
AATATGTGGCTGCTTGTGTAGCAGGAGTCTTCAATCTTGAAGATGGTTTGAGATTAATAGCTGCTAGGG
GCAGGCTGATGCAACAGTTACCTTCAGGTGGAGAAATGGTTTCTGTGATGGCTTCAGAATCTACAGTT
AGCAGACTCCTAGAACCTCACAAAAAAGAAATAGCGTTGGCGAAGCCCGCCGTAGGCATCGCAGCAA
TAAATGGACCAGAAAGCACAGTTATTTCTGGAGACTCTGTAGCAGTAACAGGTGTAGTAAATGACCTG
GAAGCAAAGGGAATAAAAACTAAAAAACTAGAGGTATCTCATGCTTTCCATTCACCATTAATGGAACC
AATGTTAGGAGAGTTTGAAGCTATAGCTAATCAACTAACCTACAATCAACCTAAAATACCTATTATATC
TAACGTTACAGGTACAAAAGCAGATAATACTATTGCTTCCCCTCAATACTGGGTTAATCATGTCGGTAA
ACCTGTAAGATTTGCCCAAGGAATGGAAACGCTACATCAACAAGGGTATGAAACCTTTATAGAAGTTG
GACCGAAACCAGTATTGTTAGGAATGGGCAGGCAATGTCTACCAGGAAATGTAGGTGTCTGGTTGCCA
TCATTACGTCCGGGAGTGGATGAGTGGCAACAAATGCTTTATAGCTTGGGAAAATTGTATGTAACAGG
AGTAAAAATAGATTGGTCAGGATTTGAGTCTGACTCTAGTCGCCAGAAAGTAGCATTGCCAACTTATC
CATTCCAGCGAGAAAGATATTGGATAGAAATAACAGAAAACAAACATAAGGAACATCAAAAGTCAGA
AAATATAAGTGACACTTCAATTGTTAAACTACTCACTCAAGGAAAAACAGAAGCTCTCACTCAACAAC
TAGAAACAGAAGCCAAATTTTCACCAGAAGAGCTTAAACTTTTACCAGAAATATTAGAGACATTAGCC
AAACAACATCAAGAACAATTAACAGCAGTAACCATCAAAAACTGGTTCTACGAAATCCAGTGGAAAC
CCTTAGCTCAAAACAACCCCAATGCAAACATTGAACCTAGTCATTGGTTAATTTTAGCCGATACCACAG
GAGTAGCAGAAAAATTAGCTCAAAAATTACAACAACAGGGTCATAAATACAGCTTAGTTTATCGAGGA
GAGAGCTATCAAAGACAAGCAACAGGTACTTATCAACTTAATCCTCAGATTCCCGAAGCATTTGAAAA
GCTGTATCAAGAAATTCAACAAAGTAGTGAAACTGCCATTACGAAGTTAATTCACTTGTGGAGTTTAG
ATGCTCCCCAATCAAAAGACTTAACCCTGGAAACCCTAGAAGAAGCTCAATTATGGGGATGTGGCAGC
GTAGTACACCTGTTACAGACCTTAGTCAAAAACTCTAGCATTCCTGAACTATGGTTAGTAACCCGTGGG
TCTCAATCAGTATTATCCCAAACAGAAAAAAATCTAACAGGACTAGCAGCGTCACCCTTGTGGGGATT
AGGTAGAGTAGTGTCTAATGAACATCCCCAATTATGGGGAGGATTAGTAGATTTAGACCCACAAGCTG
CAGCAGGAGACGAAGTAGAAATGCTGTGGCAATTATTAGTTAATGAACAAGAAGAAGATAATCTAGC
TGTACGGGGAGAAAATACCTATGTAGCTCGTCTGGCCAGGCAAGAACCTCAAGAATTTCCTGAATCCC
TATCCTTATCATCAGATGGTAGTTACCTAATAACAGGAGGGTTAGGAGCTTTAGGGTTACATACTGCCC
AATGGTTAGTATCCAAGGGAGCGAAAAATATTGTCTTAACTGGGCGTCGCCCTCCCTCAGAAAAAGTA
AGTGAATCTATAAAAAAATTAGAAGAAACAGGATGTCAAGTGAAGGTGATGTTGGGGGATGTTTCTGT
TGAAGAAGACATAGGCAAAATTCTCAAACAAATTCAGATATCAATGCCAACACTCAAAGGCATAATTC
ATACAGCAGGAGTGTTAGATGATGGAACCATACAACAAATGAATTGGGAGCGTTTTGCCAAAGTCATG
TCGCCCAAGGTAAAAGGAAGTTGGCATTTACATAAATTAACTGAAAATCAGCCATTAGATTTCTTTGTG
TGTTTCTCCTCAATAGCTTCGATGTTGGGGACTCTTGGCCAAGGAAACTATGCTGCAGCCAATGCCTTT
ATGGATGCTTTAGCTAGTTATCGTTGCAGCAGAGGATTATCAGGATTGGCGATTAATTGGGGAGCATG
GGCATCAGGGGGAATGGCTGCTCGTTTAGCAGTGGAGCATCAAAATAGGATGCACAGCAGTGGCATA
ACTGAGATGGCGACCAAAGAAGGAATGTATGCCTTAGATTTACTATTAACAAATGAATCTGCTACAGC
TCAGGTAGGTGTAGCAAGTATAGAGTGGCAGGTACTCTCAGAAAGTTGGAGTGGTATCAAAAAAAATT
CATTGCTACGAGAATTATTAGAGAAGGAGGAATGGGCAGCAAAAGATACACGACACCAAAAAGTTAA
AGCCGAATTTTTAGCAAAATTAGAGGAGGCATCACTAGAGAAACGTCAGGAAATTTTAACTGAACACA
TTCGAGTGCAAGTATCTCAGGTACTAGGTTTAAGTTCATCTAAATTACCAGAAGTAAATGTAGGTTTTG
CCTCTATGGGAATGGACTCTTTGATGACAATAGAATTAAAAAATCGACTGCAAAATCAACTAGGAACC
AAGTTGCCTGAGACAATAGCTATCGAATATCCAACTATTGCAAAATTGTCCTTAGGTATCAAAAAAAA
TTCATTGCTACGAGAATTATTAGAGAAGGAGGAATGGGCAGCAAAAGATACACGACACCAAAAAGTT
AAAGCCGAATTTTTAGCAAAATTAGAGGAGGCATCACTAGAGAAACGTCAGGAAATTTTAACTGAACA
CATTCGAGTGCAAGTATCTCAGGTACTAGGTTTAAGTTCATCTAAATTACCAGAAGTAAATGTAGGTTT
TGCCTCTATGGGAATGGACTCTTTGATGACAATAGAATTAAAAAATCGACTGCAAAATCAACTAGGAA
CCAAGTTGCCTGAGACAATAGCTATCGAATATCCAACTATTGCAAAATTGTCCTTATGTATAGAGGAAT
TAATGGGATGGAAGACAACAGAAATTGACCCATTATCTGAAGAAACAAGATTAGAGATGATGGAGCG
CTCAAGAAGTGAAGCAATAGCCATCATCGGCATAGGCTGTAGATTTCCCGGAAATGCCAACACACCAG
AAAGCTTCTGGCAATTGTTATCCAATGGCAAAGACTCCATTACAGAGATTCCCCTAGAACGTTGGGAT
CTAGATTCCTACTACGACCCCAACCCTGATACTCCAGGGAAAATGTATATCCGTCACGCAGCATTAGTA
GAAAAAGTAGATCAGTTCGACCCACGATTTTTTGGAATCTCTAACCGAGAAGCTTATAGTCTTGATCCA
CAGCAACGCTTCATTTTGGAAGTAACTTGGGAAGCTTTAGAAAGAGCTGGTATTAACCCTCAACAATT
AGAAAACACTCAGACAGGGGTGTTCCTGGGTATCGGTCAAAATGATTACGCGAATTTAGGTTTTCACC
AAGCAGCCGAAGATATAAGCCCTTACGATGCTACAGGAAATTTGTTTTGTTTTGTAGCAGGTAGGTTAT
CTTACTTTTTAGGAACGCAAGGTCCATCAATGGCAATAGATACAGCTTGTTCATCATCTCTAGTAGCTA
TCCATGAAGCTTGTGAGAGTCTGCGTCAGGGTGAGTCCAACTTGGCTTTGGCTGGAGGAGTTCAACTA
ATTCTCTCTCCCGAAGTAACAACCGCACTATCAAGATTAAAGGCTCTAGCACCTGATGGTAAGTGTAA
AACTTTTGATGCTGCTGCTGATGGTTTTGGTAGGGGAGAGGGATGTGGCATTGTGGTACTGAAGCGTTT
GTCGGATGCGCTCAAGGATGGGGACAGGATATCAGCAGTAATTCCTGGTTCGGCTGTTAACCATGATG
GACCAAGTAGCGGAATGACAGTACCGAATAAACTGGCTCAGGAAAAACTGATTCAGAAAGCTCTCAA
AGCAGCCAAGGTAAAACCGTTACAAGTGAGTTATGTGGAAGCTCATGGTACAGGAACTTCTCTAGGAG
ATCCTATGGAAGTGAGAGCTTTAGCTAGGGTGTTTGAGGAGGGACGTGATCAGGAAAATCCATTGAAC
ATCGGTTCAGTTAAAACTAATATCGGTCATCTGGAAGCAGCAGCTGGAATAGCAGGTATGATTAAGGT
GATTTTGCAATTGCAACATCAGGAAATTGTGCCCCATCTGCATTTTGCTAATCCTAACCCCTATGTTGA
TTGGGAGAATATGCCTCTACAAGTACCGACTCAACTGACTCCTTGGTTGTCGAAAGGGGAGAAAAGGG
TGGCAGGAGTTAGTTCTTTTGGTATGAGTGGTACGAATGCTCATATAGTTTTAGAAGAGGCTCCTATTG
AAGTCAGAAGGCAGAAGTCAGAAGTCAGAAGTGAAGAATATCTAGAACGTCCGGTTCATATACTAAC
TCTGTCGGCCAAGACTGAAAAAGCACTAGAAGATTTAGTTAATAGTTATGAAAGTTATTTAGAAGCCG
AAAATAATGATAATTATTTAGGGGATATTTGCTACACAGCCAACATCGGGAGAGCAAAATTTGACCAC
AAATTAGCAGTGGTTACTTCTGATAAACAAGAGTTATTAGAGAAACTCAAACAATATAAACAAGGTGA
GAATGTTGCTGGAATCTTTTCAGGAAAACAAATAAGTGAAACTAGAACAAAAATAGCCTTTATATTTA
CTGGTCAAGGTTCTCAATATCTGCAAATGGGAAGGCAGTTATACGAAACTCAACCCACTTTTCATAAA
ATTATTGACCAGTGTAGTGAAATGCTGGTAAAATATTTAGATGTTTCTTTATTAGATATACTTTATCCA
GTTGAGGTTAAAGATGAAAGTTCGACTTTGATAGACCAAACAGCTTATACTCAACCCGCTATATTTGCC
CTTGAATATGCCCTAGCTAAATTATGGGAATCATGGGGTATAAAGCCAGATGTAGTCATGGGTCACAG
TGTAGGAGAATATGTGGCAGCAACAGTAGCAGGGGTATTTAGTTTAGAAGATGGTTTAAAATTAATAG
CCATGCGGGGACAGTTGATGCAAAAGTTACCCTCCGGAGGTCAGATGGCATCTATAATGGCATCAGAA
TCTCAGGTAATAGAGGCGATAAAAGAATATAGTTCCCAAGTAACAATAGCAGCAGTTAATGGACCAG
AAATTATAGTAATTTCAGGTGAGAGTCCAGCCATTTCAAAAATTTGTAGTAAATTTGAATCAGAAGGA
GTCAAGACCAAGGCGCTACAAGTATCTCATGCTTTCCATTCCCCATTGATGGAACCAATGTTAACAGA
ATTTGAAGCAGTAGCCAAAGAAATATCCTATAATCATCCCCAAATACCACTAATATCAAATGTTACTG
GTCAAGAAGTAAATGGAGAAATAACCACTGCTGAATATTGGGTGCGTCATGTGCGTCAACCAGTAAGA
TTTGCGGAAGGGATGGAAACTTTACACAAACAAGGTGCTGAAATTTTCCTAGAAATAGGCTCAAAACC
GATACTTTTAGGTATGGGTCGTGAGTGTCTCATGGGAGAAAAAAAACTATGGTTGCCCAGTTTACGTTC
AGGAAAACCAGACTGGTTACAAATGCTACAAAGTCTGGGGCAATTGTATGTACGAGCAATAAAAATTG
ATTGGTTAGGATTTGATCGGGATTATTTCCGTAATAAGGTAGAGTTACCAACATATCCTTGGCAACGAA
AAAGGTATTGGATAACAGATATTAGACAACGGAAAAGTCAAGACAAAAAAAGCATAACATCTGAAAA
AAAAGTACAACTTGATGGAGTAAATATTCAACTAAAGGAAATACAAATGAACGACAAAATCTTACAG
CAACCAAAACTAAAATTATCAGATCCAGAATCACTATATTTATCGAATGCTGAATCAACTATAGAGGT
TCCAACAAAAGCAGTTCAAGTAAAACCTGCTGCTGATATTGAATCTGAAAATATAACTCAATTGAATA
GCCTTGATAGAGATGTGACTCAAATCATAGAAACCCTCAAAGAAAGTTTGGCAGATGCTTTATATGCA
GATATAAGTGAAATTGAAGAAGACAAAAAGTTTGTTGATTTAGGTTTAGATTCTATTGTGGGAGTAGA
ATGGATAACTAACATCAATAAAATCTACAATTTAAATATAAAAGCTACTAAACTATACGATTACCCTA
CTTTGCCAGATTTGGCTAAATATGTCGCTCACACCCTATCAGCTCAGGGTAGAAATATTGATGTAGAGC
GATCGCCATCTGACTCAAGTCAAGCAAGCATTAGCAAGCAATCTCAACTAACTGATACCCAGAGCAAT
TTCTCACAAGTCAAGGAAATTTTGAAACAACAATTAGCAGATGCTCTGTATGCGGATATTAGTGAAAT
TGAAGTAAACAAGAAGTTTGTTGATTTAGGTTTAGATTCTATTGTTGGAGTAGAATGGATAACTAATAT
CAATAAAACCTACAATTTGAATATCAAAGCCACTAAAATATACGACTACCCTACCTTATTAGATTTCGC
AAAATATATTAACCAGGAAATTTATTCTACAGGAGTAAGTAGATTTTCAACGGAGCATAAAGAATTTA
ATCAAAAAGACTATTCATCTGGAGGTTCGCAAGAGGAAATGACACAAAAATTACGATCAATATTGAAG
AAAGTGGCCAATAAAGAATTAACAGTTCAGGAAGGAAATAAAATGATTCAACAAATAAAAAATCAAT
TAAAGTAAAAATATAGTATAATTTTTAAGGAAATAAAATTTTATGAACAAAGAGCAAATATTTCAAAT
AATAAAAAAATATACCTACGAAATAGCACCAGAATTAGAAGAAGTACCTATATCACCTACTGATAGTC
TCAAAAACTTGGGGATTGATTCAGTAAATAGGGCAGAAATTATAATGATGGTAATGGAAGAGTTATCA
TTAAATATCCCGCGTATTGAATTAGCTGGATCCAAAAATATAGGAGAACTGGCTGATATATTTGCATTC
AAATTAGAAGCAATAAATTCACAAAGCTAGAAGGCCTATCAATGAGTAACATAGAAATAACAGGTAT
GGGCATTGTTACTTCCATTGGTCAAGGAGTTGCTACTTTTAAAGAAGCTCTGTTATCAGGGAAAACTCA
ATTTGCTTATCTAAAACAGCCAGGACGTGAAAGCATCAAACCATTTATTGGTGCCGAAATTCCCGATAT
TGACGCCAAAACCCTATTTCCTGAGTACAGTGGACTGTTACGTACTGCTACTAAGAGCGCCCAAGTTGC
AATAGTAGCTGTCGCTGAAGCCTGGCAAGATGCTCAACTCACCTCCAGCCAAGTTAACCCAGAACGAG
TAGGATTAGTTGTAGGTGGTTCAAATTTACAACAACGTTATCAGCAGCAAACCTGGCAACGTTATCACT
CACGTCAGGAGTTTATACGACCAACTTATGGTCTGACCTTTTGGGATACAGATATATTAGGTCTGATTT
CCCAGTGTTTTCAGATTCAAGGTGAGGGCTATAGTGTAGGTGGAGCTTCGGCCAGCGGAGCAGTTGCT
ATAATTCATGCTGCTCGTCAAATTTTAATGGGGAACAGTGATGTTTGCATTGCTCTGGGAGCTTTATCC
GATCTATCAGGGTATGAATTCCAAGCACTAATGAATTTAGGTGCAATGGGGAGCGAACGTTTTGCCGA
TCGCCCAAACCTTGCTTGTAGGCCATTTGACCAAGACCATGATGGCTTTATTTATGGAGAGGGTTGTGG
AGCAGTGATTTTAGAGAGAACTGACCGCGCTCAACAACGGGGCGCTCAATCCCATGGCCAACTTAAGG
GTTGGGGATTAACTTTAGATGGGAACCGTAGTCCCGAACCTTCCCAAAAAGGGGAAGAACGGGCTATA
AATACTGCTTTAGCCATGGCAGACCTTCAGCCAGAGAGTATTGATTATGTGAATACCCACGGTACTGGT
TCCCCCCTTGGCGACAAAACAGAAGTGGCAGCTTTCAAATCAGTAGGACTTCAGCATTGCCTATTTAAT
TCAACAAAATCTTTAATAGGCCACTGCTTAACTGCTGCTGGTGTAGTGGAAGCGATCGCCACCATATTA
CAAATGAAGTTTGGTTTTTGTCATCCAACTAAAAATTTAGTTAATCCTATTGATACCAGTCTCAATTGG
GTGAAAGAAACTTCTGTTCAAGCTGAGATTAAATATGCTATCAGCAACAGTTTTGGTTTTGGGGGTATC
AATACAGCTTTATTAATTGGACAGGAGTAAACTCATGCAACAAGTTGGAATTGAAGCACTAAATGTAT
ATGGGGGTTCAGCTAAACTAGATGTGCGAATGCTAGGCGAAGCACGTCACTTAGACATGACTCGCTTC
GATAATCTTCTGATGAAAGAGAAGACAGTTGCTATGCCCTATGAAGATCCAGTTTCTTATGCTATTAAT
GCAGCAAAACCTATTATTGATAGCCTCAGCGTTCTGGAAAAGCAACAAATCAAAATGGTGATAGCTTG
TAGCGAATCTGGCATTGATTTTGGCAAGTCTATGAGCACTTATATCCAGGATTATTTGGGACTAAATCG
TAACTGTCGAATGTTTGAAATAAAGCAAGCTTGCTACTCAGGTACAGCAGGTTTACAAATGGCATTCA
ATTTAATTTTGTCCCAAACCTGTCCAGGGGCTAAAGCTTTGGTTATAGGAACTGATATATTTCGGCCTG
TTGTTGTAGAGGGAGGAGAAGCTCTCAGCGAAGATTGGTCTTTTGTGGAACCAAGCAGTGGTGCTGGA
GCAGTAGCTATTTTAGTCAGTGATGTTCCTAAAATTTTCCAAGCTGATGTAGGATGTAATGGTTACTAT
GGCTATGAAGTCATGGATACTTGCAGACCTAACCCAGACTCAGAAGCAGGAGATGCAGATTTATCATT
GCTATCTTACCTAGATTGTTGTGAAAATGCTTACCGGGAATATCAAGATCGAGTAGAAGGAGTAGATT
ACCAAAAAACTTTTGATTACTTGAGCTTTCATACTCCTTTTGGGGGTATGGTGAAAGGGGCTCACAGAA
GTATGATGCGTAAGTTTAAAAGGGCAAAACCTGTGGAGATTGAAGAGGACTTTCAGAAACGGGTGAT
GCCGGGATTAGTCTATTGTCAACAGGTGGGTAATATTATGGGAGCTACAGTATTTTTATCTTTAGCTAG
CATGATTGATAATGCAGATTTTAGCAAAGCCCGACGAATTGGTGTATTCTCTTATGGCTCTGGTTGTTG
TTCGGAATTTTATAGTGGAGTCGTCACCCCGCAAGGAAAGGAAATTCAAGCTCAACAAAAAATTTCAT
CACAGTTAGCAATGCGCTATTCCTTAAGTATGGAAGAATATGAGCAGTTACTAAGTCATAGTTCGGCA
GTTGCCTTTGGCACTAGAAATGTTACCCTAGATTACAAACTATTTCCTGGTGTGTGGGAACAAATTGAA
GGTAAAGGTCGCTTAGTGCTGAAAAGAATCAAGGAATTTCACCGAGAATATGAATGGGTATAGCCATG
AGTTATCAAACCCTGAAAATCAGTTATCAAGATGTTGTACAAAGGATTCAGATATATCGACCTGAATC
TAACAATAGCATCAATAGTCAATTAACGATGGAATTGTTGTCAGCTTTGCAAGCTGCTGAAGCAGAGG
AAGTTGTTAAAGTAGTGATATTAGAAGGACTACCTGATGTATTTTGTACAGGGATGGATTTTGAAGAA
GTGGCAACAGCAAAACAATTTGATCCAAAAGCTAGTGCTAATGGTTACTACAATATTTTGAAACAAAT
GTCTCAAAGTAGCAAAGTAATTTTGTCACTTGTGCGTGGTAAAGTACAGGCAGGGGGAGTTGGTTTAG
TAGCAGCAAGCGATCTAGTTATTGCTGATGAAACGGCAACTTTTGTTTTATCAGAATTATTATTTGGAT
TATTACCAGCTTGTGTATTGCCTTTTTTGATTCGTCGAGTGGGATTTCAAAAAGCCTACCGTTTAGCACT
AACGACTCAAGCTATTTCAGTATCAGAGGCTGATAAGTGGGGATTAATAGATGAATATGGCAGTAATA
TTAATCAGTTAATAAGTAAATATATTCGACGTTTAAAGTATTTACCCTCATCAGGGGTGAAAGAGTTAA
AAAATTATATTAATCAGTTATGGATTATTCAGGCAGAAACTCAAGGTTTAGCCGTCAACGAGATTTCTA
GCTTAATAGCAGAACCTACTGTTCAAGAAAAAATTAAACGTTTTCAAAAAGAAGGATTATTTCCATGG
CAAACCTAAATCTTAATTTGGACTTAGTAGAGGGCAACTCTGATGTAGTACAACTGGTGGAGTTGGGT
AATGGTGTCGTGCAAATCACGATGAAAGATGAAGAAAGCTGCAATGGCTTTTCTCCTGGAATAATTGA
AGGATTATACAAATGTTTTGGTGCAGTTGCTCAAAACCAAAGTTATAAAGTAGTAATTTTGACAGGCT
ATGGAAATTATTTCTGTTCGGGAGGGACAAAAGAACGGTTAATCAGTATTTGGAAAGGAGACAGCAA
ATGTAATGATTTAGATTTTTTTAGAATAGCATTAGATTGTGAAATACCAGTAATTGCAGCTATGCAAGG
TCATAGTATTGGCGGTGGTTTGGTTTTGGGATTGTATGCAGATTTAGTAGTGTTAAGTCAAGAAAGTAT
TTACACTACTAATTTTATGAAGTATGGTTTTACTCCAGGTGTCGGATGCACGTTAATTCTCCCTGAGAA
ATTCGGTGCTTTAGGGTTTGAAATGATGTATACTGCCCAAAATTATCGAGGGAAAGAATTAGCTGAAC
GGGGTGTTTCTTTTCCAGTTGTACCGAGAAAAGATGTGCTAGAAGTGGCTAAAAATATAGCCTATGAA
ATGTCGGAAAAACCCAGACTATCTTTAATAACTTTAAAGGAACACTTAACTTCAAAAATCCGCAAAAC
GCTACCAGGATTTATAGATAAAGAAGTAGCTATGCACGAAATAACCTTTCACCAACCAGAGGTAGCAA
GTAGAATAGAGGAAAATTTTGACAAAAGGACAACGGCAAGTAATAACCCTCAAAATTTTCCTCAAGA
GGCTGTAAGAAAAGAAATAAGAACAGATTCATTGAATTGTCAACCATTTCAGTTGAAGACATTTAGTT
ATGGTTCGTTAAACAATTTAACTTTGGTACCTCTAGAACGTAGAGTTCCAAGCCCAAGTGAAGTTGAG
GTTCAAATCAAAACTGTACCAGTTAATTTTCGGGATATACTCAATGCACTTGGTATGCTCCAAGAGTAT
TACGAAAAAACATTTGGCATTGCTAATGCTGAAGATCTCACTTTTGGTTTTGAAGGTGCAGGTACTATC
GTAGCTGTTGGGGCAGAAGTATCGCAGTGGCAAGTCGGCGATGAAGTAATGGTAATGAGAATTCACG
ATGCATTTAGTAGCTTTATTATCTGCTCGCCGGACAAACTGGTGCGTAAAAATTTTAATCTGAATATGG
AAGATGGAGCTAGTATTTGGGGGCCGTTTTCGACTGCATATCACGGGTTGATTAACTTAGCCAAAATTC
AGCCAGGAGATAGAGTATTAATTCACGCTGCTTCAGGTGGGATTGGACAAGCAGCAATTCAGTTGGCT
CAACTTTTTGGGGCGGAAGTATTTGCTACTACTAGTCCAGGTAAGATGAATTATCTCCGGGAACAGGG
AATTAAGTATGTGATGAATTCTCGAACGATGGAGTTTGTAAATGATGTGATGGAATTCACTCAGGGCC
GTGGGGTAGATGTTATTCTCAATAGCCTGACTCATGGAGAATACATCCAGAAAAATCTAGAGATCCTT
GCCGATAGAGGGCGATATGTTGAACTTGGTAAGTTGGGCATTTGGAGTCACGAGCAAGTCTATCAGAA
ACGCCCAGATATCAAATATTTTACTTTTGATTTGTTAGAAGAATTTGCCAAAGATAATCAATTGTTTTCT
CAGATATGGGATAATTTGGCACTTGAATTTGACCGCGATCGCTTGAAGCCACTACCTTACAAAACATTT
CCAATAGAAGATGTTCTTAAAGCCTTTGATTATATGCGACGCGGGAAGCATTTTGGCAGGGTAGTGGT
AGTTATGCCTGACTCCTATTCTAGACAGGAACAAGAGTTGGATGCTCGGTTATCCATTGAAAACAAAA
TGACTAAAGAGGAACAGATATTATTTCAATTACAATCTGGTGAAGTTTCCTTAGAAAATGCGGAACAA
CTATTGTTAGGAAATACGGAAACAGAAACACAAGATAAAGCTATAGCAGAAAATCAAATAGATAATA
TTCAAAACAAGTTAATTAACATGGATAGCTCAGAGAAAATCTTATCTTTGATTAGTTCAGTAGAAATAT
CTTTAGAAACAGCAGAAAAATTATTATTAGAAGTAGTAGAACCAGAAGTTAAAACAGAGGTTAATGAT
GAGGTTAATCCTAGTCAAAATCATATACCAACTACAGATATAGCGATTATTGGTATTTCATGTAGATAT
CCAGGAGCGAATAACTGGAAAGAATTTTGGGAAAATTTAAAGAATGGAATTGACAGTGTAACGGAAG
CTCCTCCTGGAAGATGGGAAGAAAAAAATTGGTATCATCCAGATCCAGATAATCCAGGTACTTCCTAT
TCAAAATGTGCCGGTTTTTTAGATGAAATTGATAAATTTGACCCTTTATTTTTTCATATTTCTCCGGAAG
AAGCTTGGTTTATGGAGCCTGAGCAAAGAATATTTTTAGAAGAAGCTTACCACGCTATAGAAGATGCT
GGATATGCTACAGACTCTCTTAGAGGTAAACAATATGGAGTATTTGTGGGAGTTACGGTAAATGGTGG
TTATCTTAAGTTGTTGTCAATTTCAGGATTAGATGTTCATAGGATGGCGGCTACAGGAAATGGTCCGTC
AATGATACCAGCAAGAATTGCCTATATGCTTGACCTTCAAGGGCCAGTAGTAGCTATTGATACTGCCTG
CTCGTCGTCATTGGTAGCTGTTCATCAAGCTTGCCAAAGCATACAACGAGGAGAGAGCGAAATAGCGA
TCGCTGGAGGTATTACTCTAATGCCAACATCAGACTTCCAAATAATGTCAAGTCAGTTTCAAGTTGTAT
CTCCTGATGGACGTTGTAAGACTTTTGATGCTTCAGCATCGGGTACAGCCTGGAGCGAAGGTTGTGGTG
TTCTCTTATTAAAAAGCTACAGTCAAGCAATTCAAGACAACGACCATATTTATGGAGTAATCAAAGGA
ACAGGAGTTAATTATGATGGTAATACTAACGGGATTAGTGCCCCTAGTAGTCAATCTCAAGCCAGTTT
AGAAGAAGCAGTTTATCAGAAATTTGGAATTAACCCAGAAACTATTAGTTATGTAGAAGCTCATGGTA
CAGCAACACCTCTGGGAGACCCGATAGAAGTAGAAGCTTTAACAGAAGCCTTTTCTAAATGGACGAAC
AAAAAACAGTTTTGTGCGATTGGCTCGGTAAAAACTAATATTGGTCATTCAGCAGCGGCTGCCGGAGT
TTCTGGTTTGATCAAGACAATTTTGTGCCTCAAAAATCAAAAATTAGTTCCATCCTTACATTTTAATCA
ACCAAACCCACATATTGACTTTGAAAATAGCCCTTTTTATGTCAATACAAAATTAAAAGATTGGGAAG
TGCTTGAAGGCCAACCAAGGCAAGCTACAGTTAGTTCTTTTGGTTTTAGTGGCACTAATGCTCATATAG
TTATAGAAGAGGCTCCTTCTCAAGTTAAAAGTCAGAATATTGTTGAACGTCCGATCCATCTGTTAACTC
TATCTGCGAAAACAGAAAAGGCTCTAGAAGATTTAGTCAGTAATTATCAAAATTATTTAGAAACTAAT
CCCGAGTTACCACTAGCAGATGTATGTTATACAGCCTCTACAGGTAGAGCACATTTTAATTATCGATTA
GGAGTTATTGCTTCTGAACCAAAAGCATTAATAGAGAAACTACTTGGGTGGAAAGCTCAGGAAGAATT
AGTAGGACTATTTTCAGGAAAACGAAATAGCGAAGGTCAGAAAATAGCATTCCTGTTCACAGGTCAAG
GTTCCCAGTATGCGAATATGGGAAGGCAACTTTATGAAAAAGCACCAACTTTCCGTCAAGCTTTAGAG
GAATGTGACCAAATTTTACAACCCTATCTAGAAGTACCTCTATTAGAGGTCATATACTCTGAGGATGCA
CAAAAGTCAAGTGATAATCTATTAGACCAAACAGCTTACACCCAACCAGCTGTGTTTGCTGTTGAATAT
GCTTTGGCTAAATTATGGTCTTCATGGGGAATCAAACCATGTGTAGTCATGGGTCACAGCGTAGGAGA
ATATGTAGCAGCAACAGTAGCCGGAGTATTCAGTTTAGAAGATGGTCTGAAACTAATAGCCATGCGAG
GAAAGTTGATGCAAAAGTTACCCTCCGGTGGTGAGATGGTATCCGTAATGGCATCAGAGTCTCAGGTA
ACAGAGGCTATAAAAGAATATAGTTCCCAAGTAACAATAGCAGCGATAAATGGACCAGAAAGTATAG
TAATTTCTGGTGAGAGTGTAGCCATAAAAAATATTTGTAGTTTATTTGAATCAGAGGGAATAAAGACC
AAGCAGCTACAAGTATCCCACGCTTTCCATTCTCCAATGATGGAACCGATGTTAGCAGAGTTTGAATCA
GTAGCTAAACAAGTCACCTATAATCAACCCCAAATACCACTAATATCAAACGTCACAGGTACTGAAGT
AGATGGAGAAATAACAAATGCTGAATATTGGGTAGATCATGTGCGTCAACCAGTAAGATTTGCCCAGA
GTATGAAAACTCTAGAGTCGGAAGGATATGAAACCTTCCTAGAAATAGGACCGAGACCAATATTGCTA
GGAATGGGAAGACAATGTGTAACAGAAGATGTAGGAGAATGGCTGCCATCATTACGTCCAGGGGTGG
ATGAATGGGAACAAATGCTATCAAGCTTAGGAAAATTGTATGTAAAAGGAGCCAAAATAGACTGGTC
AGGGTTTGACTCTGATTATACTCGCCAGAAAGTAGTATTACCCACATATCCATTCCAGGGAGAACGTTA
TTGGGTAGAAACCAACAACAACTTCTGGCCTCAACGGCAATTTTCCCAAGGGGAAAACCTCCATCCCC
TATTAGGTCAAAAGCTAAATTGTGCAGGGGAACAACAAATATTTGCATCACAAATAGGAGAAAACTCA
CCCAACTATCTGAGGGACCACCGAGTATTTAATCAAGCACTATTTCCCATAACAGGCTACCTAGAAAT
AGCAATAGCAGCAGGAAATCACCAATTAAAAACATCCCAGATAGTAATAGAAGACCTAACCATAACT
AGAGGATGGATACTACCAACAGGAGAATTAACCAATGCCCAAACCATACTCACCCCAATAGATAACC
AAAGCTATAAGTTTCAAATATTTTCTCAACCAGAACAACAGGAGTGGAGACTCCACACGACAGGAAAA
ATCAGAAAAGAGTCAACCCCCCCTACTCAGACAAAAGTTGACCTAGAAAAATACAAGAGTGAATGTA
ATCAAACAATAGAAGTCAAACAACATTATCAAAAATGTCAACAAGTAGGGATAGACTACGGGAATAC
CTTCCAAGGCATCCAAGAATTGTGGTCAGGTTCAAACCAAGCATTAGGTTATATCAAACTGCCCGAAG
AATTGATAACACAAACAAGCGACTATCATTTCCATCCAGCACTATTAGATGCAGCCTTGCAAGTAATG
TTTTATGCACTGCCAGCAACGGATAATGACAAAACTTATCTGTCAGCAGGAATAGAAGAATTCAGACT
ATATAAGACTCCCGGGCTGAGTATATGGGCATATGTATCAGTAACCAGTCAAGAAGTGGAAACTCCAG
AAAGTTTGACGGCTATTGTCACCATAGTAACTCCAGAAGGAGAAATAATTGCCAACATCAAAGGTTTA
CAAGTCAAACTAGCAACAAAACAGACCCTACTGGGAACAGAAACCGAATCAATAGAAAATTGGTTAT
ATGAAGTAGAGTGGAGAAACAAAGGTATTTTAGGTAAACTACTCCCACCAGATTTCCTCATACCTCCT
ATACAAATCAACCAAAAATTAACTCCAACTCTGACAGAATTAGTAACTCAAGTAGATAATGAAACAAC
AGCGTCTTTTGAAACAAGCTTAGAAGAATTAAGCAGAGATTATATAGTACAAGCATTACAGTCAATGA
GTTGGTCATACAAACCAACAGAAAGCTTTGCATTTGATGTAGCAGCCCAAAAATTAGGTATAGTTCCT
ACCCATCGACCACTGTTTAAGCGTTTGCTGCAAATATTAACAGAGTCAGGAATACTCAACTCAAAGAA
TCAGCAGTGGGAAGTAGCACAAACCTTACCTGAAGTCAAGCCTACGGAAAAAATCAGCAGTTTACAG
AAGAAATATCCAGAAGAAACAGCAGCATTGACACTACTCTCTCGTTGTGGGTCTAAACTAAGTGGGGT
ATTACGAGGAGCAATAGACCCAGGAGAGTTAGTGTTCCCCCAAGGAGATTTGACAGCAGCGACTCAAC
TTTATGAAGACTCAACAGTAGCGAAAGTGATGAACACAATAGTAGAAAAATCCATCACCAAAGCTATA
GAAAAATCCCCGAAAAGCCGGGGGCTCAGGTTGTTGGAAATAGGAGGGGGAACAGGAGGGACTACAA
GCTATATCCTACCTCATCTAACTCCTCAGCAAACCGAATATACATTCACGGATATAGGGGCATTATTTA
CAGCCAAAGCTCAAGAGAAATTCCGGGATTATAAGTTCATAAAGTATCAAACTTTAGACATAGAAGTA
GACCCGACAACTCAAGGATTTGAGGCTCATCAATATGATGTAATTATTGCAGCTAATGTACTTCATGCA
ACGACAGATATGAAGCAGACATTATCTCATGTGCGAGAACTGTTAGCAGATGGGGGAATGTTGGTGTT
ATCTGAAGCAACAGCTAAAACACTATGGGTAGATTTAGTATTTGGGTTGTTAGAAGGATGGTGGAAAT
TCAGGGATTATGAATTACGACCAGATTATCCTTTGTTGAGTCGTGAAAAATGGCATCATGTCTTGAGAG
AAACGGGTTTTACTGAAGTAGTTACTATGCCAGAAGTGGAGGGAATGGCAGAAACATTGTCAGCACAA
ACGGTAATTGTAGCTAAAAGCTCTCAAACGAAGTTAGAACAAAGGAATGATGATTCTAAGAGTTGGTT
GATACTGGCAGACTCTGAGGGAGTAGGGCAACAGTTAGCGACCTTACTCCGCTCAGTAGGAGAGGTTT
GTACTTTAGTATTTGCCGGAGAAAGGTATCAACAGATAGCTCCGGGAGAATTTAGTATTAATCCTAATC
AAGCTAAAGATTTTGAGGAGGTAATAGAGACAGTGGCAGGGAAGTCACCATCATTGTATGGAGTGGT
ACAATGTTGGACGACTGAAGCCGGAGTGGGGAATGGGATTAATTCTGAGGAGTTAGGAAGTTTATCGA
AGTTAGGGTGTGGCACAACTCTATCTTTAGTACAAGCATTGGTGAAAGGCGGGTTATCAACTGTCCCTC
GATTATGGTTGGTGACAAATGGTGCTCAGGCGGTGCCGAATAATCATCCGGTGATACCAGGAGTAGCT
CAATCTTCGGTATGGGGAATGGGGAAAGTGATTAGCTGGGAACATCCAGAGTTGAACTGTACTCGTAT
AGATTTGGACCCAGAGGAGACTTTAGAGGGTAAAGTTGATGCCCTATTTAAAGAAATTTGGTCGGAGG
ATAGGGAAGACCAGGTAGCATGGCGTGGGGATAGTCGTTATGTAGCTCGGTTGGTGGGTAGTCATCAT
CGGCAATTAGTGGCACAACAAGCTGATGGTAAAACTCAAAAGCCCTTAAGTTTCCGTTCTGAAGCAAG
CTATTTGATTACAGGAGGTATGGGAGGTTTGGGTTTGCTGGTAGCTAGTTGGATGGTGTCAAAGGGAG
CTAAACATTTGATATTGTTAGGACGCCGTTCACCGGATGATGCTACGAGGAAAAAAATAACCGAGTTA
GAAATGGCAGGAGCGTCCGTGGTAGTGGAAAAAGCCGATGTGACTGATTTGGAGTCGATGAAAGGGG
TGTTGCAGAGGATTGAGGAGTCAAAGCGACCGTTAGTAGGAGTGATTCATTCTGTGATGGTGCTATCA
AATGGAGTGCTACGAAATCAGACTTGGTCTAGTTTTGAACAGGTGATGGAACCGAAAGTTCAAGGTGC
TTGGCACTTGCATCAATTGACTCAAAGTCAACCATTAGACTTCTTTGTGCTGTTTTCTTCAGCAACATCT
CTGTTGGGTTCACTGGGTCAGGGCATGGCAAATTATTCTGCTGCTAATGGGTTTCTTGATGGTTTAGCT
CATTATCGTCGAACTATGGGATTACCGGGATTGAGTATCCATTGGGGAGCAGTTTCTCAAGTGGGACG
AGCGCTCGGGCGAGATACAGAGACAGCCGCTATGCTCAGTAAGAATGGGATGGGTTTAATATCTCCGG
CTCAGGTATTGGAGTCTTTGGAACTATTGATGAGTAGTTCGGATGTGGAAGTAGGGGTGATGCCTATTG
AGTGGTCAGGGTGGCAGGAGAGAGTGGCACAGTGGCCGTTTTTAGTGGATTGGCAGGAAACTATATTG
GAGGTAGCCCAACCATCGAAGTCAGATTTTCTGTTAAAGTTGGAGGCTACACCACCTAATGAGCTTCG
CTTGTTGTTGGTGGCTCATGTGCGTCGTCAGGTGGCTCAAGTGTTGAGAATTAGTCATCCTGAATCAAT
TGAAATGGATACAGGGTTTTTTGATTTGGGTATAGACTCTTTGACTTCTGTGGAGTTGAGGAATAAGTT
GCAAGGTAGTTTAAAGTGTTCAGTACCTTCTACTGTAACTTTGGACTACCCTACCATTAAGGCATTGGT
AGAATATTTATATCAACAGCTATTGTTAGAACAAGTTAGTTACTCAAATACTGTACCAACTGAAGAAA
TCAATGAAGATAGGGAGGAGATTACTCTAAGCGGGACACCAGCTCGGGAAGCAAGTAGTGCCTTACG
GGATGGCTATCGTCAAGCAGGAGTCAGCGGGCGGGTTCGGAGTTATTTGGATTTATTGGCTGGTTTAA
GTGATTTTCGGGAACATTTTGATGGTAGTGATGGTTTTTCTTTAGATTTGGTGGATATGGCTGATGGTCC
CGGTGAAGTGACCGTTATTTGTTGTGCCGGTACTGCCGCTATTTCTGGTCCCCATGAATTTACCCGCTTA
GCCGGTGCTTTGCGTGGTATTGCTCCCGTGCGGGCTGTTCCACAACCAGGTTATGAAGAAGGTGAACC
CTTACCCAGTTCTATGGCCGCTGTGGCCGCTGTTCAAGCCGATGCTGTTATTCGCACCCAAGGTGATAA
ACCCTTTGTGGTTGCTGGTCATAGTGCCGGTGCTTTAATGGCCTATGCTTTGGCCACTGAATTATTGGAT
CGTGGTCATCCTCCCCGCGGTGTGGTTTTAATTGATGTGTATCCTCCCGGTCATCAAGATGCTATGAAT
GCCTGGTTAGAAGAATTGACCGCCACTTTGTTTGATCGGGAAACCGTTCGCATGGATGATACCCGCTTA
ACTGCTTTGGGTGCCTATGATCGTTTAACTGGTCAATGGCGCCCCCGTGAAACCGGTTTGCCCACTTTA
TTGGTGAGTGCTGGTGAACCTATGGGTCCCTGGCCCGATGATTCTTGGAAACCCACCTGGCCCTTTGAA
CATGATACTGTGGCCGTTCCCGGTGATCATTTTACTATGGTTCAAGAACACGCTGATGCTATTGCACGA
CACATTGATGCCTGGTTGGGCGGTGGAAACTCCTAACTCCCTATAGTGAGTCGTATTAGCGGCCGCATC
GAATATAACTTCGTATAATGTATGCTATACGAAGTTATTAGCGATGAGGACATGAGGTTGCCCCGTATT
CAGTGTCGCTGATTTGTATTGTCTGAAGTTGTTTTTACGTTAAGTTGATGCAGATCAATTAATACGATA
CCTGCGTCATAATTGATTATTTGACGTGGTTTGATGGCCTCCACGCACGTTGTGATATGTAGATGATAA
TCATTATCACTTTACGGGTCCTTTCCGGTGATCCGACAGGTTACGGGGCGGCGACCTCGCGGGTTTTCG
CTATTTATGAAAATTTTCCGGTTTAAGGCGTTTCCGTTCTTCTTCGTCATAACTTAATGTTTTTATTTAAA
ATACCCTCTGAAAAGAAAGGAAACGACAGGTGCTGAAAGCGAGGCTTTTTGGCCTCTGTCGTTTCCTTT
CTCTGTTTTTGTCCGTGGAATGAACAATGGAAGTCCTCGTCGAGGACGATCTTCCGCTGCATAACCCTG
CTTCGGGGTCATTATAGCGATTTTTTCGGTATATCCATCCTTTTTCGCACGATATACAGGATTTTGCCAA
AGGGTTCGTGTAGACTTTCCTTGGTGTATCCAACGGCGTCAGCCGGGCAGGATAGGTGAAGTAGGCCC
ACCCGCGAGCGGGTGTTCCTTCTTCACTGTCCCTTATTCGCACCTGGCGGTGCTCAACGGGAATCCTGC
TCTGCGAGGCTGGCCGGCTACCGCCGGCGTAACAGATGAGGGCAAGCGGATGGCTGATGAAACCAAG
CCAACCAGGAAGGGCAGCCCACCTATCAAGGTGTACTGCCTTCCAGACGAACGAAGAGCGATTGAGG
AAAAGGCGGCGGCGGCCGGCATGAGCCTGTCGGCCTACCTGCTGGCCGTCGGCCAGGGCTACAAAATC
ACGGGCGTCGTGGACTATGAGCACGTCCGCGAGCTGGCCCGCATCAATGGCGACCTGGGCCGCCTGGG
CGGCCTGCTGAAACTCTGGCTCACCGACGACCCGCGCACGGCGCGGTTCGGTGATGCCACGATCCTCG
CCCTGCTGGCGAAGATCGAAGAGAAGCAGGACGAGCTTGGCAAGGTCATGATGGGCGTGGTCCGCCC
GAGGGCAGAGCCATGACTTTTTTAGCCGCTAAAACGGCCGGGGGGTGCGCGTGATTGCCAAGCACGTC
CCCATGCGCTCCATCAAGAAGAGCGACTTCGCGGAGCTGGTGAAGTACATCACCGACGAGCAAGGCA
AGACGATCCCCTCGACAGCGACACACTTGCATCGGATGCAGCCCGGTTAACGTGCCGGCACGGCCTGG
GTAACCAGGTATTTTGTCCACATAACCGTGCGCAAAATGTTGTGGATAAGCAGGACACAGCAGCAATC
CACAGCAGGCATACAACCGCACACCGAGGTTACTCCGTTCTACAGGTTACGACGACATGTCAATACTT
GCCCTTGACAGGCATTGATGGAATCGTAGTCTCACGCTGATAGTCTGATCGACAATACAAGTGGGACC
GTGGTCCCAGACCGATAATCAGACCGACAACACGAGTGGGATCGTGGTCCCAGACTAATAATCAGACC
GACGATACGAGTGGGACCGTGGTCCCAGACTAATAATCAGACCGACGATACGAGTGGGACCGTGGTTC
CAGACTAATAATCAGACCGACGATACGAGTGGGACCGTGGTCCCAGACTAATAATCAGACCGACGAT
ACGAGTGGGACCATGGTCCCAGACTAATAATCAGACCGACGATACGAGTGGGACCGTGGTCCCAGTCT
GATTATCAGACCGACGATACGAGTGGGACCGTGGTCCCAGACTAATAATCAGACCGACGATACGAGTG
GGACCGTGGTCCCAGACTAATAATCAGACCGACGATACGAGTGGGACCGTGGTCCCAGTCTGATTATC
AGACCGACGATACAAGTGGAACAGTGGGCCCAGAGAGAATATTCAGGCCAGTTATGCTTTCTGGCCTG
TAACAAAGGACATTAAGTAAAGACAGATAAACGTAGACTAAAACGTGGTCGCATCAGGGTGCTGGCT
TTTCAAGTTCCTTAAGAATGGCCTCAATTTTCTCTATACACTCAGTTGGAACACGAGACCTGTCCAGGT
TAAGCACCATTTTATCGCCCTTATACAATACTGTCGCTCCAGGAGCAAACTGATGTCGTGAGCTTAAAC
TAGTTCTTGATGCAGATGACGTTTTAAGCACAGAAGTTAAAAGAGTGATAACTTCTTCAGCTTCAAATA
TCACCCCAGCTTTTTTCTGCTCATGAAGGTTAGATGCCTGCTGCTTAAGTAATTCCTCTTTATCTGTAAA
GGCTTTTTGAAGTGCATCACCTGACCGGGCAGATAGTTCACCGGGGTGAGAAAAAAGAGCAACAACTG
ATTTAGGCAATTTGGCGGTGTTGATACAGCGGGTAATAATCTTACGTGAAATATTTTCCGCATCAGCCA
GCGCAGAAATATTTCCAGCAAATTCATTCTGCAATCGGCTTGCATAACGCTGACCACGTTCATAAGCAC
TTGTTGGGCGATAATCGTTACCCAATCTGGATAATGCAGCCATCTGCTCATCATCCAGCTCGCCAACCA
GAACACGATAATCACTTTCGGTAAGTGCAGCAGCTTTACGACGGCGACTCCCATCGGCAATTTCTATG
ACACCAGATACTCTTCGACCGAACGCCGGTGTCTGTTGACCAGTCAGTAGAAAAGAAGGGATGAGATC
ATCCAGTGCGTCCTCAGTAAGCAGCTCCTGGTCACGTTCATTACCTGACCATACCCGAGAGGTCTTCTC
AACACTATCACCCCGGAGCACTTCAAGAGTAAACTTCACATCCCGACCACATACAGGCAAAGTAATGG
CATTACCGCGAGCCATTACTCCTACGCGCGCAATTAACGAATCCACCATCGGGGCAGCTGGTGTCGAT
AACGAAGTATCTTCAACCGGTTGAGTATTGAGCGTATGTTTTGGAATAACAGGCGCACGCTTCATTATC
TAATCTCCCAGCGTGGTTTAATCAGACGATCGAAAATTTCATTGCAGACAGGTTCCCAAATAGAAAGA
GCATTTCTCCAGGCACCAGTTGAAGAGCGTTGATCAATGGCCTGTTCAAAAACAGTTCTCATCCGGATC
TGACCTTTACCAACTTCATCCGTTTCACGTACAACATTTTTTAGAACCATGCTTCCCCAGGCATCCCGA
ATTTGCTCCTCCATCCACGGGGACTGAGAGCCATTACTATTGCTGTATTTGGTAAGCAAAATACGTACA
TCAGGCTCGAACCCTTTAAGATCAACGTTCTTGAGCAGATCACGAAGCATATCGAAAAACTGCAGTGC
GGAGGTGTAGTCAAACAACTCAGCAGGCGTGGGAACAATCAGCACATCAGCAGCACATACGACATTA
ATCGTGCCGATACCCAGGTTAGGCGCGCTGTCAATAACTATGACATCATAGTCATGAGCAACAGTTTC
AATGGCCAGTCGGAGCATCAGGTGTGGATCGGTGGGCAGTTTACCTTCATCAAATTTGCCCATTAACTC
AGTTTCAATACGGTGCAGAGCCAGACAGGAAGGAATAATGTCAAGCCCCGGCCAGCAAGTGGGCTTT
ATTGCATAAGTGACATCGTCCTTTTCCCCAAGATAGAAAGGCAGGAGAGTGTCTTCTGCATGAATATG
AAGATCTGGTACCCATCCGTGATACATTGAGGCTGTTCCCTGGGGGTCGTTACCTTCCACGAGCAAAAC
ACGTAGCCCCTTCAGAGCCAGATCCTGAGCAAGATGAACAGAAACTGAGGTTTTGTAAACGCCACCTT
TATGGGCAGCAACCCCGATCACCGGTGGAAATACGTCTTCAGCACGTCGCAATCGCGTACCAAACACA
TCACGCATATGATTAATTTGTTCAATTGTATAACCAACACGTTGCTCAACCCGTCCTCGAATTTCCATAT
CCGGGTGCGGTAGTCGCCCTGCTTTCTCGGCATCTCTGATAGCCTGAGAAGAAACCCCAACTAAATCC
GCTGCTTCACCTATTCTCCAGCGCCGGGTTATTTTCCTCGCTTCCGGGCTGTCATCATTAAACTGTGCAA
TGGCGATAGCCTTCGTCATTTCATGACCAGCGTTTATGCACTGGTTAAGTGTTTCCATGAGTTTCATTCT
GAACATCCTTTAATCATTGCTTTGCGTTTTTTTATTAAATCTTGCAATTTACTGCAAAGCAACAACAAA
ATCGCAAAGTCATCAAAAAACCGCAAAGTTGTTTAAAATAAGAGCAACACTACAAAAGGAGATAAGA
AGAGCACATACCTCAGTCACTTATTATCACTAGCGCTCGCCGCAGCCGTGTAACCGAGCATAGCGAGC
GAACTGGCGAGGAAGCAAAGAAGAACTGTTCTGTCAGATAGCTCTTACGCTCAGCGCAAGAAGAAAT
ATCCACCGTGGGAAAAACTCCAGGTAGAGGTACACACGCGGATAGCCAATTCAGAGTAATAAACTGT
GATAATCAACCCTCATCAATGATGACGAACTAACCCCCGATATCAGGTCACATGACGAAGGGAAAGA
GAAGGAAATCAACTGTGACAAACTGCCCTCAAATTTGGCTTCCTTAAAAATTACAGTTCAAAAAGTAT
GAGAAAATCCATGCAGGCTGAAGGAAACAGCAAAACTGTGACAAATTACCCTCAGTAGGTCAGAACA
AATGTGACGAACCACCCTCAAATCTGTGACAGATAACCCTCAGACTATCCTGTCGTCATGGAAGTGAT
ATCGCGGAAGGAAAATACGATATGAGTCGTCTGGCGGCCTTTCTTTTTCTCAATGTATGAGAGGCGCAT
TGGAGTTCTGCTGTTGATCTCATTAACACAGACCTGCAGGAAGCGGCGGCGGAAGTCAGGCATACGCT
GGTAACTTTGAGGCAGCTGGTAACGCTCTATGATCCAGTCGATTTTCAGAGAGACGATGCCTGAGCCA
TCCGGCTTACGATACTGACACAGGGATTCGTATAAACGCATGGCATACGGATTGGTGATTTCTTTTGTT
TCACTAAGCCGAAACTGCGTAAACCGGTTCTGTAACCCGATAAAGAAGGGAATGAGATATGGGTTGAT
ATGTACACTGTAAAGCCCTCTGGATGGACTGTGCGCACGTTTGATAAACCAAGGAAAAGATTCATAGC
CTTTTTCATCGCCGGCATCCTCTTCAGGGCGATAAAAAACCACTTCCTTCCCCGCGAAACTCTTCAATG
CCTGCCGTATATCCTTACTGGCTTCCGCAGAGGTCAATCCGAATATTTCAGCATATTTAGCAACATGGA
TCTCGCAGATACCGTCATGTTCCTGTAGGGTGCCATCAGATTTTCTGATCTGGTCAACGAACAGATACA
GCATACGTTTTTGATCCCGGGAGAGACTATATGCCGCCTCAGTGAGGTCGTTTGACTGGACGATTCGCG
GGCTATTTTTACGTTTCTTGTGATTGATAACCGCTGTTTCCGCCATGACAGATCCATGTGAAGTGTGAC
AAGTTTTTAGATTGTCACACTAAATAAAAAAGAGTCAATAAGCAGGGATAACTTTGTGAAAAAACAGC
TTCTTCTGAGGGCAATTTGTCACAGGGTTAAGGGCAATTTGTCACAGACAGGACTGTCATTTGAGGGTG
ATTTGTCACACTGAAAGGGCAATTTGTCACAACACCTTCTCTAGAACCAGCATGGATAAAGGCCTACA
AGGCGCTCTAAAAAAGAAGATCTAAAAACTATAAAAAAAATAATTATAAAAATATCCCCGTGGATAA
GTGGATAACCCCAAGGGAAGTTTTTTCAGGCATCGTGTGTAAGCAGAATATATAAGTGCTGTTCCCTG
GTGCTTCCTCGCTCACTCGAGGGCTTCGCCCTGTCGCTCGACTGCGGCGAGCCTACTGGCTGTAAAAGG
ACAGACCACATCATGGTTCTGTGTTCATTAGGTTGTTCTGTCCATTGCTGACATAATCCGCTCCACTTCA
ACGTAACACCGCACGAAGATTTCTATTGTTCCTGAAGGCATATTCAAATCGTTTTCGTTACCGCTTGCA
GGCATCATGACAGAACACTACTTCCTATAAACGCTACACAGGCTCCTGAGATTAATAATGCGGATCTC
TACGATAATGGGAGATTTTCCCGACTGTTTCGTTCGCTTCTCAGTGGATAACAGCCAGCTTCTCTGTTTA
ACAGACAAAAACAGCATATCCACTCAGTTCCACATTTCCATATAAAGGCCAAGGCATTTATTCTCAGG
ATAATTGTTTCAGCATCGCAACCGCATCAGACTCCGGCATCGCAAACTGCACCCGGTGCCGGGCAGCC
ACATCCAGCGCAAAAACCTTCGTGTAGACTTCCGTTGAACTGATGGACTTATGTCCCATCAGGCTTTGC
AGAACTTTCAGCGGTATACCGGCATACAGCATGTGCATCGCATAGGAATGGCGGAACGTATGTGGTGT
GACCGGAACAGAGAACGTCACACCGTCAGCAGCAGCGGCGGCAACCGCCTCCCCAATCCAGGTCCTG
ACCGTTCTGTCCGTCACTTCCCAGATCCGCGCTTTCTCTGTCCTTCCTGTGCGACGGTTACGCCGCTCCA
TGAGCTT

### REFERENCES

1. Rohr, J., Angew Chem Int Ed Engl 2000, 39, 2847-2849.
2. Yu, M. J.; Zheng, W.; Seletsky, B. M., Nat Prod Rep 2013, 30, 1158-1164.
3. Eissler, S.; Nahrwold, M.; Neumann, B.; Stammler, H. G.; Sewald, N., Org Lett 2007, 9, 817-819.
4. Shao, N.; Rodriguez, J.; Quintard, A., Org Lett 2022, 36, 6537-6542.
5. Luesch, H.; Yoshida, W. Y.; Moore, R. E.; Paul, V. J.; Corbett, T. H., J Am Chem Soc 2001, 123, 5418-5423.
6. Liu, Y.; Law, B. K.; Luesch, H., Mol Pharmacol 2009, 76, 91-104.
7. Paatero, A. O.; Kellosalo, J.; Dunyak, B. M.; Almaliti, J.; Gestwicki, J. E.; Gerwick, W. H.; Taunton, J.; Paavilainen, V. O., Cell Chem Biol 2016, 23, 561-566.
8. Chen, Q. Y.; Liu, Y.; Luesch, H., ACS Med Chem Lett 2011, 2, 861-865.
9. Chen, Q. Y.; Liu, Y. X.; Cai, W. J.; Luesch, H., J Med Chem 2014, 57, 3011-3029.
10. Qiu, B.; Tan, A.; Veluchamy, A. B.; Li, Y.; Murray, H.; Cheng, W.; Liu, C.; Busoy, J. M.; Chen, Q. Y.; Sistla, S.; Hunziker, W.; Cheung, C. M. G.; Wong, T. Y.; Hong, W.; Luesch, H.; Wang, X., Invest Ophthalmol Vis Sci 2019, 60, 3254-3263.
11. Pohl, M. O.; Martin-Sancho, L.; Ratnayake, R.; White, K. M.; Riva, L.; Chen, Q. Y.; Lieber, G.; Busnadiego, I.; Yin, X.; Lin, S.; Pu, Y.; Pache, L.; Rosales, R.; Dejosez, M.; Qin, Y.; De Jesus, P. D.; Beall, A.; Yoh, S.; Hale, B. G.; Zwaka, T. P.; Matsunaga, N.; Garcia-Sastre, A.; Stertz, S.; Chanda, S. K.; Luesch, H., ACS Infect Dis 2022, 8, 1265-1279.
12. Zhang, J. J.; Tang, X.; Moore, B. S., Nat Prod Rep 2019, 36, 1313-1332.
13. Dhakal, D.; Chen, M.; Luesch, H.; Ding, Y., J Ind Microbiol Biotechnol 2021, 48, kuab003.
14. Yang, G.; Cozad, M. A.; Holland, D. A.; Zhang, Y.; Luesch, H.; Ding, Y., ACS Syn Biol 2018,7, 664-671.
15. Videau, P.; Wells, K. N.; Singh, A. J.; Gerwick, W. H.; Philmus, B., ACS Synth Biol 2016, 5, 978-88.
16. Taton, A.; Ecker, A.; Diaz, B.; Moss, N. A.; Anderson, B.; Reher, R.; Leao, T. F.; Simkovsky, R.; Dorrestein, P. C.; Gerwick, L.; Gerwick, W. H.; Golden, J. W., ACS Synth Biol 2020, 9, 3364-3376.
17. Taton, A.; Rohrer, S.; Diaz, B.; Reher, R.; Caraballo Rodriguez, A. M.; Pierce, M. L.; Dorrestein, P. C.; Gerwick, L.; Gerwick, W. H.; Golden, J. W., ACS Chem Biol 2022, 17, 1910-1923.
18. Grindberg, R. V.; Ishoey, T.; Brinza, D.; Esquenazi, E.; Coates, R. C.; Liu, W. T.; Gerwick, L.; Dorrestein, P. C.; Pevzner, P.; Lasken, R.; Gerwick, W. H., PloS One 2011, 6, e18565.
19. Gu, L.; Geders, T. W.; Wang, B.; Gerwick, W. H.; Hakansson, K.; Smith, J. L.; Sherman, D. H., Science 2007, 318, 970-974.
20. Buchholz, T. J.; Rath, C. M.; Lopanik, N. B.; Gardner, N. P.; Hakansson, K.; Sherman, D. H., Chem Biol 2010, 17, 1092-1100.
21. Matthew, S.; Schupp, P. J.; Luesch, H., J Nat Prod 2008, 71, 1113-1116.
22. Wu, P.; Cai, W.; Chen, Q. Y.; Xu, S.; Yin, R.; Li, Y.; Zhang, W.; Luesch, H., Org Lett 2016, 18, 5400-5403.
23. Wolk, C. P.; Fan, Q.; Zhou, R.; Huang, G.; Lechno-Yossef, S.; Kuritz, T.; Wojciuch, E., Arch Microbiol 2007, 188, 551-563.
24. Hagen, A.; Poust, S.; de Rond, T.; Fortman, J. L.; Katz, L.; Petzold, C. J.; Keasling, J. D., ACS Synth Biol 2016, 5, 21-27.
25. Thiel, T.; Wolk, C. P., Methods Enzymol 1987, 153, 232-243.
26. Enghiad, B.; Huang, C.; Guo, F.; Jiang, G.; Wang, B.; Tabatabaei, S. K.; Martin, T. A.; Zhao, H., Nat Commun 2021, 12, 1171.
27. Zhang, Y.; Muyrers, J. P.; Testa, G.; Stewart, A. F., Nat Biotechnol 2000, 18, 1314-1317.
28. Bankevich, A.; Nurk, S.; Antipov, D.; Gurevich, A. A.; Dvorkin, M.; Kulikov, A. S.; Lesin, V. M.; Nikolenko, S. I.; Pham, S.; Prjibelski, A. D.; Pyshkin, A. V.; Sirotkin, A. V.; Vyahhi, N.; Tesler, G.; Alekseyev, M. A.; Pevzner, P. A., J Comput Biol 2012, 19, 455-477.
29. Enghiad, B.; Huang, C.; Guo, F.; Jiang, G.; Wang, B.; Tabatabaei, S. K.; Martin, T. A.; Zhao, H., Nat Commun 2021, 12, 1171.
30. Thiel, T.; Wolk, C. P., Methods Enzymol 1987, 153, 232-243.
31. Yang, G.; Cozad, M. A.; Holland, D. A.; Zhang, Y.; Luesch, H.; Ding, Y., ACS Syn Biol 2018, 7,664-671.
32. Chen, Q. Y.; Liu, Y. X.; Cai, W. J.; Luesch, H., J Med Chem 2014, 57, 3011-3029.
33. Luesch, H.; Yoshida, W. Y.; Moore, R. E.; Paul, V. J.; Corbett, T. H. J Am Chem Soc 2001, 123, 5418-5423.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A recombinant cyanobacterial cell comprising a nucleic acid construct comprising a nucleic acid sequence encoding a heterologous polyketide synthase (PKS); and a nucleic acid sequence encoding a type-II thioesterase (TE-II).

2. The recombinant cyanobacterial cell of claim 1, wherein the nucleic acid construct further comprises a nucleic acid sequence encoding a second heterologous polyketide synthase (PKS).

3. The recombinant cyanobacterial cell of any one of claims 1 or 2, wherein the first heterologous PKS is an apratoxin A PKS, optionally wherein the PKS is AprA.

4. The recombinant cyanobacterial cell of any one of claims 1 to 3, wherein the second heterologous PKS is an apratoxin A PKS, optionally wherein the PKS is AprB.

5. The recombinant cyanobacterial cell of any one of claims 1 to 4, wherein the nucleic acid construct further comprises a nucleic acid sequence encoding one or more hydroxymethylglutaryl (HMG)-CoA synthase (HCS)-like proteins.

6. The recombinant cyanobacterial cell of claim 5, wherein the one or more (HCS)-like proteins comprise AprC, AprD, AprE, and/or AprF.

7. The recombinant cyanobacterial cell of any one of claims 1 to 6, wherein the nucleic acid construct further comprises a nucleic acid sequence encoding a third heterologous polyketide synthase (PKS).

8. The recombinant cyanobacterial cell of claim 7, wherein the third heterologous PKS is an apratoxin A PKS, optionally wherein the PKS is AprG.

9. The recombinant cyanobacterial cell of any one of claims 1 to 8, wherein each of the heterologous PKS are Moorena bouillonii PKS.

10. The recombinant cyanobacterial cell of any one of claims 1 to 9, wherein the nucleic acid construct further comprises a promoter operably linked to the nucleic acid sequence encoding the first heterologous PKS.

11. The recombinant cyanobacterial cell of any one of claim 10, wherein the promoter is an inducible promoter.

12. The recombinant cyanobacterial cell of any one of claims 10 or 11, wherein the promoter comprises a PcoaT or Prha promoter.

13. The recombinant cyanobacterial cell of any one of claims 1 to 12, wherein the nucleic acid construct is a plasmid.

14. A recombinant cyanobacterial cell comprising an expression construct comprising an inducible promoter operably linked to a nucleic acid sequence encoding, in the following order:
Moorena bouillonii AprA, AprB, AprC, AprD, AprE, AprF, AprG; and
a type-II thioesterase (TE-II) from an erythromycin cluster.

15. The recombinant cyanobacterial cell of any one of claims 1 to 14, wherein the nucleic acid sequence encoding TE-II comprises SEQ ID NO: 64.

16. The recombinant cyanobacterial cell of any one of claims 1-15, wherein the cell is Anabaena sp.

17. The recombinant cyanobacterial cell of any one of claims 1-16, comprising pRL838-Apra.

18. The recombinant cyanobacterial cell of any one of claims 1-17, comprising pRL838-cApra.

19. The recombinant cyanobacterial cell of any one of claims 1-18, comprising pRL838-rApra.

20. An expression construct comprising a promotor operably linked to a nucleic acid sequence encoding, in the following order:
Moorena bouillonii AprA, AprB, AprC, AprD, AprE, AprF, AprG; and
a type-II thioesterase (TE-II) from an erythromycin cluster.

21. The expression construct of claim 20, wherein the promoter is an inducible promoter.

22. The expression construct of any one of claims 20 or 21, wherein the promoter comprises a PcoaT or Prha promoter.

23. A method of making a chemical compound comprising:
culturing a recombinant cyanobacterial cell of any one of claims 1-19 under conditions permitting the production of the chemical compound; and
isolating or recovering the chemical compound.

24. The method of making a chemical compound of claim 23 wherein the culturing of the recombinant cyanobacterial cell of claim 1 further comprises inducing with CoCl₂.

25. The method of making a chemical compound of claim 23 wherein the culturing of the recombinant cyanobacterial cell of claim 1 further comprises inducing with rhamnose.

26. The method of making a chemical compound of any one of claims 23-25, wherein the compound is a polyketide.

27. The method of making a chemical compound of any one of claims 23-26, wherein the compound is a polyketide fragment contained in a natural product.

28. The method of making a chemical compound of any one of claims 23-27, wherein the compound is a polyketide fragment contained in an apratoxin natural product.

29. The method of making a chemical compound of any one of claims 23-28, wherein the compound is Compound (I): 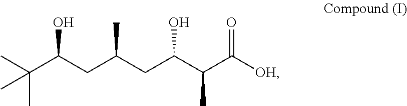 [SMILES: C[C@@H](C[C@H](O)[C@H](C)C(=O)O)C[C@H](O)C(C)(C)C]
or a salt thereof.

30. A recombinant cyanobacterial cell comprising at least one nucleic acid construct that provides for production of Compound (I): 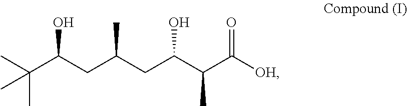 [SMILES: C[C@@H](C[C@H](O)[C@H](C)C(=O)O)C[C@H](O)C(C)(C)C]
or a salt thereof.
